(19) United States
(12) Patent Application Publication
(10) Pub. No.: US 2023/0348467 A1
(43) Pub. Date: Nov. 2, 2023

(54) Heteroaryl Derivative, Preparation Method Therefor, And Use Thereof

(71) Applicants: Zhejiang Hisun Pharmaceutical Co., Ltd., Taizhou (CN); Hisun AccuRay Therapeutics Co., Ltd., Shanghai (CN)

(72) Inventors: Xiangui Huang, Shanghai (CN); Yanghui Guo, Shanghai (CN); Zongxing Qiu, Shanghai (CN); Pingyan Bie, Shanghai (CN); Weiwei Liao, Shanghai (CN); Qingyan Yan, Shanghai (CN); Weichao Shen, Shanghai (CN); Hai Cao, Shanghai (CN); Qingna Xing, Shanghai (CN); Xin Wang, Shanghai (CN); Qi Cao, Shanghai (CN); Lichen Meng, Shanghai (CN); Nuoyi Wu, Shanghai (CN); Wenpeng Li, Shanghai (CN); Cheng Ye, Shanghai (CN); Taishan Hu, Shanghai (CN); Lei Chen, Taizhou (CN)

(73) Assignees: Zhejiang Hisun Pharmaceutical Co., Ltd., Taizhou (CN); Hisun AccuRay Therapeutics Co., Ltd., Shanghai (CN)

(21) Appl. No.: 17/791,283

(22) PCT Filed: Jan. 12, 2021
(86) PCT No.: PCT/CN2021/071297

§ 371 Date: Jul. 7, 2022

(30) Foreign Application Priority Data
Jan. 16, 2020 (CN) 202010046221.8
Jul. 10, 2020 (CN) 202010660519.8

(51) Int. Cl. C07D 487/04; C07D 519/00; A61P 35/00

(52) U.S. Cl. CPC C07D 487/04; C07D 519/00; A61P 35/00

(57) ABSTRACT

The present invention relates to heteroaryl derivatives, preparation methods therefor, and applications thereof in medicine. Specifically, the present invention relates to a heteroaryl derivative represented by general formula (AI), a preparation method therefor, and a pharmaceutically acceptable salt, and use thereof as a therapeutic agent, in particular, as an SHP2 allosteric inhibitor, wherein the definition of substituents in general formula (AI) is the same as that in the description.

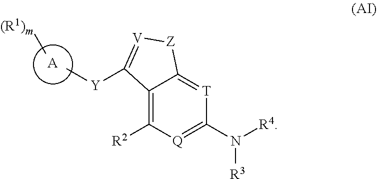

### CROSS-REFERENCE TO RELATED APPLICATION

The present application is a U.S. national phase entry under 35 U.S.C. § 371 of International Application No. PCT/CN2021/071297 filed Jan. 12, 2021, which claims priority from Chinese Application No. 202010046221.8 filed on Jan. 16, 2020 and Chinese Application No. 202010660519.8 filed on Jul. 10, 2020, all of which are hereby incorporated by reference in its entirety.

### TECHNICAL FIELD

The present invention relates to novel heteroaryl derivatives, preparation methods thereof, pharmaceutical compositions containing the derivatives and uses thereof as therapeutic agents, in particular, as a SHP2 allosteric inhibitor.

### BACKGROUND

Src Homology-2 Domain-Containing Protein Tyrosine Phosphatase (SHP2) is an important member of Protein Tyrosine Phosphatase (PTP) family, which is encoded by Protein Tyrosine Phosphatase, Non-Receptor Type 11 (PTPN11) gene and catalyzes the dephosphorylation of tyrosine in protein. An N-terminal of SHP2 contains two SH2 domains, which control the subcellular localization and function regulation of SHP2, and a C-terminal contains a PTP domain with catalytic activity and two tyrosine residues related to activities thereof. Normally, SHP2 is in a state of self-inhibition. When stimulated by growth factors, cytokines or inflammatory factors, such as Platelet-Derived Growth Factors PDGF and FGF, catalytic sites are exposed, leading to the activation of SHP2 enzymes.

SHP2 is widely present in human body, and participates in Rat Sarcoma (RAS)-Extracellular Signal-related Kinase (ERK), Phosphatidylinositol 3 Kinase (PI3K)-protein kinase B and NF-KB, and activates multiple signalling channels like fibroblast growth factors, epidermal growth factors and Mitogen-Activated Protein Kinase (MAPK/ERK) downstream of insulin receptors, so as to regulate cell proliferation, differentiation, migration and apoptosis. At present, it has been found that activating mutation of SHP2 is closely related to the occurrence of Noonan syndrome, Leopard spot syndrome, monocytic leukemia, melanoma, solid tumor, cardiovascular diseases, immune disorder, fibrosis or visual disorder. Over-expression of SHP2 will increase the risks of chronic granulocytic leukemia, mastocytosis, glioblastoma, lung cancer, breast cancer and other cancers, indicating that SHP2 plays an important role in different types of cancers and different stages of the cancers. Due to the multiple functions of SHP2 in tumors, the research on SHP2 target inhibitors also brings new hope and orientation for tumor therapy.

According to different mechanisms of action, SHP2 inhibitors may be divided into competitive inhibitors (including tautomycin, phenylpyrazolyl hydrazine sulfonate and NSC-87877), noncompetitive inhibitors (including indole salicylic acid and fumostone) and irreversible inhibitors (including sodium antimonyl gluconate and cryptotanshinone). As an irreversible SHP2 inhibitor, it is reported that cryptotanshinone can inhibit the proliferation of rhabdomyosarcoma, melanoma, colon cancer and breast cancer in vitro, while in vivo studies have shown that cryptotanshinone can inhibit the proliferation of prostate cancer in mice, and whether cryptotanshinone can further become a clinically effective drug needs many tests to verify.

At present, RMC-4630, a compound developed by REVOLUTION Medicines Inc, has entered clinical phase II for the treatment of solid tumors, and meanwhile, RMC-4550, which is in preclinical phase, is included. Meanwhile, there are also three compounds JAB-3068, JAB-3312 and TNO-155 of clinical phase I, developed by Jacobio (Jacobio Pharmaceuticals Co Ltd) and Novartis (Novartis AG), respectively. Meanwhile, Novartis preclinical drug SHP-099 is included. REVOLUTION Medicines Inc and Novartis AG have disclosed a series of SHP2 inhibitor patents, including WO-2019075265, WO-2018136265, WO-2018136264, WO-2017216706 and WO-2018013597, and the like. Although some progress has been made in SHP2 research, there are still no proven drugs on the market, so it is still necessary to continue research and development of new SHP2 inhibitors.

### SUMMARY

In view of the above technical problems, the present invention provides a novel heteroaryl compound represented by general formula (AI) or a stereoisomer or a tautomer thereof, or a pharmaceutically acceptable salt thereof:

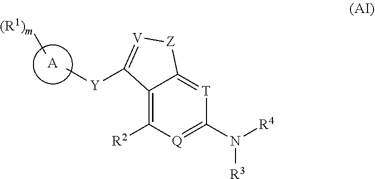

wherein:
Y is selected from a chemical bond or —S—;
when Z is selected from —NH—, V is selected from —N— or —CH—; alternatively, when Z is selected from —O—, V is selected from —N—;
Q and T are each independently selected from N or CH; wherein at least one of Q and T is selected from N;
ring A is selected from aryl, heteroaryl or bicyclic fused ring, wherein the aryl is monocyclic aryl, the heteroaryl is a 5-6 membered monocyclic heteroaryl, and the bicyclic fused ring is preferably a fused ring of aryl or heteroaryl with monocyclic heterocyclyl or monocyclic cycloalkyl;
R¹ are the same or different, and are each independently selected from hydrogen atom, alkyl, alkenyl, alkynyl, cyano, halogen, nitro, cycloalkyl, heterocyclyl, —OR⁵, —C(O)R⁵, —SO₂R⁵, —NR⁶R⁷, —SO₂NR⁶R⁷, —NHSO₂R⁵ or —C(O)NR⁶R⁷, wherein the alkyl, alkenyl, alkynyl, cycloalkyl or heterocyclyl is optionally further substituted by one or more substituents selected from halogen, nitro, cyano, alkyl, cycloalkyl, heterocyclyl, aryl, heteroaryl, —OR⁵, —C(O)R⁵, —C(O)OR⁵, —OC(O)R⁵, —SO₂R⁵, —NR⁶R⁷, —SO₂NR⁶R⁷, —NHSO₂R⁵ or —C(O)NR⁶R⁷;
R² is selected from cyano, tetrazolyl, —C(O)R⁵, —C(O)OR⁵ or —C(O)NR⁶R⁷;
R³ and R⁴ together with the N atom bound therewith form a 4-11 membered heterocyclyl, preferably a 5-11 membered heterocyclyl, wherein the heterocyclyl internally contains one or more N, O, S or SO₂ atoms, and the heterocyclyl is optionally further substituted by one or more substituents selected from halogen, nitro, cyano, alkyl, alkenyl, alkynyl, cycloalkyl, heterocyclyl, aryl, heteroaryl, —CH₂R⁵, —CH(OH)R⁵, —CH₂OR⁵, ═O, —OR⁵, —SR⁵, —SOR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷, wherein the alkyl, alkenyl, alkynyl, cycloalkyl, heterocyclyl, aryl or heteroaryl is optionally further substituted by one or more substituents selected from hydroxy, amino, halogen, nitro, cyano, alkyl, haloalkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
alternatively, R³ and R⁴ together with the N atom bound therewith form a group:

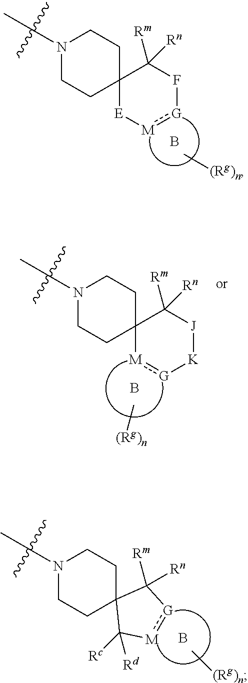

[symbol] is a single bond or double bond;
when [symbol] represents a single bond, G and M are each independently selected from N or CR^{j};
when [symbol] represents a double bond, G and M are each independently selected from C;
ring B is selected from cycloalkyl, heterocyclyl aryl or heteroaryl;
E is selected from NR^{k}, (CR^{p}R^{q})ₚ, O or S;
F is selected from (CR^{p}R^{q})_{q};
the condition is that when E is selected from (CR^{p}R^{q})ₚ, p is 1 and q is 1; alternatively, p is 2 and q is 0; and when E is selected from NR^{k}, O or S, q is 1;
J is selected from CR^{p}R^{q};
K is selected from NR^{k}, (CR^{p}R^{q})ᵣ, O or S;
r is 0 or 1;
R^{m}, Rⁿ, R^{p} and R^{q} are the same or different, and are each independently selected from R^{A};
alternatively, R^{p} and R^{q} together with the carbon atom bound therewith form R^{B};
R^{c} and R^{d} are the same or different, and are each independently selected from hydrogen atom, halogen, alkyl or —OR⁵, wherein the alkyl is optionally further substituted by a substituent of hydroxy, halogen, alkoxy, cycloalkyl or —NR⁶R⁷;
alternatively, R^{c} and R^{d} together with the carbon atom bound therewith form R^{B};
R^{g} are the same or different, and are each independently selected from hydrogen atom, halogen, nitro, alkyl, alkenyl, alkynyl, cyano, cycloalkyl, heterocyclyl, aryl, heteroaryl, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷, wherein the alkyl, alkenyl, alkynyl, cycloalkyl, heterocyclyl, aryl or heteroaryl is optionally further substituted by a substituent of hydroxy, halogen, alkyl, alkoxy, cycloalkyl or —NR⁶R⁷;
alternatively, two R^{g} together with the same carbon atom bound therewith form C═O;
R^{j} and R^{k} are the same or different, and are each independently selected from hydrogen atom or alkyl;
R^{A} are the same or different, and are each independently selected from hydrogen atom, halogen, nitro, alkyl, alkenyl, alkynyl, cyano, cycloalkyl, heterocyclyl, aryl, heteroaryl, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷, wherein the alkyl, alkenyl, alkynyl, cycloalkyl, heterocyclyl, aryl or heteroaryl is optionally further substituted by a substituent of hydroxy, halogen, alkyl, alkoxy, cycloalkyl or —NR⁶R⁷;
R^{B} are the same or different, and are each independently selected from 3-10 membered cycloalkyl or 3-10 membered heterocyclyl, wherein the cycloalkyl or heterocyclyl is optionally further substituted by one or more substituents selected from halogen, cyano, nitro, alkyl, cycloalkyl, heterocyclyl, aryl, heteroaryl, ═O, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷;
R⁵, R⁶ and R⁷ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, amino, halogen, nitro, cyano, alkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
alternatively, R⁶ and R⁷ together with the N atom bound therewith form a 3-8 membered heterocyclyl, wherein the 3-8 membered heterocyclyl internally contains one or more N, O, S or SO₂ atoms, and the 3-8 membered heterocyclyl is optionally further substituted by one or more substituents selected from hydroxy, halogen, amino, alkyl or alkoxy;
R⁸, R⁹ and R¹⁰ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, halogen, nitro, cyano, alkyl, alkoxy, cycloalkyl, heterocyclyl, aryl, heteroaryl, carboxyl or carboxylate;
m is 0, 1, 2, 3, 4 or 5;
n is selected from 0, 1, 2, 3 or 4; and
p is selected from 1 or 2.

In a preferred embodiment of the present invention, the compound represented by general formula (AI) or the stereoisomer or the tautomer thereof, or the pharmaceutically acceptable salt thereof, is a compound represented by general formula (AII) or a stereoisomer or a tautomer thereof, or a pharmaceutically acceptable salt thereof:

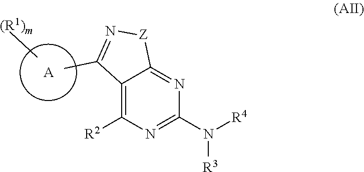

wherein: ring A, m, Z and R¹-R⁴ are defined as in general formula (AI).

In a preferred embodiment of the present invention, the compound represented by general formula (AI) or the stereoisomer or the tautomer thereof, or the pharmaceutically acceptable salt thereof, is a compound represented by general formula (I) or a stereoisomer or a tautomer thereof, or a pharmaceutically acceptable salt thereof:

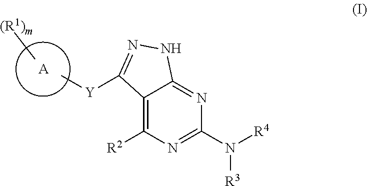

wherein: ring A, Y, m, and R¹-R⁴ are defined as in general formula (AI).

In a preferred embodiment of the present invention, the compound represented by general formula (AI) or the stereoisomer or the tautomer thereof, or the pharmaceutically acceptable salt thereof, is a compound represented by general formula (II) or a stereoisomer or a tautomer thereof, or a pharmaceutically acceptable salt thereof:

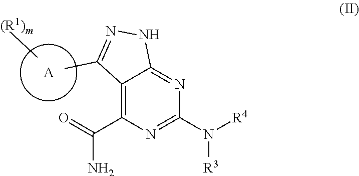

wherein: ring A, m, R¹, R³ and R⁴ are defined as in general formula (AI).

In a preferred embodiment of the present invention, in the compound represented by general formula (AI), (AII), (I) or (II), or the stereoisomer or the tautomer thereof, or the pharmaceutically acceptable salt thereof, wherein:
R³ and R⁴ together with the N atom bound therewith form a 4-8 membered monocyclic heterocyclyl, preferably a 5-6 membered monocyclic heterocyclyl, more preferably piperidinyl, wherein the monocyclic heterocyclyl is optionally further substituted by one or more substituents selected from methyl, amino, cycloalkyl, phenyl, halophenyl, heteroaryl, —CH₂NH₂, —CH₂OH, —NHC(═NH)NH₂, ═O or —OR⁵; wherein the methyl, cycloalkyl, phenyl or heteroaryl is optionally further substituted by substituents selected from one or more of mesyl, hydroxy, amino, halogen, haloalkyl, alkoxy, haloalkoxy, pyridinyl, or pyrimidinyl; wherein the heteroaryl is preferably pyridinyl, pyrimidinomethylbenzopyrazolyl, pyrrolyl, furanyl, thienyl, pyrazolyl, imidazolyl, thiazolyl, benzimidazolyl, benzofuranyl or benzoxazolyl; and
R⁵ is defined as in general formula (AI).

In a preferred embodiment of the present invention, in the compound represented by general formula (AI), (AII), (I) or (II), or the stereoisomer or the tautomer thereof, or the pharmaceutically acceptable salt thereof, wherein:
R³ and R⁴ together with the N atom bound therewith form a 7-11 membered spiroheterocyclyl, wherein the spiroheterocyclyl is optionally further substituted by one or more substituents selected from methyl, amino, —CH₂NH₂, —CH₂OH, —NHC(═NH)NH₂, ═O or —OR⁵; R⁵ is defined as in general formula (AI); and preferably, the spiroheterocyclyl is selected from:

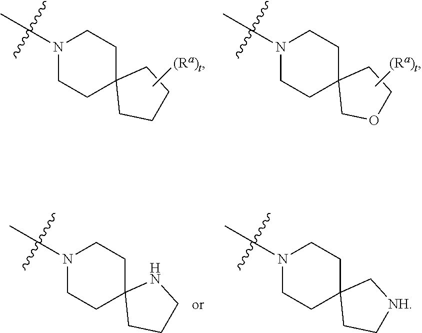

R^{a} are the same or different, and are each independently selected from methyl, amino, —CH₂NH₂, —CH₂OH, —NHC(═NH)NH₂ or —OR⁵; or two R^{a} together with the same carbon atom bound therewith form C═O; t is 1, 2 or 3; and R⁵ is defined as in general formula (AI).

In a preferred embodiment of the present invention, in the compound represented by general formula (AI), (AII), (I) or (II), or the stereoisomer or the tautomer thereof, or the pharmaceutically acceptable salt thereof, R³ and R⁴ together with the N atom bound therewith form a 7-11 membered bridged heterocyclyl, wherein the bridged heterocyclyl is optionally further substituted by one or more substituents selected from methyl, amino, —CH₂NH₂, —CH₂OH, —NHC(═NH)NH₂, ═O or —OR⁵; and R⁵ is defined as in general formula (AI).

In a preferred embodiment of the present invention, in the compound represented by general formula (I) or (II), or the stereoisomer or the tautomer thereof, or the pharmaceutically acceptable salt thereof, R³ and R⁴ together with the N atom bound therewith form a 7-11 membered fused heterocyclyl, wherein the fused heterocyclyl is optionally further substituted by one or more substituents selected from methyl, amino, —CH₂NH₂, —CH₂OH, —NHC(═NH)NH₂, ═O or —OR⁵; and R⁵ is defined as in general formula (AI).

In a preferred embodiment of the present invention, the compound represented by general formula (I) or the stereoisomer or the tautomer thereof, or the pharmaceutically acceptable salt thereof, is a compound represented by general formula (III) or a stereoisomer or a tautomer thereof, or a pharmaceutically acceptable salt thereof:

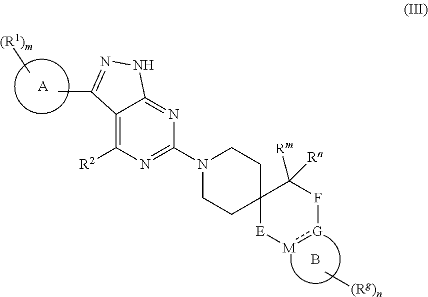

wherein:
ring B is selected from phenyl, 3-8 membered cycloalkyl, 4-8 membered heterocyclyl or 5-6 membered heteroaryl;
E is selected from NR^{k}, (CR^{p}R^{q})ₚ, O or S;
F is selected from ((CR^{p}R^{q})_{q};
the condition is that when E is selected from (CR^{p}R^{q})ₚ, p is 1 and q is 1; alternatively, p is 2 and q is 0; and when E is selected from NR^{k}, O or S, q is 1;
R^{m} is selected from amino, —CH₂NH₂ or —NHC(═NH)NH₂;
Rⁿ is selected from hydrogen atom, methyl or —CH₂OH;
R^{p} and R^{q} are each independently selected from hydrogen atom, halogen, amino, C₁-C₄ alkyls, hydroxy C₁-C₄ alkyls, amino C₁-C₄ alkyls or —OR⁵; and [symbol] , ring A, G, M, m, n, R¹-R², R⁵, R^{k} and R^{g} are defined as in general formula (I).

In a preferred embodiment of the present invention, the compound represented by general formula (I) or the stereoisomer or the tautomer thereof, or the pharmaceutically acceptable salt thereof, is a compound represented by general formula (IV) or a stereoisomer or a tautomer thereof, or a pharmaceutically acceptable salt thereof:

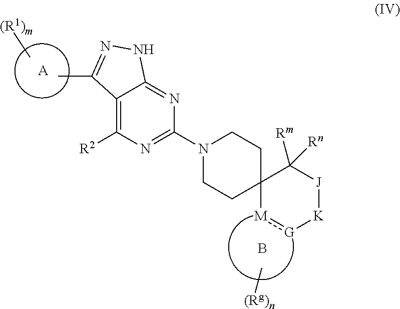

wherein:
ring B is selected from phenyl, 3-8 membered cycloalkyl, 4-8 membered heterocyclyl or 5-6 membered heteroaryl;
J is selected from CR^{p}R^{q};
K is selected from NR^{k}, (CR^{p}R^{q})ᵣ, O or S;
r is 0 or 1;
R^{m} is selected from amino, —CH₂NH₂ or —NHC(═NH)NH₂;
Rⁿ is selected from hydrogen atom, methyl or —CH₂OH;
R^{p} and R^{q} are each independently selected from hydrogen atom, halogen, amino, C₁-C₄ alkyls, hydroxy C₁-C₄ alkyls, amino C₁-C₄ alkyls or —OR⁵; and
[symbol] , ring A, G, M, m, n, R¹-R², R⁵, R^{k} and R^{g} are defined as in general formula (I).

In a preferred embodiment of the present invention, the compound represented by general formula (I) or the stereoisomer or the tautomer thereof, or the pharmaceutically acceptable salt thereof, is a compound represented by general formula (V) or a stereoisomer or a tautomer thereof, or a pharmaceutically acceptable salt thereof:

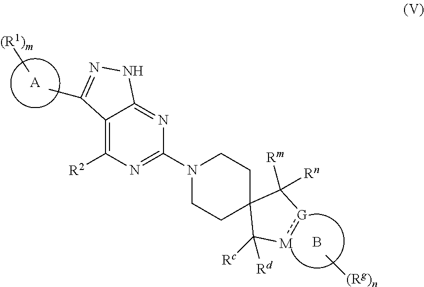

wherein:
ring B is selected from phenyl, 3-8 membered cycloalkyl, 4-8 membered heterocyclyl or 5-6 membered heteroaryl;
R^{c} and R^{d} together with the atom bound therewith form a 3-8 membered cycloalkyl;
R^{m} is selected from amino, —CH₂NH₂ or —NHC(═NH)NH₂;
Rⁿ is selected from hydrogen atom, methyl or —CH₂OH; and
[symbol] , ring A, G, M, m, n, R¹-R² and R^{g} are defined as in general formula (I).

In a preferred embodiment of the present invention, the compound represented by general formula (AI) or the stereoisomer or the tautomer thereof, or the pharmaceutically acceptable salt thereof, is a compound represented by general formula (VI) or a stereoisomer or a tautomer thereof, or a pharmaceutically acceptable salt thereof:

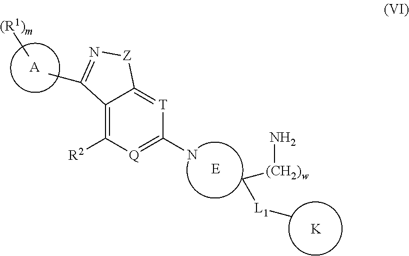

wherein:
L₁ is absent, or selected from —(C═O)— and —(CR^{w}R^{v})—, wherein any one of —(CR^{w}R^{v})— is optionally further replaced by —N(R^{z})—, —O—, —S—, —SO— and —SO₂—;
each R^{w} and R^{v} are the same or different, and are each independently selected from hydrogen atom, halogen, hydroxy, alkyl or alkoxy;
each R^{z} are the same or different, and are each independently selected from hydrogen atom or alkyl;
ring E is selected from 4-11 membered monocyclic heterocyclyl containing N, 4-11 membered fused heterocyclyl containing N or 4-11 membered bridged heterocyclyl containing N, wherein the monocyclic heterocyclyl, fused heterocyclyl or bridged heterocyclyl is optionally further substituted by one or more substituents selected from halogen, alkyl, —OR⁵ or ═O;
ring K is absent, or selected from cycloalkyl, aryl or heteroaryl, wherein the cycloalkyl, aryl or heteroaryl is optionally further substituted by one or more substituents selected from hydroxy, amino, halogen, nitro, cyano, alkyl, alkoxy, haloalkyl, cycloalkyl, heterocyclyl, aryl, heteroaryl, —C(O)R⁸, —C(O)OR⁸, —OC(O)R⁸, —SO₂R, —NR⁹R¹⁰, —C(O)NR⁹R¹⁰, —SO₂NR⁹R¹⁰ or —NR⁹C(O)R¹⁰;
wherein -L₁-ring K and —(CH₂)_{w}—NH₂ are bound to the same carbon atom of ring E;
w is 0, 1 or 2;
u is 0, 1, 2 or 3; and
ring A, Z, Q, T, m, R¹-R², R⁵, and R⁸-R¹⁰ are defined as in general formula (AI).

In a preferred embodiment of the present invention, in the compound represented by general formula (AI), (AII), (I), (II), (III), (IV), (V) or (VI) or the stereoisomer or the tautomer thereof, or the pharmaceutically acceptable salt thereof, R¹ is selected from hydrogen atom, F, Cl, Br, amino, hydroxy, cyano, nitro, methoxy, ethoxy, methyl, ethyl, trifluoromethyl, cyclopropyloxy, ethynyl, ethenyl, —NHCH₃ or —N(CH₃)₂.

In a preferred embodiment of the present invention, in the compound represented by general formula (III), (IV), (V) or (VI) or the stereoisomer or the tautomer thereof, or the pharmaceutically acceptable salt thereof, R² is selected from —C(O)NH₂ or —C(O)OH.

In a preferred embodiment of the present invention, in the compound represented by general formula (AI), (AII), (I), (II), (III), (IV), (V) or (VI) or the stereoisomer or the tautomer thereof, or the pharmaceutically acceptable salt thereof, R⁵ is selected from hydrogen atom or alkyl.

In a preferred embodiment of the present invention, in the compound represented by general formula (AI), (All), (I), (II), (III), (IV), (V) or (VI) or the stereoisomer or the tautomer thereof, or the pharmaceutically acceptable salt thereof, ring A is selected from phenyl, pyridinyl or pyrimidinyl.

In a preferred embodiment of the present invention, in the compound represented by general formula (III), (IV) or (V) or the stereoisomer or the tautomer thereof, or the pharmaceutically acceptable salt thereof, ring B is selected from:

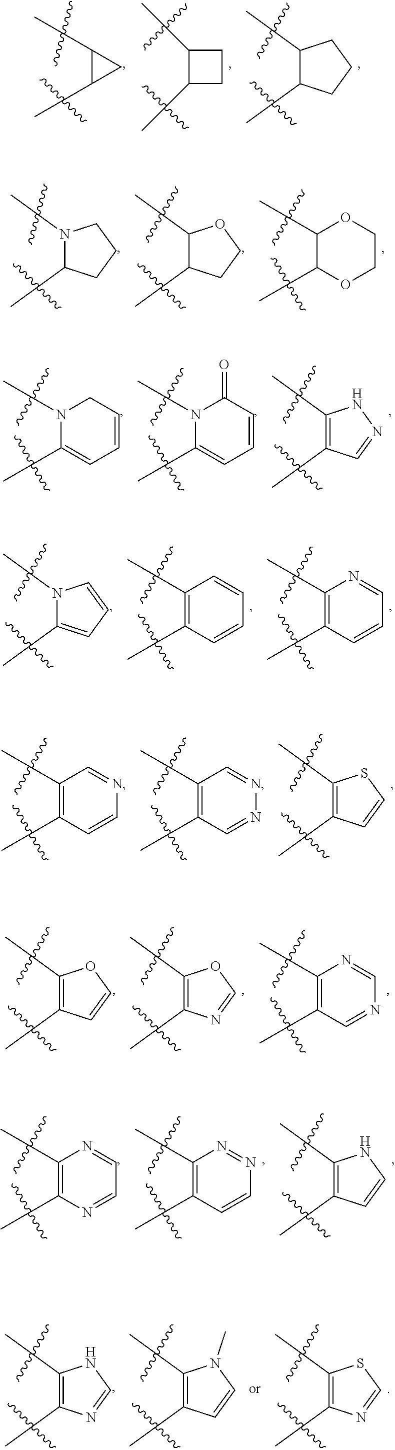

In a preferred embodiment of the present invention, in the compound represented by general formula (III), (IV) or (V) or the stereoisomer or the tautomer thereof, or the pharmaceutically acceptable salt thereof, R^{g} are the same or different, and are each independently selected from hydrogen atom, F, Cl, Br, amino, hydroxy, cyano, nitro, methoxy, ethoxy, methyl, ethyl, ethynyl, ethenyl, —NHCH₃ or —N(CH₃)₂; and
alternatively, two R^{g} together with the same carbon atom bound therewith form C═O.

In a preferred embodiment of the present invention, in the compound represented by general formula (VI) or the stereoisomer or the tautomer thereof, or the pharmaceutically acceptable salt thereof, ring E is selected from:

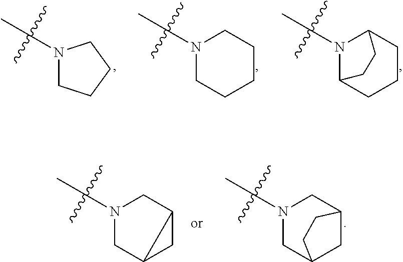

In a preferred embodiment of the present invention, the compound represented by general (AI) is selected from:

| Compound | | |
|---|---|---|
| No. | Structure | Name |
| Example 1 | 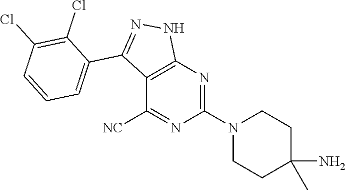 [SMILES: CC1(N)CCN(c2nc(C#N)c3c(-c4cccc(Cl)c4Cl)n[nH]c3n2)CC1] | 6-(4-Amino-4-methylpiperidin-1- yl)-3-(2,3-dichlorophenyl)-1H- pyrazolo[3,4-d]pyrimidine-4- carbonitrile |
| Example 2 | 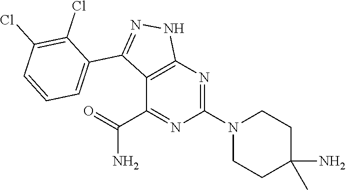 [SMILES: CC1(N)CCN(c2nc(C(N)=O)c3c(-c4cccc(Cl)c4Cl)n[nH]c3n2)CC1] | 6-(4-Amino-4-methylpiperidin-1- yl)-3-(2,3-dichlorophenyl)-1H- pyrazolo[3,4-d]pyrimidine-4- carboxamide |
| Example 3 | 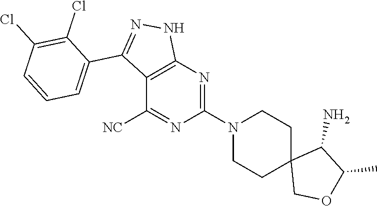 [SMILES: C[C@@H]1OCC2(CCN(c3nc(C#N)c4c(-c5cccc(Cl)c5Cl)n[nH]c4n3)CC2)[C@@H]1N] | 6-((3S,4S)-4-Amino-3-methyl-2- oxa-8-azaspiro[4.5]decan-8-yl)-3- (2,3-dichlorophenyl)-1H- pyrazolo[3,4-d]pyrimidine-4- carbonitrile |
| Example 4 | 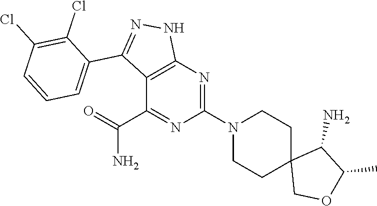 [SMILES: C[C@@H]1OCC2(CCN(c3nc(C(N)=O)c4c(-c5cccc(Cl)c5Cl)n[nH]c4n3)CC2)[C@@H]1N] | 6-((3S,4S)-4-amino-3-methyl-2- oxa-8-azaspiro[4.5]decan-8-yl)-3- (2,3-dichlorophenyl)-1H- pyrazolo[3,4-d]pyrimidine-4- carboxamide |
| Example 5 | 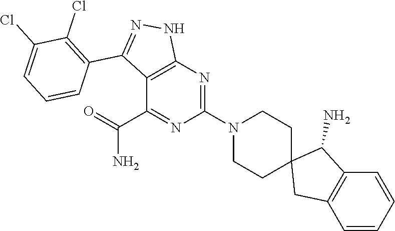 [SMILES: NC(=O)c1nc(N2CCC3(CC2)Cc2ccccc2[C@H]3N)nc2[nH]nc(-c3cccc(Cl)c3Cl)c12] | (S)-6-(1-amino-1,3- dihydrospiro[indene-2,4′-piperidin]- 1′-yl)-3-(2,3-dichlorophenyl)-1H- pyrazolo[3,4-d]pyrimidine-4- carboxamide |
| Example 6 | 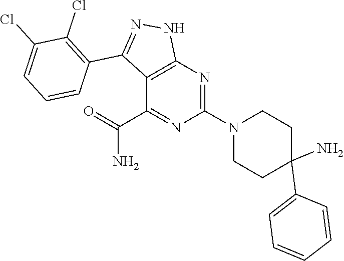 [SMILES: NC(=O)c1nc(N2CCC(N)(c3ccccc3)CC2)nc2[nH]nc(-c3cccc(Cl)c3Cl)c12] | 6-(4-Amino-4-phenylpiperidin-1- yl)-3-(2,3-dichlorophenyl)-1H- pyrazolo[3,4-d]pyrimidine-4- carboxamide |
| Example 7 | 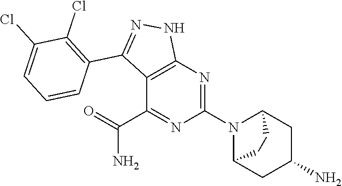 [SMILES: NC(=O)c1nc(N2[C@@H]3CC[C@H]2C[C@@H](N)C3)nc2[nH]nc(-c3cccc(Cl)c3Cl)c12] | 6-((Endo)-3-amino-8- azabicyclo[3.2.1]octan-8-yl)-3-(2,3- dichlorophenyl)-1H-pyrazolo[3,4- d]pyrimidine-4-carboxamide |
| Example 8 | 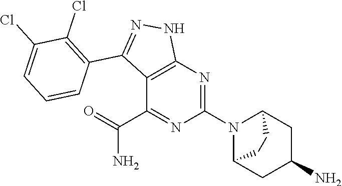 [SMILES: NC(=O)c1nc(N2[C@@H]3CC[C@H]2C[C@H](N)C3)nc2[nH]nc(-c3cccc(Cl)c3Cl)c12] | 6-((Exo)-3-amino-8- azabicyclo[3.2.1]octan-8-yl)-3-(2,3- dichlorophenyl)-1H-pyrazolo[3,4- d]pyrimidine-4-carboxamide |
| Example 9 | 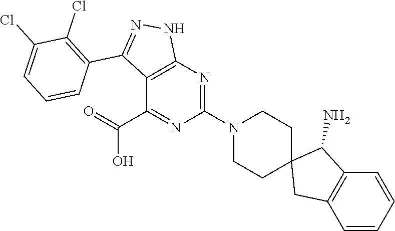 [SMILES: N[C@@H]1c2ccccc2CC12CCN(c1nc(C(=O)O)c3c(-c4cccc(Cl)c4Cl)n[nH]c3n1)CC2] | (S)-6-(1-Amino-1,3- dihydrospiro[indene-2,4′-piperidin]- 1′-yl)-3-(2,3-dichlorophenyl)-1H- pyrazolo[3,4-d]pyrimidine-4- carboxylic acid |
| Example 10 | 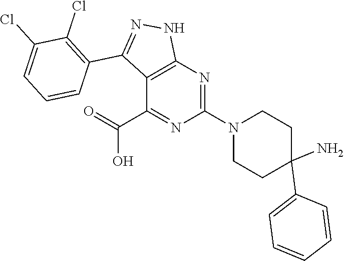 [SMILES: NC1(c2ccccc2)CCN(c2nc(C(=O)O)c3c(-c4cccc(Cl)c4Cl)n[nH]c3n2)CC1] | 6-(4-Amino-4-phenylpiperidin-1- yl)-3-(2,3-dichlorophenyl)-1H- pyrazolo[3,4-d]pyrimidine-4- carboxylic acid |
| Example 11 | 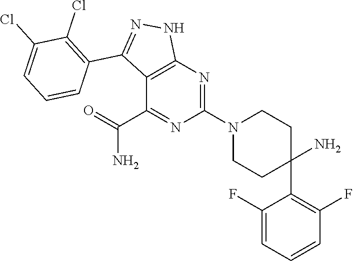 [SMILES: NC(=O)c1nc(N2CCC(N)(c3c(F)cccc3F)CC2)nc2[nH]nc(-c3cccc(Cl)c3Cl)c12] | 6-(4-Amino-4-(2,6- difluorophenyl)piperidin-1-yl)-3- (2,3-dichlorophenyl)-1H- pyrazolo[3,4-d]pyrimidine-4- carboxamide |
| Example 12 | 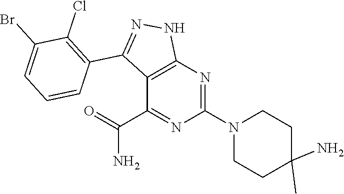 [SMILES: CC1(N)CCN(c2nc(C(N)=O)c3c(-c4cccc(Br)c4Cl)n[nH]c3n2)CC1] | 6-(4-Amino-4-methylpiperidin-1- yl)-3-(3-bromo-2-chlorophenyl)- 1H-pyrazolo[3,4-d]pyrimidine-4- carboxamide |
| Example 13 | 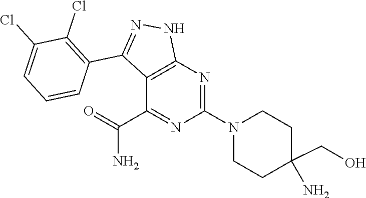 [SMILES: NC(=O)c1nc(N2CCC(N)(CO)CC2)nc2[nH]nc(-c3cccc(Cl)c3Cl)c12] | 6-(4-Amino-4- (hydroxymethyl)piperidin-1-yl)-3- (2,3-dichlorophenyl)-1H- pyrazolo[3,4-d]pyrimidine-4- carboxamide |
| Example 14 | 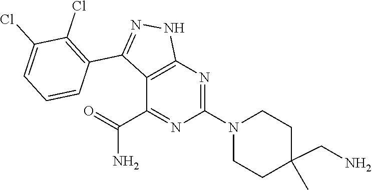 [SMILES: CC1(CN)CCN(c2nc(C(N)=O)c3c(-c4cccc(Cl)c4Cl)n[nH]c3n2)CC1] | 6-(4-(Aminomethyl)-4- methylpiperidin-1-yl)-3-(2,3- dichlorophenyl)-1H-pyrazolo[3,4- d]pyrimidine-4-carboxamide |
| Example 15 | 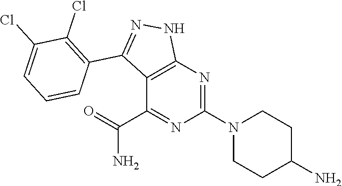 [SMILES: NC(=O)c1nc(N2CCC(N)CC2)nc2[nH]nc(-c3cccc(Cl)c3Cl)c12] | 6-(4-Aminopiperidin-1-yl)-3-(2,3- dichlorophenyl)-1H-pyrazolo[3,4- d]pyrimidine-4-carboxamide |
| Example 16 | 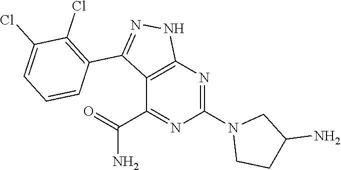 [SMILES: NC(=O)c1nc(N2CCC(N)C2)nc2[nH]nc(-c3cccc(Cl)c3Cl)c12] | 6-(3-Aminopyrrolidin-1-yl)-3-(2,3- dichlorophenyl)-1H-pyrazolo[3,4- d]pyrimidine-4-carboxamide |
| Example 17 | 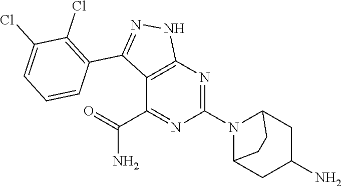 [SMILES: NC(=O)c1nc(N2C3CCC2CC(N)C3)nc2[nH]nc(-c3cccc(Cl)c3Cl)c12] | 6-(3-Amino-8- azabicyclo[3.2.1]octan-8-yl)-3-(2,3- dichlorophenyl)-1H-pyrazolo[3,4- d]pyrimidine-4-carboxamide |
| Example 18 | 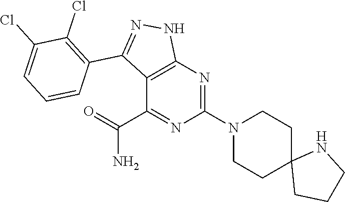 [SMILES: NC(=O)c1nc(N2CCC3(CCCN3)CC2)nc2[nH]nc(-c3cccc(Cl)c3Cl)c12] | 3-(2,3-Dichlorophenyl)-6-(1,8- diazaspiro[4.5]decan-8-yl)-1H- pyrazolo[3,4-d]pyrimidine-4- carboxamide |
| Example 19 | 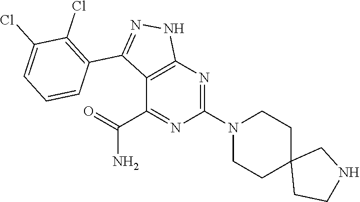 [SMILES: NC(=O)c1nc(N2CCC3(CCNC3)CC2)nc2[nH]nc(-c3cccc(Cl)c3Cl)c12] | 3-(2,3-Dichlorophenyl)-6-(2,8- diazaspiro[4.5]decan-8-yl)-1H- pyrazolo[3,4-d]pyrimidine-4- carboxamide |
| Example 20 | 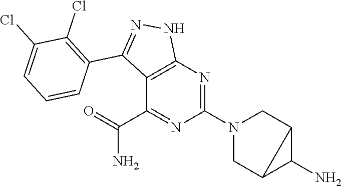 [SMILES: NC(=O)c1nc(N2CC3C(N)C3C2)nc2[nH]nc(-c3cccc(Cl)c3Cl)c12] | 6-(6-Amino-3- azabicyclo[3.1.0]hexan-3-yl)-3- (2,3-dichlorophenyl)-1H- pyrazolo[3,4-d]pyrimidine-4- carboxamide |
| Example 21 | 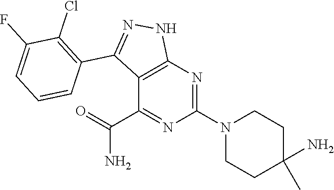 [SMILES: CC1(N)CCN(c2nc(C(N)=O)c3c(-c4cccc(F)c4Cl)n[nH]c3n2)CC1] | 6-(4-Amino-4-methylpiperidin-1- yl)-3-(2-chloro-3-fluorophenyl)-1H- pyrazolo[3,4-d]pyrimidine-4- carboxamide |
| Example 22 | 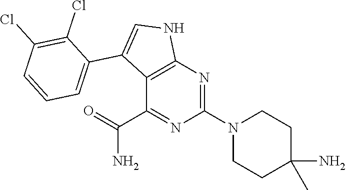 [SMILES: CC1(N)CCN(c2nc(C(N)=O)c3c(-c4cccc(Cl)c4Cl)c[nH]c3n2)CC1] | 2-(4-Amino-4-methylpiperidin-1- yl)-5-(2,3-dichlorophenyl)-7H- pyrrolo[2,3-d]pyrimidine-4- carboxamide |
| Example 23 | 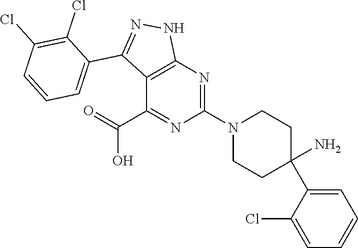 [SMILES: NC1(c2ccccc2Cl)CCN(c2nc(C(=O)O)c3c(-c4cccc(Cl)c4Cl)n[nH]c3n2)CC1] | 6-(4-Amino-4-(2- chlorophenyl)piperidin-1-yl)-3-(2,3- dichlorophenyl)-1H-pyrazolo[3,4- d]pyrimidine-4-carboxylic acid |
| Example 24 | 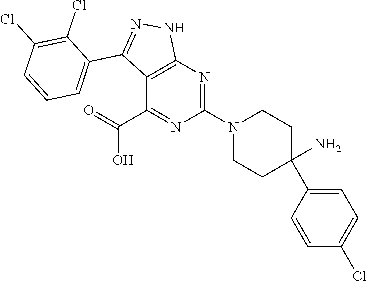 [SMILES: NC1(c2ccc(Cl)cc2)CCN(c2nc(C(=O)O)c3c(-c4cccc(Cl)c4Cl)n[nH]c3n2)CC1] | 6-(4-Amino-4-(4- chlorophenyl)piperidin-1-yl)-3-(2,3- dichlorophenyl)-1H-pyrazolo[3,4- d]pyrimidine-4-carboxylic acid |
| Example 25 | 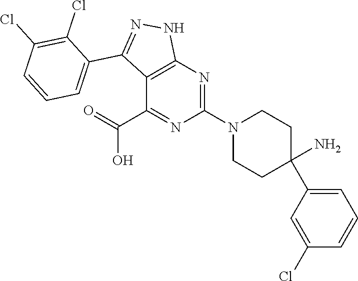 [SMILES: NC1(c2cccc(Cl)c2)CCN(c2nc(C(=O)O)c3c(-c4cccc(Cl)c4Cl)n[nH]c3n2)CC1] | 6-(4-Amino-4-(3- chlorophenyl)piperidin-1-yl)-3-(2,3- dichlorophenyl)-1H-pyrazolo[3,4- d]pyrimidine-4-carboxylic acid |
| Example 26 | 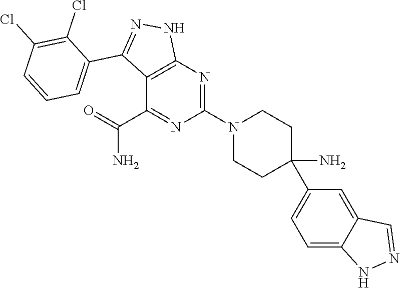 [SMILES: NC(=O)c1nc(N2CCC(N)(c3ccc4[nH]ncc4c3)CC2)nc2[nH]nc(-c3cccc(Cl)c3Cl)c12] | 6-(4-Amino-4-(1H-indazole-5- yl)piperidin-1-yl)-3-(2,3- dichlorophenyl)-1H-pyrazolo[3,4- d]pyrimidine-4-carboxamide |
| Example 27 | 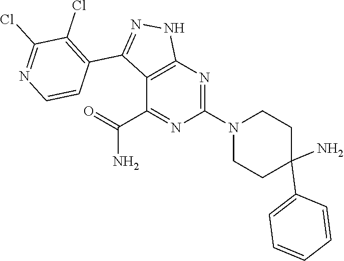 [SMILES: NC(=O)c1nc(N2CCC(N)(c3ccccc3)CC2)nc2[nH]nc(-c3ccnc(Cl)c3Cl)c12] | 6-(4-Amino-4-phenylpiperidin-1- yl)-3-(2,3-dichloropyridin-4-yl)-1H- pyrazolo[3,4-d]pyrimidine-4- carboxamide |
| Example 28 | 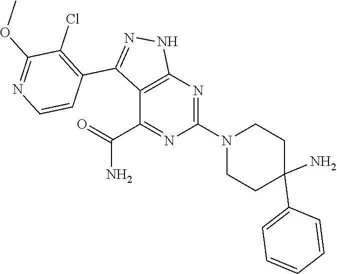 [SMILES: COc1nccc(-c2n[nH]c3nc(N4CCC(N)(c5ccccc5)CC4)nc(C(N)=O)c23)c1Cl] | 6-(4-Amino-4-phenylpiperidin-1- yl)-3-(3-chloro-2-methoxypyridin- 4-yl)-1H-pyrazolo[3,4- d]pyrimidine-4-carboxamide |
| Example 29 | 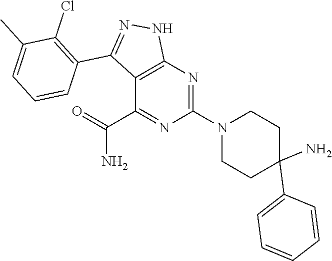 [SMILES: Cc1cccc(-c2n[nH]c3nc(N4CCC(N)(c5ccccc5)CC4)nc(C(N)=O)c23)c1Cl] | 6-(4-Amino-4-phenylpiperidin-1- yl)-3-(2-chloro-3-methylphenyl)- 1H-pyrazolo[3,4-d]pyrimidine-4- carboxamide |
| Example 30 | 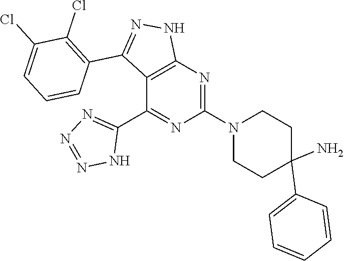 [SMILES: NC1(c2ccccc2)CCN(c2nc(-c3nnn[nH]3)c3c(-c4cccc(Cl)c4Cl)n[nH]c3n2)CC1] | 1-(3-(2,3-Dichlorophenyl)-4-(1H- tetrazol-5-yl)-1H-pyrazolo[3,4- d]pyrimidin-6-yl)-4- phenylpiperidin-4-amine |
| Example 32 | 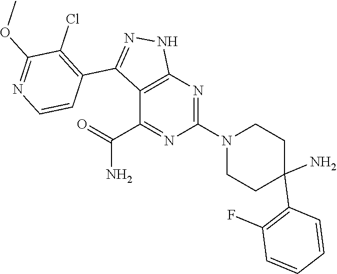 [SMILES: COc1nccc(-c2n[nH]c3nc(N4CCC(N)(c5ccccc5F)CC4)nc(C(N)=O)c23)c1Cl] | 6-(4-Amino-4-(2- fluorophenyl)piperidin-1-yl)-3-(3- chloro-2-methoxypyridin-4-yl)-1H- pyrazolo[3,4-d]pyrimidine-4- carboxamide |
| Example 33 | 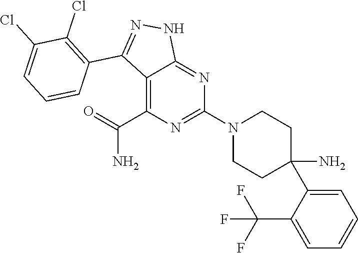 [SMILES: NC(=O)c1nc(N2CCC(N)(c3ccccc3C(F)(F)F)CC2)nc2[nH]nc(-c3cccc(Cl)c3Cl)c12] | 6-(4-Amino-4-(2- (trifluoromethyl)phenyl)piperidin-1- yl)-3-(2,3-dichlorophenyl)-1H- pyrazolo[3,4-d]pyrimidine-4- carboxamide |
| Example 34 | 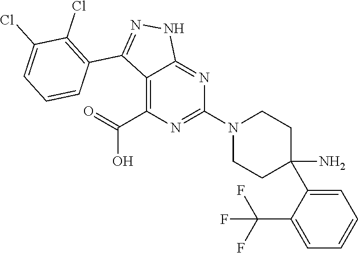 [SMILES: NC1(c2ccccc2C(F)(F)F)CCN(c2nc(C(=O)O)c3c(-c4cccc(Cl)c4Cl)n[nH]c3n2)CC1] | 6-(4-Amino-4-(2- (trifluoromethyl)phenyl)piperidin-1- yl)-3-(2,3-dichlorophenyl)-1H- pyrazolo[3,4-d]pyrimidine-4- carboxylic acid |
| Example 35 | 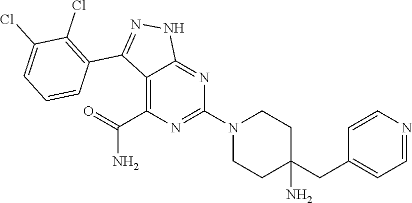 [SMILES: NC(=O)c1nc(N2CCC(N)(Cc3ccncc3)CC2)nc2[nH]nc(-c3cccc(Cl)c3Cl)c12] | 6-(4-Amino-4-(pyridin-4- ylmethyl)piperidin-1-yl)-3-(2,3- dichlorophenyl)-1H-pyrazolo[3,4- d]pyrimidine-4-carboxamide |
| Example 36 | 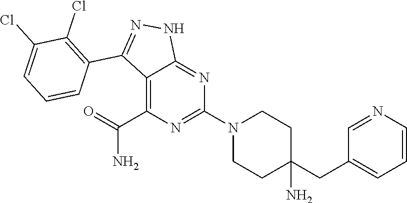 [SMILES: NC(=O)c1nc(N2CCC(N)(Cc3cccnc3)CC2)nc2[nH]nc(-c3cccc(Cl)c3Cl)c12] | 6-(4-Amino-4-(pyridin-3- ylmethyl)piperidin-1-yl)-3-(2,3- dichlorophenyl)-1H-pyrazolo[3,4- d]pyrimidine-4-carboxamide |
| Example 37 | 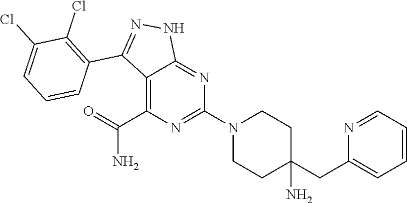 [SMILES: NC(=O)c1nc(N2CCC(N)(Cc3ccccn3)CC2)nc2[nH]nc(-c3cccc(Cl)c3Cl)c12] | 6-(4-Amino-4-(pyridin-2- ylmethyl)piperidin-1-yl)-3-(2,3- dichlorophenyl)-1H-pyrazolo[3,4- d]pyrimidine-4-carboxamide |
| Example 38 | 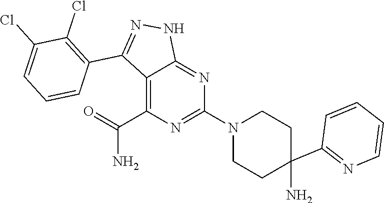 [SMILES: NC(=O)c1nc(N2CCC(N)(c3ccccn3)CC2)nc2[nH]nc(-c3cccc(Cl)c3Cl)c12] | 6-(4-Amino-4-(pyridin-2- yl)piperidin-1-yl)-3-(2,3- dichlorophenyl)-1H-pyrazolo[3,4- d]pyrimidine-4-carboxamide |
| Example 39 | 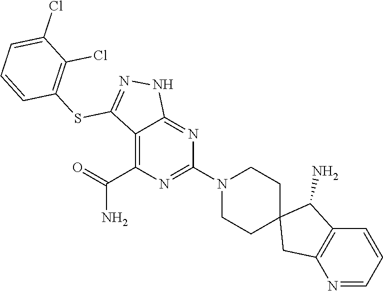 [SMILES: NC(=O)c1nc(N2CCC3(CC2)Cc2ncccc2[C@H]3N)nc2[nH]nc(Sc3cccc(Cl)c3Cl)c12] | (S)-6-(5-amino-5,7- dihydrospiro[cyclopenta[b]pyridine- 6,4′-piperidin]-1′-yl)-3-((2,3- dichlorophenyl)thio)-1H- pyrazolo[3,4-d]pyrimidine-4- carboxamide |
| Example 40 | 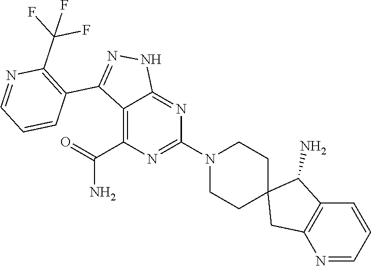 [SMILES: NC(=O)c1nc(N2CCC3(CC2)Cc2ncccc2[C@H]3N)nc2[nH]nc(-c3cccnc3C(F)(F)F)c12] | (S)-6-(5-amino-5,7- dihydrospiro[cyclopenta[b]pyridine- 6,4′-piperidin]-1′-yl)-3-(2- (trifluoromethyl)pyridin-3-yl)-1H- pyrazolo[3,4-d]pyrimidine-4- carboxamide |
| Example 41 | 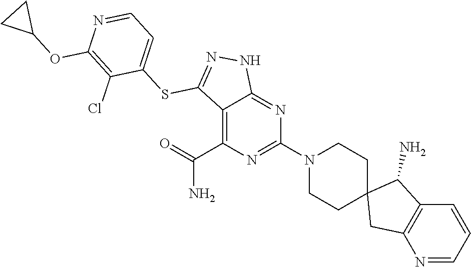 [SMILES: NC(=O)c1nc(N2CCC3(CC2)Cc2ncccc2[C@H]3N)nc2[nH]nc(Sc3ccnc(OC4CC4)c3Cl)c12] | (S)-6-(5-amino-5,7- dihydrospiro[cyclopenta[b]pyridine- 6,4′-piperidin]-1′-yl)-3-((3-chloro- 2-cyclopropoxypyridin-4-yl)thio)- 1H-pyrazolo[3,4-d]pyrimidine-4- carboxamide |
| Example 42 | 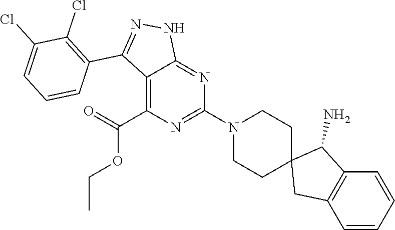 [SMILES: CCOC(=O)c1nc(N2CCC3(CC2)Cc2ccccc2[C@H]3N)nc2[nH]nc(-c3cccc(Cl)c3Cl)c12] | Ethyl (S)-6-(1-amino-1,3- dihydrospiro[indene-2,4′-piperidin]- 1′-yl)-3-(2,3-dichlorophenyl)-1H- pyrazolo[3,4-d]pyrimidine-4- carboxylate |
| Example 43 | 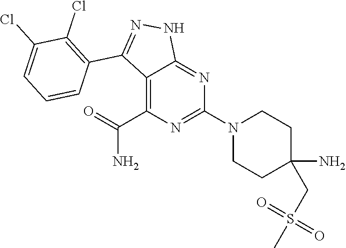 [SMILES: CS(=O)(=O)CC1(N)CCN(c2nc(C(N)=O)c3c(-c4cccc(Cl)c4Cl)n[nH]c3n2)CC1] | 6-(4-Amino-4- ((methylsulfonyl)methyl)piperidin- 1-yl)-3-(2,3-dichlorophenyl)-1H- pyrazolo[3,4-d]pyrimidine-4- carboxamide |
| Example 44 | 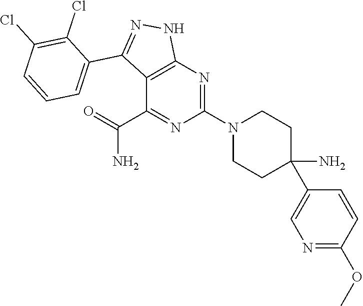 [SMILES: COc1ccc(C2(N)CCN(c3nc(C(N)=O)c4c(-c5cccc(Cl)c5Cl)n[nH]c4n3)CC2)cn1] | 6-(4-Amino-4-(6-methoxypyridin- 3-yl)piperidin-1-yl)-3-(2,3- dichlorophenyl)-1H-pyrazolo[3,4- d]pyrimidine-4-carboxamide |
| Example 45 | 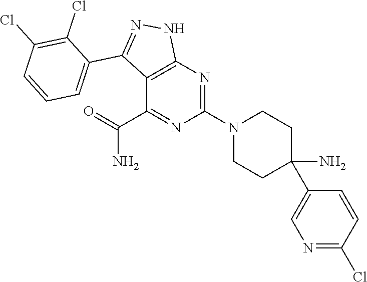 [SMILES: NC(=O)c1nc(N2CCC(N)(c3ccc(Cl)nc3)CC2)nc2[nH]nc(-c3cccc(Cl)c3Cl)c12] | 6-(4-Amino-4-(6-chloropyridin-3- yl)piperidin-1-yl)-3-(2,3- dichlorophenyl)-1H-pyrazolo[3,4- d]pyrimidine-4-carboxamide |
| Example 46 | 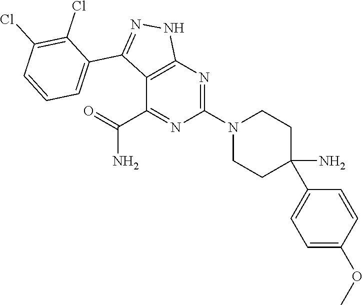 [SMILES: COc1ccc(C2(N)CCN(c3nc(C(N)=O)c4c(-c5cccc(Cl)c5Cl)n[nH]c4n3)CC2)cc1] | 6-(4-Amino-4-(4- methoxyphenyl)piperidin-1-yl)-3- (2,3-dichlorophenyl)-1H- pyrazolo[3,4-d]pyrimidine-4- carboxamide |
| Example 47 | 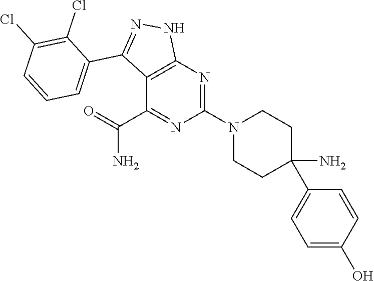 [SMILES: NC(=O)c1nc(N2CCC(N)(c3ccc(O)cc3)CC2)nc2[nH]nc(-c3cccc(Cl)c3Cl)c12] | 6-(4-Amino-4-(4- hydroxyphenyl)piperidin-1-yl)-3- (2,3-dichlorophenyl)-1H- pyrazolo[3,4-d]pyrimidine-4- carboxamide |
| Example 48 | 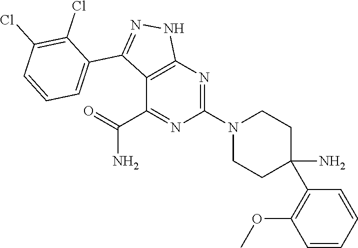 [SMILES: COc1ccccc1C1(N)CCN(c2nc(C(N)=O)c3c(-c4cccc(Cl)c4Cl)n[nH]c3n2)CC1] | 6-(4-Amino-4-(2- methoxyphenyl)piperidin-1-yl)-3- (2,3-dichlorophenyl)-1H- pyrazolo[3,4-d]pyrimidine-4- carboxamide |
| Example 49 | 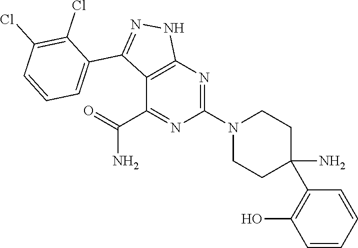 [SMILES: NC(=O)c1nc(N2CCC(N)(c3ccccc3O)CC2)nc2[nH]nc(-c3cccc(Cl)c3Cl)c12] | 6-(4-Amino-4-(2- hydroxyphenyl)piperidin-1-yl)-3- (2,3-dichlorophenyl)-1H- pyrazolo[3,4-d]pyrimidine-4- carboxamide |
| Example 50 | 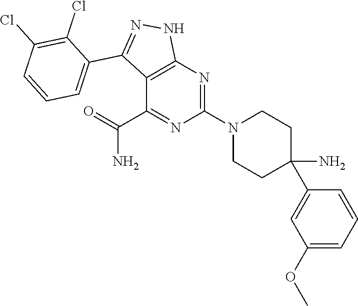 [SMILES: COc1cccc(C2(N)CCN(c3nc(C(N)=O)c4c(-c5cccc(Cl)c5Cl)n[nH]c4n3)CC2)c1] | 6-(4-Amino-4-(3- methoxyphenyl)piperidin-1-yl)-3- (2,3-dichlorophenyl)-1H- pyrazolo[3,4-d]pyrimidine-4- carboxamide |
| Example 51 | 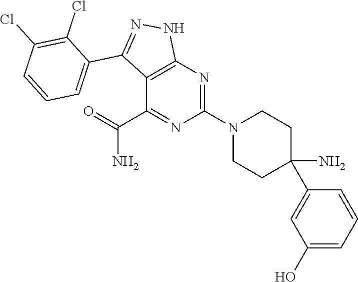 [SMILES: NC(=O)c1nc(N2CCC(N)(c3cccc(O)c3)CC2)nc2[nH]nc(-c3cccc(Cl)c3Cl)c12] | 6-(4-Amino-4-(3- hydroxyphenyl)piperidin-1-yl)-3- (2,3-dichlorophenyl)-1H- pyrazolo[3,4-d]pyrimidine-4- carboxamide |
| Example 52 | 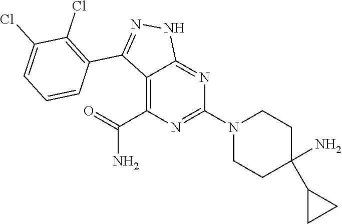 [SMILES: NC(=O)c1nc(N2CCC(N)(C3CC3)CC2)nc2[nH]nc(-c3cccc(Cl)c3Cl)c12] | 6-(4-Amino-4- cyclopropylpiperidin-1-yl)-3-(2,3- dichlorophenyl)-1H-pyrazolo[3,4- d]pyrimidine-4-carboxamide |
| Example 53 | 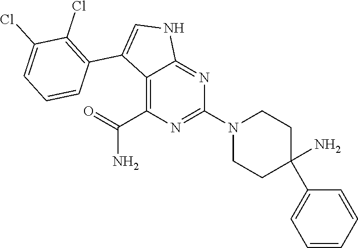 [SMILES: NC(=O)c1nc(N2CCC(N)(c3ccccc3)CC2)nc2[nH]cc(-c3cccc(Cl)c3Cl)c12] | 2-(4-Amino-4-phenylpiperidin-1- yl)-5-(2,3-dichlorophenyl)-7H- pyrrolo[2,3-d]pyrimidine-4- carboxamide |
| Example 54 | 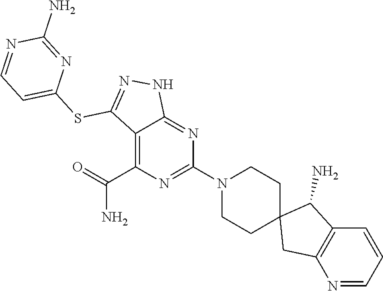 [SMILES: NC(=O)c1nc(N2CCC3(CC2)Cc2ncccc2[C@H]3N)nc2[nH]nc(Sc3ccnc(N)n3)c12] | (S)-6-(5-amino-5,7- dihydrospiro[cyclopenta[b]pyridine- 6,4′-piperidin]-1′-yl)-3-((2- aminopyrimidin-4-yl)thio)-1H- pyrazolo[3,4-d]pyrimidine-4- carboxamide |
| Example 55 | 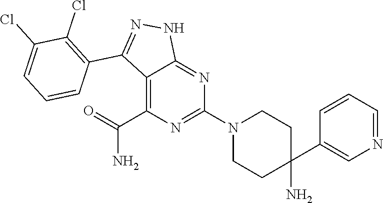 [SMILES: NC(=O)c1nc(N2CCC(N)(c3cccnc3)CC2)nc2[nH]nc(-c3cccc(Cl)c3Cl)c12] | 6-(4-Amino-4-(pyridin-3- yl)piperidin-1-yl)-3-(2,3- dichlorophenyl)-1H-pyrazolo[3,4- d]pyrimidine-4-carboxamide |
| Example 56 | 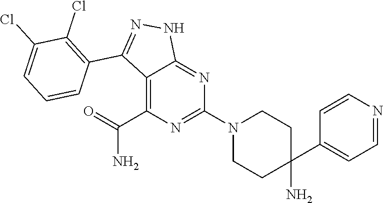 [SMILES: NC(=O)c1nc(N2CCC(N)(c3ccncc3)CC2)nc2[nH]nc(-c3cccc(Cl)c3Cl)c12] | 6-(4-Amino-4-(pyridin-4- yl)piperidin-1-yl)-3-(2,3- dichlorophenyl)-1H-pyrazolo[3,4- d]pyrimidine-4-carboxamide |
| Example 57 | 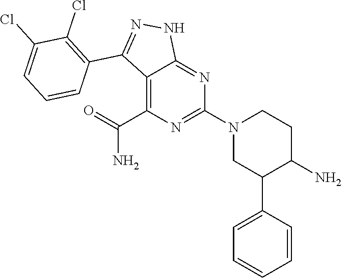 [SMILES: NC(=O)c1nc(N2CCC(N)C(c3ccccc3)C2)nc2[nH]nc(-c3cccc(Cl)c3Cl)c12] | 6-(4-Amino-3-phenylpiperidin-1- yl)-3-(2,3-dichlorophenyl)-1H- pyrazolo[3,4-d]pyrimidine-4- carboxamide |
| Example 58 | 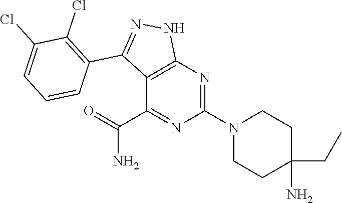 [SMILES: CCC1(N)CCN(c2nc(C(N)=O)c3c(-c4cccc(Cl)c4Cl)n[nH]c3n2)CC1] | 6-(4-Amino-4-ethylpiperidin-1-yl)- 3-(2,3-dichlorophenyl)-1H- pyrazolo[3,4-d]pyrimidine-4- carboxamide |

or a stereoisomer, tautomer thereof or a pharmaceutically acceptable salt thereof.

Further, the present invention provides a preparation method for the compound represented by general formula (I) or the stereoisomer or the tautomer thereof, wherein the method comprises:

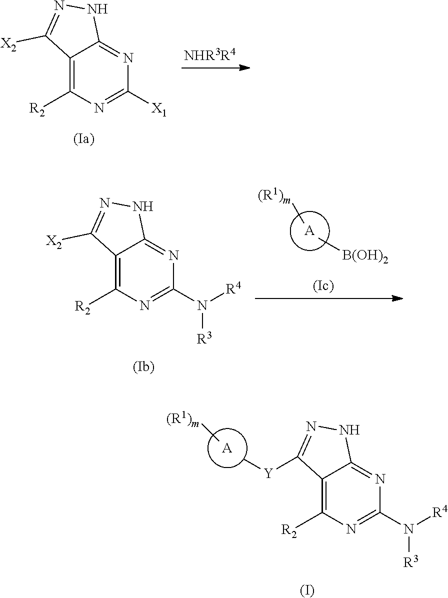

subjecting the compound represented by general formula (Ia) and NHR³R⁴ to a nucleophilic substitution reaction under alkaline condition to obtain the compound represented by general formula (Ib); and subjecting the compound represented by general formula (Ib) and the compound represented by general formula (Ic) to a Suzuki reaction in the presence of palladium catalyst and alkaline condition, and optionally further removing a protecting group of the obtained compound to obtain the compound represented by general formula (I); wherein:
Y is selected from chemical bond;
X₁ is selected from leaving group, wherein the leaving group is selected from halogen or —SO₂R^{t};
X₂ is selected from halogen;
R^{t} is selected from alkyl; and
ring A, m, and R¹-R⁴ are defined as in general formula (I).

Further, the present invention provides a preparation method for the compound represented by general formula (I) or the stereoisomer or the tautomer thereof, wherein the method comprises:

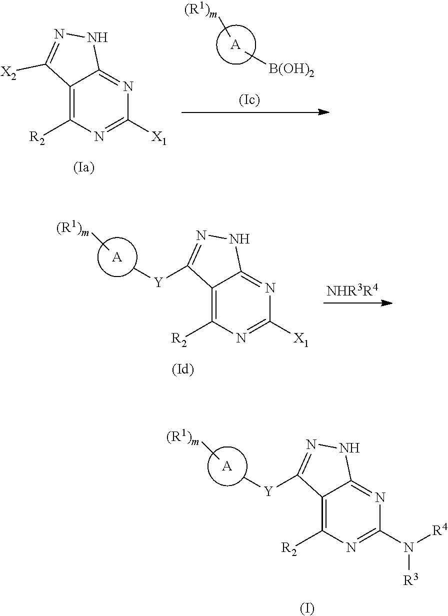

subjecting the compound represented by general formula (Ia) and the compound represented by general formula (Ic) to a Suzuki reaction in the presence of palladium catalyst and alkaline condition, to obtain the compound represented by general formula (Id); and subjecting the compound represented by general formula (Id) and NHR³R⁴ to a nucleophilic substitution reaction under alkaline condition, the protecting group of the obtained compound is removed to obtain the compound represented by general formula (I); wherein:
Y is selected from chemical bond;
X₁ is selected from leaving group, wherein the leaving group is selected from halogen or SO₂R^{t};
X₂ is selected from halogen;
R^{t} is selected from alkyl; and
ring A, m, and R¹-R⁴ are defined as in general formula (I).

Further, the present invention provides a compound represented by general formula (Ia) or a stereoisomer or a tautomer thereof, which is an intermediate for preparing a compound represented by general formula (I):

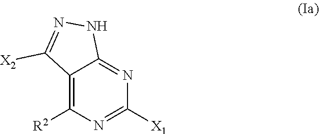

wherein:
X₁ is selected from leaving group, wherein the leaving group is selected from halogen or SO₂R^{t};
X₂ is selected from halogen;
R^{t} is selected from alkyl; and
R² is selected from cyano, tetrazolyl, —C(O)R⁵, —C(O)OR⁵ or —C(O)NR⁶R⁷;
R⁵, R⁶ and R⁷ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, amino, halogen, nitro, cyano, alkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
alternatively, R⁶ and R⁷ together with the N atom bound therewith form a 3-8 membered heterocyclyl, wherein the 3-8 membered heterocyclyl internally contains one or more N, O, S or SO₂ atoms, and the 3-8 membered heterocyclyl is optionally further substituted by one or more substituents selected from hydroxy, halogen, amino, alkyl or alkoxy; and
R⁸, R⁹ and R¹⁰ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, halogen, nitro, cyano, alkyl, alkoxy, cycloalkyl, heterocyclyl, aryl, heteroaryl, carboxyl or carboxylate.

Further, the present invention provides a preparation method for the compound represented by general formula (Ia) or the stereoisomer or the tautomer thereof, wherein the method comprises:

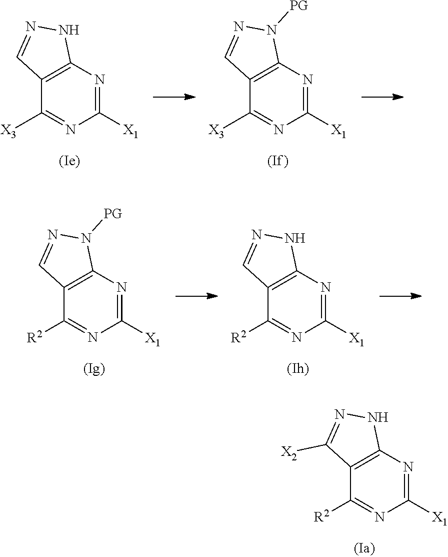

protecting the amino of the compound represented by general formula (Ie) to obtain the compound represented by general formula (If); subjecting the compound represented by general formula (If) to a coupling reaction under the action of palladium catalysts to obtain the compound represented by general formula (Ig); removing the protecting group PG from the compound represented by general formula (Ig) to obtain the compound represented by general formula (Ih); and subjecting the compound represented by general formula (Ih) to a halogenating reaction to obtain the compound represented by general formula (Ia); wherein:
PG is the protecting group, preferably

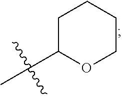 [SMILES: *C1CCCCO1]

X₃ is selected from halogen; and
X₁, X₂ and R² are as defined in general formula (Ia).

Further, the present invention provides a pharmaceutical composition, wherein the pharmaceutical composition comprises an effective dose of the compound represented by general formula (AI), (All), (I), (II), (III), (IV), (V) or (VI) or the stereoisomer or the tautomer thereof, or the pharmaceutically acceptable salt thereof, and a pharmaceutically acceptable carrier, an excipient or a combination thereof.

The present invention provides use of the compound represented by general formula (AI), (AII), (I), (II), (III), (IV), (V) or (VI) or the stereoisomer or the tautomer thereof, or the pharmaceutically acceptable salt thereof, or the pharmaceutical composition thereof in preparing a SHP2 allosteric inhibitor.

The present invention also provides use of the compound represented by general formula (AI), (AII), (I), (II), (III), (IV), (V) or (VI) or the stereoisomer or the tautomer thereof, or the pharmaceutically acceptable salt thereof, or the pharmaceutical composition thereof in preparing a medicament for treating a disease mediated by SHP2, wherein the disease mediated by SHP2 is preferably cancer, cancerometastasis, cardiovascular disease, immune disorder, fibrosis or visual disorder; wherein the disease mediated by SHP2 is preferably selected from Noonan syndrome, Leopard spot syndrome, juvenile myelomonocytic leukemia, neuroblastoma, melanoma, acute myeloid leukemia, breast cancer, esophagus cancer, lung cancer, colon cancer, head cancer, neuroblastoma, squamous cell carcinoma of head and neck, gastric cancer, anaplastic large cell lymphoma and glioblastoma.

The present invention further provides use of the compound represented by general formula (AI), (AII), (I), (II), (III), (IV), (V) or (VI) or the stereoisomer or the tautomer thereof, or the pharmaceutically acceptable salt thereof, or the pharmaceutical composition thereof in preparing a medicament for treating cancer, cancerometastasis, cardiovascular disease, immune disorder, fibrosis or visual disorder.

The present invention provides use of the compound represented by general formula (AI), (AII), (I), (II), (III), (IV), (V) or (VI) or the stereoisomer or the tautomer thereof, or the pharmaceutically acceptable salt thereof, or the pharmaceutical composition thereof in preparing a medicament for treating Noonan syndrome, Leopard spot syndrome, juvenile myelomonocytic leukemia, neuroblastoma, melanoma, acute myeloid leukemia, breast cancer, esophagus cancer, lung cancer, colon cancer, head cancer, neuroblastoma, squamous cell carcinoma of head and neck, gastric cancer, anaplastic large cell lymphoma and glioblastoma.

The present invention provides a method for inhibiting a SHP2 receptor in vitro, wherein the method comprises the step of contacting the SHP2 receptor with the compound represented by general formula (AI), (AII), (I), (II), (III), (IV), (V) or (VI) or the stereoisomer or the tautomer thereof, or the pharmaceutically acceptable salt thereof, or the pharmaceutical composition thereof.

The present invention provides a method for treating a disease mediated by SHP2, wherein the method comprises the steps of administering to a patient in need of treatment an effective dose of the compound represented by general formula (AI), (AII), (I), (II), (III), (IV), (V) or (VI) or the stereoisomer or the tautomer thereof, or the pharmaceutically acceptable salt thereof, or the pharmaceutical composition thereof, wherein the disease mediated by SHP2 is preferably cancer, cancerometastasis, cardiovascular disease, immune disorder, fibrosis or visual disorder; wherein the disease mediated by SHP2 is more preferably selected from Noonan syndrome, Leopard spot syndrome, juvenile myelomonocytic leukemia, neuroblastoma, melanoma, acute myeloid leukemia, breast cancer, esophagus cancer, lung cancer, colon cancer, head cancer, neuroblastoma, squamous cell carcinoma of head and neck, gastric cancer, anaplastic large cell lymphoma and glioblastoma.

### DETAILED DESCRIPTION OF THE INVENTION

Unless stated to the contrary, some terms used in the specification and claims of the present invention are defined as follows:

“Alkyl”, when regarded as a group or a part of a group, means to include C₁-C₂₀ linear chain or branched aliphatic hydrocarbon groups. It is preferably C₁-C₁₀ alkyl, and more preferably C₁-C₆ alkyl. Examples of alkyls include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, or the like. The alkyl may be substituted or unsubstituted.

“Alkenyl” refers to an alkyl as defined above consisting of at least two carbon atoms and at least one carbon-carbon double bond, representative examples of which comprise but are not limited to ethenyl, 1-propenyl, 2-propenyl, 1-, 2- or 3-butenyl, or the like. The alkenyl may also be substituted or unsubstituted.

“Alkynyl” refers to an aliphatic hydrocarbon group with one carbon-carbon triple bond, which may be a linear chain or branched chain. Preferably, C₂-C₁₀ alkynyl, more preferably C₂-C₆ alkynyl, and most preferably C₂-C₄ alkynyl. Examples of alkynyl groups comprise, but are not limited to, ethynyl, 1-propynyl, 2-propynyl, 1-, 2- or 3-butynyl, or the like. The alkynyl may be substituted or unsubstituted “Cycloalkyl” refers to saturated or partially saturated monocyclic, fused, bridged and spirocyclic carbocycles. Preferably, C₃-C₁₂ cycloalkyl, more preferably C₃-C₈ cycloalkyl, and most preferably C₃-C₆ cycloalkyl. Examples of monocyclic cycloalkyl comprise but are not limited to cyclopropyl, cyclobutyl, cyclopentyl, cyclopentenyl, cyclohexyl, cyclohexenyl, cyclohexadienyl, cycloheptyl, cyclohepttrienyl, cyclooctyl, or the like, and cyclopropyl and cyclohexenyl are preferred. The cycloalkyl may be optionally substituted or unsubstituted.

“Spirocycloalkyl” refers to a 5-18 membered polycyclic group with two or more cyclic structures, and single rings share one carbon atom (called spiro atom) with each other. The ring contains one or more double bonds, but none of the rings has a completely conjugated a electron aromatic system. Preferably, 6-14 membered, and more preferably 7-10 membered. According to the number of spiro atoms shared between rings, the spirocycloalkyl may be classified into mono-spiro, di-spiro or multi-spiro-cycloalkyls, preferably mono-spiro and di-spiro-cycloalkyls, and preferably 4 membered/5 membered, 4 membered/6 membered, 5 membered/5 membered, or 5 membered/6 membered. Non-limiting examples of “spirocycloalkyl” comprise, but are not limited to, spiro[4.5]decyl, spiro[4.4]nonyl, spiro[3.5]nonyl, and spiro[2.4]heptyl.

“Fused cycloalkyl” refers to a 5-18 membered all-carbon polycyclic group with two or more cyclic structures sharing a pair of carbon atoms, and one or more rings may contain one or more double bonds, but none of the rings has a completely conjugated a electron aromatic system. The fused acycloalkyl is preferably 6-12 membered, and more preferably 7-10 membered. According to the number of constituent rings, fused cycloalkyl may be classified into bicyclic, tricyclic, tetracyclic or polycyclic fused cycloalkyls, preferably bicyclic or tricyclic, and more preferably 5 membered/5 membered or 5 membered/6 membered bicycloalkyl. Non-limiting examples of “fused cycloalkyl” comprise, but are not limited to, bicyclo[3.1.0]hexyl, bicyclo[3.2.0]heptyl-1-alkenyl, bicyclo[3.2.0]heptyl, decalinyl or tetradecahydrophenanthryl.

“Bridged cycloalkyl” refers to a 5-18 membered all-carbon polycyclic group with two or more cyclic structures sharing two carbon atoms that are not directly bound with each other, one or more rings may contain one or more double bonds, but none of the rings has a completely conjugated a electron aromatic system. The bridged cycloalkyl is preferably 6-12 membered, and more preferably 7-10 membered. It is preferably 6-14 membered, and more preferably 7-10 membered. According to the number of constituent rings, it may be classified into bicyclic, tricyclic, tetracyclic or polycyclic bridged cycloalkyls, preferably bicyclic, tricyclic or tetracyclic, and more preferably bicyclic or tricyclic. Non-limiting examples of “bridged cycloalkyl” comprise, but are not limited to, (1s,4s)-bicyclo[2.2.1]heptyl, bicyclo[3.2.1]octyl, (1s,5s)-bicyclo[3.3.1]nonyl, bicyclo[2.2.2]octyl, and (1r,5r)-bicyclo[3.3.2]decyl.

“Heterocyclyl”, “heterocycle” or “heterocyclic” are used interchangeably in this application, and all refer to non-aromatic heterocyclyls, wherein one or more ring-forming atoms are heteroatoms, such as oxygen, nitrogen, sulfur atoms, or the like, comprising monocyclic ring, polycyclic ring, fused ring, bridged ring and spiro. Preferably having a 5-7 membered monocyclic ring or a 7-10 membered bicyclic or tricyclic ring, which may contain 1, 2 or 3 atoms selected from nitrogen, oxygen and/or sulfur. Examples of “heterocyclyl” comprise but are not limited to morpholinyl, oxetanyl, thiomorpholinyl, tetrahydropyranyl, 1,1-dioxo-thiomorpholinyl, piperidinyl, 2-oxo-piperidinyl, pyrrolidinyl, 2-oxo-pyrrolidinyl, piperazine-2-one, 8-oxa-3-aza-bicyclo[3.2.1]octyl, piperazinyl,

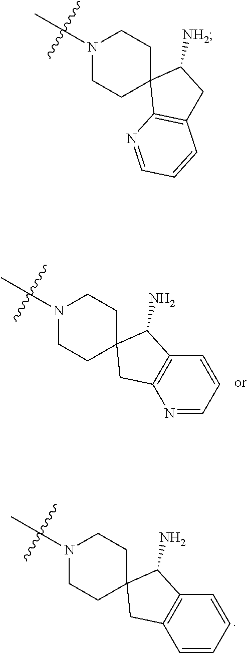 [SMILES: *N1CCC2(CC1)c1ncccc1C[C@H]2N ; *N1CCC2(CC1)Cc1ncccc1[C@H]2N ; *N1CCC2(CC1)Cc1ccccc1[C@H]2N]

The heterocyclyl may be substituted or unsubstituted.

“Spiroheterocyclyl” refers to a 5-18 membered polycyclic group with two or more cyclic structures, and single rings share one atom with each other. The ring contains one or more double bonds, but none of the rings has a completely conjugated a electron aromatic system, wherein one or more ring atoms are selected from heteroatoms of nitrogen, oxygen or S(O). (wherein n is selected from 0, 1 or 2), and the remaining ring atoms are carbon. It is preferably 6-14 membered, and more preferably 7-10 membered. According to the number of spiro atoms shared between rings, the spirocycloalkyl may be classified into mono-spiroheterocyclyl, bi-spiroheterocyclyl or multi-spiroheterocyclyl, preferably mono-spiroheterocyclyl and bi-spiroheterocyclyl. Preferably a 4 membered/4 membered, 4 membered/5 membered, 4 membered/6 membered, 5 membered/5 membered, or 5 membered/6 membered mono-spiroheterocyclyl. Non-limiting examples of “spiroheterocyclyl” comprise, but are not limited to: 1,7-dioxaspiro[4.5]decyl, 2-oxa-7-azaspiro[4.4]nonyl, 7-oxaspiro[3.5]nonyl, 5-oxaspiro[2.4]heptyl,

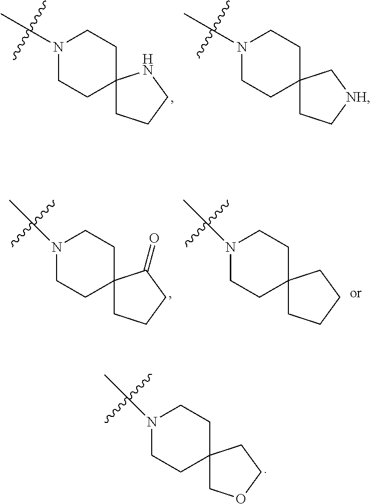 [SMILES: *N1CCC2(CCCN2)CC1 ; *N1CCC2(CCNC2)CC1 ; *N1CCC2(CCCC2=O)CC1 ; *N1CCC2(CCCC2)CC1 ; *N1CCC2(CCOC2)CC1]

“Fused heterocyclyl” refers to a polycyclic group with two or more cyclic structures sharing a pair of atoms, and one or more rings may contain one or more double bonds, but none of the rings has a completely conjugated a electron aromatic system, wherein one or more ring atoms are selected from heteroatoms of nitrogen, oxygen or S(O)ₙ (wherein n is selected from 0, 1 or 2), and the remaining ring atoms are carbon. Preferably 6-14 membered, and more preferably 7-10 membered. According to the number of constituent rings, fused heterocyclyl may be classified into bicyclic, tricyclic, tetracyclic or polycyclic fused heterocyclyls, preferably bicyclic or tricyclic, and more preferably 5 membered/5 membered or 5 membered/6 membered bicyclic fused heterocyclyl. Non-limiting examples of “fused heterocyclyl” comprise, but are not limited to: octahydropyrrolo[3,4-c]pyrrolyl, octahydro-1H-isoindolyl, 3-azabicyclo[3.1.0]hexyl, octahydrobenzo[b][1,4]dioxine (dioxine) and

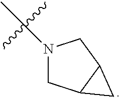 [SMILES: *N1CC2CC2C1]

“Bridged heterocyclyl” refers to a 5-14 membered or 5-18 membered polycyclic group with two or more cyclic structures sharing two atoms that are not directly bound with each other. One or more rings may contain one or more double bonds, but none of the rings has a completely conjugated a electron aromatic system, wherein one or more ring atoms are selected from heteroatoms of nitrogen, oxygen or S(O)ₙ (wherein n is selected from 0, 1 or 2), and the remaining ring atoms are carbon. It is preferably 6-14 membered, and more preferably 7-10 membered. According to the number of constituent rings, bridged heterocyclyl may be classified into bicyclic, tricyclic, tetracyclic or polycyclic bridged heterocyclyls, preferably bicyclic, tricyclic or tetracyclic, and more preferably bicyclic or tricyclic. Non-limiting examples of “bridged heterocyclyl” comprise, but are not limited to, 2-azabicyclo[2.2.1]heptyl, 2-azabicyclo[2.2.2]octyl, 2-azabicyclo[3.3.2]decyl,

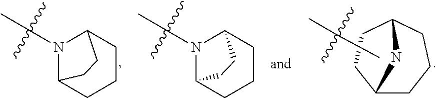

“Aryl” refers to a carbocyclic aromatic system containing one or two rings, wherein the rings may be bound together in a fused manner. The term “aryl” comprises monocyclic or bicyclic aryls, such as aromatic groups of phenyl, naphthyl, and tetrahydronaphthyl. The aryl may be substituted or unsubstituted.

“Heteroaryl” refers to a 5-6 membered monocyclic ring or a 8-10 membered bicyclic ring, which may contain 1 to 4 atoms selected from nitrogen, oxygen and/or sulfur. Examples of “heteroaryl” comprise but are not limited to, furanyl, pyridinyl, 2-oxo-1,2-dihydropyridyl, pyridazinyl, pyrimidinyl, pyrazinyl, thienyl, isoxazolyl, oxazolyl, oxadiazolyl, imidazolyl, pyrrolyl, pyrazolyl, triazolyl, tetrazolyl, thiazolyl, isothiazolyl, 1,2,3-thiadiazolyl, benzodioxolyl, benzothienyl, benzimidazolyl, indolyl, isoindolyl, 1,3-dioxo-isoindolyl, quinolinyl, indazolyl, benzisothiazolyl, benzoxazolyl, and benzisoxazolyl,

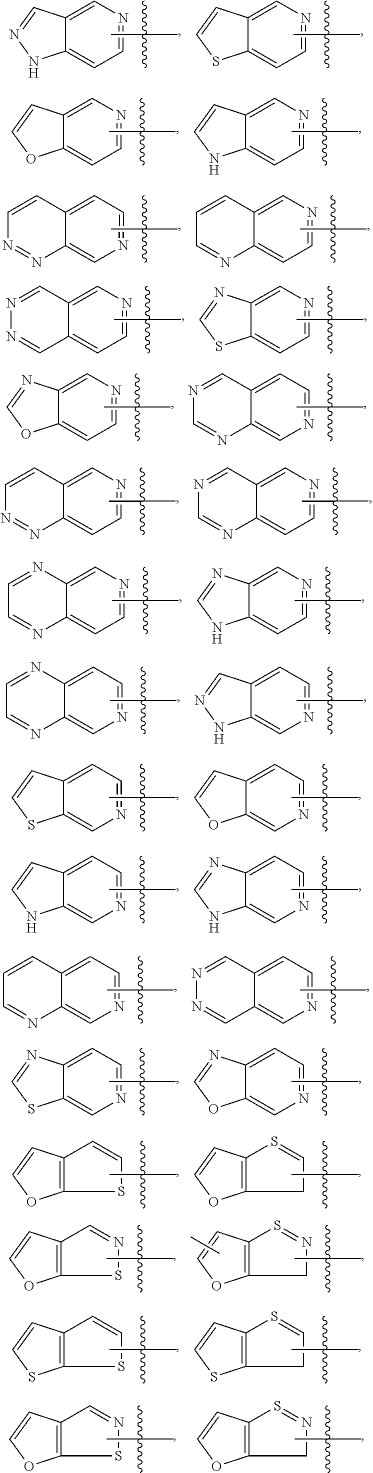

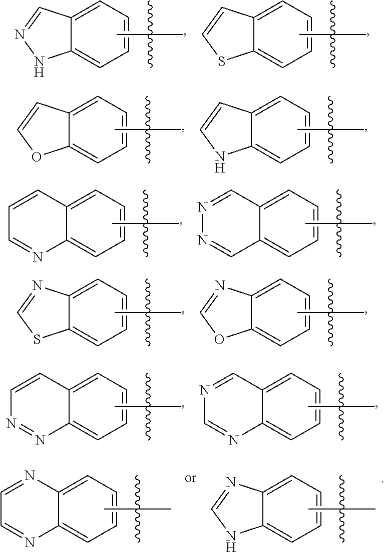

The heteroaryl may be substituted or unsubstituted.

“Fused ring” refers to a polycyclic group with two or more cyclic structures sharing a pair of atoms with each other. One or more rings may contain one or more double bonds, but at least one ring does not have a completely conjugated a electron aromatic system, and meanwhile, at least one ring has a completely conjugated a electron aromatic system, wherein zero, one or more ring atoms are selected from heteroatoms of nitrogen, oxygen or S(O)ₙ (wherein n is selected from 0, 1 or 2), and the remaining ring atoms are carbon. The fused ring is preferably a bicyclic or tricyclic fused ring, wherein the bicyclic fused ring is preferably a fused ring of aryl or heteroaryl and monocyclic heterocyclyl or monocyclic cycloalkyl. Preferably 7-14 membered, and more preferably 8-10 membered. Examples of “fused ring” comprise, but are not limited to:

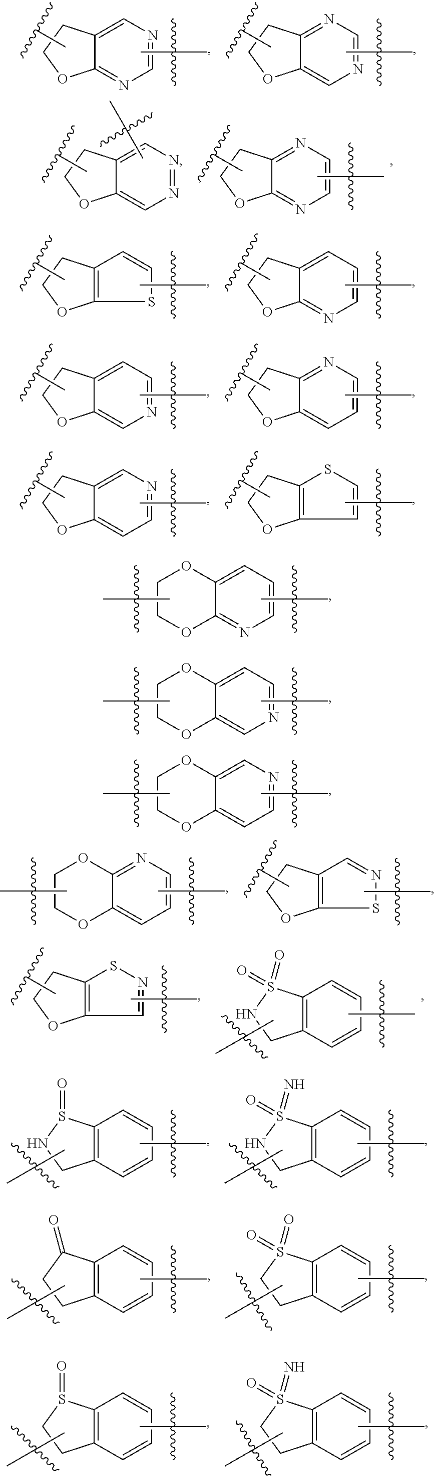

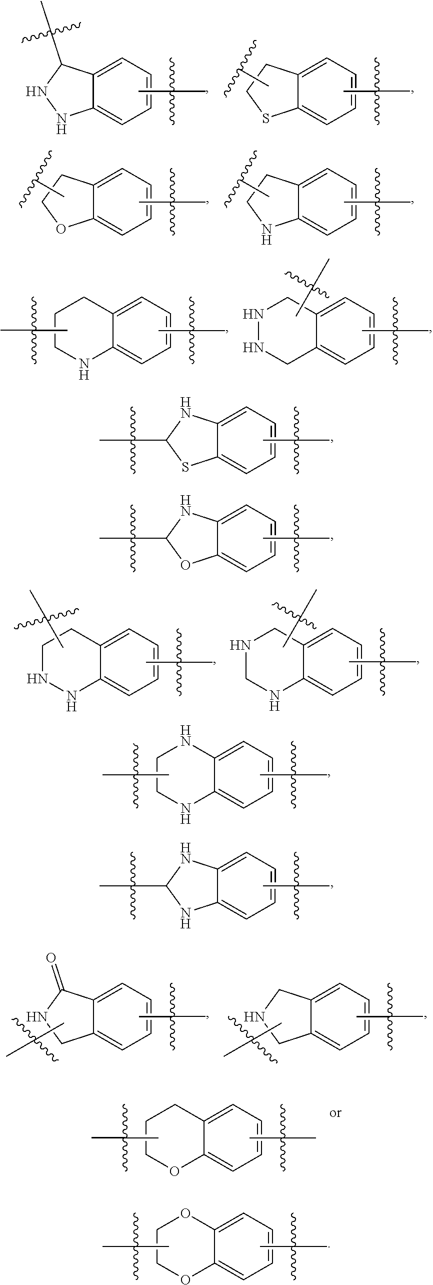

“Alkoxy” refers to a group of (alkyl-O—), wherein, the alkyl is defined herein. C₁-C₆ alkoxy is preferred. Examples of such alkoxy comprise, but are not limited to, methoxy, ethoxy, n-propoxy, isopropoxy, n-butoxy, isobutoxy, tert-butoxy, and the like.
“Hydroxy” refers to an —OH group.
“Halogen” refers to fluorine, chlorine, bromine and iodine.
“Amino” refers to —NH₂.
“Cyano” refers to —CN.
“Nitro” refers to —NO₂.
“Benzyl” and “Bn” refer to —CH₂— phenyl.
“Carboxyl” refers to —C(O)OH.
“Carboxylate” refers to —C(O)O-alkyl or —C(O)O-cycloalkyl, wherein the definitions of the alkyl and the cycloalkyl are as above.
“DMSO” refers to dimethyl sulfoxide.
“BOC” refers to tert-butoxycarbonyl.
“TFA” refers to trifluoroacetic acid.
“Ts” refers to p-toluenesulfonyl.
“Hydroxy C₁-C₄ alkyl” refers to a C₁-C₄ alkyl substituted by hydroxy.
“Amino C₁-C₄ alkyl” refers to a C₁-C₄ alkyl substituted by amino.
“Leaving group”, is an atom or functional group separated from a larger molecule in chemical reaction, which is a term used in nucleophilic substitution reaction and elimination reaction. In nucleophilic substitution reaction, a reactant attacked by a nucleophilic reagent is called substrate, while an atom or atomic group broken away with a pair of electrons in the substrate molecule is called leaving group. A group that accepts electrons easily and has strong ability of bearing negative charges is a good leaving group. When the pKa of a conjugate acid of the leaving group is smaller, it is easier for the leaving group to separate from other molecules. The reason is that when the pKa of the conjugated acid of the leaving group is smaller, the corresponding leaving group does not need to be combined with other atoms, and the tendency to exist in the form of anions (or electrically neutral leaving group) is enhanced. Common leaving groups comprise but are not limited to, halogen, mesyl, —OTs or —OH.

“Substituted” means that one or more hydrogen atoms in a group, preferably at most 5, more preferably 1 to 3 hydrogen atoms, are independently replaced by a corresponding number of substituents. Obviously, substituents are only in their possible chemical positions, and those skilled in the art can determine (through experiments or theories) possible or impossible substitutions without going through much effort. For example, amino or hydroxy with free hydrogen may be unstable when combined with carbon atoms with unsaturated (e.g., olefinic) bonds.

As used in this specification, “substitute” or “substituted”, unless otherwise specified, means that a group may be substituted by one or more groups selected from the following: alkyl, alkenyl, alkynyl, alkoxy, alkylthio, alkylamino, halogen, mercapto, hydroxy, nitro, cyano, cycloalkyl, heterocyclic, aryl, heteroaryl, cycloalkoxy, heterocyclic alkoxy, cycloalkylthio, heterocycloalkylthio, amino, haloalkyl, hydroxyalkyl, carboxyl, carboxylate, ═O, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷;

R⁵, R⁶ and R⁷ are each independently selected from hydrogen atom, alkyl, cycloalkyl or heterocyclyl, wherein the alkyl, cycloalkyl or heterocyclyl is optionally further substituted by one or more substituents selected from hydroxy, amino, halogen, nitro, cyano, alkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR₉R¹⁰, —SO₂NR⁹R¹⁰ or —NR⁹C(O)R¹⁰;
alternatively, R⁶ and R⁷ together with the N atom bound therewith form a 3-8 membered heterocyclyl, wherein the 3-8 membered heterocyclyl internally contains one or more N, O, S or SO₅ atoms, and the 3-8 membered heterocyclyl is further substituted by one or more substituents selected from hydroxy, halogen, amino, alkyl or alkoxy; and
R⁸, R⁹ and R⁰ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, halogen, nitro, cyano, alkyl, alkoxy, cycloalkyl, heterocyclyl, aryl, heteroaryl, carboxyl or carboxylate.

“Pharmaceutically acceptable salts” refers to some salts of the above-mentioned compounds which can keep the original biological activity and are suitable for medical use. The pharmaceutically acceptable salt of a compound represented by general formula (I) may be a metal salt, an amine salt formed by a suitable acid.

“Pharmaceutical composition” represents a mixture containing one or more compounds described herein or physiologically acceptable salts or prodrugs thereof and other chemical components, as well as other components such as physiologically acceptable carriers and excipients. The object of the pharmaceutical composition is to promote the administration to organisms and facilitate the absorption of active ingredients to exert biological activity.

### Synthesis Methods of the Compounds of the Present Invention

In order to achieve the objects of the present invention, the following technical solutions are adopted by the present invention.

The present invention provides a preparation method for a compound represented by general formula (I) or a stereoisomer or a tautomer thereof, or a pharmaceutically acceptable salt thereof, wherein the method comprises:

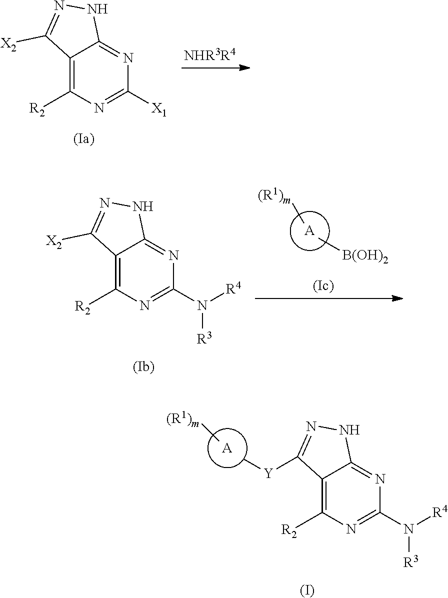

subjecting the compound represented by general formula (Ia) and NHR³R⁴ to a nucleophilic substitution reaction under alkaline condition to obtain the compound represented by general formula (Ib); and subjecting the compound represented by general formula (Ib) and the compound represented by general formula (Ic) to a Suzuki reaction in the presence of palladium catalyst and alkaline condition, and optionally further removing a protecting group of the obtained compound to obtain the compound represented by general formula (I);
   wherein:
Y is selected from chemical bond;
X₁ is selected from leaving group, wherein the leaving group is selected from halogen or —SO₂R^{t};
X₂ is selected from halogen;
R^{t} is selected from alkyl; and
ring A, m, and R¹-R⁴ are defined as in general formula (I).

The present invention provides a preparation method for a compound represented by general formula (I) or a stereoisomer or a tautomer thereof, or a pharmaceutically acceptable salt thereof, wherein the method comprises:

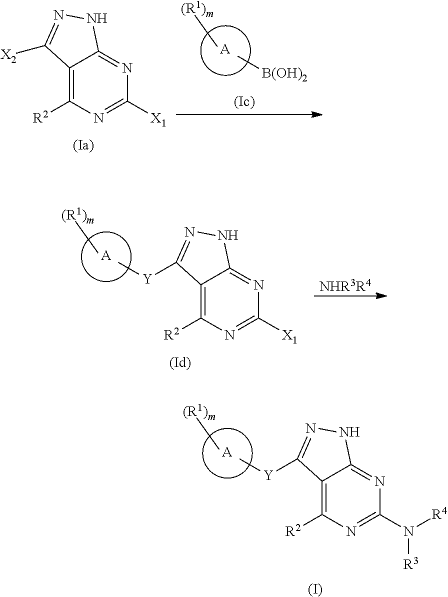

subjecting the compound represented by general formula (Ia) and the compound represented by general formula (Ic) to a Suzuki reaction in the presence of palladium catalyst and alkaline condition, to obtain the compound represented by general formula (Id); and subjecting the compound represented by general formula (Id) and NHR³R⁴ to a nucleophilic substitution reaction under alkaline condition, and further removing a protecting group from the obtained compound to obtain the compound represented by general formula (I);
   wherein:
Y is selected from chemical bond;
X₁ is selected from leaving group, wherein the leaving group is selected from halogen or —SO₂R^{t};
X₂ is selected from halogen;
R^{t} is selected from alkyl; and
ring A, m, and R¹-R⁴ are defined as in general formula (I).

The present invention provides a preparation method for a compound of general formula (II) or a stereoisomer or a tautomer thereof or a pharmaceutically acceptable salt thereof, wherein the method comprises:

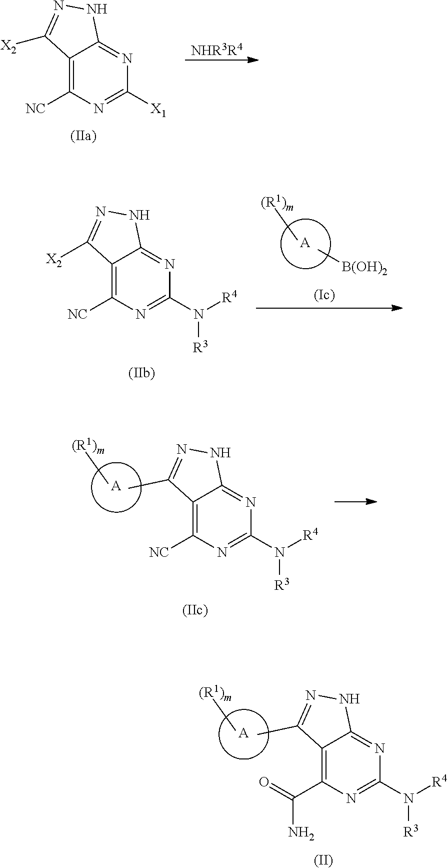

subjecting the compound represented by general formula (IIa) and NHR³R⁴ to a nucleophilic substitution reaction under alkaline condition to obtain the compound represented by general formula (Ib); subjecting the compound represented by general formula (IIb) and the compound represented by general formula (Ic) to a Suzuki reaction in the presence of palladium catalyst and alkaline condition, and optionally further removing a protecting group from the obtained compound to obtain the compound represented by general formula (IIc); and hydrolyzing the compound represented by general formula (IIc)under the condition of a sodium hydroxide solution to obtain the compound represented by general formula (II);
wherein:
X₁ is selected from leaving group, wherein the leaving group is selected from halogen or —SO₂R^{t};
X₂ is selected from halogen;
R^{t} is selected from alkyl; and
ring A, m, R¹, R³ and R⁴ are defined as in general formula (II).

The present invention provides a preparation method for a compound of general formula (II) or a stereoisomer or a tautomer thereof or a pharmaceutically acceptable salt thereof, wherein the method comprises:

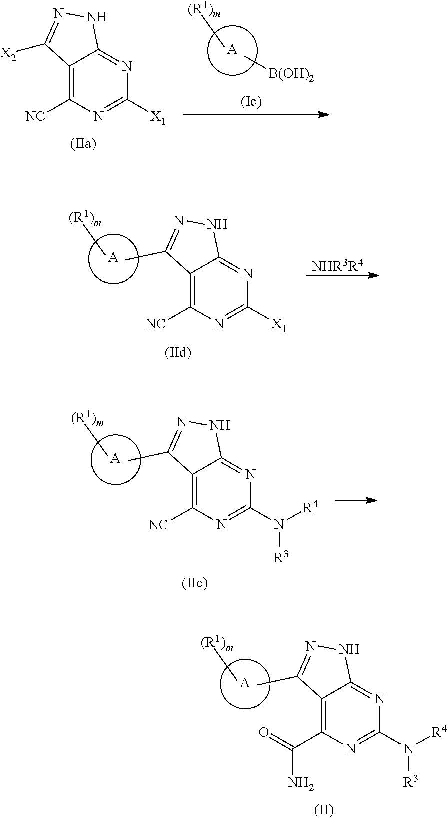

subjecting the compound represented by general formula (IIa) and the compound represented by general formula (Ic) to a Suzuki reaction in the presence of palladium catalyst and alkaline condition, to obtain the compound represented by general formula (IId); subjecting the compound represented by general formula (IId) and NHR³R⁴ to a nucleophilic substitution reaction under alkaline condition to obtain the compound represented by general formula (IIc); and hydrolyzing the compound represented by general formula (IIc) under the condition of a sodium hydroxide solution to obtain the compound represented by general formula (II);
wherein:
X₁ is selected from leaving group, wherein the leaving group is selected from halogen or —SO₂R^{t};
X₂ is selected from halogen;
R^{t} is selected from alkyl; and
ring A, m, R¹, R³ and R⁴ are defined as in general formula (II).

### EMBODIMENTS

The following examples are used to further describe the present invention, but these examples do not limit the scope of the present invention.

### EXAMPLES

The examples show the preparation of representative compounds represented by formula (I) and related structural identification data. It should be noted that the following examples are only used to illustrate the present invention, but not to limit the present invention. ¹H NMR spectrum was measured by Bruker instrument (400 MHz), and chemical shift was expressed in ppm. Tetramethylsilane internal standard (0.00 ppm) was employed. ¹H NMR was expressed as follows: s=singlet, d=doublet, t=triplet, m=multiplet, br=broadened, dd=doublet of doublets, and dt=doublet of triplets. If a coupling constant was provided, it was in the unit of Hz.

A mass spectrum was determined by LC/MS, and an ionization method may be ESI or APCI.

Yantai Huanghai HSGF254 or Qingdao GF254 silica gel plates were used as silica gel plates for thin layer chromatography. The silica gel plates used for thin layer chromatography (TLC) had a specification of 0.15 mm to 0.2 mm, and products separated and purified by TLC had a specification of 0.4 mm to 0.5 mm.

In general, Yantai Huanghai silica gel with 200-300 meshes was used as a carrier for column chromatography.

In the following examples, unless otherwise specified, all temperatures are in Celsius. Unless otherwise specified, various starting materials and reagents are commercially available or synthesized according to known methods, and the commercially available materials and reagents are directly used without further purification. Unless otherwise specified, the commercially available manufacturers include but are not limited to Aldrich Chemical Company, ABCR GmbH & Co.KG, Acros Organics, Guangzan Chemical Science and Technology Ltd., Jingyan Chemical Science and Technology Ltd., and the like.

CD₃OD: Methanol-d4.

CDCl₃: Chloroform-d.

DMSO-d₆: Dimethyl sulfoxide-d6.

Argon atmosphere refers to that a reaction flask is connected with an argon balloon with a volume of about 1 L.

Unless otherwise specified in the examples, a solution in the reaction refers to an aqueous solution.

The compounds were purified by a silica gel column chromatography eluent system and thin layer chromatography, wherein the eluent system was selected from A: petroleum ether and ethyl acetate system; B: dichloromethane and methanol system; and C: dichloromethane and ethyl acetate system. The volume ratios of the solvents varied according to the polarity of the compounds, and may also be adjusted by adding a small amount of acidic or basic reagents, such as acetic acid or triethylamine, or the like.

### Example 1

### 6-(4-amino-4-methylpiperidin-1-yl)-3-(2,3-dichlorophenyl)-1H-pyrazolo[3,4-d]pyrimidine-4-carbonitrile

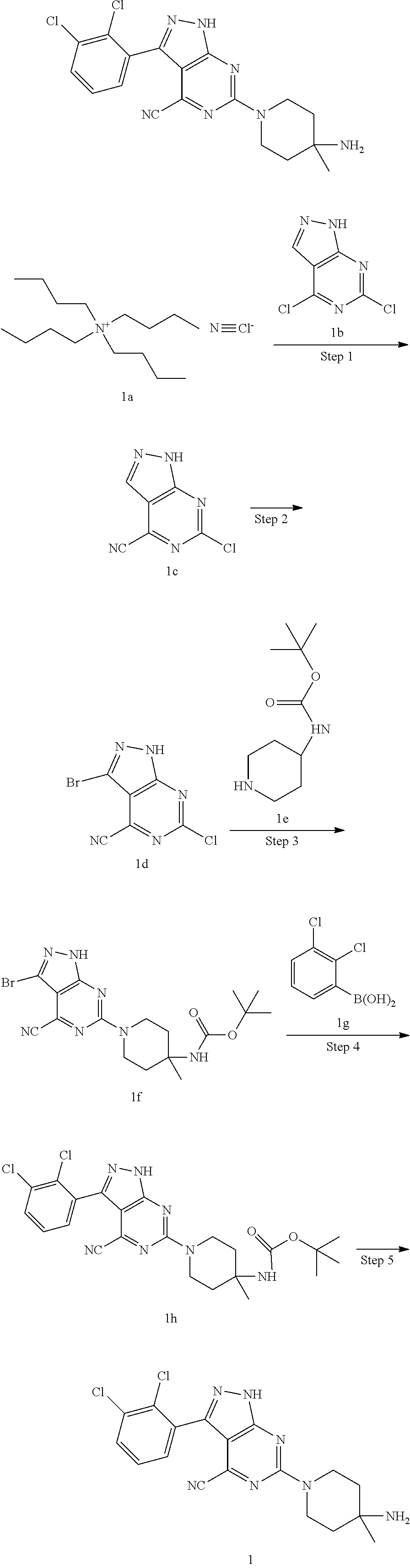

### Step 1

### 6-chloro-1H-pyrazolo[3,4-d]pyrimidine-4-carbonitrile

Tetrabutylammonium cyanide 1a (2.81 g, 10.48 mmol), 4,6-dichloro-1H-pyrazolo[3,4-d]pyrimidine 1b (1.8 g, 9.52 mmol) and triethylene diamine (213.65 mg, 1.90 mmol) were added to dichloromethane (30 mL) in turn, and continuously stirred for 2 hours at room temperature. After the reaction was completed, the reaction solution was concentrated under reduced pressure. The obtained residue was further analyzed and purified by silica gel column chromatography (eluent: system B), and a small amount of triethylamine was added to the system to obtain 6-chloro-1H-pyrazolo[3,4-d]pyrimidine-4-carbonitrile 1c (741 mg) with a yield of 43.33%.

MS m/z (ESI): 179.9 [M+1]

### Step 2

### 3-bromo-6-chloro-1H-pyrazolo[3,4-d]pyrimidine-4-carbonitrile

6-chloro-1H-pyrazolo[3,4-d]pyrimidine-4-carbonitrile 1c (200 mg, 1.11 mmol) and N-bromosuccinimide (218.06 mg, mmol) were added to acetonitrile (4 mL) in turn, heated to 90° C., and reacted for 4 hours. After the reaction was completed, the reaction solution was concentrated under reduced pressure. The obtained residue was further analyzed and purified by silica gel column chromatography (eluent: system B) to obtain 3-bromo-6-chloro-1H-pyrazolo[3,4-d]pyrimidine-4-carbonitrile 1d (280 mg) with a yield of 97.26%.

MS m/z (ESI): 257.7 [M+1]

### Step 3

### tert-butyl N-[1-(3-bromo-4-cyano-1H-pyrazolo[3,4-d]pyrimidin-6-yl)-4-methyl-4-piperidinyl]carbamate

3-bromo-6-chloro-1H-pyrazolo[3,4-d]pyrimidine-4-carbonitrile 1d (200 mg, 773.81 μmol), tert-butyl N-(4-methyl-4-piperidinyl) carbamate 1e (248.74 mg, 1.16 mmol) and diisopropylethylamine (600.04 mg, 4.64 mmol, 810.87 uL) were added to N-methyl pyrrolidone (3 mL) in turn, heated to 110° C., and reacted for 6 hours. After the reaction was completed, the reaction solution was added with 30 mL of ethyl acetate and 15 mL of water for liquid separation and extraction to separate the aqueous layer, then organic phases were washed with a saturated sodium chloride solution (10 mL×2) in turn, and concentrated under reduced pressure. The obtained residue was separated and purified by silica gel column chromatography (eluent: system B) to obtain tert-butyl N-[1-(3-bromo-4-cyano-1H-pyrazolo[3,4-d]pyrimidin-6-yl)-4-methyl-4-piperidinyl] carbamate if (230 mg) with a yield of 68.12%.

MS m/z (ESI): 436.1 [M+1]

### Step 4

### Tert-butyl N-[1-(4-cyano-3-(2,3-dichlorophenyl)-1H-pyrazolo[3,4-d]pyrimidin-6-yl)-4-methyl-4-piperidinyl) carbamate

Under the protection of argon gas, tert-butyl N-[1-(3-bromo-4-cyano-1H-pyrazolo[3,4-d]pyrimidin-6-yl)-4-methyl-4-piperidinyl] carbamate if (230 mg, 527.15 μmol), (2,3-dichlorophenyl)boronic acid 1g (150.89 mg, 790.73 μmol), methanesulfonato(2-dicyclohexylphosphino-2′,6′-di-isopropoxy-1,1′-biphenyl)(2′-amino-1,1′-biphenyl-2-yl)palladium (88.25 mg, 105.43 μmol), potassium phosphate (335.27 mg, 1.58 mmol) and 2-dicyclohexylphosphino-2′,6′-di-isopropoxy-1,1′-biphenyl (98.26 mg, 210.86 μmol) were added to 6 mL of mixed solution (1,4-dioxane:water=5:1) in turn, heated to 120° C., and reacted for 6 hours. After the reaction was completed, the reaction solution was added with 15 mL of water and 30 mL of ethyl acetate for liquid separation and extraction, then organic phases were washed with 10 mL of saturated salt solution, and concentrated under reduced pressure. The obtained residue was further separated and purified by silica gel column chromatography (eluent: system A) to obtain tert-butyl N-[1-[4-cyano-3-(2,3-dichlorophenyl)-1H-pyrazolo[3,4-d]pyrimidin-6-yl)-4-methyl-4-piperidinyl] carbamate 1h (143 mg) with a yield of 53.99%.

MS m/z (ESI): 502.0 [M+1]

### Step 5

### 6-(4-amino-4-methylpiperidin-1-yl)-3-(2,3-dichlorophenyl)-1H-pyrazolo[3,4-d]pyrimidine-4-carbonitrile

tert-butyl N-[1-[4-cyano-3-(2,3-dichlorophenyl)-1H-pyrazolo[3,4-d]pyrimidin-6-yl)-4-methyl-4-piperidinyl] carbamate 1h (140 mg, 278.67 μmol) and trifluoroacetic acid (1.53 g, 13.42 mmol, 1 mL) were added to dichloromethane (3 mL) in turn, and reacted at room temperature for 6 hours. After the reaction was completed, a saturated sodium carbonate solution was added slowly dropwise to the reaction solution to adjust the pH to be 8, and then concentrated under reduced pressure. 30 mL of ethyl acetate and 15 mL of water were added to the residue for liquid separation and extraction, then organic phases were washed with 10 mL of saturated salt water, and concentrated under reduced pressure. The obtained residue was further separated and purified by silica gel column chromatography (eluent: system A) to obtain the target product 6-(4-amino-4-methylpiperidin-1-yl)-3-(2,3-dichlorophenyl)-1H-pyrazolo[3,4-d]pyrimidine-4-carbonitrile 1 (70 mg) with a yield of 47.92%.

MS m/z (ESI): 401.9 [M+1]

### Example 2

### 6-(4-amino-4-methylpiperidin-1-yl)-3-(2,3-dichlorophenyl)-1H-pyrazolo[3,4-d]pyrimidine-4-carboxamide

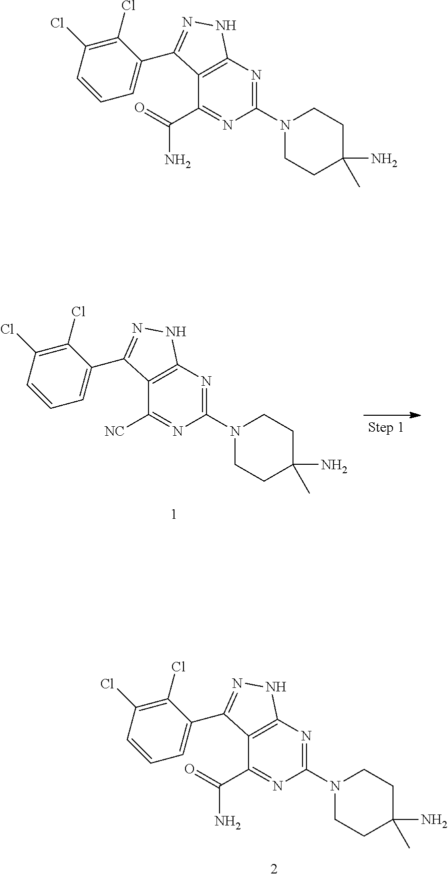 [SMILES: CC1(N)CCN(c2nc(C(N)=O)c3c(-c4cccc(Cl)c4Cl)n[nH]c3n2)CC1 ; CC1(N)CCN(c2nc(C#N)c3c(-c4cccc(Cl)c4Cl)n[nH]c3n2)CC1 ; CC1(N)CCN(c2nc(C(N)=O)c3c(-c4cccc(Cl)c4Cl)n[nH]c3n2)CC1]

### Step 1

### 6-(4-amino-4-methylpiperidin-1-yl)-3-(2,3-dichlorophenyl)-1H-pyrazolo[3,4-d]pyrimidine-4-carboxamide

6-(4-amino-4-methylpiperidin-1-yl)-3-(2,3-dichlorophenyl)-1H-pyrazolo[3,4-d]pyrimidine-4-carbonitrile 1 (65 mg, 125.90 μmol) and 6 M sodium hydroxide solution (0.5 mL) were added to 2 mL of ethanol in turn, heated to 80° C., and reacted for 3 hours. After the reaction was completed, trifluoroacetic acid was slowly added dropwise in the reaction solution to adjust the pH to be 5, and then concentrated under reduced pressure. The obtained residue was subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain the target product 6-(4-amino-4-methylpiperidin-1-yl)-3-(2,3-dichlorophenyl)-1H-pyrazolo[3,4-d]pyrimidine-4-carboxamide 2 (10 mg) with a yield of 13.80%.

MS m/z (ESI): 421.9 [M+1]

¹H NMR (400 MHz, CD₃OD) δ7.56 (dd, J=7.6, 2.0 Hz, 1H), 7.38-7.31 (m, 2H), 4.64-4.60 (m, 2H), 3.59-3.52 (m, 2H), 1.89-1.82 (m, 4H), 1.51 (s, 3H).

### Example 3 and Example 4

### 6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carbonitrile 3

6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carboxamide 4

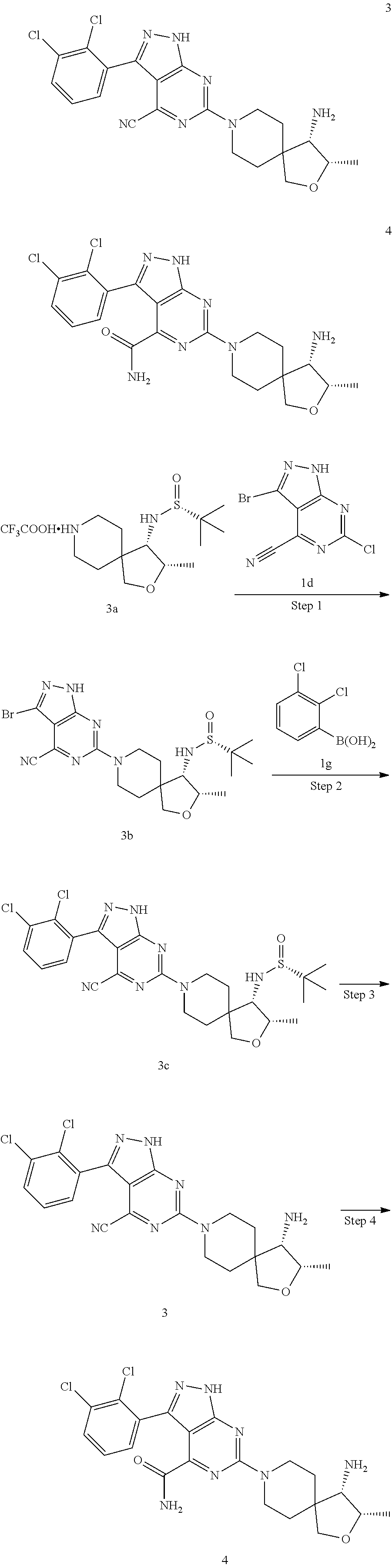

### Step 1

### (R)—N-((3S,4S)-8-(3-bromo-4-cyano-1H-pyrazolo[3,4-d]pyrimidin-6-yl)-3-methyl-2-oxa-8-azaspiro[4.5]decan-4-yl)-2-methylpropane-2-sulfinamide

3-bromo-6-chloro-1H-pyrazolo[3,4-d]pyrimidine-4-carbonitrile 1d (300 mg, 1.16 mmol), diisopropylethylamine (750 mg, 5.8 mmol, 1.0 mL) and (R)-2-methyl-N-((3S,4S)-3-methyl-2-oxa-8-azaspiro[4.5]decan-4-yl)propane-2-sulfinamide 2,2,2-trifluoroacetate 3a (478 mg, 1.74 mmol) were added to N-methyl pyrrolidone (5 mL) in turn, heated to 110° C., and reacted for 2 hours. After the reaction was completed, the reaction solution was added with 30 mL of ethyl acetate and 15 mL of water for liquid separation and extraction to separate the aqueous layer, then organic phases were washed with a saturated sodium chloride solution (10 mL×2) in turn, and concentrated under reduced pressure. The obtained residue was separated and purified by silica gel column chromatography (eluent: system B) to obtain the product (R)—N-((3S,4S)-8-(3-bromo-4-cyano-1H-pyrazolo[3,4-d]pyrimidin-6-yl)-3-methyl-2-oxa-8-azaspiro[4.5]decan-4-yl)-2-methylpropane-2-sulfinamide 3b (230 mg) with a yield of 40%.

MS m/z (ESI): 495.9 [M+1]

### Step 2

### (R)—N-((3S,4S)-8-(3-(2,3-dichlorophenyl)-4-cyano-1H-pyrazolo[3,4-d]pyrimidin-6-yl)-3-methyl-2-oxa-8-azaspiro[4.5]decan-4-yl)-2-methylpropane-2-sulfinamide

Under the protection of argon gas, (R)—N-((3S,4S)-8-(3-bromo-4-cyano-1H-pyrazolo[3,4-d]pyrimidin-6-yl)-3-methyl-2-oxa-8-azaspiro[4.5]decan-4-yl)-2-methylpropane-2-sulfinamide 3b (230 mg, 463 μmol), (2,3-dichlorophenyl)boronic acid 1g (142 mg, 745 μmol), methanesulfonato(2-dicyclohexylphosphino-2′,6′-di-isopropoxy-1,1′-biphenyl)(2-amino-1,1′-biphenyl-2-yl)palladium (77 mg, 93 μmol), potassium phosphate (295 mg, 1.39 mmol) and 2-dicyclohexylphosphino-2′,6′-di-isopropoxy-1,1′-biphenyl (87 mg, 186 μmol) were added to 6 mL of mixed solution of 1,4-dioxane and water (V_{1,4-dionxane}:V_{water}=5:1) in turn, heated to 100° C., and reacted for 6 hours. After the reaction was completed, the reaction solution was added with 15 mL of water and 30 mL of ethyl acetate for liquid separation and extraction, then organic phases were washed with 10 mL of saturated salt water, and concentrated under reduced pressure. The obtained residue was further separated and purified by silica gel column chromatography (eluent: system A) to obtain the product (R)—N-((3S,4S)-8-(3-(2,3-dichlorophenyl)-4-cyano-1H-pyrazolo[3,4-d]pyrimidin-6-yl)-3-methyl-2-oxa-8-azaspiro[4.5]decan-4-yl)-2-methylpropane-2-sulfinamide 3c (74 mg) with a yield of 28%.

MS m/z (ESI): 561.9 [M+1]

### Step 3

### 6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carbonitrile

(R)—N-((3S,4S)-8-(3-(2,3-dichlorophenyl)-4-cyano-1H-pyrazolo[3,4-d]pyrimidin-6-yl)-3-methyl-2-oxa-8-azaspiro[4.5]decan-4-yl)-2-methylpropane-2-sulfinamide 3c (74 mg, 132 μmol) and bromosuccinimide (26 mg, 145 μmol) were added to 1 mL of N,N-dimethylformamide in turn, and reacted for 2 hours at room temperature. After the reaction was completed, the reaction solution was concentrated under reduced pressure, and the obtained residue was further separated and purified by silic agel column chromatography (eluent: system A) to obtain the product 6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carbonitrile 3 (20 mg) with a yield of 33%.

MS m/z (ESI): 457.9 [M+1]

### Step 4

### 6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carboxamide

6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carbonitrile 3 (20 mg, 43.6 μmol) and 6 M sodium hydroxide solution (0.5 mL) were added to 2 mL of ethanol in turn, heated to 80° C., and reacted for 1 hour.

After the reaction was completed, trifluoroacetic acid was slowly added dropwise to the reaction solution to adjust the pH to be 5, and then concentrated under reduced pressure. The obtained residue was subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain the target product 6-((3S,4S)-4-amino-3-methyl-2-oxa-8-azaspiro[4.5]decan-8-yl)-3-(2,3-dichlorophenyl)-1H-pyrazolo[3,4-d]pyrimidine-4-carboxamide 4 (4.5 mg) with a yield of 22%.

MS m/z (ESI): 475.9 [M+1]

### Example 5

### (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxamide

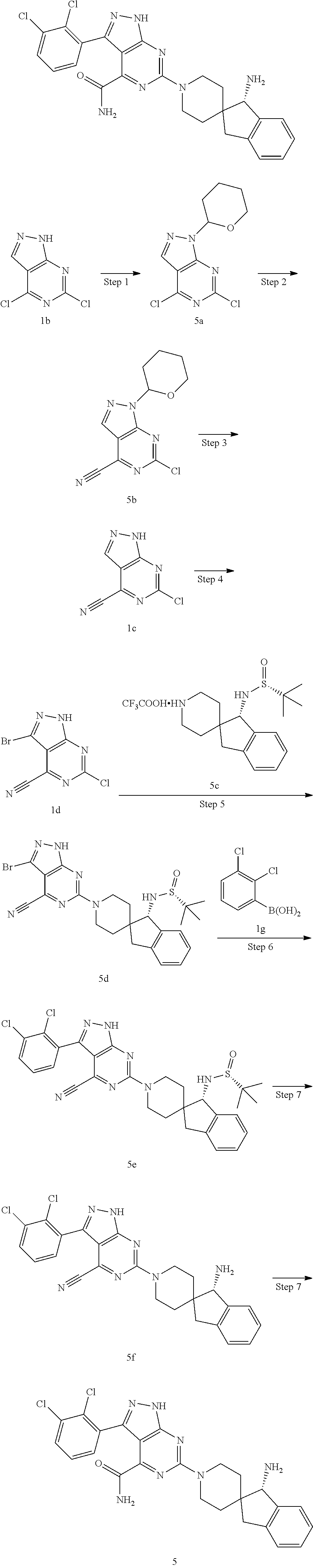

4,6-dichloro-1-(tetrahydro-2H-pyran-2-yl)-1H-pyrazolo[3,4-d]pyrimidine 3,4-dihydro-2H-pyrane (14.69 g, 174.60 mmol), 4,6-dichloro-1H-pyrazolo[3,4-d]pyrimidine 1b (11 g, 58.20 mmol) and p-toluenesulfonic acid (1.00 g, 5.82 mmol) were added to tetrahydrofuran (100 mL), heated to 60° C., and reacted for 2 hours. After the reaction was completed, the reaction solution was concentrated under reduced pressure. The obtained residue was further analyzed and purified by silica gel column chromatography (eluent: system A) to obtain 4,6-dichloro-1-(tetrahydro-2H-pyran-2-yl)-1H-pyrazolo[3,4-d]pyrimidine 5a (14.2 g) with a yield of 89.3%.

MS m/z (ESI): 272.9 [M+1]

### Step 2

### 6-chloro-1-(tetrahydro-2H-pyran-2-yl)-1H-pyrazolo[3,4-d]pyrimidine-4-carbonitrile

Under the protection of argon gas, 4,6-dichloro-1-(tetrahydro-2H-pyran-2-yl)-1H-pyrazolo[3,4-d]pyrimidine 5a (2 g, 7.32 mmol), tetrakis(triphenylphosphine)palladium (845.79 mg, 732.28 μmol) and zinc cyanide (1.72 g, 14.65 mmol) were added to N,N-dimethylformamide (20 mL), heated to 110° C., and reacted for 2 hours. After the reaction was completed, the reaction solution was concentrated under reduced pressure, and added with ethyl acetate (50 mL) for fully dissolved to filter insolubles. The filtrate was concentrated under reduced pressure. The obtained residue was further analyzed and purified by silica gel column chromatography (eluent: system A) to obtain 6-chloro-1-(tetrahydro-2H-pyran-2-yl)-1H-pyrazolo[3,4-d]pyrimidine-4-carbonitrile 5b (0.8 g) with a yield of 41.4%.

### Step 3

### 6-chloro-1H-pyrazolo[3,4-d]pyrimidine-4-carbonitrile

At room temperature, 6-chloro-1-(tetrahydro-2H-pyran-2-yl)-1H-pyrazolo[3,4-d]pyrimidine-4-carbonitrile 5b (1 g, 3.79 mmol) was added to trifluoroacetic acid (10 mL) and water (1 mL), and reacted for 4 hours at room temperature. After the reaction was completed, the reaction solution was concentrated under reduced pressure, and subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain 6-chloro-1H-pyrazolo[3,4-d]pyrimidine-4-carbonitrile 1c (321 mg) with a yield of 47.1%.

MS m/z (ESI): 179.9 [M+1]

### Step 4

### 3-bromo-6-chloro-1H-pyrazolo[3,4-d]pyrimidine-4-carbonitrile

Bromosuccinimide (475.77 mg, 2.67 mmol) and 6-chloro-1H-pyrazolo[3,4-d]pyrimidine-4-carbonitrile 1c (320 mg, 1.78 mmol) was added to acetonitrile (10 mL) for heated and refluxed for 2 hours. After the reaction was completed, the reaction solution was concentrated under reduced pressure, and subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain 3-bromo-6-chloro-1H-pyrazolo[3,4-d]pyrimidine-4-carbonitrile 1d (380 mg) with a yield of 82.5%.

MS m/z (ESI): 259.8 [M+1]

### Step 5

### (R)—N—((S)-1′-(3-bromo-4-cyano-1H-pyrazolo[3,4-d]pyrimidin-6-yl)-1,3-dihydrospiro[indene-2,4′-piperidin]-1-yl)-2-methylpropane-2-sulfinamide

At room temperature, (R)—N—((S)-1,3-dihydrospiro[indene-2,4′-piperidin]-1-yl)-2-methylpropane-2-sulfinamide 2,2,2,-trifluoroacetate 5c (110.87 mg, 361.75 μmol), N,N-diisopropylethylamine (127.51 mg, 986.60 μmol, 162.93 μL) and 3-bromo-6-chloro-1H-pyrazolo[3,4-d]pyrimidine-4-carbonitrile 1d (85 mg, 328.87 μmol) were added to N-methyl pyrrolidone (5 mL), heated to 100° C. and reacted for 3 hours. After the reaction was completed, the reaction solution was concentrated under reduced pressure, and subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain (R)—N—((S)-1′-(3-bromo-4-cyano-1H-pyrazolo[3,4-d]pyrimidine-6-yl)-1,3-dihydrospiro[indene-2,4′-piperidin]-1-yl)-2-methylpropane-2-sulfinamide 5d (151 mg) with a yield of 86.9%.

MS m/z (ESI): 527.8 [M+1].

### Step 6

### (R)—N—((S)-1′-(4-cyano-3-(2,3-dichlorophenyl)-1H-pyrazolo[3,4-d]pyrimidin-6-yl)-1,3-dihydrospiro[indene-2,4′-piperidin]-1-yl)-2-methylpropane-2-sulfinamide

Under the protection of argon gas, (R)—N—((S)-1′-(3-bromo-4-cyano-1H-pyrazolo[3,4-d]pyrimidin-6-yl)-1,3-dihydrospiro[indene-2,4′-piperidin]-1-yl)-2-methylpropane-2-sulfinamide 5d (150 mg, 283.84 μmol), (2,3-dichlorophenyl)boronic acid 1g (162.49 mg, 851.52 μmol), 2-dicyclohexylphosphino-2′,6′-di-isopropoxy-1,1′-biphenyl (52.98 mg, 113.54 μmol), methanesulfonato(2-dicyclohexylphosphino-2′,6′-di-isopropoxy-1,1′-biphenyl)(2-amino-1,1′-biphenyl-2-yl)palladium (47.54 mg, 56.77 μmol) and potassium phosphate (180.75 mg, 851.52 μmol) were added to 12 mL of mixed solution (1,4-dioxane:water=5:1) in turn, heated to 100° C., and reacted for 16 hours. After the reaction was completed, the reaction solution was concentrated under reduced pressure, and added with ethyl acetate (10 mL) and water (10 mL) for extraction and liquid separation, and then aqueous phases were extracted with ethyl acetate (20 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain (R)—N—((S)-1′-(4-cyano-3-(2,3-dichlorophenyl)-1H-pyrazolo[3,4-d]pyrimidin-6-yl)-1,3-dihydrospiro[indene-2,4′-piperidin]-1-yl)-2-methylpropane-2-sulfinamide 5e (115 mg) with a yield of 68.14%.

MS m/z (ESI): 593.8 [M+1]

### Step 7

### (S)-6-(1-amino-1,3-dihydrospiro[indene-2,4′-piperidin]-1′-yl)-3-(2,3-dichlorophenyl)-1H-pyrazolo[3,4-d]pyrimidine-4-carbonitrile

(R)—N—((S)-1′-(4-cyano-3-(2,3-dichlorophenyl)-1H-pyrazolo[3,4-d]pyrimidin-6-yl)-1,3-dihydrospiro[indene-2,4′-piperidin]-1-yl)-2-methylpropane-2-sulfinamide 5e (115 mg, 193.42 μmol) was added to a hydrochloric acid dioxane solution (5 mL), and reacted for 1 hour at room temperature. After the reaction was completed, the reaction solution was concentrated under reduced pressure to obtain crude product (S)-6-(1-amino-1,3-dihydrospiro[indene-2,4′-piperidin]-1′-yl)-3-(2,3-dichlorophenyl)-1H-pyrazolo[3,4-d]pyrimidine-4-carbonitrile 5f (94 mg) with a yield of 92.2%, which was directly used for the next reaction without purification.

MS m/z (ESI): 472.9 [M−16]

### Step 8

### (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxamide

At room temperature, (S)-6-(1-amino-1,3-dihydrospiro[indene-2,4′-piperidin]-1′-yl)-3-(2,3-dichlorophenyl)-1H-pyrazolo[3,4-d]pyrimidine-4-carbonitrile 5f (94 mg, 155.52 μmol) was added to a mixed solution of aqueous sodium hydroxide (5 M, 1 mL), 30% hydrogen peroxide (1 mL) and methanol (1 mL), and reacted for 3 hours at room temperature. After the reaction was completed, the reaction solution was concentrated under reduced pressure, and subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxamide 5 (21 mg) with a yield of 20.4%.

MS m/z (ESI): 508.1 [M+1]

¹HNMR (400 MHz, DMSO-d₆) δ 8.24 (s, 3H), 8.12 (s, 1H), 7.65-7.70 (m, 2H), 7.50-7.55 (m, 1H), 7.30-7.42 (m, 5H), 4.52-5.00 (m, 2H), 4.39 (s, 1H), 3.18-3.35 (m, 3H), 3.00-3.10 (m, 1H), 1.65-1.82 (m, 2H), 1.47-1.60 (m, 2H).

### Example 6

### 6-(4-amino-4-phenylpiperidin-1-yl)-3-(2,3-dichlorophenyl)-1H-pyrazolo[3,4-d]pyrimidine-4-carboxamide

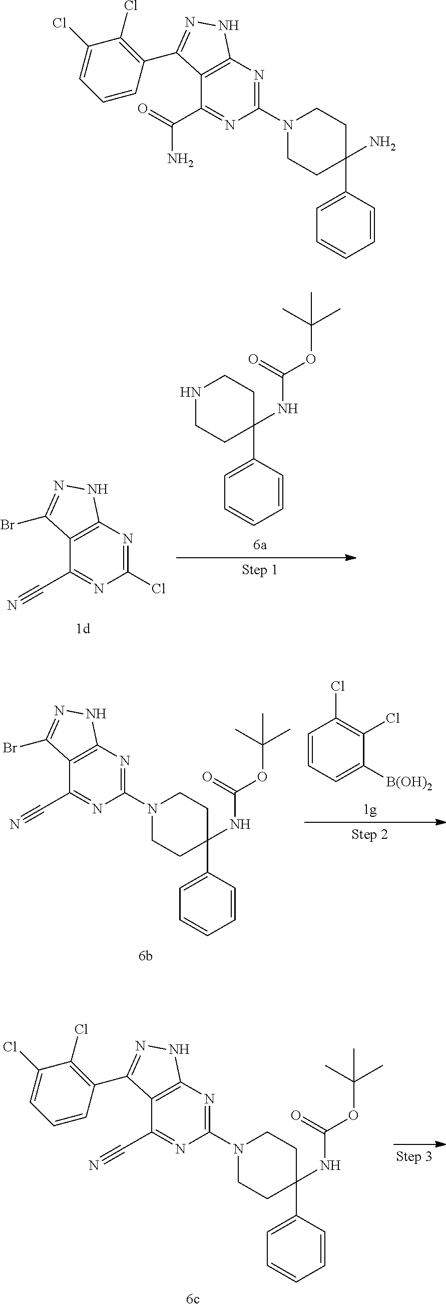

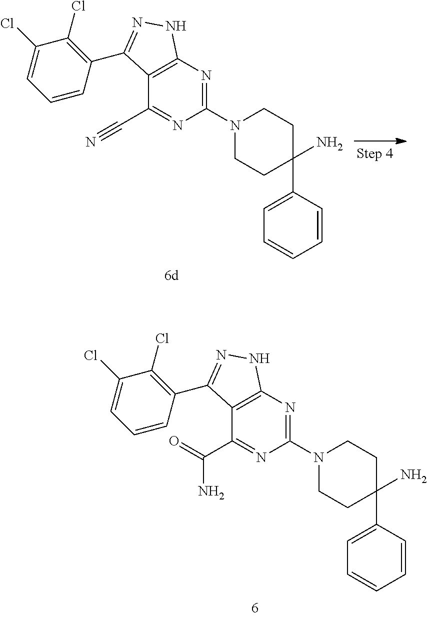 [SMILES: N#Cc1nc(N2CCC(N)(c3ccccc3)CC2)nc2[nH]nc(-c3cccc(Cl)c3Cl)c12 ; NC(=O)c1nc(N2CCC(N)(c3ccccc3)CC2)nc2[nH]nc(-c3cccc(Cl)c3Cl)c12]

### Step 1

### Tert-butyl (1-(3-bromo-4-cyano-1H-pyrazolo[3,4-d]pyrimidin-6-yl)-4-phenylpiperidine-4-yl)carbamate

At room temperature, tert-butyl (4-phenylpiperidin-4-yl)carbamate 6a (117.62 mg, 425.59 μmol), 3-bromo-6-chloro-1H-pyrazolo[3,4-d]pyrimidine-4-carbonitrile 1d (100 mg, 386.90 μmol) and N,N-diisopropylethylamine (150.01 mg, 1.16 mmol, 191.68 μL) were added to N-methyl pyrrolidone (5 mL), heated to 110° C., and reacted for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.1% TFA+H₂O, mobile phase B: CH₃CN) to obtain tert-butyl (1-(3-bromo-4-cyano-1H-pyrazolo[3,4-d]pyrimidin-6-yl)-4-phenylpiperidin-4-yl)carbamate 6b (153 mg) with a yield of 79.35%.

MS m/z (ESI): 497.8 [M+1].

### Step 2

### tert-butyl (1-(3-(2,3-dichlorophenyl)-4-cyano-1H-pyrazolo[3,4-d]pyrimidin-6-yl)-4-phenylpiperidin-4-yl) carbamate

Under the protection of argon gas, tert-butyl (1-(3-bromo-4-cyano-1H-pyrazolo[3,4-d]pyrimidin-6-yl)-4-phenylpiperidin-4-yl)carbamate 6b (153 mg, 307.00 μmol), (2,3-dichlorophenyl)boronic acid 1g (175.74 mg, 920.99 μmol), 2-dicyclohexylphosphino-2′,6′-di-isopropoxy-1,1′-biphenyl (57.30 mg, 122.80 μmol), methanesulfonato(2-dicyclohexylphosphino-2′,6′-di-isopropoxy-1,1′-biphenyl)(2-amino-1,1′-biphenyl-2-yl)palladium (51.41 mg, 61.40 μmol) and potassium phosphate (195.50 mg, 920.99 μmol) were added to 11 mL of mixed solution (1,4-dioxane:water=10:1) in turn, heated to 100° C., and reacted for 16 hours. After the reaction was completed, the reaction solution was concentrated under reduced pressure, and added with ethyl acetate (10 mL) and water (10 mL) for extraction and separation, and then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain tert-butyl (1-(3-(2,3-dichlorophenyl)-4-cyano-1H-pyrazolo[3,4-d]pyrimidin-6-yl)-4-phenylpiperidin-4-yl) carbamate 6c (128 mg) with a yield of 73.86%.

MS m/z (ESI): 563.8 [M+1]

### Step 3

### 6-(4-amino-4-phenylpiperidin-1-yl)-3-(2,3-dichlorophenyl)-1H-pyrazolo[3,4-d]pyrimidine-4-carbonitrile

Tert-butyl (1-(3-(2,3-dichlorophenyl)-4-cyano-1H-pyrazolo[3,4-d]pyrimidin-6-yl)-4-phenylpiperidin-4-yl) carbamate 6c (120 mg, 212.59 μmol) and trifluoroacetic acid (1 mL) were added to dichloromethane (3 mL), and reacted at room temperature for 1 hour. The reaction solution was concentrated under reduced pressure to obtain crude product 6-(4-amino-4-phenylpiperidin-1-yl)-3-(2,3-dichlorophenyl)-1H-pyrazolo[3,4-d]pyrimidine-4-carbonitrile 6d (89.94 mg) with a yield of 91.3%, which was directly used for the next reaction without purification.

MS m/z (ESI): 447.1 [M−16]

### Step 4

### 6-(4-amino-4-phenylpiperidin-1-yl)-3-(2,3-dichlorophenyl)-1H-pyrazolo[3,4-d]pyrimidine-4-carboxamide

6-(4-amino-4-phenylpiperidin-1-yl)-3-(2,3-dichlorophenyl)-1H-pyrazolo[3,4-d]pyrimidine-4-carbonitrile 6d (89.94 mg, 193.68 μmol) was added to a mixed solution of methanol (1.00 mL), aqueous sodium hydroxide (5 M, 1.00 mL) and 30% hydrogen peroxide (0.5 mL), and reacted for 3 hours at room temperature. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-phenylpiperidin-1-yl)-3-(2,3-dichlorophenyl)-1H-pyrazolo[3,4-d]pyrimidine-4-carboxamide 6 (18 mg) with a yield of 14.65%.

MS m/z (ESI): 481.9 [M+1]

¹HNMR (400 MHz, DMSO-d₆) δ 8.25-8.50 (m, 3H), 8.05-8.23 (m, 1H), 7.60-7.80 (m, 4H), 7.50-7.60 (m, 2H), 7.38-7.50 (m, 3H), 4.15-4.60 (m, 4H), 1.93-2.18 (m, 2H), 1.20-1.50 (m, 2H).

### Example 7

### 6-((endo)-3-amino-8-azabicyclo[3.2.1]octan-8-yl)-3-(2,3-dichlorophenyl)-1H-pyrazolo[3,4-d]pyrimidine-4-carboxamide

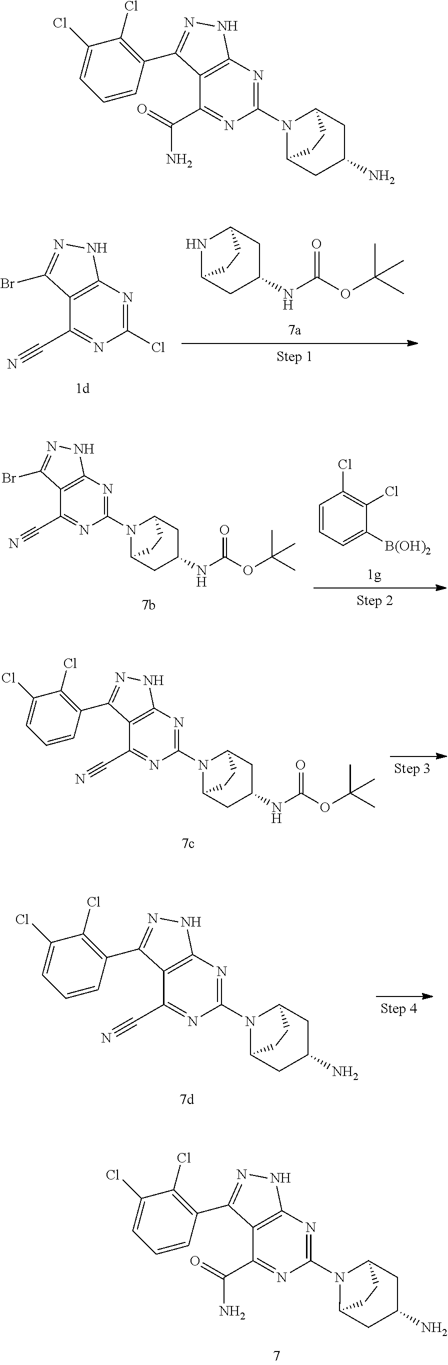

### Step 1

### Tert-butyl ((endo)-8-(3-bromo-4-cyano-1H-pyrazolo[3,4-d]pyrimidin-6-yl)-8-azabicyclo[3.2.1]octan-3-yl)carbamate

At room temperature, tert-butyl((endo)-8-azabicyclo[3.2.1]octan-3-yl)carbamate 7a (144 mg, 636 μmol), 3-bromo-6-chloro-1H-pyrazolo[3,4-d]pyrimidine-4-carbonitrile 1d (150 mg, 580 μmol) and N,N-diisopropylethylamine (225 mg, 1.74 mmol) were added to N-methyl pyrrolidone (5 mL), heated to 110° C., and reacted for 16 hours. After the reaction was completed, the reaction solution was concentrated under reduced pressure, and separated on a C₁₈ reversed phase column (C₁₈ separation column 20-45 μm; mobile phase A: H₂O, mobile phase B: CH₃CN) to obtain tert-butyl ((endo)-8-(3-bromo-4-cyano-1H-pyrazolo[3,4-d]pyrimidin-6-yl)-8-azabicyclo[3.2.1]octan-3-yl)carbamate 7b (150 mg) with a yield of 57.7%.

MS m/z (ESI): 448.0 [M+1].

### Step 2

### Tert-butyl ((endo)-8-(4-cyano-3-(2,3-dichlorophenyl)-1H-pyrazolo[3,4-d]pyrimidin-6-yl)-8-azabicyclo[3.2.1]octan-3-yl) carbamate

Under the protection of argon gas, tert-butyl((endo)-8-(3-bromo-4-cyano-1H-pyrazolo[3,4-d]pyrimidin-6-yl)-8-azabicyclo[3.2.1]octan-3-yl) carbamate 7b (150 mg, 335 μmol), (2,3-dichlorophenyl)boronic acid 1g (255 mg, 1.34 mmol), 2-dicyclohexylphosphino-2′,6′-di-isopropoxy-1,1′-biphenyl (62.5 mg, 134 mmol), methanesulfonato(2-dicyclohexylphosphino-2′,6′-di-isopropoxy-1,1′-biphenyl)(2-amino-1,1′-biphenyl-2-yl)palladium (56 mg, 67 μmol) and potassium phosphate (213 mg, 1.00 mmol) were added to 12 mL of mixed solution (1,4-dioxane:water=5:1) in turn, heated to 100° C., and reacted for 16 hours. After the reaction was completed, the reaction solution was concentrated under reduced pressure, and added with ethyl acetate (10 mL) and water (10 mL) for extraction and separation, and then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain tert-butyl((endo)-8-(4-cyano-3-(2,3-dichlorophenyl)-1H-pyrazolo[3,4-d]pyrimidin-6-yl)-8-azabicyclo[3.2.1]octan-3-yl) carbamate 7c (45 mg) with a yield of 26.1%.

MS m/z (ESI): 513.8 [M+1]

### Step 3

### 6-((endo)-3-amino-8-azabicyclo[3.2.1]octan-8-yl)-3-(2,3-dichlorophenyl)-1H-pyrazolo[3,4-d]pyrimidine-4-carbonitrile

Tert-butyl((endo)-8-(4-cyano-3-(2,3-dichlorophenyl)-1H-pyrazolo[3,4-d]pyrimidin-6-yl)-8-azabicyclo[3.2.1]octan-3-yl) carbamate 7c (45 mg, 87.75 μmol) and trifluoroacetic acid (1 mL) were added to dichloromethane (3 mL), and reacted at room temperature for 1 hour. The reaction solution was concentrated under reduced pressure to obtain crude product 6-((endo)-3-amino-8-azabicyclo[3.2.1]octan-8-yl)-3-(2,3-dichlorophenyl)-1H-pyrazolo[3,4-d]pyrimidine-4-carbonitrile 7d (35.9 mg) with a yield of 99%, which was directly used for the next reaction without purification.

MS m/z (ESI): 413.6 [M+1]

### Step 4

### 6-((endo)-3-amino-8-azabicyclo[3.2.1]octan-8-yl)-3-(2,3-dichlorophenyl)-1H-pyrazolo[3,4-d]pyrimidine-4-carboxamide

6-((endo)-3-amino-8-azabicyclo[3.2.1]octan-8-yl)-3-(2,3-dichlorophenyl)-1H-pyrazolo[3,4-d]pyrimidine-4-carbonitrile 7d (35.9 mg, 86.6 μmol) was added to a mixed solution of methanol (1.00 mL), aqueous sodium hydroxide (5 M, 1.00 mL) and 30% hydrogen peroxide (0.5 mL), and reacted for 3 hours at room temperature. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-((endo)-3-amino-8-azabicyclo[3.2.1]octan-8-yl)-3-(2,3-dichlorophenyl)-1H-pyrazolo[3,4-d]pyrimidine-4-carboxamide 7 (18 mg) with a yield of 48%.

MS m/z (ESI): 431.9 [M+1]

### Example 8

### 6-((exo)-3-amino-8-azabicyclo[3.2.1]octan-8-yl)-3-(2,3-dichlorophenyl)-1H-pyrazolo[3,4-d]pyrimidine-4-carboxamide

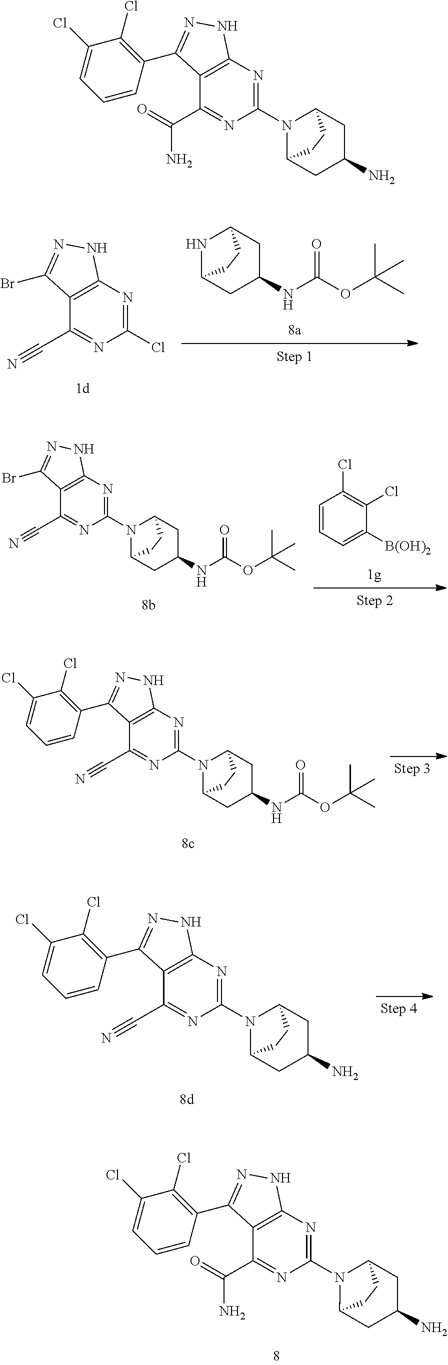

### Step 1

### Tert-butyl ((exo)-8-(3-bromo-4-cyano-1H-pyrazolo[3,4-d]pyrimidin-6-yl)-8-azabicyclo[3.2.1]octan-3-yl)carbamate

At room temperature, tert-butyl((exo)-8-azabicyclo[3.2.1]octan-3-yl)carbamate 8a (144 mg, 636 μmol), 3-bromo-6-chloro-1H-pyrazolo[3,4-d]pyrimidine-4-carbonitrile 1d (150 mg, 580 μmol) and N,N-diisopropylethylamine (225 mg, 1.74 mmol) were added to N-methyl pyrrolidone (5 mL), heated to 110° C., and reacted for 16 hours. After the reaction was completed, the reaction solution was concentrated under reduced pressure and separated on a C₁₈ reversed phase column (C₁₈ separation column 20-45 μm; mobile phase A: H₂O, mobile phase B: CH₃CN) to obtain tert-butyl ((exo)-8-(3-bromo-4-cyano-1H-pyrazolo[3,4-d]pyrimidin-6-yl)-8-azabicyclo[3.2.1]octan-3-yl) carbamate 8b (90 mg) with a yield of 34.6%.

MS m/z (ESI): 448.0 [M+1]

### Step 2

### Tert-butyl ((exo)-8-(4-cyano-3-(2,3-dichlorophenyl)-1H-pyrazolo[3,4-d]pyrimidin-6-yl)-8-azabicyclo[3.2.1]octan-3-yl) carbamate

Under the protection of argon gas, tert-butyl ((exo)-8-(3-bromo-4-cyano-1H-pyrazolo[3,4-d]pyrimidin-6-yl)-8-azabicyclo[3.2.1]octan-3-yl)carbamate 8b (90 mg, 200 μmol), (2,3-dichlorophenyl)boronic acid 1g (153 mg, 0.8 mmol), 2-dicyclohexylphosphino-2′,6′-di-isopropoxy-1,1′-biphenyl (37 mg, 80 μmol), methanesulfonato(2-dicyclohexylphosphino-2′,6′-di-isopropoxy-1,1′-biphenyl)(2-amino-1,1′-biphenyl-2-yl)palladium (33 mg, 40 μmol) and potassium phosphate (127 mg, 0.6 mmol) were added to 12 mL of mixed solution (1,4-dioxane:water=5:1) in turn, heated to 100° C., and reacted for 16 hours. After the reaction was completed, the reaction solution was concentrated under reduced pressure, and added with ethyl acetate (10 mL) and water (10 mL) for extraction and separation, and then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain tert-butyl ((exo)-8-(4-cyano-3-(2,3-dichlorophenyl)-1H-pyrazolo[3,4-d]pyrimidin-6-yl)-8-azabicyclo[3.2.1]octan-3-yl) carbamate Sc (25 mg) with a yield of 24.3%.

MS m/z (ESI): 513.8 [M+1]

### Step 3

### 6-((exo)-3-amino-8-azabicyclo[3.2.1]octan-8-yl)-3-(2,3-dichlorophenyl)-1H-pyrazolo[3,4-d]pyrimidine-4-carbonitrile

Tert-butyl ((exo)-8-(4-cyano-3-(2,3-dichlorophenyl)-1H-pyrazolo[3,4-d]pyrimidin-6-yl)-8-azabicyclo[3.2.1]octan-3-yl)carbamate 8c (25 mg, 49 μmol) and trifluoroacetic acid (1 mL) were added to dichloromethane (3 mL), and reacted at room temperature for 1 hour. The reaction solution was concentrated under reduced pressure to obtain crude product 6-((exo)-3-amino-8-azabicyclo[3.2.1]octan-8-yl)-3-(2,3-dichlorophenyl)-1H-pyrazolo[3,4-d]pyrimidine-4-carbonitrile 8d (20 mg) with a yield of 98%, which was directly used for the next reaction without purification.

MS m/z (ESI): 413.6 [M+1]

### Step 4

### 6-((exo)-3-amino-8-azabicyclo[3.2.1]octan-8-yl)-3-(2,3-dichlorophenyl)-1H-pyrazolo[3,4-d]pyrimidine-4-carboxamide

6-((exo)-3-amino-8-azabicyclo[3.2.1]octan-8-yl)-3-(2,3-dichlorophenyl)-1H-pyrazolo[3,4-d]pyrimidine-4-carbonitrile 8d (20 mg, 48 μmol) was added to a mixed solution of methanol (1.00 mL), aqueous sodium hydroxide (5 M, 1.00 mL) and 30% hydrogen peroxide (0.5 mL), and reacted for 3 hours at room temperature. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-((exo)-3-amino-8-azabicyclo[3.2.1]octan-8-yl)-3-(2,3-dichlorophenyl)-1H-pyrazolo[3,4-d]pyrimidine-4-carboxamide 8 (9 mg) with a yield of 43.5%.

MS m/z (ESI): 431.9 [M+1]

¹H NMR (400 MHz, DMSO-d₆) δ 8.02-8.14 (m, 1H), 7.87 (br, 3H), 7.55-7.73 (m, 2H), 7.30-7.47 (m, 2H), 4.90-5.16 (br, 1H), 4.49-4.76 (br, 1H), 3.16-3.27 (m, 1H), 2.29-2.48 (m, 2H), 2.06-2.23 (m, 2H), 1.83-2.01 (m, 2H), 1.56-1.80 (m, 2H).

### Example 9

### (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxylic acid

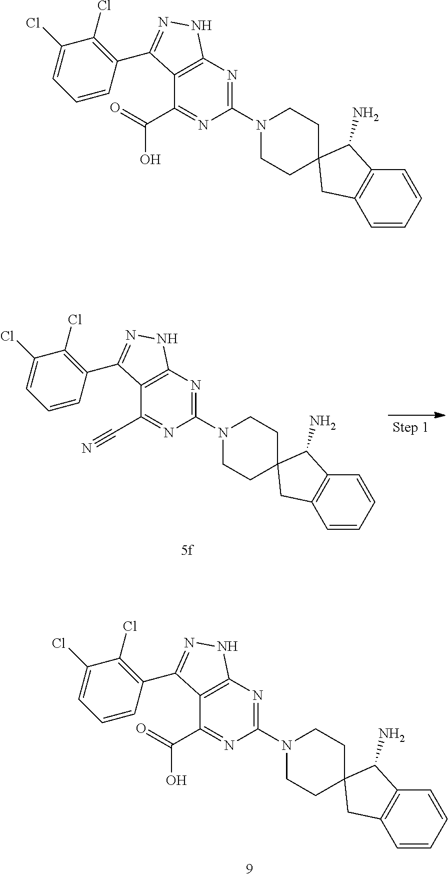 [SMILES: N[C@@H]1c2ccccc2CC12CCN(c1nc(C(=O)O)c3c(-c4cccc(Cl)c4Cl)n[nH]c3n1)CC2 ; N#Cc1nc(N2CCC3(CC2)Cc2ccccc2[C@H]3N)nc2[nH]nc(-c3cccc(Cl)c3Cl)c12 ; N[C@@H]1c2ccccc2CC12CCN(c1nc(C(=O)O)c3c(-c4cccc(Cl)c4Cl)n[nH]c3n1)CC2]

### Step 1

(S)-6-(1-amino-1,3-dihydrospiro[indene-2,4′-piperidin]-1′-yl)-3-(2,3-dichlorophenyl)-1H-pyrazolo[3,4-d]pyrimidine-4-carbonitrile 5f (63 mg, 128.47 μmol) was added to 5 mL of aqueous hydrochloric acid solution, and heated and refluxed for 3 hours. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxylic acid 9 (21 mg) with a yield of 25.43%.

MS m/z (ESI): 509.1 [M+1]

¹HNMR (400 MHz, DMSO-d₆) δ 13.64 (s, 1H), 8.24 (s, 3H), 7.63-7.75 (m, 1H), 7.49-7.54 (m, 1H), 7.40-7.48 (m, 2H), 7.28-7.40 (m, 3H), 4.47-4.88 (m, 2H), 4.39 (s, 1H), 3.18-3.40 (m, 3H), 3.00-3.10 (m, 1H), 1.61-1.82 (m, 2H), 1.44-1.60 (m, 2H).

### Example 10

### 6-(4-amino-4-phenylpiperidin-1-yl)-3-(2,3-dichlorophenyl)-1H-pyrazolo[3,4-d]pyrimidine-4-carboxylic acid

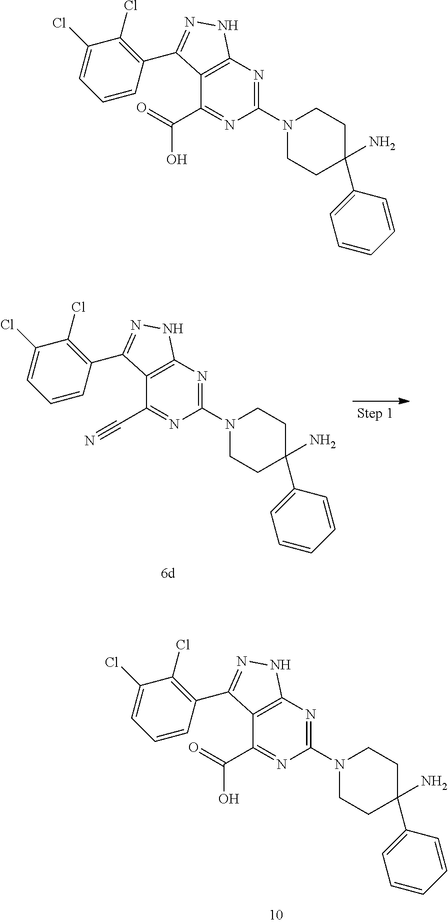 [SMILES: NC1(c2ccccc2)CCN(c2nc(C(=O)O)c3c(-c4cccc(Cl)c4Cl)n[nH]c3n2)CC1 ; N#Cc1nc(N2CCC(N)(c3ccccc3)CC2)nc2[nH]nc(-c3cccc(Cl)c3Cl)c12 ; NC1(c2ccccc2)CCN(c2nc(C(=O)O)c3c(-c4cccc(Cl)c4Cl)n[nH]c3n2)CC1]

### Step 1

6-(4-amino-4-phenylpiperidin-1-yl)-3-(2,3-dichlorophenyl)-1H-pyrazolo[3,4-d]pyrimidine-4-carbonitrile 6d (50 mg, 107.68 μmol) was added to 3 mL of concentrated hydrochloric acid, and heated and refluxed for 1 hour. After the reaction was completed, the reaction solution was added with a potassium carbonate solution to adjust the pH to be 9-10, concentrated under reduced pressure, and then subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-phenylpiperidin-1-yl)-3-(2,3-dichlorophenyl)-1H-pyrazolo[3,4-d]pyrimidine-4-carboxylic acid 10 (21 mg) with a yield of 32.65%.

MS m/z (ESI): 465.8 [M−16]

¹HNMR (400 MHz, DMSO-d₆) δ 13.70 (s, 1H), 8.25-8.45 (m, 3H), 7.68-7.76 (m, 3H), 7.50-7.60 (m, 2H), 7.40-7.50 (m, 3H), 4.15-4.50 (m, 2H), 3.43-3.60 (m, 2H), 2.54-2.64 (m, 2H), 2.00-2.15 (m, 2H).

### Example 11

### 6-(4-amino-4-(2,6-difluorophenyl)piperidin-1-yl)-3-(2,3-dichlorophenyl)-1H-pyrazolo[3,4-d]pyrimidine-4-carboxamide

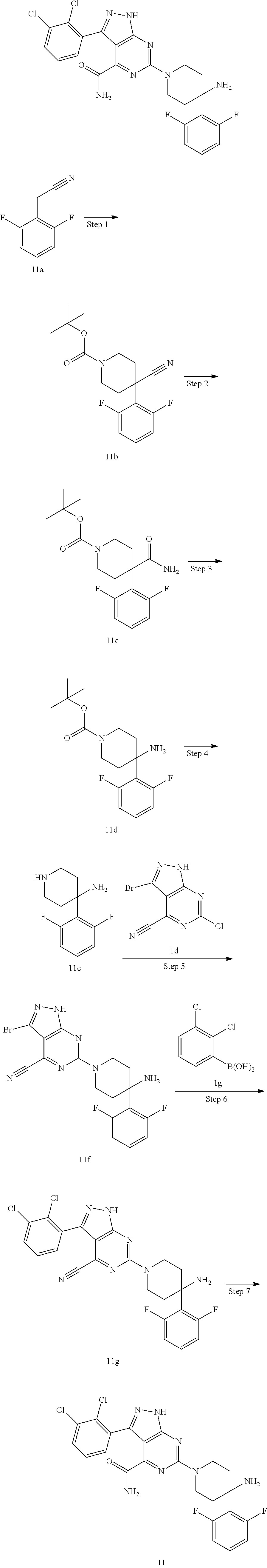

### Step 1

### Tert-butyl 4-cyano-4-(2,6-difluorophenyl)piperidine-1-carboxylate

At 0° C., sodium hydride (2.40 g, 59.92 mmol), 2-(2,6-difluorophenyl)acetonitrile 11a (1.53 g, 9.99 mmol) and tert-butyl N,N-bis(2-chloroethyl)carbamate (2.66 g, 10.99 mmol) were added to 15 mL of N,N-dimethylformamide, stirred for 1 hour in an ice bath, heated to 60° C., and then stirred for 16 hours. After the reaction was completed, the reaction solution was quenched by adding a saturated aqueous ammonium chloride solution (30 mL), and added with ethyl acetate (30 mL) for extraction and separation, and then aqueous phases were extracted with ethyl acetate (30 mL×2), and organic phases were combined, and washed with saturated brine, dried by anhydrous sodium sulfate, and concentrated under reduced pressure. The obtained residue was further separated and purified by silica gel column chromatography (eluent: system A) to obtain tert-butyl 4-cyano-4-(2,6-difluorophenyl)piperidine-1-carboxylate 11b (0.67 g) with a yield of 20.81%.

MS m/z (ESI): 222.8 [M−99]

### Step 2

### Tert-butyl 4-carbamoyl-4-(2,6-difluorophenyl)piperidine-1-carboxylate

Potassium hydroxide (233.25 mg, 4.16 mmol) and tert-butyl 4-cyano-4-(2,6-difluorophenyl)piperidine-1-carboxylate 11b (0.67 g, 2.08 mmol) were added to a solution of dimethyl sulfoxide (5 mL), hydrogen peroxide (2 mL, 30%) was slowly added to the reaction solution in an ice bath. After the dropwise addition was completed, the reaction solution was heated to room temperature, and stirred for 30 minutes. After the reaction was completed, the reaction solution was added with water (100 mL) to precipitate a white solid, and filtered, then the filter cake was washed with water, and dried in vacuum to obtain tert-butyl 4-carbamoyl-4-(2,6-difluorophenyl)piperidine-1-carboxylate 11c (0.64 g) with a yield of 90.47%.

MS m/z (ESI): 285.1 [M−55]

### Step 3

### Tert-butyl 4-amino-4-(2,6-difluorophenyl)piperidine-1-carboxylate

[Bis(trifluoroacetoxy)iodo]benzene (985.51 mg, 2.29 mmol) was added to 20 mL of mixed solution (acetonitrile:water=1:1) containing tert-butyl 4-carbamoyl-4-(2,6-difluorophenyl)piperidine-1-carboxylate 11c (0.64 g, 1.88 mmol), and stirred for 16 hours at room temperature. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain tert-butyl 4-amino-4-(2,6-difluorophenyl)piperidine-1-carboxylate 11d (251 mg) with a yield of 30.83%.

MS m/z (ESI): 239.9 [M−72]

### Step 4

### 4-(2,6-difluorophenyl)piperidin-4-amine

Tert-butyl 4-amino-4-(2,6-difluorophenyl)piperidine-1-carboxylate 11d (270 mg, 691.61 μmol) and trifluoroacetic acid (1 mL) were added to dichloromethane (3 mL), and reacted at room temperature for 1 hour. The reaction solution was concentrated under reduced pressure to obtain 4-(2,6-difluorophenyl)piperidin-4-amine lie (138.85 mg) with a yield of 94.7%, which was directly used for the next reaction without purification.

MS m/z (ESI): 160.1 [M−52]

### Step 5

### 6-(4-amino-4-(2,6-difluorophenyl)piperidin-1-yl)-3-bromo-1H-pyrazolo[3,4-d]pyrimidine-4-carbonitrile

At room temperature, 4-(2,6-difluorophenyl)piperidin-4-amine lie (138.85 mg, 425.59 μmol), 3-bromo-6-chloro-1H-pyrazolo[3,4-d]pyrimidine-4-carbonitrile 1d (100 mg, 386.90 μmol) and N,N-diisopropylethylamine (150 mg, 1.16 mmol) were added to N-methyl pyrrolidone (5 mL), heated to 110° C., and stirred for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2,6-difluorophenyl)piperidin-1-yl)-3-bromo-1H-pyrazolo[3,4-d]pyrimidine-4-carbonitrile 11f (181 mg) with a yield of 85.33%.

MS m/z (ESI): 417.0 [M−15]

### Step 6

### 6-(4-amino-4-(2,6-difluorophenyl)piperidin-1-yl)-3-(2,3-dichlorophenyl)-1H-pyrazolo[3,4-d]pyrimidine-4-carbonitrile

Under the protection of argon gas, 6-(4-amino-4-(2,6-difluorophenyl)piperidin-1-yl)-3-bromo-1H-pyrazolo[3,4-d]pyrimidine-4-carbonitrile 11f (150 mg, 345.43 μmol), (2,3-dichlorophenyl)boronic acid 1g (263.66 mg, 1.38 mmol), 2-dicyclohexylphosphino-2′,6′-di-isopropoxy-1,1′-biphenyl (64.48 mg, 138.17 μmol), methanesulfonato(2-dicyclohexylphosphino-2′,6′-di-isopropoxy-1,1′-biphenyl)(2-amino-1,1′-biphenyl-2-yl)palladium (57.85 mg, 69.09 μmol) and potassium phosphate (219.97 mg, 1.04 mmol) were added to 11 mL of mixed solution (1,4-dioxane:water=10:1) in turn, heated to 100° C., and reacted for 16 hours. After the reaction was completed, the reaction solution was concentrated under reduced pressure, added with ethyl acetate (10 mL) and water (10 mL) for extraction and separation, and then aqueous phases were extracted with ethyl acetate (10 mL×2), and organic phases were combined, and washed with saturated salt water, dried by anhydrous sodium sulfate, concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2,6-difluorophenyl)piperidin-1-yl)-3-(2,3-dichlorophenyl)-1H-pyrazolo[3,4-d]pyrimidine-4-carbonitrile 11g (130 mg) with a yield of 61.26%.

MS m/z (ESI): 483.0 [M−15]

### Step 7

### 6-(4-amino-4-(2,6-difluorophenyl)piperidin-1-yl)-3-(2,3-dichlorophenyl)-1H-pyrazolo[3,4-d]pyrimidine-4-carboxamide

6-(4-amino-4-(2,6-difluorophenyl)piperidin-1-yl)-3-(2,3-dichlorophenyl)-1H-pyrazolo[3,4-d]pyrimidine-4-carbonitrile 11g (50 mg, 81.39 μmol) was added to a mixed solution of methanol (1.00 mL), aqueous sodium hydroxide (5 M, 1.00 mL) and 30% hydrogen peroxide (0.5 mL), and reacted for 3 hours at room temperature. After the reaction was completed, the reaction solution was concentrated under reduced pressure, and subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2,6-difluorophenyl)piperidin-1-yl)-3-(2,3-dichlorophenyl)-1H-pyrazolo[3,4-d]pyrimidine-4-carboxamide 11 (15 mg) with a yield of 35.7%.

MS m/z (ESI): 517.8 [M+1]

¹HNMR (400 MHz, DMSO-d₆) δ 8.50-8.70 (m, 3H), 8.16 (s, 1H), 7.55-7.70 (m, 3H), 7.37-7.42 (m, 2H), 7.22-7.33 (m, 2H), 4.00-4.60 (m, 2H), 3.60-3.90 (m, 2H), 2.62-2.81 (m, 2H), 2.00-2.25 (m, 2H).

### Example 12

### 6-(4-amino-4-methylpiperidin-1-yl)-3-(3-bromo-2-chlorophenyl)-1H-pyrazolo[3,4-d]pyrimidine-4-carboxamide

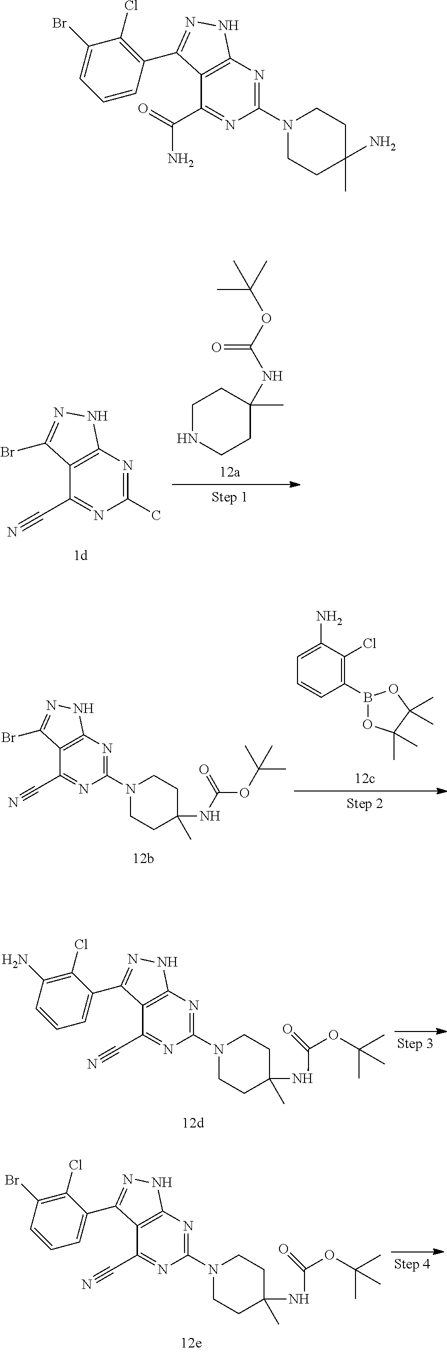

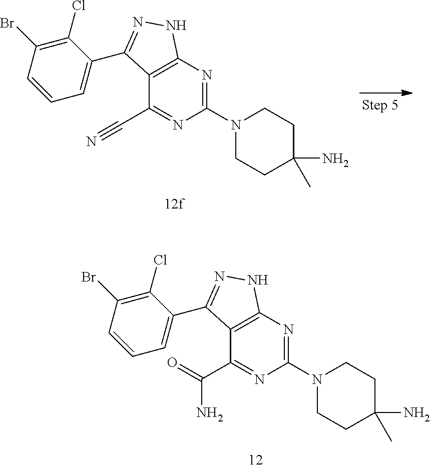 [SMILES: CC1(N)CCN(c2nc(C#N)c3c(-c4cccc(Br)c4Cl)n[nH]c3n2)CC1 ; CC1(N)CCN(c2nc(C(N)=O)c3c(-c4cccc(Br)c4Cl)n[nH]c3n2)CC1]

### Step 1

### Tert-butyl(1-(3-bromo-4-cyano-1H-pyrazolo[3,4-d]pyrimidin-6-yl)-4-methylpiperidin-4-yl)carbamate

3-bromo-6-chloro-1H-pyrazolo[3,4-d]pyrimidine-4-carbonitrile 1d (800 mg, 3.10 mmol), tert-butyl (4-methylpiperidin-4-yl)carbamate 12a (729.65 mg, 3.40 mmol) and diisopropylethylamine (1.20 g, 9.29 mmol, 1.53 mL) were added to N-methyl pyrrolidone (5 mL) in turn, heated to 110° C., and reacted for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain tert-butyl (1-(3-bromo-4-cyano-1H-pyrazolo[3,4-d]pyrimidin-6-yl)-4-methylpiperidin-4-yl)carbamate 12b (810 mg) with a yield of 59.98%.

MS m/z (ESI): 435.9 [M+1]

### Step 2

### Tert-butyl(1-(3-(3-amino-2-chlorophenyl)-4-cyano-1H-pyrazolo[3,4-d]pyrimidin-6-yl)-4-methylpiperidin-4-yl] carbamate

Under the protection of argon gas, tert-butyl (1-(3-bromo-4-cyano-1H-pyrazolo[3,4-d]pyrimidin-6-yl)-4-methylpiperidin-4-yl)carbamate 12b (100 mg, 181.71 μmol), 2-chloro-3-(4,4,5,5,-tetramethyl-1,3,2-dioxaborolan-2-yl)aniline 12c (138.21 mg, 545.13 μmol), methanesulfonato(2-dicyclohexylphosphino-2′,6′-di-isopropoxy-1,1′-biphenyl)(2-amino-1,1′-biphenyl-2-yl)palladium (30.42 mg, 36.34 μmol), potassium phosphate (55.43 mg, 261.15 μmol) and 2-dicyclohexylphosphino-2′,6′-di-isopropoxy-1,1′-biphenyl (33.92 mg, 72.68 μmol) were added to 11 mL of mixed solution (1,4-dioxane:water=10:1) in turn, and heated and refluxed for 16 hours. After the reaction was completed, the reaction solution was added with 10 mL of water and 10 mL of ethyl acetate for liquid separation and extraction, and then aqueous phases were extracted with ethyl acetate (10 mL×2), and organic phases were combined, and washed with 10 mL of saturated salt water, and concentrated under reduced pressure. The obtained residue was further separated and purified by silica gel column chromatography (eluent: system A) to obtain tert-butyl (1-(3-(3-amino-2-chlorophenyl)-4-cyano-1H-pyrazolo[3,4-d]pyrimidin-6-yl)-4-methylpiperidin-4-yl]carbamate 12d (51 mg) with a yield of 58.11%.

MS m/z (ESI): 482.9 [M+1]

### Step 3

### Tert-butyl(1-(3-(3-bromo-2-chlorophenyl)-4-cyano-1H-pyrazolo[3,4-d]pyrimidin-6-yl)-4-methylpiperidin-4-yl]carbamate

Under the protection of argon gas, tert-butyl (1-(3-(3-amino-2-chlorophenyl)-4-cyano-1H-pyrazolo[3,4-d]pyrimidin-6-yl)-4-methylpiperidin-4-yl]carbamate 12d (51 mg, 105.60 μmol) and cuprous bromide (30.30 mg, 211.20 μmol) were added to 5 mL of acetonitrile, cooled to 0-10° C. in an ice water bath, then added with tert-butyl nitrite (22.0 mg, 211.20 μmol), and reacted for 2 hours. After the reaction was completed, the reaction solution was extracted with ethyl acetate (10 mL×2), then organic phases were combined, and organic phases were washed with 10 mL of saturated salt water, and concentrated under reduced pressure. The obtained residue was further separated and purified by silica gel column chromatography (eluent: system A) to obtain tert-butyl (1-(3-(3-bromo-2-chlorophenyl)-4-cyano-1H-pyrazolo[3,4-d]pyrimidin-6-yl)-4-methylpiperidin-4-yl]carbamate 12e (42 mg) with a yield of 72.73%.

MS m/z (ESI): 545.8 [M+1]

### Step 4

### 6-(4-amino-4-methylpiperidin-1-yl)-3-(3-bromo-2-chlorophenyl)-1H-pyrazolo[3,4-d]pyrimidine-4-carbonitrile

Tert-butyl(1-(3-(3-bromo-2-chlorophenyl)-4-cyano-1H-pyrazolo[3,4-d]pyrimidin-6-yl)-4-methylpiperidin-4-yl]carbamate 12e (42 mg, 76.80 μmol) and trifluoroacetic acid (1 mL) were added to dichloromethane (3 mL), and reacted at room temperature for 1 hour. The reaction solution was concentrated under reduced pressure to obtain crude product 6-(4-amino-4-methylpiperidin-1-yl)-3-(3-bromo-2-chlorophenyl)-1H-pyrazolo[3,4-d]pyrimidine-4-carbonitrile 12f, which was directly used for the next reaction without purification.

MS m/z (ESI): 446.0 [M+1]

### Step 5

### 6-(4-amino-4-methylpiperidin-1-yl)-3-(3-bromo-2-chlorophenyl)-1H-pyrazolo[3,4-d]pyrimidine-4-carboxamide

6-(4-amino-4-methylpiperidin-1-yl)-3-(3-bromo-2-chlorophenyl)-1H-pyrazolo[3,4-d]pyrimidine-4-carbonitrile 12f (50 mg, 81.39 μmol) was added to a mixed solution of methanol (1.00 mL), aqueous sodium hydroxide (5 M, 1.00 mL) and 30% hydrogen peroxide (0.5 mL), and reacted for 3 hours at room temperature. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-methylpiperidin-1-yl)-3-(3-bromo-2-chlorophenyl)-1H-pyrazolo[3,4-d]pyrimidine-4-carboxamide 12 (2.1 mg) with a yield of 4.4%.

MS m/z (ESI): 463.8 [M+1]

¹HNMR (400 MHz, DMSO-d₆) δ 8.11 (s, 1H), 8.01 (s, 3H), 7.79 (d, J=8.0 Hz, 1H), 7.65 (s, 1H), 7.35-7.48 (m, 1H), 7.23-7.35 (m, 1H), 4.30-4.55 (m, 2H), 3.48-3.55 (m, 2H), 1.68-1.79 (m, 4H), 1.41 (s, 3H).

### Examples 13-21

Referring to the operation steps of step 1 to step 4 of Example 6, different starting materials were used to obtain the compounds of Examples 13-21.

| Example | | MS m/z | |
|---|---|---|---|
| No. | Product structure | (ESI) | ¹HNMR (400 MHz, DMSO-d₆₎ |
| 13 | 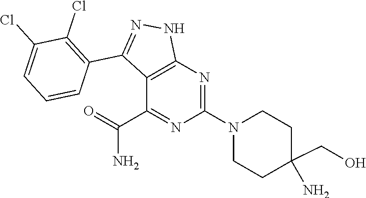 [SMILES: NC(=O)c1nc(N2CCC(N)(CO)CC2)nc2[nH]nc(-c3cccc(Cl)c3Cl)c12] | 436.1 [M + 1] | δ 8.13 (s, 1H), 8.01-7.76 (m, 3H), 7.70-7.61 (m, 2H), 7.40 (d, J = 8.0 Hz, 2H), 5.59 (s, 1H), 4.40-4.12 (m, 2H), 3.70-3.60 (m, 4H), 1.90-1.74 (m, 2H), 1.74-1.60 (m, 2H) |
| 14 | 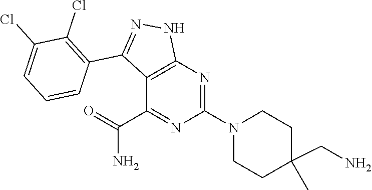 [SMILES: CC1(CN)CCN(c2nc(C(N)=O)c3c(-c4cccc(Cl)c4Cl)n[nH]c3n2)CC1] | 434.1 [M + 1] | δ 8.06 (s, 1H), 7.72 (s, 3H), 7.67-7.61 (m, 2H), 7.38 (d, J = 8.0 Hz, 2H), 4.40-4.20 (m, 2H), 3.62-3.45 (m, 2H), 2.80 (s, 2H), 1.58-1.47 (m, 2H), 1.47-1.36 (m, 2H), 1.10 (s, 3H) |
| 15 | 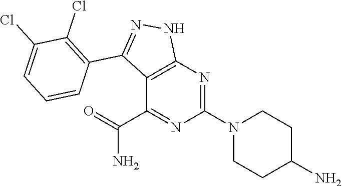 [SMILES: NC(=O)c1nc(N2CCC(N)CC2)nc2[nH]nc(-c3cccc(Cl)c3Cl)c12] | 405.9 [M + 1] | δ 8.12 (s, 1H), 7.95 (s, 3H), 7.75-7.55 (m, 2H), 7.45-7.35 (m, 2H), 5.0-4.60 (m, 2H), 3.42-3.32 (m, 1H), 3.06 (t, J = 16 Hz, 2H), 2.08-1.91 (m, 2H), 1.60-1.38 (m, 2H) |
| 16 | 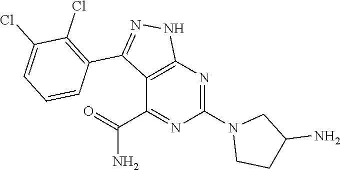 [SMILES: NC(=O)c1nc(N2CCC(N)C2)nc2[nH]nc(-c3cccc(Cl)c3Cl)c12] | 391.9 [M + 1] | δ 8.08 (s, 1H), 7.77 (s, 3H), 7.68-7.60 (m, 2H), 7.38 (d, J = 4.0 Hz, 2H), 4.08-3.56 (m, 6H), 2.41-2.26 (m, 1H) |
| 17 | 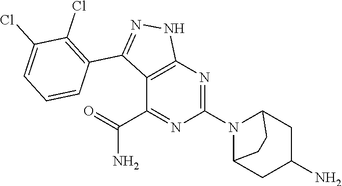 [SMILES: NC(=O)c1nc(N2C3CCC2CC(N)C3)nc2[nH]nc(-c3cccc(Cl)c3Cl)c12] | 431.1 [M + 1] | δ 8.12 (s, 1H), 7.80-7.60 (m, 5H), 7.45-7.35 (m, 2H), 5.07 (s, 1H), 4.67 (s, 1H), 3.42-3.32 (m, 1H), 2.06 (s, 2H), 2.0-1.73 (m, 5H), 1.73-1.58 (m, 1H) |
| 18 | 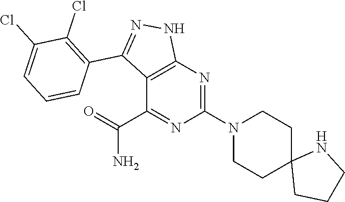 [SMILES: NC(=O)c1nc(N2CCC3(CCCN3)CC2)nc2[nH]nc(-c3cccc(Cl)c3Cl)c12] | 445.9 [M + 1] | δ 8.88 (s, 2H), 8.12 (s, 1H), 7.70-7.60 (m, 2H), 7.45-7.35 (m, 2H), 4.60-4.20 (m, 2H), 3.42-3.32 (m, 3H), 2.13-1.93 (m, 5H), 1.93-1.78 (m, 4H) |
| 19 | 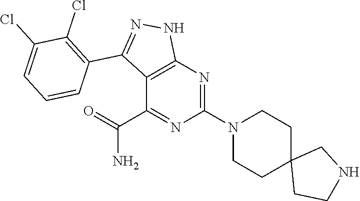 [SMILES: NC(=O)c1nc(N2CCC3(CCNC3)CC2)nc2[nH]nc(-c3cccc(Cl)c3Cl)c12] | 445.9 [M + 1] | δ 8.88 (s, 2H), 7.94 (s, 1H), 7.53-7.45 (m, 2H), 7.33-7.16 (m, 2H), 3.90-3.80 (m, 2H), 3.20-3.0 (m, 4H), 3.0-2.80 (m, 2H), 1.73 (t, J = 4.0 Hz, 2H), 1.57-1.36 (m, 4H) |
| 20 | 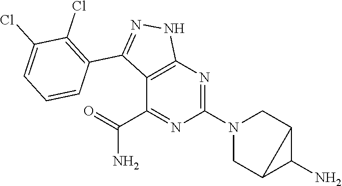 [SMILES: NC(=O)c1nc(N2CC3C(N)C3C2)nc2[nH]nc(-c3cccc(Cl)c3Cl)c12] | 403.9 [M + 1] | δ 8.25-8.20 (m, 1H), 8.0-7.80 (m, 3H), 7.75-7.61 (m, 2H), 7.48-7.36 (m, 2H), 4.0-3.77 (m, 4H), 2.15-2.0 (m, 3H) |
| 21 | 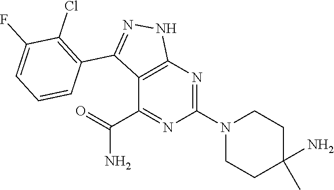 [SMILES: CC1(N)CCN(c2nc(C(N)=O)c3c(-c4cccc(F)c4Cl)n[nH]c3n2)CC1] | 403.9 [M + 1] | δ 8.11 (s, 1H), 8.01 (s, 3H), 7.65 (s, 1H), 7.48-7.35 (m, 2H), 7.25-7.33 (m, 1H), 4.55-4.30 (m, 2H), 4.0-3.7 (m, 2H), 1.74 (s, 4H), 1.41 (s, 3H) |

### Example 22

### 2-(4-amino-4-methylpiperidin-1-yl)-5-(2,3-dichlorophenyl)-7H-pyrrolo[2,3-d]pyrimidine-4-carboxamide

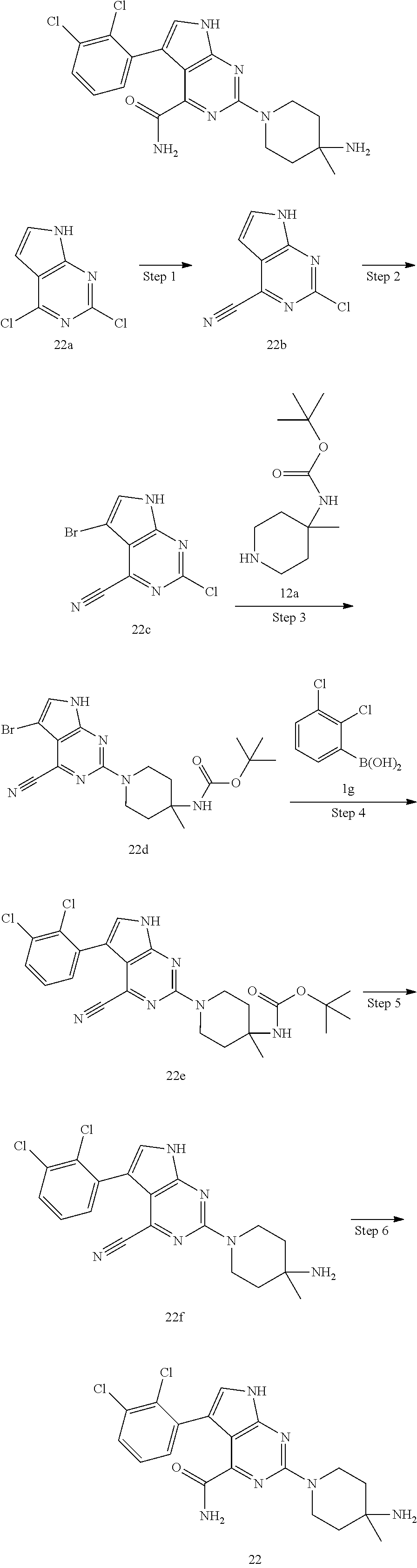

### Step 1

### 2-chloro-7H-pyrrolo[2,3-d]pyrimidine-4-carbonitrile

2,4-dichloro-7H-pyrrolo[2,3-d]pyrimidine 22a (1 g, 5.32 mmol), tetrakis(triphenylphosphine)palladium (614.32 mg, 531.88 μmol) and zinc cyanide (1.25 g, 10.64 mmol) were added to N,N-dimethylformamide (20 mL), heated to 110° C. and stirred for 4 hours after argon gas displacement. After the reaction was completed, the filtrate was concentrated under reduced pressure, and the obtained residue was further separated and purified by silicagel column chromatography (eluent: system A) to obtain 2-chloro-7H-pyrrolo[2,3-d]pyrimidine-4-carbonitrile 22b (850 mg) with a yield of 89.49%.

MS m/z (ESI): 178.9 [M+1]

### Step 2

### 5-bromo-2-chloro-7H-pyrrolo[2,3-d]pyrimidine-4-carbonitrile

2-chloro-7H-pyrrolo[2,3-d]pyrimidine-4-carbonitrile 22b (850 mg, 4.76 mmol) was added to a solution of acetonitrile (20 mL), added with bromosuccinimide (1,270 mg, 7.14 mmol), heated and refluxed, and stirred for 2 hours. After the reaction was completed, the filtrate was concentrated under reduced pressure, and the obtained residue was further separated and purified by silica gel column chromatography (eluent: system A) to obtain 5-bromo-2-chloro-7H-pyrrolo[2,3-d]pyrimidine-4-carbonitrile 22c (1.05 g) with a yield of 85.68%.

MS m/z (ESI): 256.8 [M+1]

### Step 3

### Tert-butyl(1-(5-bromo-4-cyano-7H-pyrrolo[2,3-d]pyrimidin-2-yl)-4-methylpiperidin-4-yl)carbamate

At room temperature, tert-butyl (4-methylpiperidin-4-yl)carbamate 12a (174.79 mg, 815.61 μmol), N-methyl pyrrolidone (10 mL) and N,N-diisopropylethylamine (301.17 mg, 2.33 mmol, 384.83 μL) were added to a 100 mL single-necked round-bottom flask and shaken for 1 minute, then added with 5-bromo-2-chloro-7H-pyrrolo[2,3-d]pyrimidine-4-carbonitrile 22c (200 mg, 776.78 μmol), heated to 110° C., and stirred for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain tert-butyl (1-(5-bromo-4-cyano-7H-pyrrolo[2,3-d]pyrimidin-2-yl)-4-methylpiperidin-4-yl) carbamate 22d (186 mg) with a yield of 55.01%.

MS m/z (ESI): 434.9 [M+1]

### Step 4

### Tert-butyl (1-(5-(2,3-dichlorophenyl)-4-cyano-7H-pyrrolo[2,3-d]pyrimidin-2-yl)-4-methylpiperidin-4-yl) carbamate

At room temperature, tert-butyl(1-(5-bromo-4-cyano-7H-pyrrolo[2,3-d]pyrimidin-2-yl)-4-methylpiperidin-4-yl) carbamate 22d (186 mg, 427.27 μmol), (2,3-dichlorophenyl)boronic acid 1g (326.13 mg, 1.71 mmol), 2-dicyclohexylphosphino-2′,6′-di-isopropoxy-1,1′-biphenyl (79.75 mg, 170.91 μmol), methanesulfonato(2-dicyclohexylphosphino-2′,6′-di-isopropoxy-1,1′-biphenyl)(2-amino-1,1′-biphenyl-2-yl)palladium (71.56 mg, 85.45 μmol) and potassium phosphate (272.09 mg, 1.28 mmol) were added to a 50 mL double-necked round-bottom flask, and finally added with 11 mL of mixed solution (1,4-dioxane:water=10:1), subjected to argon gas displacement thrice, heated to 100° C., and reacted for 16 hours. After the reaction was completed, the reaction solution was concentrated under reduced pressure, and then added with ethyl acetate (10 mL) and water (10 mL) for extraction and separation, and then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain tert-butyl (1-(5-(2,3-dichlorophenyl)-4-cyano-7H-pyrrolo[2,3-d]pyrimidin-2-yl)-4-methylpiperidin-4-yl)carbamate 22e (159 mg) with a yield of 74.22%.

MS m/z (ESI): 500.9 [M+1]

### Step 5

### 2-(4-amino-4-methylpiperidin-1-yl)-5-(2,3-dichlorophenyl)-7H-pyrrolo[2,3-d]pyrimidine-4-carbonitrile

Tert-butyl (1-(5-(2,3-dichlorophenyl)-4-cyano-7H-pyrrolo[2,3-d]pyrimidin-2-yl)-4-methylpiperidin-4-yl)carbamate 22e (159 mg, 317.11 μmol) and trifluoroacetic acid (1 mL) were added to a solution of dichloromethane (3 mL), and stirred at room temperature for 1 hour. After the reaction was completed, the reaction solution was concentrated under reduced pressure to obtain 2-(4-amino-4-methylpiperidin-1-yl)-5-(2,3-dichlorophenyl)-7H-pyrrolo[2,3-d]pyrimidine-4-carbonitrile 22f, which was directly used for the next reaction without purification.

MS m/z (ESI): 400.7 [M+1]

### Step 6

### 2-(4-amino-4-methylpiperidin-1-yl)-5-(2,3-dichlorophenyl)-7H-pyrrolo[2,3-d]pyrimidine-4-carboxamide

2-(4-amino-4-methylpiperidin-1-yl)-5-(2,3-dichlorophenyl)-7H-pyrrolo[2,3-d]pyrimidine-4-carbonitrile 22f (160 mg, 310.49 μmol) was dissolved in a mixed solution of methanol (1.00 mL), aqueous sodium hydroxide (5 M, 1.00 mL) and 30% hydrogen peroxide (0.5 mL), and the reaction solution was stirred for 0.5 hour at room temperature. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 2-(4-amino-4-methylpiperidin-1-yl)-5-(2,3-dichlorophenyl)-7H-pyrrolo[2,3-d]pyrimidine-4-carboxamide 22 (48 mg) with a yield of 28.99%.

MS m/z (ESI): 418.8 [M+1]

¹HNMR (400 MHz, DMSO-d₆) δ 8.0-7.80 (m, 4H), 7.54-7.48 (m, 1H), 7.48-7.43 (m, 1H), 7.33-7.28 (m, 3H), 4.45-4.35 (m, 2H), 3.45-3.35 (m, 2H), 1.78-1.65 (m, 4H), 1.41 (s, 3H).

### Example 23

### 6-(4-amino-4-(2-chlorophenyl)piperidin-1-yl)-3-(2,3-dichlorophenyl)-1H-pyrazolo[3,4-d]pyrimidine-4-carboxylic acid

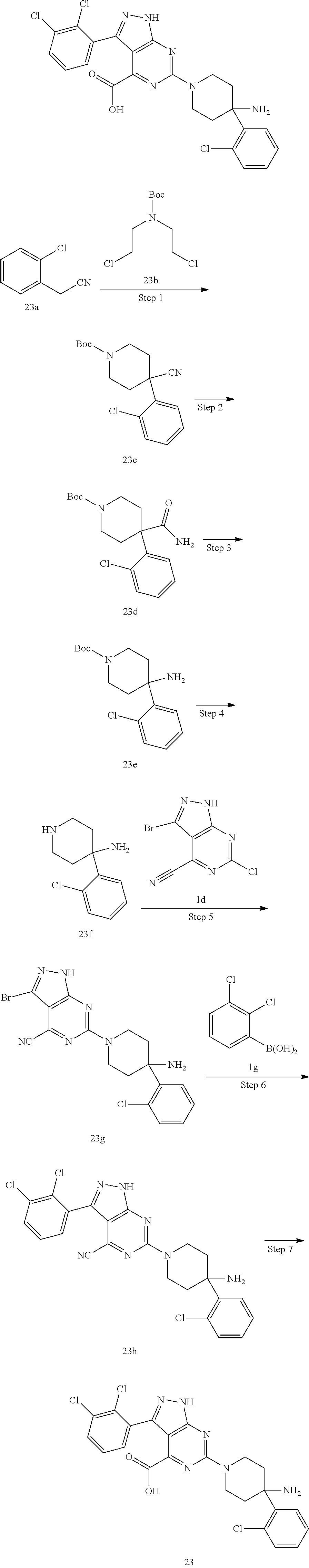

### Step 1

### Tert-butyl 4-(2-chlorophenyl)-4-cyanopiperidine-1-carboxylate

In an ice water bath, sodium hydride (2.40 g, 60 mmol, 60%) was added to a solution of N,N-dimethylformamide (15 mL) containing 2-(2-chlorophenyl)acetonitrile 23a (1.52 g, 10 mmol) and tert-butyl bis(2-chloroethyl)carbamate 23b (2.66 g, 11 mmol), stirred for 1 hour, heated to 60° C., and then stirred overnight. After the reaction was completed, the reaction solution was cooled to room temperature, quenched with a saturated aqueous ammonium chloride solution (30 mL), and added with ethyl acetate (30 mL) for extraction and separation, then aqueous phases were extracted with ethyl acetate (30 mL×2), and organic phases were combined, and washed with saturated salt water, dried by anhydrous sodium sulfate, and concentrated under reduced pressure to obtain crude product. The crude product was beaten with 100 mL of mixed solvent (ethyl acetate:petroleum ether=10:90), filtered and dried to obtain the product tert-butyl 4-(2-chlorophenyl)-4-cyanopiperidine-1-carboxylate 23c (2.1 g) with a yield of 65.5%.

MS m/z (ESI): 338.1 [M+18]

### Step 2

### Tert-butyl 4-carbamoyl-4-(2-chlorophenyl)piperidine-1-carboxylate

Potassium hydroxide (735 mg, 13.1 mmol) and tert-butyl 4-(2-chlorophenyl)-4-cyanopiperidine-1-carboxylate 23c (2.1 g, 6.55 mmol) were added to a solution of dimethylsulphoxide (15 mL), and hydrogen peroxide (30%, 6.5 mL) was slowly added dropwise to the reaction solution. After the dropwise addition was completed, the reaction solution was stirred for 1 hour. After the reaction was completed, the reaction solution was added with 50 mL of water to precipitate a yellow solid, and filtered, then the filter cake was washed with water, and dried in vacuum to obtain the product tert-butyl 4-carbamoyl-4-(2-chlorophenyl)piperidine-1-carboxylate 23d (1.9 g) with a yield of 85.7%.

MS m/z (ESI): 282.9 [M−55]

### Step 3

### Tert-butyl 4-amino-4-(2-chlorophenyl)piperidine-1-carboxylate

Tert-butyl 4-carbamoyl-4-(2-chlorophenyl)piperidine-1-carboxylate 23d (1.9 g, 5.61 mmol) was added to a mixed solution of acetonitrile (10 mL) and water (40 mL) containing potassium hydroxide (1.42 g, 25.23 mmol), and then added with 1,3-dibromo-5,5-dimethylhydantoin (882 mg, 3.08 mmol) in batches, and stirred at room temperature for 1 hour. After the reaction was completed, the reaction solution was added with sodium sulfite (70.6 mg, 0.56 mmol) and stirred for 15 minutes, then added with ethyl acetate (20 mL) and potassium phosphate (1.31 g, 6.17 mmol) for liquid separation, aqueous phases were extracted with ethyl acetate (50 mL×2), organic phases were combined and washed with a saturated brine solution, dried, and concentrated to obtain tert-butyl 4-amino-4-(2-chlorophenyl)piperidine-1-carboxylate 23e (1.62 g) with a yield of 93.0%.

MS m/z (ESI): 311.0 [M+1]

### Step 4

### 4-(2-chlorophenyl)piperidin-4-amine

Trifluoroacetic acid (1 mL) was dropwise added to 3 mL of dichloromethane solution containing tert-butyl 4-amino-4-(2-chlorophenyl)piperidine-1-carboxylate 23e (200 mg, 643 μmol), and reacted at room temperature for 1 hour. The reaction solution was concentrated under reduced pressure to obtain 4-(2-chlorophenyl)piperidine-4-amine 23f, which was directly used for the next reaction without purification.

MS m/z (ESI): 211.0 [M+1]

### Step 5

### 6-(4-amino-4-(2-chlorophenyl)piperidin-1-yl)-3-bromo-1H-pyrazolo[3,4-d]pyrimidine-4-carbonitrile

Diisopropylethylamine (91.4 mg, 707 μmol) and the above-mentioned crude product 4-(2-chlorophenyl)piperidine-4-amine 23f were added to a solution of N-methyl pyrrolidone (5 mL) containing 3-bromo-6-chloro-1H-pyrazolo[3,4-d]pyrimidine-4-carbonitrile 1d (150 mg, 580 μmol), heated to 100° C., and stirred for 1 hour. After the reaction was completed, the reaction solution was separated on a C₁₈ reversed phase column (C₁₈ separation column 20-45 μm; mobile phase A: H₂O, mobile phase B: CH₃CN) to obtain the product 6-(4-amino-4-(2-chlorophenyl)piperidin-1-yl)-3-bromo-1H-pyrazolo[3,4-d]pyrimidine-4-carbonitrile 23g (200 mg) with a yield of 65.4%.

MS m/z (ESI): 414.8 [M−16]

### Step 6

### 6-(4-amino-4-(2-chlorophenyl)piperidin-1-yl)-3-(2,3-dichlorophenyl)-1H-pyrazolo[3,4-d]pyrimidine-4-carbonitrile

6-(4-amino-4-(2-chlorophenyl)piperidin-1-yl)-3-bromo-1H-pyrazolo[3,4-d]pyrimidine-4-carbonitrile 23g (200 mg, 462 μmol), (2,3-dichlorophenyl)boronic acid 1g (353 mg, 1.85 mmol), methanesulfonato(2-dicyclohexylphosphino-2′,6′-di-isopropoxy-1,1′-biphenyl)(2-amino-1,1′-biphenyl-2-yl)palladium (77 mg, 92 μmol), 2-dicyclohexylphosphino-2′,6′-di-isopropoxy-1,1′-biphenyl (86 mg, 185 μmol) and potassium phosphate (294 mg, 1.39 mmol) were added to a mixed solution of 1,4-dioxane (5 mL) and water (1 mL), subjected to argon gas displacement thrice, then heated to 100° C., and reacted overnight. After the reaction was completed, the reaction solution was concentrated under reduced pressure, and added with ethyl acetate (10 mL) and water (10 mL) for extraction and separation, then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain 6-(4-amino-4-(2-chlorophenyl)piperidin-1-yl)-3-(2,3-dichlorophenyl)-1H-pyrazolo[3,4-d]pyrimidine-4-carbonitrile 23h (90 mg) with a yield of 39.0%.

MS m/z (ESI): 480.8 [M−16]

### Step 7

### 6-(4-amino-4-(2-chlorophenyl)piperidin-1-yl)-3-(2,3-dichlorophenyl)-1H-pyrazolo[3,4-d]pyrimidine-4-carboxylic acid

6-(4-amino-4-(2-chlorophenyl)piperidin-1-yl)-3-(2,3-dichlorophenyl)-1H-pyrazolo[3,4-d]pyrimidine-4-carbonitrile 23h (90 mg, 180 μmol) was added to 3 mL of concentrated hydrochloric acid, and heated and refluxed for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2-chlorophenyl)piperidin-1-yl)-3-(2,3-dichlorophenyl)-1H-pyrazolo[3,4-d]pyrimidine-4-carboxylic acid 23 (12 mg) with a yield of 10%.

MS m/z (ESI): 516.8 [M+1]

¹HNMR (400 MHz, CD₃OD) δ 7.72-7.82 (m 1H), 7.57-7.67 (m, 2H), 7.46-7.56 (m, 2H), 7.33-7.45 (m, 2H), 4.21-4.52 (m, 2H), 3.78-4.03 (m, 2H), 2.87-3.04 (m, 2H), 2.23-2.39 (m, 2H).

### Example 24

### 6-(4-amino-4-(4-chlorophenyl)piperidin-1-yl)-3-(2,3-dichlorophenyl)-1H-pyrazolo[3,4-d]pyrimidine-4-carboxylic acid

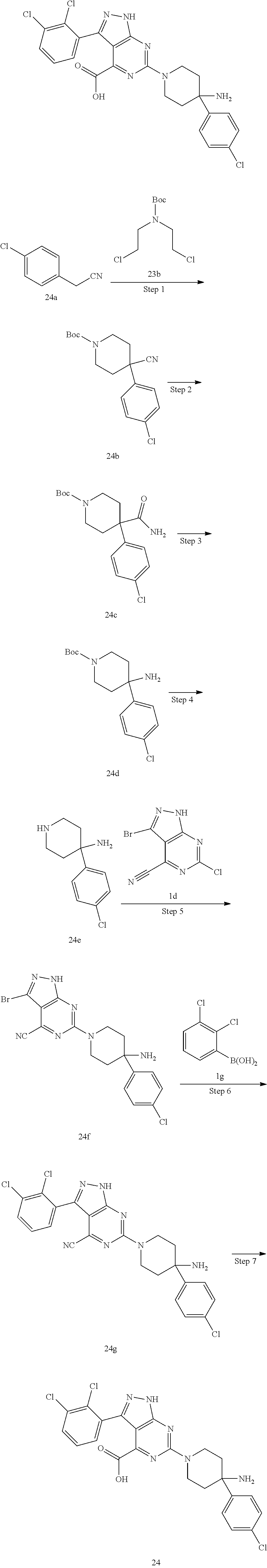

### Step 1

### Tert-butyl 4-(4-chlorophenyl)-4-cyanopiperidine-1-carboxylate

In an ice water bath, sodium hydride (2.41 g, 60.2 mmol, 60%) was added to a solution of N,N-dimethylformamide (15 mL) containing 2-(4-chlorophenyl)acetonitrile 24a (1.52 g, 10 mmol) and tert-butyl bis(2-chloroethyl)carbamate 23b (2.67 g, 11 mmol), stirred for 1 hour, heated to 60° C., and then stirred overnight. After the reaction was completed, the reaction solution was cooled to room temperature, quenched with a saturated aqueous ammonium chloride solution (30 mL), then added with ethyl acetate (30 mL) for extraction and separation, then aqueous phases were washed with ethyl acetate (30 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain the product tert-butyl 4-(4-chlorophenyl)-4-cyanopiperidine-1-carboxylate 24b (2.4 g) with a yield of 74.6%.

MS m/z (ESI): 338.1 [M+18]

### Step 2

### Tert-butyl 4-carbamoyl-4-(4-chlorophenyl)piperidine-1-carboxylate

Potassium hydroxide (840 mg, 15.0 mmol) and tert-butyl 4-(4-chlorophenyl)-4-cyanopiperidine-1-carboxylate 24b (2.40 g, 7.48 mmol) were added to a solution of dimethylsulphoxide (15 mL), and hydrogen peroxide (30%, 6.5 mL) was slowly added dropwise to the reaction solution. After the dropwise addition was completed, the reaction solution was stirred for 1 hour. After the reaction was completed, the reaction solution was added with 50 mL of water to precipitate a yellow solid, and filtered, then the filter cake was washed with water, and dried in vacuum to obtain the product tert-butyl 4-carbamoyl-4-(4-chlorophenyl)piperidine-1-carboxylate 24c (2.2 g) with a yield of 86.8%.

MS m/z (ESI): 283.1 [M−55]

### Step 3

### Tert-butyl 4-amino-4-(4-chlorophenyl)piperidine-1-carboxylate

Tert-butyl 4-carbamoyl-4-(4-chlorophenyl)piperidine-1-carboxylate 24c (1.0 g, 2.95 mmol) was added to a mixed solution of acetonitrile (2.5 mL) and water (10 mL) containing potassium hydroxide (745 mg, 13.3 mmol), and then added with dibromohydantion (464 mg, 1.62 mmol) in batches, and stirred at room temperature for 1 hour. After the reaction was completed, the reaction solution was added with sodium sulfite (37.8 mg, 0.3 mmol) and stirred for 15 minutes, then added with ethyl acetate (5 mL) and potassium phosphate (688 mg, 3.25 mmol) for liquid separation, aqueous phases were extracted with ethyl acetate (5 mL×2), organic phases were combined and washed with a saturated salt water, dried, and concentrated to obtain tert-butyl 4-amino-4-(4-chlorophenyl)piperidine-1-carboxylate 24d (0.85 g) with a yield of 92.7%.

MS m/z (ESI): 237.9 [M−72]

### Step 4

### 4-(4-chlorophenyl)piperidin-4-amine

Trifluoroacetic acid (1 mL) was dropwise added to 3 mL of dichloromethane solution containing tert-butyl 4-amino-4-(4-chlorophenyl)piperidine-1-carboxylate 24d (200 mg, 643 μmol), and reacted at room temperature for 1 hour. The reaction solution was concentrated under reduced pressure to obtain 4-(4-chlorophenyl)piperidin-4-amine 24e, which was directly used for the next reaction without purification.

MS m/z (ESI): 211.0 [M+1]

### Step 5

### 6-(4-amino-4-(4-chlorophenyl)piperidin-1-yl)-3-bromo-1H-pyrazolo[3,4-d]pyrimidine-4-carbonitrile

Diisopropylethylamine (91.4 mg, 707 μmol) and the above-mentioned crude product 4-(4-chlorophenyl)piperidin-4-amine 24e were added to a solution of N-methyl pyrrolidone (5 mL) containing 3-bromo-6-chloro-1H-pyrazolo[3,4-d]pyrimidine-4-carbonitrile 1d (150 mg, 580 μmol), heated to 100° C., and stirred for 1 hour. After the reaction was completed, the reaction solution was separated on a C₁₈ reversed phase column (C₁₈ separation column 20-45 μm; mobile phase A: H₂O, mobile phase B: CH₃CN) to obtain the product 6-(4-amino-4-(4-chlorophenyl)piperidin-1-yl)-3-bromo-1H-pyrazolo[3,4-d]pyrimidine-4-carbonitrile 24f (225 mg) with a yield of 73.5%.

MS m/z (ESI): 414.8 [M−16]

### Step 6

### 6-(4-amino-4-(4-chlorophenyl)piperidin-1-yl)-3-(2,3-dichlorophenyl)-1H-pyrazolo[3,4-d]pyrimidine-4-carbonitrile

6-(4-amino-4-(4-chlorophenyl)piperidin-1-yl)-3-bromo-1H-pyrazolo[3,4-d]pyrimidine-4-carbonitrile 24f (225 mg, 519.99 μmol), (2,3-dichlorophenyl)boronic acid 1g (396.89 mg, 2.08 mmol), methanesulfonato(2-dicyclohexylphosphino-2′,6′-di-isopropoxy-1,1′-biphenyl)(2-amino-1,1′-biphenyl-2-yl)palladium (87.08 mg, 104.00 μmol), 2-dicyclohexylphosphino-2′,6′-di-isopropoxy-1,1′-biphenyl (97.06 mg, 207.99 μmol) and potassium phosphate (331 mg, 1.56 mmol) were added to a mixed solution of 1,4-dioxane (5 mL) and water (1 mL), subjected to argon gas displacement thrice, then heated to 100° C., and reacted overnight. After the reaction was completed, the reaction solution was concentrated under reduced pressure, and added with ethyl acetate (10 mL) and water (10 mL) for extraction and separation, then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain 6-(4-amino-4-(4-chlorophenyl)piperidin-1-yl)-3-(2,3-dichlorophenyl)-1H-pyrazolo[3,4-d]pyrimidine-4-carbonitrile 24g (95 mg) with a yield of 36.6%.

MS m/z (ESI): 480.8 [M−16]

### Step 7

### 6-(4-amino-4-(4-chlorophenyl)piperidin-1-yl)-3-(2,3-dichlorophenyl)-1H-pyrazolo[3,4-d]pyrimidine-4-carboxylic acid

6-(4-amino-4-(4-chlorophenyl)piperidin-1-yl)-3-(2,3-dichlorophenyl)-1H-pyrazolo[3,4-d]pyrimidine-4-carbonitrile 24g (95 mg, 190 μmol) was added to 3 mL of concentrated hydrochloric acid, and heated and refluxed for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(4-chlorophenyl)piperidin-1-yl)-3-(2,3-dichlorophenyl)-1H-pyrazolo[3,4-d]pyrimidine-4-carboxylic acid 24 (15 mg) with a yield of 8.8%.

MS m/z (ESI): 516.8 [M+1]

### Example 25

### 6-(4-amino-4-(3-chlorophenyl)piperidin-1-yl)-3-(2,3-dichlorophenyl)-1H-pyrazolo[3,4-d]pyrimidine-4-carboxylic acid

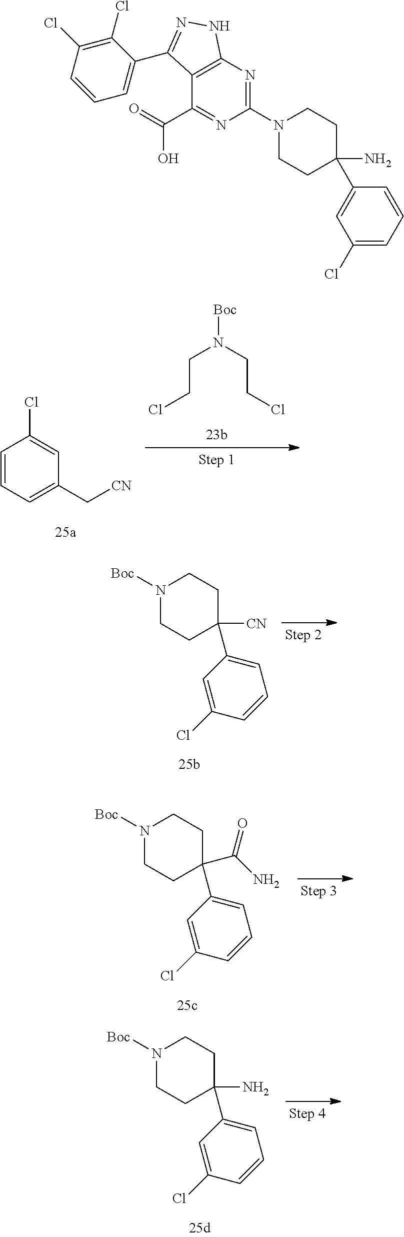 [SMILES: NC1(c2cccc(Cl)c2)CCN(c2nc(C(=O)O)c3c(-c4cccc(Cl)c4Cl)n[nH]c3n2)CC1 ; N#CCc1cccc(Cl)c1 ; CC(C)(C)OC(=O)N(CCCl)CCCl ; CC(C)(C)OC(=O)N1CCC(C#N)(c2cccc(Cl)c2)CC1 ; CC(C)(C)OC(=O)N1CCC(C(N)=O)(c2cccc(Cl)c2)CC1 ; CC(C)(C)OC(=O)N1CCC(N)(c2cccc(Cl)c2)CC1]

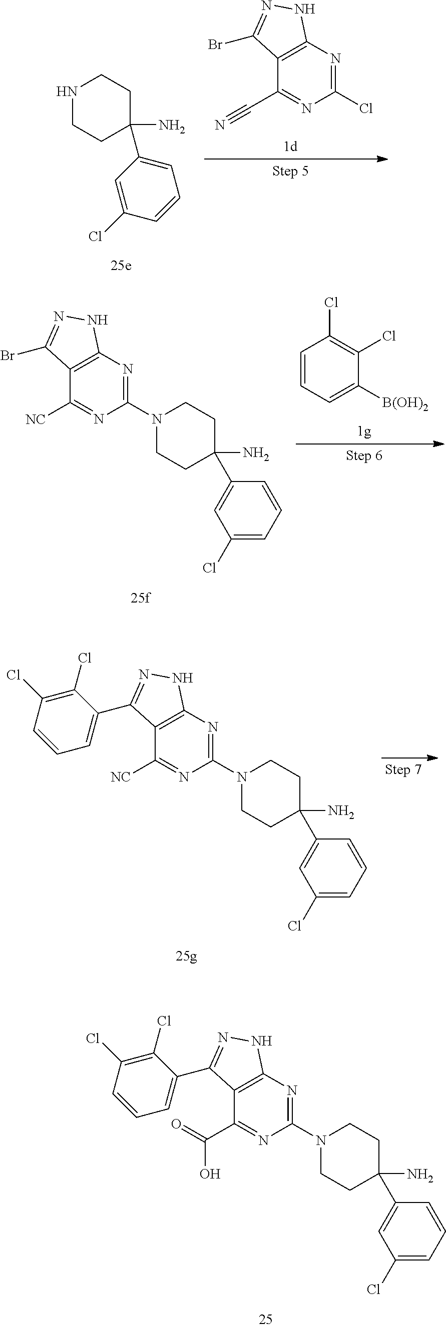 [SMILES: NC1(c2cccc(Cl)c2)CCNCC1 ; N#Cc1nc(Cl)nc2[nH]nc(Br)c12 ; N#Cc1nc(N2CCC(N)(c3cccc(Cl)c3)CC2)nc2[nH]nc(Br)c12 ; OB(O)c1cccc(Cl)c1Cl ; N#Cc1nc(N2CCC(N)(c3cccc(Cl)c3)CC2)nc2[nH]nc(-c3cccc(Cl)c3Cl)c12 ; NC1(c2cccc(Cl)c2)CCN(c2nc(C(=O)O)c3c(-c4cccc(Cl)c4Cl)n[nH]c3n2)CC1]

### Step 1

### Tert-butyl 4-(3-chlorophenyl)-4-cyanopiperidine-1-carboxylate

In an ice water bath, sodium hydride (2.4 g, 60 mmol, 60%) was added to a solution of N,N-dimethylformamide (15 mL) containing 2-(3-chlorophenyl)acetonitrile 25a (1.52 g, 10 mmol) and tert-butyl bis(2-chloroethyl)carbamate 23b (2.66 g, 11 mmol), stirred for 1 hour, heated to 60° C., and then stirred overnight. After the reaction was completed, the reaction solution was cooled to room temperature, quenched with a saturated aqueous ammonium chloride solution (30 mL), then added with ethyl acetate (30 mL) for extraction and separation, then aqueous phases were washed with ethyl acetate (30 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain the product tert-butyl 4-(3-chlorophenyl)-4-cyanopiperidine-1-carboxylate 25b (2.19 g) with a yield of 68.3%.

MS m/z (ESI): 338.1 [M+18]

### Step 2

### Tert-butyl 4-carbamoyl-4-(3-chlorophenyl)piperidine-1-carboxylate

Potassium hydroxide (770 mg, 13.7 mmol) and tert-butyl 4-(3-chlorophenyl)-4-cyanopiperidine-1-carboxylate 25b (2.2 g, 6.86 mmol) were added to a solution of dimethylsulphoxide (15 mL), and hydrogen peroxide (30%, 6.5 mL) was slowly added dropwise to the reaction solution. After the dropwise addition was completed, the reaction solution was stirred for 1 hour. After the reaction was completed, the reaction solution was added with 50 mL of water to precipitate a yellow solid, and filtered, then the filter cake was washed with water, and dried in vacuum to obtain the product tert-butyl 4-carbamoyl-4-(3-chlorophenyl)piperidine-1-carboxylate 25c (2.28 g) with a yield of 98.1%.

MS m/z (ESI): 260.9 [M+23]

### Step 3

### Tert-butyl 4-amino-4-(3-chlorophenyl)piperidine-1-carboxylate

Tert-butyl 4-carbamoyl-4-(3-chlorophenyl)piperidine-1-carboxylate 25c (2.28 g, 6.73 mmol) and [Bis(trifluoroacetoxy)iodo]benzene (3.18 g, 7.40 mmol) were added to a mixed solution of acetonitrile (15 mL) and water (15 mL) containing potassium hydroxide (566 mg, 10 mmol), and reacted at room temperature overnight. After the reaction was completed, the reaction solution was separated on a C₁₈ reversed phase column (C₁₈ separation column 20-45 μm; mobile phase A: H₂O, mobile phase B: CH₃CN) to obtain tert-butyl 4-amino-4-(3-chlorophenyl)piperidine-1-carboxylate 25d (0.9 g) with a yield of 43.1%.

MS m/z (ESI): 237.9 [M−72]

### Step 4

### 4-(3-chlorophenyl)piperidin-4-amine

Trifluoroacetic acid (1 mL) was dropwise added to 3 mL of dichloromethane solution containing tert-butyl 4-amino-4-(3-chlorophenyl)piperidine-1-carboxylate 25d (200 mg, 643 μmol), and reacted at room temperature for 1 hour. The reaction solution was concentrated under reduced pressure to obtain 4-(3-chlorophenyl)piperidin-4-amine 25e, which was directly used for the next reaction without purification.

MS m/z (ESI): 211.0 [M+1]

### Step 5

### 6-(4-amino-4-(3-chlorophenyl)piperidin-1-yl)-3-bromo-1H-pyrazolo[3,4-d]pyrimidine-4-carbonitrile

Diisopropylethylamine (91.4 mg, 707 μmol) and the above-mentioned crude product 4-(3-chlorophenyl)piperidin-4-amine 25e were added to a solution of N-methyl pyrrolidone (5 mL) containing 3-bromo-6-chloro-1H-pyrazolo[3,4-d]pyrimidine-4-carbonitrile 1d (150 mg, 580 μmol), heated to 100° C., and stirred for 1 hour. After the reaction was completed, the reaction solution was separated on a C₁₈ reversed phase column (C₁₈ separation column 20-45 μm; mobile phase A: H₂O, mobile phase B: CH₃CN) to obtain the product 6-(4-amino-4-(3-chlorophenyl)piperidin-1-yl)-3-bromo-1H-pyrazolo[3,4-d]pyrimidine-4-carbonitrile 25f (230 mg) with a yield of 75.2%.

MS m/z (ESI): 414.8 [M−16]

### Step 6

### 6-(4-amino-4-(3-chlorophenyl)piperidin-1-yl)-3-(2,3-dichlorophenyl)-1H-pyrazolo[3,4-d]pyrimidine-4-carbonitrile

6-(4-amino-4-(3-chlorophenyl)piperidin-1-yl)-3-bromo-1H-pyrazolo[3,4-d]pyrimidine-4-carbonitrile 25f (100 mg, 231 μmol), (2,3-dichlorophenyl)boronic acid 1g (176 mg, 924 μmol), methanesulfonato(2-dicyclohexylphosphino-2′,6′-di-isopropoxy-1,1′-biphenyl)(2-amino-1,1′-biphenyl-2-yl)palladium (38.7 mg, 46.2 μmol), 2-dicyclohexylphosphino-2′,6′-di-isopropoxy-1,1′-biphenyl (43 mg, 92 μmol) and potassium phosphate (147 mg, 693 μmol) were added to a mixed solution of 1,4-dioxane (5 mL) and water (1 mL), subjected to argon gas displacement thrice, then heated to 100° C., and reacted overnight. After the reaction was completed, the reaction solution was concentrated under reduced pressure, and added with ethyl acetate (10 mL) and water (10 mL) for extraction and separation, then aqueous phases were extracted with ethyl acetate (10 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain 6-(4-amino-4-(3-chlorophenyl)piperidin-1-yl)-3-(2,3-dichlorophenyl)-1H-pyrazolo[3,4-d]pyrimidine-4-carbonitrile 25g (75 mg) with a yield of 65.1%.

MS m/z (ESI): 480.8 [M−16]

### Step 7

### 6-(4-amino-4-(3-chlorophenyl)piperidin-1-yl)-3-(2,3-dichlorophenyl)-1H-pyrazolo[3,4-d]pyrimidine-4-carboxylic acid

6-(4-amino-4-(3-chlorophenyl)piperidin-1-yl)-3-(2,3-dichlorophenyl)-1H-pyrazolo[3,4-d]pyrimidine-4-carbonitrile 25g (75 mg, 150 μmol) was added to 3 mL of concentrated hydrochloric acid, and heated and refluxed for 1 hour. After the reaction was completed, the reaction solution was concentrated under reduced pressure, and subjected to liquid phase separation (separation column: AKZONOBEL Kromasil; 250×21.2 mm I.D.; 5 μM, 20 mL/min; mobile phase A: 0.05% TFA+H₂O, mobile phase B: CH₃CN) to obtain 6-(4-amino-4-(3-chlorophenyl)piperidin-1-yl)-3-(2,3-dichlorophenyl)-1H-pyrazolo[3,4-d]pyrimidine-4-carboxylic acid 25 (7 mg) with a yield of 4.7%.

MS m/z (ESI): 516.8 [M+1]

### Example 26

### 6-(4-amino-4-(1H-indazole-5-yl)piperidin-1-yl)-3-(2,3-dichlorophenyl)-1H-pyrazolo[3,4-d]pyrimidine-4-carboxamide

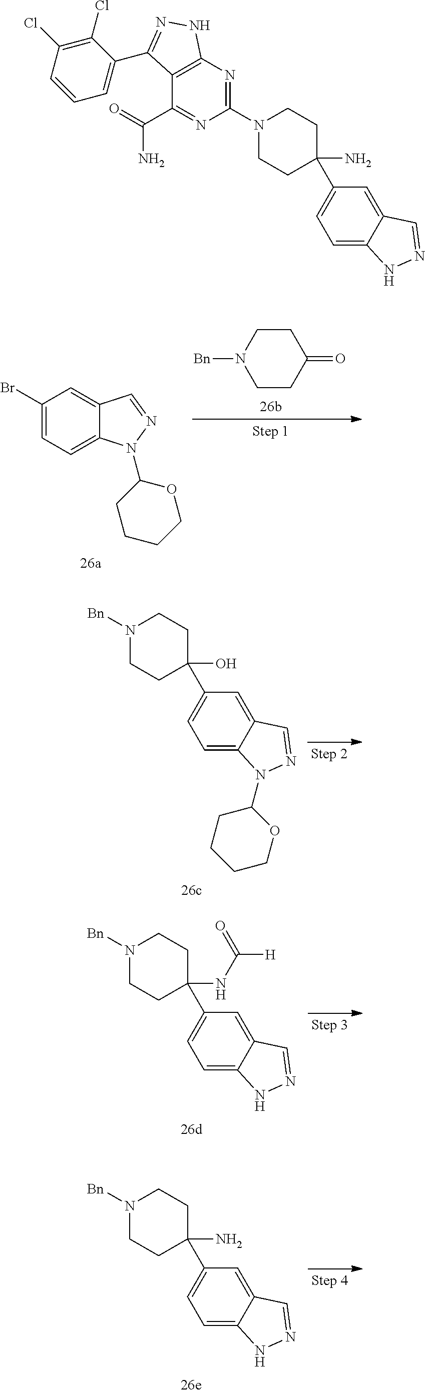 [SMILES: NC(=O)c1nc(N2CCC(N)(c3ccc4[nH]ncc4c3)CC2)nc2[nH]nc(-c3cccc(Cl)c3Cl)c12 ; O=C1CCN(Cc2ccccc2)CC1 ; Brc1ccc2c(cnn2C2CCCCO2)c1 ; OC1(c2ccc3c(cnn3C3CCCCO3)c2)CCN(Cc2ccccc2)CC1 ; O=CNC1(c2ccc3[nH]ncc3c2)CCN(Cc2ccccc2)CC1 ; NC1(c2ccc3[nH]ncc3c2)CCN(Cc2ccccc2)CC1]

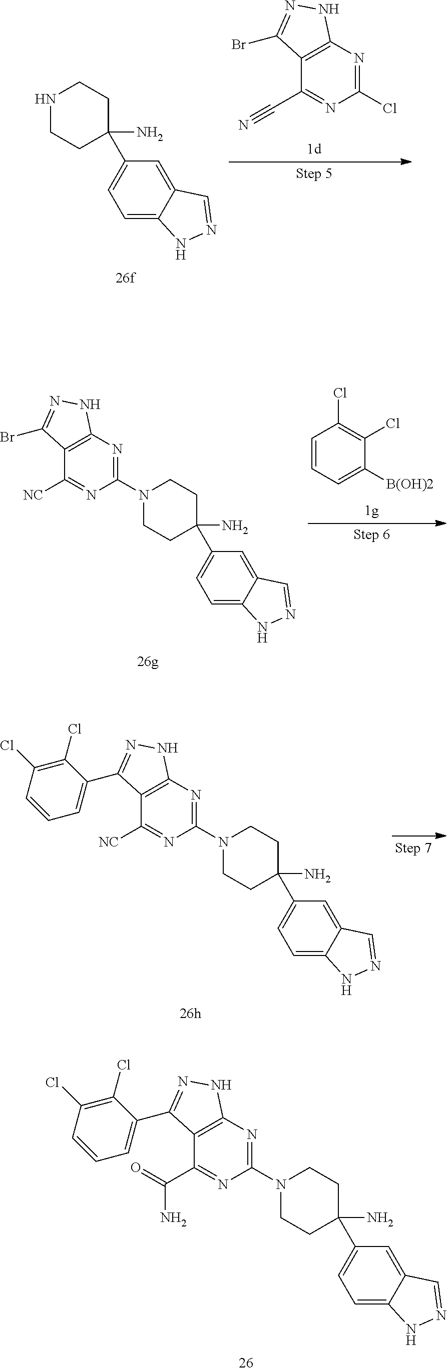 [SMILES: NC1(c2ccc3[nH]ncc3c2)CCNCC1 ; N#Cc1nc(Cl)nc2[nH]nc(Br)c12 ; N#Cc1nc(N2CCC(N)(c3ccc4[nH]ncc4c3)CC2)nc2[nH]nc(Br)c12 ; OB(O)c1cccc(Cl)c1Cl ; N#Cc1nc(N2CCC(N)(c3ccc4[nH]ncc4c3)CC2)nc2[nH]nc(-c3cccc(Cl)c3Cl)c12 ; NC(=O)c1nc(N2CCC(N)(c3ccc4[nH]ncc4c3)CC2)nc2[nH]nc(-c3cccc(Cl)c3Cl)c12]

### Step 1

### 1-benzyl-4-(1-(tetrahydro-2H-pyran-2-yl)-1H-indazole-5-yl)piperidin-4-ol

5-bromo-1-(tetrahydro-2H-pyran-2-yl)-1H-indazole 26a (321.16 mg, 1.70 mmol, prepared according to patent WO 2017060326) was added to 5 mL of tetrahydrofuran, cooled to −78° C., then dropwise added with n-butyl lithium (2.5 M, 783.22 μL), stirred at −78° C. for 0.5 hour, added with 1-benzylpiperidine-4-one 26b (321.16 mg, 1.70 mmol), stirred at −78° C. for 1 hour, and then heated to −20° C. After the reaction was completed, the reaction solution was quenched with a saturated ammonium chloride solution, extracted with ethyl acetate (10 mL×2), then organic phases were combined, dried with anhydrous sodium sulfate, filtered, and concentrated under reduced pressure. The obtained residue was further purified by silica gel column chromatography (eluent: system A) to obtain 1-benzyl-4-(1-(tetrahydro-2H-pyran-2-yl)-1H-indazole-5-yl)piperidin-4-ol 26c (350 mg) with a yield of 68.49%.

MS m/z (ESI): 392.1 [M+1]

### Step 2

### N-(1-benzyl-4-(1H-indazole-5-yl)piperidin-4-yl)carboxamide

1-benzyl-4-(1-(tetrahydro-2H-pyran-2-yl)-1H-indazole-5-yl)piperidin-4-ol 26c (1 g, 2.55 mmol) was added to 10 mL of trifluoroacetic acid, cooled to −15° C., added with trimethylsilyl cyanide 1.01 g, 10.22 mmol, 1.28 mL) and 2 mL of concentrated sulfuric acid, slowly heated to room temperature, and stirred overnight. After the reaction was completed, the reaction solution was poured into ice water to adjust the pH to be 7-8 with a 6N sodium hydroxide solution, extracted with dichloromethane (30 mL×3), dried and filtered, and concentrated under reduced pressure. The obtained residue was purified by silica gel column chromatography (eluent: system B) to obtain N-(1-benzyl-4-(1H-indazole-5-yl)piperidin-4-yl)carboxamide 26d (530 mg) with a yield of 62.05%.

MS m/z (ESI): 335.1 [M+1]

### Step 3

### 1-benzyl-4-(1H-indazole-5-yl)piperidine-4-amine

N-(1-benzyl-4-(1H-indazole-5-yl)piperidin-4-yl)carboxamide 26d (530 mg, 1.58 mmol) and 1 mL of concentrated hydrochloric acid were added to 2 mL of methanol, heated to 80° C., and reacted for 2 hours. The reaction solution was cooled to room temperature, and then concentrated under reduced pressure to obtain 1-benzyl-4-(1H-indazole-5-yl)piperidine-4-amine 26e (450 mg) with a yield of 92.67%, which was directly used for the next reaction without purification.

MS m/z (ESI): 307.0 [M+1]

### Step 4

### 4-(1H-indazole-5-yl)piperidine-4-amine

1-benzyl-4-(1H-indazole-5-yl)piperidine-4-amine 26e (500 mg, 1.63 mmol) and palladium hydroxide/carbon (180 mg, 1.63 mmol) were added to 15 mL of methanol, subjected to vacuum replacement thrice, and stirred at room temperature overnight. After the reaction was completed, the reaction solution is filtered through diatomite, and then concentrated under reduced pressure to obtain 4-(1H-indazole-5-yl)piperidine-4-amine 26f (200 mg) with a yield of 56.67%, which was directly used for the next reaction without purification.

MS m/z (ESI): 200.0 [M−16]

### Step 5

### 6-(4-amino-4-(1H-indazole-5-yl)piperidin-1-yl)-3-bromo-1H-pyrazolo[3,4-d]pyrimidine-4-carbonitrile

4-(1H-indazole-5-yl)piperidine-4-amine 26f (80.33 mg, 371.43 μmol), 3-bromo-6-chloro-1H-pyrazolo[3,4-d]pyrimidine-4-carbonitrile 1d (80 mg, 309.52 μmol) and N,N-diisopropylethylamine (120.01 mg, 928.57 μmol) were added to 2 mL of dimethylacetamide, heated to 95° C., and stirred for 2 hours. After the reaction was completed, the reaction solution was cooled to room temperature and concentrated under reduced pressure. The obtained residue was purified by silica gel column chromatography (eluent: system B) to obtain 6-(4-amino-4-(1H-indazole-5-yl)piperidin-1-yl)-3-bromo-1H-pyrazolo[3,4-d]pyrimidine-4-carbonitrile 26g (120 mg) with a yield of 88.46%.

MS m/z (ESI): 421.1 [M−16]

### Step 6

### 6-(4-amino-4-(1H-indazole-5-yl)piperidin-1-yl)-3-(2,3-dichlorophenyl)-1H-pyrazolo[3,4-d]pyrimidine-4-carbonitrile

6-(4-amino-4-(1H-indazole-5-yl)piperidin-1-yl)-3-bromo-1H-pyrazolo[3,4-d]pyrimidine-4-carbonitrile 26g (120 mg, 273.80 μmol), (2,3-dichlorophenyl)boronic acid 1g (208.98 mg, 1.10 mmol), methanesulfonato(2-dicyclohexylphosphino-2′,6′-di-isopropoxy-1,1′-biphenyl)(2-amino-1,1′-biphenyl-2-yl)palladium (45.85 mg, 54.76 μmol), 2-dicyclohexylphosphino-2′,6′-di-isopropoxy-1,1′-biphenyl (51.04 mg, 109.52 μmol) and potassium phosphate (174.13 mg, 821.39 μmol) were added to 5.5 mL of mixed solution (1,4-dioxane:water=10:1) in turn, subjected to argon gas displacement thrice, heated to 100° C., and stirred overnight. After the reaction was completed, the reaction solution was cooled to room temperature and concentrated under reduced pressure. The obtained residue was purified by silica gel column chromatography (eluent: system B) to obtain 6-(4-amino-4-(1H-indazole-5-yl)piperidin-1-yl)-3-(2,3-dichlorophenyl)-1H-pyrazolo[3,4-d]pyrimidine-4-carbonitrile 26h (80 mg) with a yield of 57.93%.

MS m/z (ESI): 487.0 [M−16]

### Step 7

### 6-(4-amino-4-(1H-indazole-5-yl)piperidin-1-yl)-3-(2,3-dichlorophenyl)-1H-pyrazolo[3,4-d]pyrimidine-4-carboxamide

6-(4-amino-4-(1H-indazole-5-yl)piperidin-1-yl)-3-(2,3-dichlorophenyl)-1H-pyrazolo[3,4-d]pyrimidine-4-carbonitrile 26h (80 mg, 158.61 μmol) and potassium hydroxide (17.80 mg, 317.23 μmol) were added to 2 mL of dimethyl sulfoxide, added with 1 mL of hydrogen peroxide in an ice water bath, heated to room temperature and stirred for 3 hours. After the reaction was completed, a small amount of trifluoroacetic acid was added in the reaction solution to adjust the pH to be 7-8, and then the reaction solution was concentrated under reduced pressure. The obtained residue was subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain the product 6-(4-amino-4-(1H-indazole-5-yl)piperidin-1-yl)-3-(2,3-dichlorophenyl)-1H-pyrazolo[3,4-d]pyrimidine-4-carboxamide 26 (19 mg) with a yield of 17.84%.

MS m/z (ESI): 505.1 [M−16]

### Example 27

### 6-(4-amino-4-phenylpiperidin-1-yl)-3-(2,3-dichloropyridin-4-yl)-1H-pyrazolo[3,4-d]pyrimidine-4-carboxamide

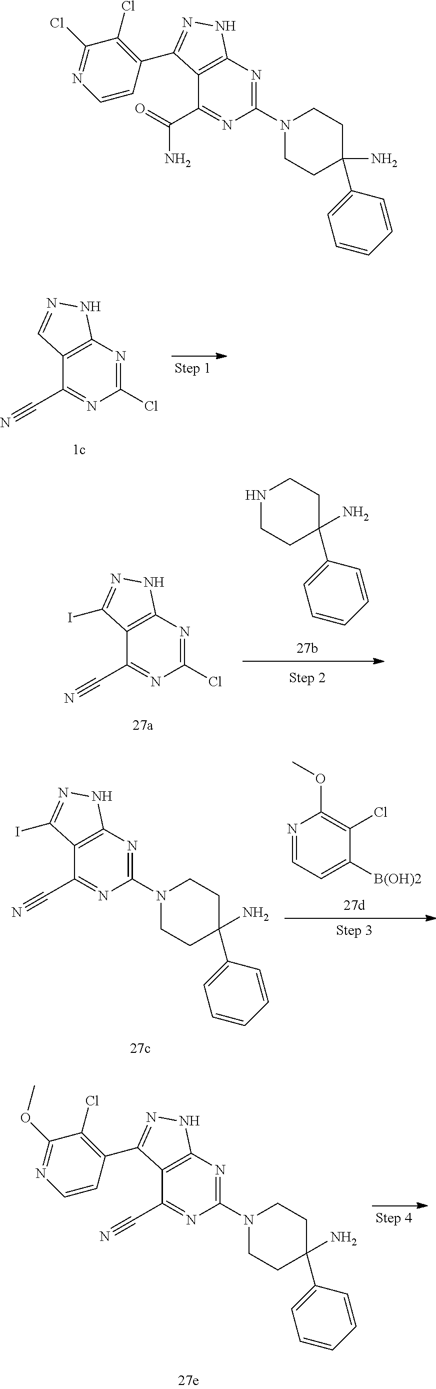

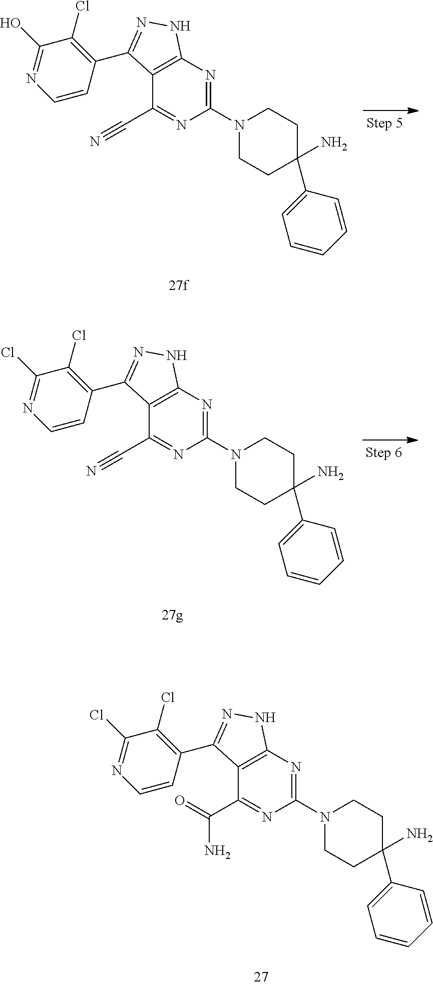 [SMILES: N#Cc1nc(N2CCC(N)(c3ccccc3)CC2)nc2[nH]nc(-c3ccnc(O)c3Cl)c12 ; N#Cc1nc(N2CCC(N)(c3ccccc3)CC2)nc2[nH]nc(-c3ccnc(Cl)c3Cl)c12 ; NC(=O)c1nc(N2CCC(N)(c3ccccc3)CC2)nc2[nH]nc(-c3ccnc(Cl)c3Cl)c12]

### Step 1

### 3-iodine-6-chloro-1H-pyrazolo[3,4-d]pyrimidine-4-carbonitrile

6-chloro-1H-pyrazolo[3,4-d]pyrimidine-4-carbonitrile 1c (290 mg, 1.62 mmol) and iodosuccinimide (726.69 mg, 3.23 mmol) were added to 10 mL of dichloroethane, heated to 80° C., and reacted for 4 hours. After the reaction was completed, the reaction solution was concentrated under reduced pressure. The obtained residue was purified by silica gel column chromatography (eluent: system A) to obtain 3-iodine-6-chloro-1H-pyrazolo[3,4-d]pyrimidine-4-carbonitrile 27a (250 mg) with a yield of 50.68%.

### Step 2

### 6-(4-amino-4-phenylpiperidin-1-yl)-3-iodine-1H-pyrazolo[3,4-d]pyrimidine-4-carbonitrile

4-phenylpiperidine-4-amine 27b (144.25 mg, 818.43 μmol), 3-iodine-6-chloro-1H-pyrazolo[3,4-d]pyrimidine-4-carbonitrile 27a (250 mg, 818.43 μmol) and N,N-diisopropylethylamine (317.32 mg, 2.46 mmol) were added to 3 mL of N,N-dimethylacetamide, heated to 90° C., and stirred for 2 hours. After the reaction was completed, the reaction solution was cooled to room temperature, and poured into ice water to precipitate a solid, then stirred for 10 minutes to collect the solid. Then the solid was dried under vacuum to obtain 6-(4-amino-4-phenylpiperidin-1-yl)-3-iodine-1H-pyrazolo[3,4-d]pyrimidine-4-carbonitrile 27c (300 mg) with a yield of 82.32%.

MS m/z (ESI): 428.8 [M−16]

### Step 3

### 6-(4-amino-4-phenylpiperidin-1-yl)-3-(3-chloro-2-methoxypyridin-4-yl)-1H-pyrazolo[3,4-d]pyrimidine-4-carbonitrile

6-(4-amino-4-phenylpiperidin-1-yl)-3-iodine-1H-pyrazolo[3,4-d]pyrimidine-4-carbonitrile 27c (150 mg, 336.88 μmol), (3-chloro-2-methoxypyridin-4-yl)boronic acid 27d (157.82 mg, 842.21 μmol), methanesulfonato(2-dicyclohexylphosphino-2′,6′-di-isopropoxy-1,1′-biphenyl)(2-amino-1,1′-biphenyl-2-yl)palladium (56.42 mg, 67.38 μmol), 2-dicyclohexylphosphino-2′,6′-di-isopropoxy-1,1′-biphenyl (62.79 mg, 134.75 μmol) and potassium phosphate (214.26 mg, 1.01 mmol) were added to 3.3 mL of mixed solution of (1,4-dioxane:water=10:1) in turn, subjected to argon gas displacement trice, heated to 100° C., and reacted for 2 hours. After the reaction was completed, the reaction solution was concentrated under reduced pressure. The obtained residue was purified by silica gel column chromatography (eluent: system B) to obtain 6-(4-amino-4-phenylpiperidin-1-yl)-3-(3-chloro-2-methoxypyridin-4-yl)-1H-pyrazolo[3,4-d]pyrimidine-4-carbonitrile 27e (40 mg) with a yield of 25.76%.

MS m/z (ESI): 443.8 [M−16]

### Step 4

### 6-(4-amino-4-phenylpiperidin-1-yl)-3-(3-chloro-2-hydroxypyridin-4-yl)-1H-pyrazolo[3,4-d]pyrimidine-4-carbonitrile

6-(4-amino-4-phenylpiperidin-1-yl)-3-(3-chloro-2-methoxypyridin-4-yl)-1H-pyrazolo[3,4-d]pyrimidine-4-carbonitrile 27e (40 mg, 86.78 μmol) and 2 mL of 4M hydrochloric acid dioxane solution were added to 1 mL of dichloromethane, heated to 50° C. and stirred for 4 hours. After the reaction was completed, the reaction solution was separated on a C₁₈ reversed phase column (C₁₈ separation column 20-45 μm; mobile phase A: H₂O, mobile phase B: CH₃CN) to obtain 6-(4-amino-4-phenylpiperidin-1-yl)-3-(3-chloro-2-hydroxypyridin-4-yl)-1H-pyrazolo[3,4-d]pyrimidine-4-carbonitrile 27f (30 mg) with a yield of 77.35%.

MS m/z (ESI): 429.9 [M−16]

### Step 5

### 6-(4-amino-4-phenylpiperidin-1-yl)-3-(2,3-dichloropyridin-4-yl)-1H-pyrazolo[3,4-d]pyrimidine-4-carbonitrile

6-(4-amino-4-phenylpiperidin-1-yl)-3-(3-chloro-2-hydroxypyridin-4-yl)-1H-pyrazolo[3,4-d]pyrimidine-4-carbonitrile 27f (30 mg, 67.13 μmol) was added to 1.5 mL of phosphorus oxychloride, heated to 110° C., and stirred overnight. After the reaction was completed, the reaction solution was cooled to room temperature, and poured to ice water, stirred for 0.5 hour, extracted with ethyl acetate (10 mL×3), then organic phases were combined and washed with a saturated sodium bicarbonate solution, dried with anhydrous sodium sulfate, filtered, and concentrated under reduced pressure to obtain 6-(4-amino-4-phenylpiperidin-1-yl)-3-(2,3-dichloropyridin-4-yl)-1H-pyrazolo[3,4-d]pyrimidine-4-carbonitrile 27g (30 mg) with a yield of 96.04%, which was directly used for the next reaction without purification.

MS m/z (ESI): 448.1 [M−16]

### Step 6

### 6-(4-amino-4-phenylpiperidin-1-yl)-3-(2,3-dichloropyridin-4-yl)-1H-pyrazolo[3,4-d]pyrimidine-4-carboxamide

6-(4-amino-4-phenylpiperidin-1-yl)-3-(2,3-dichloropyridin-4-yl)-1H-pyrazolo[3,4-d]pyrimidine-4-carbonitrile 27g (30 mg, 64.47 μmol), sodium hydroxide (5.16 mg, 128.94 μmol) and 0.5 mL of hydrogen peroxide were added to 1 mL of dimethyl sulfoxide, and stirred at room temperature for 1 hour. After the reaction was completed, a small amount of trifluoroacetic acid was added in the reaction solution to adjust the pH to be 7-8, and then the reaction solution was concentrated under reduced pressure. The obtained residue was subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain the product 6-(4-amino-4-phenylpiperidin-1-yl)-3-(2,3-dichloropyridin-4-yl)-1H-pyrazolo[3,4-d]pyrimidine-4-carboxamide 27 (4.1 mg) with a yield of 10.01%.

MS m/z (ESI): 482.8 [M+1]

¹HNMR (400 MHz, CD₃OD) δ 8.36 (d, J=4.9 Hz, 1H), 7.72 (d, J=7.7 Hz, 2H), 7.58 (t, J=7.6 Hz, 2H), 7.50 (s, 1H), 7.44 (d, J=4.8 Hz, 1H), 4.69 (d, J=14.2 Hz, 2H), 3.47 (t, J=12.2 Hz, 2H), 2.77 (d, J=13.8 Hz, 2H), 2.15 (ddd, J=14.2, 10.9, 3.8 Hz, 2H).

### Example 28

### 6-(4-amino-4-phenylpiperidin-1-yl)-3-(3-chloro-2-methoxypyridin-4-yl)-1H-pyrazolo[3,4-d]pyrimidine-4-carboxamide

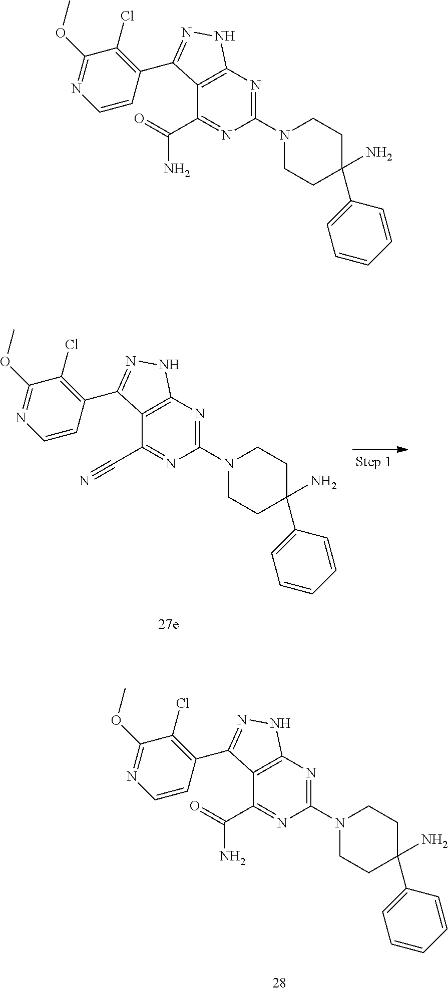 [SMILES: COc1nccc(-c2n[nH]c3nc(N4CCC(N)(c5ccccc5)CC4)nc(C(N)=O)c23)c1Cl ; COc1nccc(-c2n[nH]c3nc(N4CCC(N)(c5ccccc5)CC4)nc(C#N)c23)c1Cl ; COc1nccc(-c2n[nH]c3nc(N4CCC(N)(c5ccccc5)CC4)nc(C(N)=O)c23)c1Cl]

### Step 1

### 6-(4-amino-4-phenylpiperidin-1-yl)-3-(3-chloro-2-methoxypyridin-4-yl)-1H-pyrazolo[3,4-d]pyrimidine-4-carboxamide

6-(4-amino-4-phenylpiperidin-1-yl)-3-(3-chloro-2-methoxypyridin-4-yl)-1H-pyrazolo[3,4-d]pyrimidine-4-carbonitrile 27e was dissolved in methanol (10 mL), added with sodium hydroxide (99.82 mg, 2.50 mmol) and hydrogen peroxide (1 mL) in turn, and then stirred at room temperature for 2 hours. After the reaction was completed, a trifluoroacetic acid was used to adjust the pH to be 7, then the reaction solution was concentrated under reduced pressure, and subjected to separation to obtain 6-(4-amino-4-phenylpiperidin-1-yl)-3-(3-chloro-2-methoxypyridin-4-yl)-1H-pyrazolo[3,4-d]pyrimidine-4-carboxamide 28 (32 mg) with a yield of 10.38%.

MS m/z (ESI): 478.9 [M+1]

¹HNMR (400 MHz, CD₃OD) δ 8.06-8.12 (m, 1H), 7.72 (d, J=7.8 Hz, 2H), 7.57 (t, J=7.6 Hz, 2H), 7.51 (d, J=7.2 Hz, 1H), 6.99-7.05 (m, 1H), 4.68 (d, J=14.2 Hz, 2H), 4.01 (d, J=1.3 Hz, 3H), 3.46 (t, J=12.2 Hz, 2H), 2.77 (d, J=14.0 Hz, 2H), 2.08-2.23 (m, 2H).

### Example 29

### 6-(4-amino-4-phenylpiperidin-1-yl)-3-(2-chloro-3-methylphenyl)-1H-pyrazolo[3,4-d]pyrimidine-4-carboxamide

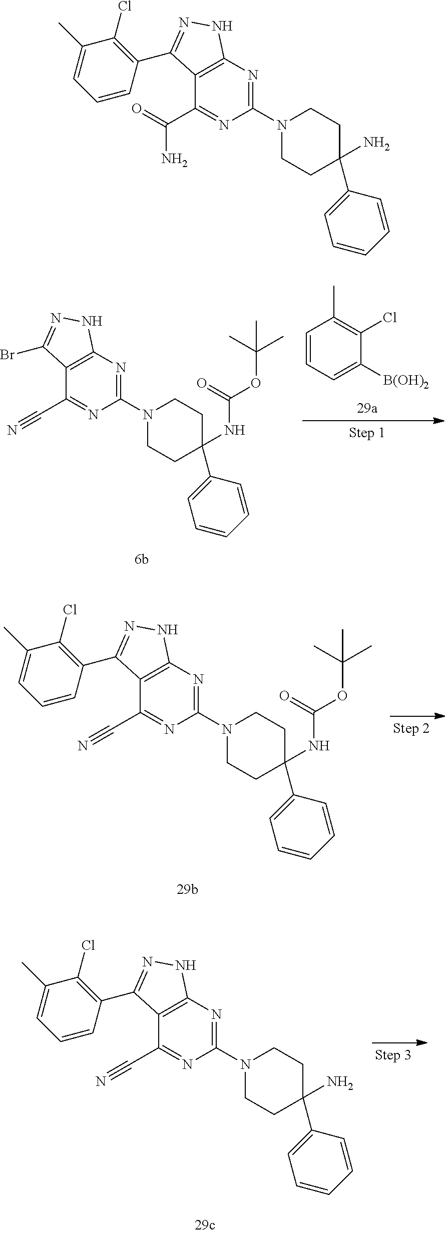

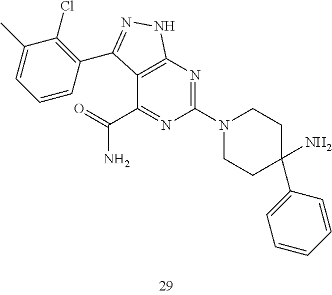 [SMILES: Cc1cccc(-c2n[nH]c3nc(N4CCC(N)(c5ccccc5)CC4)nc(C(N)=O)c23)c1Cl]

### Step 1

### Tert-butyl(1-(3-(2-chloro-3-methylphenyl)-4-cyano-1H-pyrazolo[3,4-d]pyrimidin-6-yl)-4-phenylpiperidin-4-yl)carbamate

Under the protection of argon gas, tert-butyl (1-(3-bromo-4-cyano-1H-pyrazolo[3,4-d]pyrimidin-6-yl)-4-phenylpiperidin-4-yl)carbamate 6b (100 mg, 200.65 μmol), (2-chloro-3-methylphenyl)boronic acid 29a (136.77 mg, 802.61 μmol), methanesulfonato(2-dicyclohexylphosphino-2′,6′-di-isopropoxy-1,1′-biphenyl)(2-amino-1,1′-biphenyl-2-yl)palladium (33.60 mg, 40.13 μmol), 2-dicyclohexylphosphino-2′,6′-di-isopropoxy-1,1′-biphenyl (37.40 mg, 80.26 μmol) and potassium phosphate (212.69 mg, 1.00 mmol) were added to 11 mL of mixed solution (1,4-dioxane:water=10:1) in turn, heated to 130° C., and reacted for 3 hours. After the reaction was completed, the reaction solution was concentrated under reduced pressure. The obtained residue was further analyzed and purified by silica gel column chromatography (eluent: system A) to obtain tert-butyl (1-(3-(2-chloro-3-methylphenyl)-4-cyano-1H-pyrazolo[3,4-d]pyrimidin-6-yl)-4-phenylpiperidin-4-yl)carbamate 29b (100 mg) with a yield of 91.61%.

MS m/z (ESI): 543.9 [M+1]

### Step 2

### 6-(4-amino-4-phenylpiperidin-1-yl)-3-(2-chloro-3-methylphenyl)-1H-pyrazolo[3,4-d]pyrimidine-4-carbonitrile

Tert-butyl (1-(3-(2-chloro-3-methylphenyl)-4-cyano-1H-pyrazolo[3,4-d]pyrimidin-6-yl)-4-phenylpiperidin-4-yl)carbamate 29b (100 mg, 183.81 μmol) and trifluoroacetic acid (0.5 mL) were added to dichloromethane (1.5 mL) in turn, and reacted at room temperature for 1 hour. After the reaction was completed, the reaction solution was concentrated under reduced pressure to obtain 6-(4-amino-4-phenylpiperidin-1-yl)-3-(2-chloro-3-methylphenyl)-1H-pyrazolo[3,4-d]pyrimidine-4-carbonitrile 29c (81 mg) with a yield of 99.27%, which was directly used for the next reaction without purification.

MS m/z (ESI): 426.9 [M−16]

### Step 3

### 6-(4-amino-4-phenylpiperidin-1-yl)-3-(2-chloro-3-methylphenyl)-1H-pyrazolo[3,4-d]pyrimidine-4-carboxamide

6-(4-amino-4-phenylpiperidin-1-yl)-3-(2-chloro-3-methylphenyl)-1H-pyrazolo[3,4-d]pyrimidine-4-carbonitrile 29c (81 mg, 182.46 μmol) and sodium hydroxide (43.79 mg, 1.09 mmol) were added to methanol (2 mL), added with hydrogen peroxide (0.2 mL), and stirred at room temperature for 2 hours. After the reaction was completed, a trifluoroacetic acid was used to adjust the pH to be 7, then the reaction solution was concentrated under reduced pressure, and subjected to separation to obtain 6-(4-amino-4-phenylpiperidin-1-yl)-3-(2-chloro-3-methylphenyl)-1H-pyrazolo[3,4-d]pyrimidine-4-carboxamide 29 (50 mg) with a yield of 47.37%.

MS m/z (ESI): 462.2 [M+1]

¹HNMR (400 MHz, CD₃OD) δ 7.72 (d, J=7.8 Hz, 2H), 7.57 (t, J=7.6 Hz, 2H), 7.50 (d, J=7.3 Hz, 1H), 7.34 (d, J=7.1 Hz, 1H), 7.21-7.30 (m, 2H), 4.67 (d, J=14.4 Hz, 2H), 3.45 (t, J=12.0 Hz, 2H), 2.76 (d, J=13.9 Hz, 2H), 2.40 (s, 3H), 2.16 (td, J=10.2, 5.0 Hz, 2H).

### Example 30

### 1-(3-(2,3-dichlorophenyl)-4-(1H-tetrazol-5-yl)-1H-pyrazolo[3,4-d]pyrimidin-6-yl)-4-phenylpiperidin-4-amine

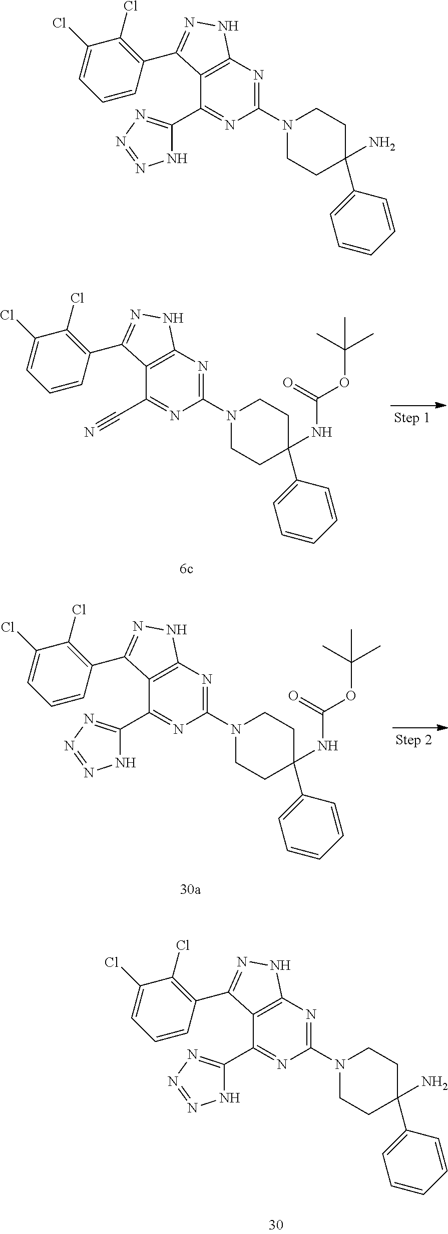 [SMILES: NC1(c2ccccc2)CCN(c2nc(-c3nnn[nH]3)c3c(-c4cccc(Cl)c4Cl)n[nH]c3n2)CC1 ; CC(C)(C)OC(=O)NC1(c2ccccc2)CCN(c2nc(C#N)c3c(-c4cccc(Cl)c4Cl)n[nH]c3n2)CC1 ; CC(C)(C)OC(=O)NC1(c2ccccc2)CCN(c2nc(-c3nnn[nH]3)c3c(-c4cccc(Cl)c4Cl)n[nH]c3n2)CC1 ; NC1(c2ccccc2)CCN(c2nc(-c3nnn[nH]3)c3c(-c4cccc(Cl)c4Cl)n[nH]c3n2)CC1]

### Step 1

### Tert-butyl (1-(3-(2,3-dichlorophenyl)-4-(1H-tetrazol-5-yl)-1H-pyrazolo[3,4-d]pyrimidin-6-yl)-4-phenylpiperidin-4-yl) carbamate

Tert-butyl(1-(4-cyano-3-(2,3-dichlorophenyl)-1H-pyrazolo[3,4-d]pyrimidin-6-yl)-4-phenylpiperidin-4-yl)carbamate 6c (200 mg, 354.32 μmol), sodium azide (230.31 mg, 3.54 mmol) and ammonium chloride (191.33 mg, 3.54 mmol) were added to N,N-dimethylformamide (3 mL), heated to 130° C., and reacted for 6 hours. After the reaction was completed, the reaction solution was cooled to room temperature, then an appropriate amount of water was added to the reaction solution to quench the reaction, a solid was precipitated, then the solid was filtered and dried to obtain tert-butyl (1-(3-(2,3-dichlorophenyl)-4-(1H-tetrazol-5-yl)-1H-pyrazolo[3,4-d]pyrimidin-6-yl)-4-phenylpiperidine-4-yl)carbamate 30a (150 mg) with a yield of 69.69%, which was directly used for the next reaction without purification.

MS m/z (ESI): 607.2 [M+1]

### Step 2

### 1-(3-(2,3-dichlorophenyl)-4-(1H-tetrazol-5-yl)-1H-pyrazolo[3,4-d]pyrimidin-6-yl)-4-phenylpiperidin-4-amine

Tert-butyl (1-(3-(2,3-dichlorophenyl)-4-(1H-tetrazol-5-yl)-1H-pyrazolo[3,4-d]pyrimidin-6-yl)-4-phenylpiperidin-4-yl)carbamate 30a (150 mg, 246.92 μmol) and trifluoroacetic acid (0.5 mL) were added to dichloromethane (2 mL), and stirred at room temperature for 2 hours. After the reaction was completed, the reaction solution was concentrated under reduced pressure, and subjected to separation to obtain 1-(3-(2,3-dichlorophenyl)-4-(1H-tetrazol-5-yl)-1H-pyrazolo[3,4-d]pyrimidin-6-yl)-4-phenylpiperidin-4-amine 30 (24 mg) with a yield of 15.33%.

MS m/z (ESI): 507.1 [M+1]

¹H NMR (400 MHz, CD₃OD) δ 7.73 (d, J=7.9 Hz, 2H), 7.59 (q, J=8.1, 7.6 Hz, 3H), 7.51 (d, J=7.3 Hz, 1H), 7.43 (d, J=7.6 Hz, 1H), 7.38 (d, J=7.8 Hz, 1H), 4.73 (d, J=14.1 Hz, 2H), 3.49 (t, J=12.1 Hz, 2H), 2.78 (d, J=14.0 Hz, 2H), 2.18 (td, J=11.0, 10.6, 5.5 Hz, 2H).

### Example 32

### 6-(4-amino-4-(2-fluorophenyl)piperidin-1-yl)-3-(3-chloro-2-methoxypyridin-4-yl)-1H-pyrazolo[3,4-d]pyrimidine-4-carboxamide

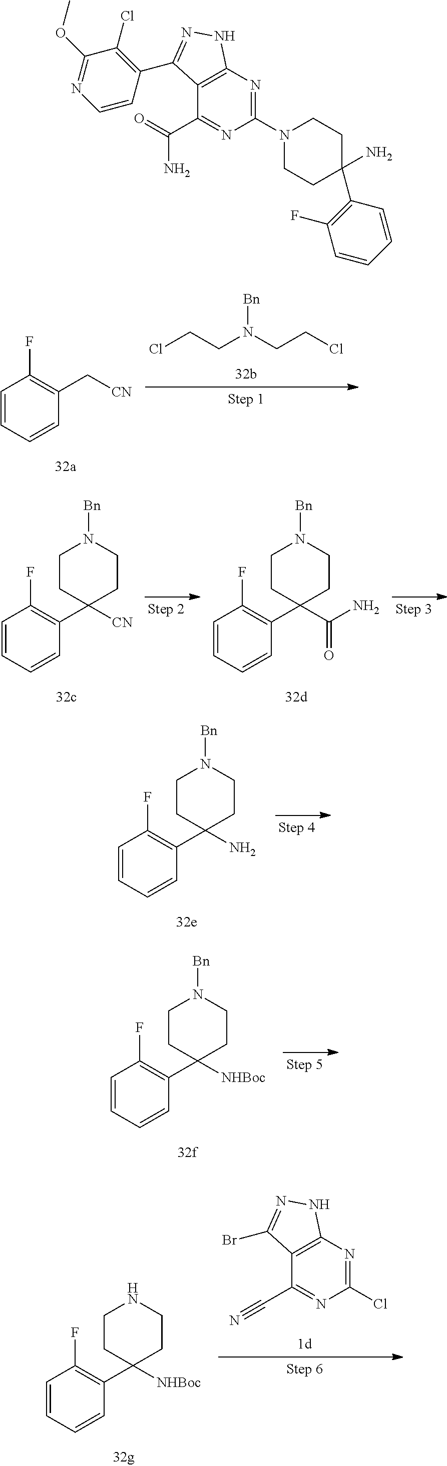

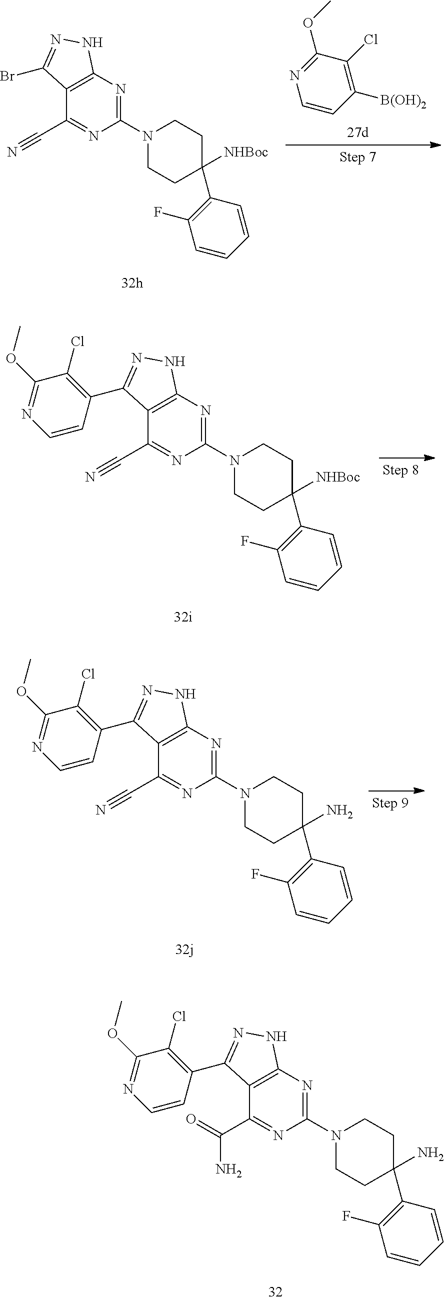 [SMILES: CC(C)(C)OC(=O)NC1(c2ccccc2F)CCN(c2nc(C#N)c3c(Br)n[nH]c3n2)CC1 ; COc1nccc(B(O)O)c1Cl ; COc1nccc(-c2n[nH]c3nc(N4CCC(NC(=O)OC(C)(C)C)(c5ccccc5F)CC4)nc(C#N)c23)c1Cl ; COc1nccc(-c2n[nH]c3nc(N4CCC(N)(c5ccccc5F)CC4)nc(C#N)c23)c1Cl ; COc1nccc(-c2n[nH]c3nc(N4CCC(N)(c5ccccc5F)CC4)nc(C(N)=O)c23)c1Cl]

### Step 1

### 1-benzyl-4-(2-fluorophenyl) piperidine-4-carbonitrile

In an ice water bath, sodium hydride (3.55 g, 88.80 mmol) was added to N,N-dimethylformamide (10 mL) containing 2-(2-fluorophenyl)acetonitrile 32a (2 g, 14.80 mmol) and N-benzyl-2-chloro-N-(2-chloroethyl)ethan-1-amine 32b (3.78 g, 16.28 mmol), stirred in an ice bath for 1 hour, heated to 60° C., and then stirred for 16 hours. After the reaction was completed, the reaction solution was poured to 100 mL of ice water, and stirred for 0.5 hour to collect a solid, then beated with petroleum ether, filtered and dried to obtain a solid. The mothor liquor was extracted with ethyl acetate, dried by anhydrous sodium sulfate, and then concentrated under reduced pressure. The obtained residue was further analyzed and purified by silica gel column chromatography (eluent: system A) to obtain 1-benzyl-4-(2-fluorophenyl) piperidine-4-carbonitrile 32c (4.1 g) with a yield of 94.11%.

MS m/z (ESI): 295.0 [M+1]

### Step 2

### 1-benzyl-4-(2-fluorophenyl)piperidine-4-carboxamide

Potassium hydroxide (1.56 g, 27.86 mmol) and 1-benzyl-4-(2-fluorophenyl) piperidine-4-carbonitrile 32c (4.1 g, 13.93 mmol) were added to a solution of dimethyl sulfoxide (10 mL), and hydrogen peroxide (10 mL) was slowly added dropwise to the reaction solution. After the dropwise addition was completed, the reaction solution was stirred at room temperature for 5 hours. After the reaction was completed, the reaction solution was added with water (50 mL) to precipitate a yellow solid, and filtered, then the filter cake was washed with water, and dried in vacuum to obtain 1-benzyl-4-(2-fluorophenyl)piperidine-4-carboxamide 32d (3.1 g) with a yield of 71.25%.

MS m/z (ESI): 313.1 [M+1]

### Step 3

### 1-benzyl-4-(2-fluorophenyl)piperidin-4-amine

Potassium hydroxide (2.51 g, 44.66 mmol) was added to a mixed solution of acetonitrile (20 mL) and water (30 mL) containing 1-benzyl-4-(2-fluorophenyl)piperidine-4-carboxamide 32d (3.1 g, 9.92 mmol) and then added with 1,3-dibromo-5,5-dimethylhydantoin (1.56 g, 5.46 mmol) in a water bath in batches, and stirred at room temperature for 1 hour. After the reaction was completed, the reaction solution was added with sodium sulfite (125.04 mg, 992.38 μmol) and stirred at room temperature for 15 minutes, then added with ethyl acetate (50 mL) and potassium phosphate (1.32 g, 10.92 mmol) for liquid separation, aqueous phases were extracted with ethyl acetate (50 mL×2), organic phases were combined and washed with a sodium chloride solution, and dried. The obtained residue was further analyzed and purified by silica gel column chromatography (eluent: system A) to obtain 1-benzyl-4-(2-fluorophenyl)piperidin-4-amine 32e (2.4 g) with a yield of 85.05%.

MS m/z (ESI): 285.1 [M+1]

### Step 4

### Tert-butyl (1-benzyl-4-(2-fluorophenyl)piperidin-4-yl)carbamate

1-benzyl-4-(2-fluorophenyl)piperidin-4-amine 32e (2.4 g, 8.44 mmol), sodium hydroxide (405.11 mg, 10.13 mmol) and di-tert-butyl dicarbonate (3.68 g, 16.88 mmol) were added to a mixed solution of water (5 mL) and 1,4-dioxane (10 mL), and stirred at room temperature for 3 hours. After the reaction was completed, the reaction solution was filtered, and the filtrate was extracted with ethyl acetate (50 mL×3), dried with anhydrous sodium sulfate, and then concentrated under reduced pressure. The obtained residue was further analyzed and purified by silica gel column chromatography (eluent: system A) to obtain tert-butyl (1-benzyl-4-(2-fluorophenyl)piperidin-4-yl)carbamate 32f (1.9 g) with a yield of 58.55%.

MS m/z (ESI): 385.1 [M+1]

### Step 5

### Tert-butyl (4-(2-fluorophenyl)piperidin-4-yl)carbamate

Tert-butyl (1-benzyl-4-(2-fluorophenyl)piperidin-4-yl)carbamate 32f (900 mg, 2.34 mmol) and 10% palladium carbon (450 mg) were added to methanol (30 mL), and hydrogenated at room temperature and pressure in hydrogen for 18 hours. After the reaction was completed, the reaction solution was filtered by diatomite to remove Pd/C, rinsed with methanol, and then concentrated under reduced pressure to obtain tert-butyl (4-(2-fluorophenyl)piperidin-4-yl)carbamate 32g (689 mg) with a yield of 99.99%, which was directly used for the next reaction without purification.

MS m/z (ESI): 295.0 [M+1]

### Step 6

### Tert-butyl (1-(3-bromo-4-cyano-1H-pyrazolo[3,4-d]pyrimidin-6-yl)-4-(2-fluorophenyl)piperidin-4-yl)carbamate

N,N-diisopropylethylamine (737.60 mg, 5.71 mmol, 943.22 μL), tert-butyl (4-(2-fluorophenyl)piperidin-4-yl) carbamate 32g (560 mg, 1.90 mmol) and 3-bromo-6-chloro-1H-pyrazolo[3,4-d]pyrimidine-4-carbonitrile 1d (491.70 mg, 1.90 mmol) were added to a solution of N,N-dimethylacetamide (3 mL), heated to 100° C., and stirred for 1 hour. After the reaction was completed, the mixed solution was poured with 100 mL of water and extracted with ethyl acetate (50 mL×3), then organic phases were combined, dried with anhydrous sodium sulfate, filtered, and concentrated under reduced pressure. The obtained residue was further analyzed and purified by silica gel column chromatography (eluent: system A) to obtain tert-butyl (1-(3-bromo-4-cyano-1H-pyrazolo[3,4-d]pyrimidin-6-yl)-4-(2-fluorophenyl)piperidin-4-yl)carbamate 32h (600 mg) with a yield of 61.08%.

MS m/z (ESI): 516.1 [M+1]

### Step 7

### Tert-butyl(1-(3-(3-chloro-2-methoxypyridin-4-yl)-4-cyano-1H-pyrazolo[3,4-d]pyrimidin-6-yl)-4-(2-fluorophenyl)piperidin-4-yl)carbamate

Tert-butyl(1-(3-bromo-4-cyano-1H-pyrazolo[3,4-d]pyrimidin-6-yl)-4-(2-fluorophenyl)piperidin-4-yl) carbamate 32h (250 mg, 484.15 μmol), (3-chloro-2-methoxypyridin-4-yl)boronic acid 27d (272.17 mg, 1.45 mmol), methanesulfonato(2-dicyclohexylphosphino-2′,6′-di-isopropoxy-1,1′-biphenyl)(2-amino-1,1′-biphenyl-2-yl)palladium (162.17 mg, 193.66 μmol), 2-dicyclohexylphosphino-2′,6′-di-isopropoxy-1,1′-biphenyl (180.49 mg, 387.32 μmol) and potassium phosphate (513.20 mg, 2.42 mmol) were added to a mixed solution of 1,4-dioxane (6 mL) and water (0.6 mL), subjected to argon gas displacement thrice, heated to 130° C., and reacted for 4.5 hours. After the reaction was completed, the reaction solution was concentrated under reduced pressure. The obtained residue was further analyzed and purified by silica gel column chromatography (eluent: system A) to obtain tert-butyl(1-(3-(3-chloro-2-methoxypyridin-4-yl)-4-cyano-1H-pyrazolo[3,4-d]pyrimidin-6-yl)-4-(2-fluorophenyl)piperidin-4-yl)carbamate 32i (160 mg) with a yield of 57.07%.

MS m/z (ESI): 578.9 [M+1]

### Step 8

### 6-(4-amino-4-(2-fluorophenyl)piperidin-1-yl)-3-(3-chloro-2-methoxypyridin-4-yl)-1H-pyrazolo[3,4-d]pyrimidine-4-carbonitrile

Tert-butyl(1-(3-(3-chloro-2-methoxypyridin-4-yl)-4-cyano-1H-pyrazolo[3,4-d]pyrimidin-6-yl)-4-(2-fluorophenyl)piperidin-4-yl)tert-butyl carbamate 32i (30 mg, 51.81 μmol) and trifluoroacetic acid (1 mL) were added to a solution of dichloromethane (3 mL), and stirred at room temperature for 1 hour. After the reaction was completed, the reaction solution was concentrated under reduced pressure to obtain 6-(4-amino-4-(2-fluorophenyl)piperidin-1-yl)-3-(3-chloro-2-methoxypyridin-4-yl)-1H-pyrazolo[3,4-d]pyrimidine-4-carbonitrile 32j, which was directly used for the next reaction without purification.

MS m/z (ESI): 462.1 [M−16]

### Step 9

### 6-(4-amino-4-(2-fluorophenyl)piperidin-1-yl)-3-(3-chloro-2-methoxypyridin-4-yl)-1H-pyrazolo[3,4-d]pyrimidine-4-carboxamide

The above-mentioned crude product 6-(4-amino-4-(2-fluorophenyl)piperidin-1-yl)-3-(3-chloro-2-methoxypyridin-4-yl)-1H-pyrazolo[3,4-d]pyrimidine-4-carbonitrile 32j was dissolved in methanol (3 mL), added with sodium hydroxide (10.36 mg, 258.92 μmol) and hydrogen peroxide (0.3 mL) in turn, and then stirred at room temperature for 2 hours. After the reaction was completed, a trifluoroacetic acid was used to adjust the pH to be 7, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2-fluorophenyl)piperidin-1-yl)-3-(3-chloro-2-methoxypyridin-4-yl)-1H-pyrazolo[3,4-d]pyrimidine-4-carboxamide 32 (7 mg) with a yield of 27.2%.

MS m/z (ESI): 497.2 [M+1]

¹H NMR (400 MHz, CD₃OD) δ 8.01 (d, J=5.0 Hz, 1H), 7.61 (s, 1H), 7.47 (dd, J=5.9, 2.0 Hz, 1H), 7.17-7.32 (m, 2H), 6.94 (d, J=5.1 Hz, 1H), 4.35-4.45 (m, 2H), 3.92 (s, 3H), 3.64 (ddd, J=13.6, 9.7, 3.1 Hz, 2H), 2.64-2.73 (m, 2H), 2.06-2.15 (m, 2H).

### Example 33

### 6-(4-amino-4-(2-(trifluoromethyl)phenyl)piperidin-1-yl)-3-(2,3-dichlorophenyl)-1H-pyrazolo[3,4-d]pyrimidine-4-carboxamide

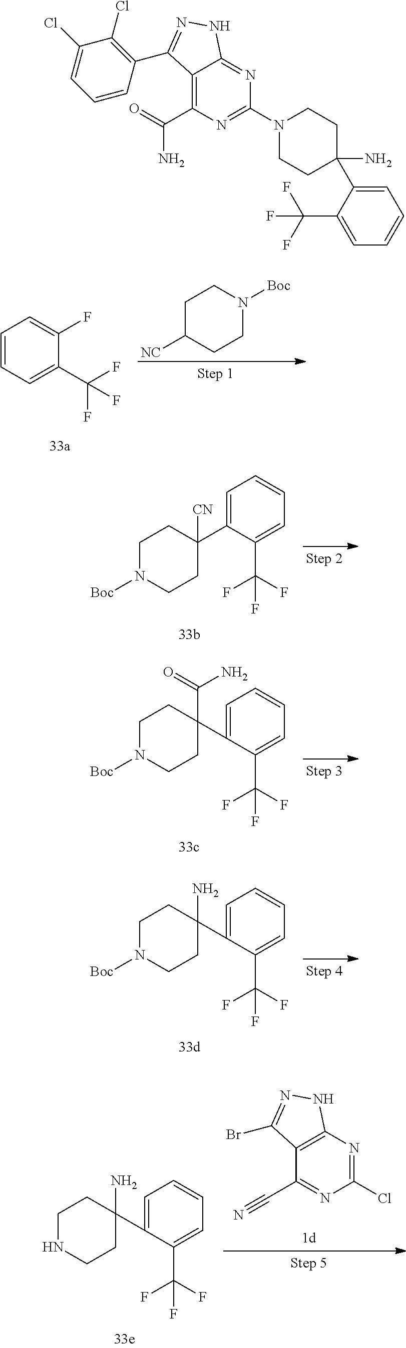

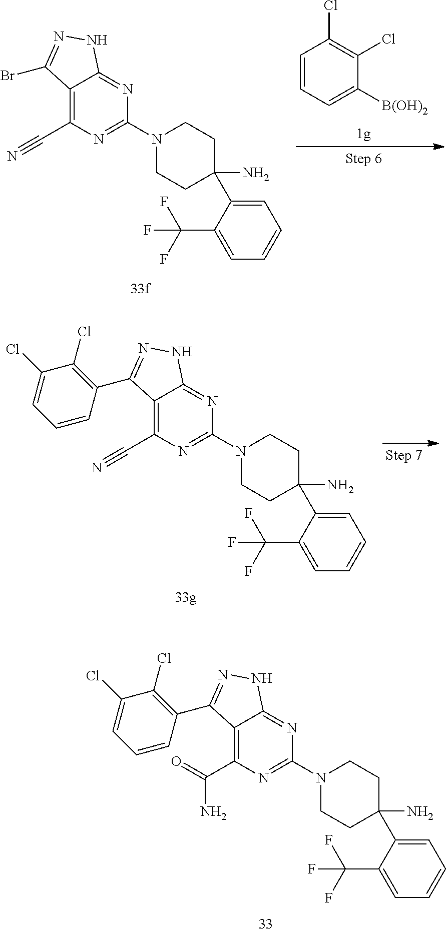 [SMILES: N#Cc1nc(N2CCC(N)(c3ccccc3C(F)(F)F)CC2)nc2[nH]nc(Br)c12 ; OB(O)c1cccc(Cl)c1Cl ; N#Cc1nc(N2CCC(N)(c3ccccc3C(F)(F)F)CC2)nc2[nH]nc(-c3cccc(Cl)c3Cl)c12 ; NC(=O)c1nc(N2CCC(N)(c3ccccc3C(F)(F)F)CC2)nc2[nH]nc(-c3cccc(Cl)c3Cl)c12]

### Step 1

### Tert-butyl 4-cyano-4-(2-(trifluoromethyl)phenyl)piperidine-1-carboxylate

1-fluoro-2-(trifluoromethyl)benzene 33a (1.56 g, 9.51 mmol), tert-butyl 4-cyanopiperidine-1-carboxylate (2 g, 9.51 mmol) and a 1 M tetrahydrofuran solution (2.85 g, 14.27 mmol) containing potassium bis(trimethylsilyl)amide were added to 20 mL of toluene, heated to 70° C., and reacted overnight. After the reaction was completed, the reaction solution was added with 20 mL of water and ethyl acetate (20 mL×3) for extraction, and then washed with a saturated sodium chloride solution (20 mL). Organic phases were dried with anhydrous sodium sulfate, filtered, concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, and mobile phase B: CH₃CN) to obtain tert-butyl 4-cyano-4-(2-(trifluoromethyl)phenyl)piperidine-1-tert-carboxylate 33b (410 mg) with a yield of 12.16%.

MS m/z (ESI): 255.1 [M−99]

### Step 2

### Tert-butyl 4-carbamoyl-4-(2-(trifluoromethyl)phenyl)piperidine-1-carboxylate

Potassium hydroxide (129.52 mg, 2.31 mmol) and tert-butyl 4-cyano-4-(2-(trifluoromethyl)phenyl)piperidine-1-carboxylate 33b (409 mg, 1.15 mmol) were added to a solution of dimethyl sulfoxide (2 mL), and hydrogen peroxide (0.8 mL) was slowly added to the reaction solution in a water bath, then the reaction solution was heated to room temperature, and reacted for 30 minutes. After the reaction was completed, the reaction solution was added with 100 mL of water to precipitate a large amount of solids, and the liquid was removed by filtration to obtain tert-butyl 4-carbamoyl-4-(2-(trifluoromethyl)phenyl)piperidine-1-carboxylate 33c (400 mg) with a yield of 93.07%.

MS m/z (ESI): 317.1 [M−55]

### Step 3

### Tert-butyl 4-amino-4-(2-(trifluoromethyl)phenyl)piperidine-1-carboxylate

Potassium hydroxide (13.56 mg, 241.69 μmol) and tert-butyl 4-carbamoyl-4-(2-(trifluoromethyl)phenyl)piperidine-1-carboxylate 33c (20 mg, 53.71 μmol) were added to a mixed solution of acetonitrile (5 mL) and water (5 mL), added with 1,3-dibromo-5,5-dimethylhydantoin (8.45 mg, 29.54 μmol) in a water bath in batches, and stirred at room temperature for 1 hour. After the reaction was completed, the reaction solution was concentrated under reduced pressure, and separated on a C₁₈ reversed phase chromatographic column (C₁₈ separation column 20-45 μm; mobile phase A: H₂O, mobile phase B: CH₃CN) to obtain tert-butyl 4-amino-4-(2-(trifluoromethyl)phenyl)piperidine-1-carboxylate 33d (18.5 mg) with a yield of 100%.

MS m/z (ESI): 289.1 [M−55]

### Step 4

### 4-(2-(trifluoromethyl)phenyl)piperidin-4-amine

Tert-butyl 4-amino-4-(2-(trifluoromethyl)phenyl)piperidine-1-carboxylate 33d (199 mg, 577.87 μmol) and 0.5 mL of trifluoroacetic acid were added to 2 mL of dichloromethane solution, and continuously reacted for 1 hour. After the reaction was completed, the reaction solution was concentrated under reduced pressure to obtain 4-(2-(trifluoromethyl)phenyl)piperidin-4-amine 33e (141 mg) with a yield of 99.9%, which was directly used for the next reaction without purification.

MS m/z (ESI): 228.1 [M−16]

### Step 5

### 6-(4-amino-4-(2-(trifluoromethyl)phenyl)piperidin-1-yl)-3-bromo-1H-pyrazolo[3,4-d]pyrimidine-4-carbonitrile

3-bromo-6-chloro-1H-pyrazolo[3,4-d]pyrimidine-4-carbonitrile 1d (136 mg, 526.19 μmol) was added to N-methyl pyrrolidone (2 mL), added with N,N-dimethylacetamide (203.47 mg, 1.57 mmol) and 4-(2-(trifluoromethyl)phenyl)piperidin-4-amine 33e (141 mg, 577.26 μmol), heated to 80° C., and reacted for 1 hour. After the reaction was completed, the reaction solution was concentrated under reduced pressure. The obtained residue was purified by silica gel column chromatography (eluent: system B) to obtain 6-(4-amino-4-(2-(trifluoromethyl)phenyl)piperidin-1-yl)-3-bromo-1H-pyrazolo[3,4-d]pyrimidine-4-carbonitrile 33f (190 mg) with a yield of 77.65%.

MS m/z (ESI): 449.0 [M−16]

### Step 6

### 6-(4-amino-4-(2-(trifluoromethyl)phenyl)piperidin-1-yl)-3-(2,3-dichlorophenyl)-1H-pyrazolo[3,4-d]pyrimidine-4-carbonitrile

6-(4-amino-4-(2-(trifluoromethyl)phenyl)piperidin-1-yl)-3-bromo-1H-pyrazolo[3,4-d]pyrimidine-4-carbonitrile 33f (190 mg, 407.50 μmol), (2,3-dichlorophenyl)boronic acid 1g (311.04 mg, 1.63 mmol), methanesulfonato(2-dicyclohexylphosphino-2′,6′-di-isopropoxy-1,1′-biphenyl)(2-amino-1,1′-biphenyl-2-yl)palladium (68.25 mg, 81.50 μmol), 2-dicyclohexylphosphino-2′,6′-di-isopropoxy-1,1′-biphenyl (76.06 mg, 163.00 μmol) and potassium phosphate (259.62 mg, 1.22 mmol) were added to a mixed solution of 1,4-dioxane (8 mL) and water (0.8 mL), subjected to argon gas displacement thrice, heated to 100° C., and reacted for 16 hours. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2-(trifluoromethyl)phenyl)piperidine-1-yl)-3-(2,3-dichlorophenyl)-1H-pyrazolo[3,4-d]pyrimidine-4-carbonitrile 33g (75 mg) with a yield of 34.57%.

MS m/z (ESI): 515.0 [M−16]

### Step 7

### 6-(4-amino-4-(2-(trifluoromethyl)phenyl)piperidin-1-yl)-3-(2,3-dichlorophenyl)-1H-pyrazolo[3,4-d]pyrimidine-4-carboxamide

5 M sodium hydroxide (0.5 mL) was added to a solution of methanol (1 mL) containing 6-(4-amino-4-(2-(trifluoromethyl)phenyl)piperidin-1-yl)-3-(2,3-dichlorophenyl)-1H-pyrazolo[3,4-d]pyrimidine-4-carbonitrile 33g (55 mg, 103.32 μmol), then added with hydrogen peroxide (0.5 mL), and stirred at room temperature for 1 hour. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2-(trifluoromethyl)phenyl)piperidin-1-yl)-3-(2,3-dichlorophenyl)-1H-pyrazolo[3,4-d]pyrimidine-4-carboxamide 33 (2 mg) with a yield of 3.5%.

MS m/z (ESI): 550.1 [M+1]
¹H NMR (400 MHz, DMSO-d₆) δ 13.58 (s, 1H), 8.48 (s, 2H), 8.14 (s, 1H), 7.97 (t, J=8.1 Hz, 2H), 7.85 (t, J=7.7 Hz, 1H), 7.73 (t, J=7.7 Hz, 1H), 7.61-7.68 (m, 2H), 7.40 (d, J=4.3 Hz, 2H), 4.24 (s, 2H), 3.75 (s, 2H), 2.67 (s, 2H), 2.22 (s, 2H).

### Example 34

### 6-(4-amino-4-(2-(trifluoromethyl)phenyl)piperidin-1-yl)-3-(2,3-dichlorophenyl)-1H-pyrazolo[3,4-d]pyrimidine-4-carboxylic acid

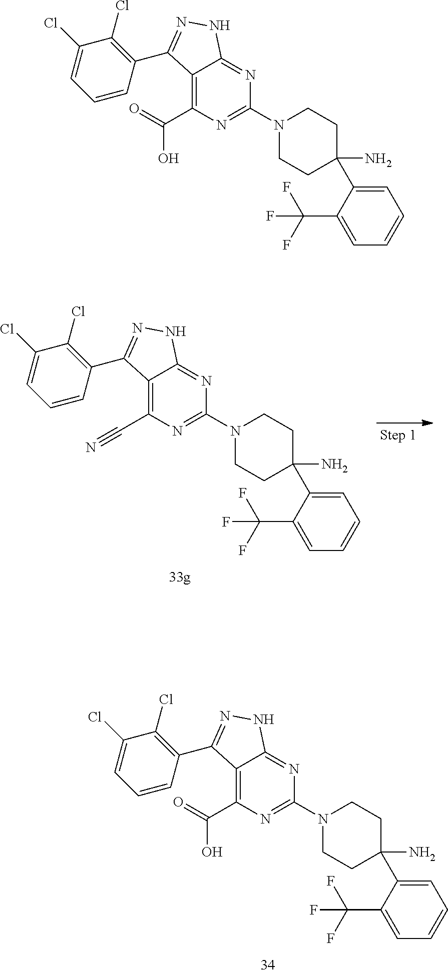 [SMILES: NC1(c2ccccc2C(F)(F)F)CCN(c2nc(C(=O)O)c3c(-c4cccc(Cl)c4Cl)n[nH]c3n2)CC1 ; N#Cc1nc(N2CCC(N)(c3ccccc3C(F)(F)F)CC2)nc2[nH]nc(-c3cccc(Cl)c3Cl)c12 ; NC1(c2ccccc2C(F)(F)F)CCN(c2nc(C(=O)O)c3c(-c4cccc(Cl)c4Cl)n[nH]c3n2)CC1]

6-(4-amino-4-(2-(trifluoromethyl)phenyl)piperidin-1-yl)-3-(2,3-dichlorophenyl)-1H-pyrazolo[3,4-d]pyrimidine-4-carbonitrile 33g (20 mg, 37.57 μmol) was added to concentrated hydrochloric acid (0.7 mL), sealed, heated to 100° C., and reacted for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2-(trifluoromethyl)phenyl)piperidin-1-yl)-3-(2,3-dichlorophenyl)-1H-pyrazolo[3,4-d]pyrimidine-4-carboxylic acid 34 (3 mg) with a yield of 14.4%.

MS m/z (ESI): 551.1 [M+1]

¹H NMR (400 MHz, DMSO-d₆) δ 13.67 (s, 1H), 8.50 (s, 2H), 7.97 (d, J=7.9 Hz, 2H), 7.82 (s, 1H), 7.71 (dd, J=6.3, 3.4 Hz, 3H), 7.45 (q, J=3.4, 2.6 Hz, 2H), 4.15 (s, 2H), 3.73 (s, 2H), 2.67 (s, 2H), 2.20 (s, 2H).

### Example 35

### 6-(4-amino-4-(pyridin-4-ylmethyl)piperidin-1-yl)-3-(2,3-dichlorophenyl)-1H-pyrazolo[3,4-d]pyrimidine-4-carboxamide

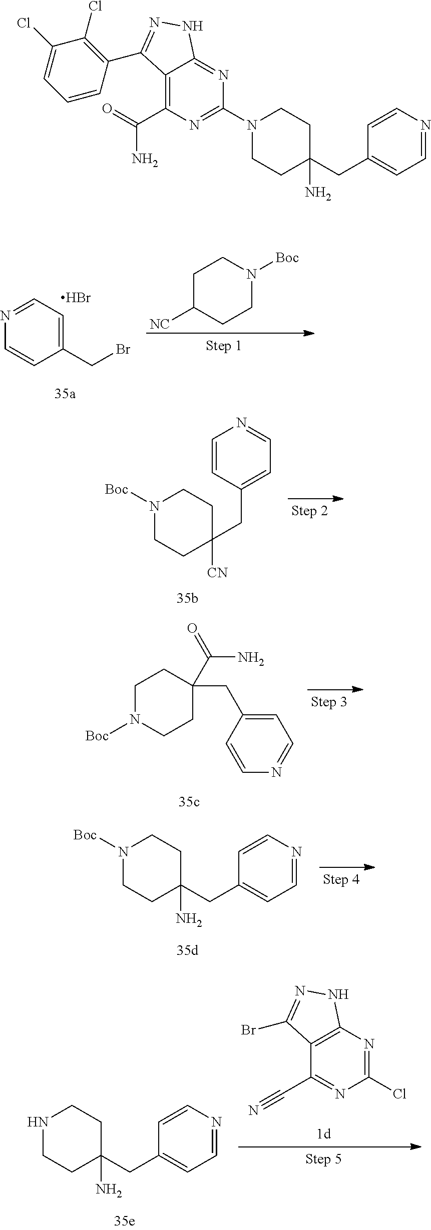

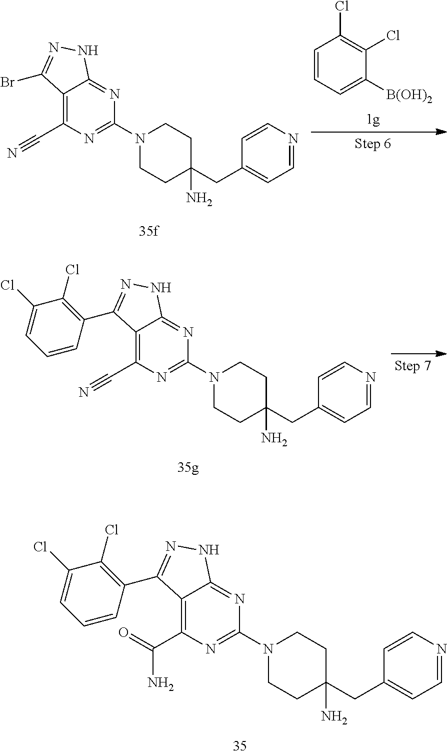 [SMILES: N#Cc1nc(N2CCC(N)(Cc3ccncc3)CC2)nc2[nH]nc(Br)c12 ; OB(O)c1cccc(Cl)c1Cl ; N#Cc1nc(N2CCC(N)(Cc3ccncc3)CC2)nc2[nH]nc(-c3cccc(Cl)c3Cl)c12 ; NC(=O)c1nc(N2CCC(N)(Cc3ccncc3)CC2)nc2[nH]nc(-c3cccc(Cl)c3Cl)c12]

### Step 1

### Tert-butyl 4-cyano-4-(pyridin-4-ylmethyl)piperidine-1l-carboxylate

4-(bromomethyl)pyridine hydrobromide 35a (1.06 g, 4.19 mmol),tert-butyl 4-cyanopiperidine-1-carboxylate (800 mg, 3.80 mmol) and N,N-diisopropylethylamine (639.22 mg, 4.95 mmol) were added to toluene (2 mL), stirred at room temperature for 15 minutes, cooled to 0° C., dropwise added with a 1 M tetrahydrofuran solution (834.83 mg, 4.19 mmol) containing potassium bis(trimethylsilyl)amide, then heated to room temperature, and reacted for 16 hours. After the reaction was completed, the reaction solution was added with 20 mL of saturated sodium chloride solution and extracted with ethyl acetate (20 mL×3), and then washed with a saturated sodium chloride solution (20 mL). Organic phases were dried with anhydrous sodium sulfate, filtered, concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, and mobile phase B: CH₃CN) to obtain tert-butyl 4-cyano-4-(pyridin-4-ylmethyl)piperidine-1-carboxylate 35b (630 mg) with a yield of 54.94%.

MS m/z (ESI): 302.0 [M+1]

### Step 2

### Tert-butyl 4-carbamoyl-4-(pyridin-4-ylmethyl)piperidine-1-carboxylate

Potassium hydroxide (234.58 mg, 4.18 mmol) and tert-butyl 4-cyano-4-(pyridin-4-ylmethyl)piperidine-1-carboxylate 35b (630 mg, 2.09 mmol) were added to dimethyl sulfoxide (2.3 mL), and hydrogen peroxide (1 mL) was slowly added to the reaction solution in a water bath, then the reaction solution was heated to room temperature, and reacted for 30 minutes. After the reaction was completed, the reaction solution was added with 100 mL of water, and extracted with ethyl acetate (20 mL×2), washed with a saturated sodium chloride solution (20 mL), dried with anhydrous sodium sulfate, and concentrated under reduced pressure to obtain tert-butyl 4-carbamoyl-4-(pyridin-4-ylmethyl)piperidine-1-carboxylate 35c (667 mg) with a yield of 99.90%, which was directly used for the next reaction without purification.

MS m/z (ESI): 320.0 [M+1]

### Step 3

### Tert-butyl 4-amino-4-(pyridin-4-ylmethyl)piperidine-1-carboxylate

Potassium hydroxide (527.29 mg, 9.40 mmol) and tert-butyl 4-carbamoyl-4-(pyridin-4-ylmethyl)piperidine-1-carboxylate 35c (667 mg, 2.09 mmol) were added to a mixed solution of acetonitrile (2 mL) and water (2 mL), added with 1,3-dibromo-5,5-dimethylhydantoin (328.40 mg, 1.15 mmol) in a water bath in batches, and stirred at room temperature for 1 hour. After the reaction was completed, the reaction solution was concentrated under reduced pressure, and separated on a C₁₈ reversed phase chromatographic column (C₁₈ separation column 20-45 μm; mobile phase A: H₂O, mobile phase B: CH₃CN) to obtain tert-butyl 4-amino-4-(pyridin-4-ylmethyl)piperidine-1-carboxylate 35d (500 mg) with a yield of 82.17%.

MS m/z (ESI): 292.2 [M+1]

### Step 4

### 4-(pyridin-4-ylmethyl)piperidin-4-amine

Tert-butyl 4-amino-4-(pyridin-4-ylmethyl)piperidine-1-carboxylate 35d (200 mg, 686.37 μmol) and 0.5 mL of trifluoroacetic acid were added to 2 mL of dichloromethane, and reacted for 1 hour. After the reaction was completed, the reaction solution was concentrated under reduced pressure to obtain 4-(pyridin-4-ylmethyl)piperidin-4-amine 35e (131 mg) with a yield of 99.78%, which was directly used for the next reaction without purification.

MS m/z (ESI): 192.1 [M+1]

### Step 5

### 6-(4-amino-4-(pyridin-4-ylmethyl)piperidin-1-yl)-3-bromo-1H-pyrazolo[3,4-d]pyrimidine-4-carbonitrile

3-bromo-6-chloro-1H-pyrazolo[3,4-d]pyrimidine-4-carbonitrile 1d (147.51 mg, 570.74 μmol) was added to N,N-dimethylacetamide (3 mL), added with N,N-diisopropylethylamine (221.29 mg, 1.71 mmol) and 4-(pyridin-4-ylmethyl)piperidin-4-amine 35e (131 mg, 684.89 μmol), heated to 80° C., and reacted for 1 hour. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-4-ylmethyl)piperidin-1-yl)-3-bromo-1H-pyrazolo[3,4-d]pyrimidine-4-carbonitrile 35f (120 mg) with a yield of 50.88%.

MS m/z (ESI): 413.1 [M+1]

### Step 6

### 6-(4-amino-4-(pyridin-4-ylmethyl)piperidin-1-yl)-3-(2,3-dichlorophenyl)-1H-pyrazolo[3,4-d]pyrimidine-4-carbonitrile

6-(4-amino-4-(pyridin-4-ylmethyl)piperidin-1-yl)-3-bromo-1H-pyrazolo[3,4-d]pyrimidine-4-carbonitrile 35f (70.17 mg, 169.79 μmol), (2,3-dichlorophenyl)boronic acid 1g (129.59 mg, 679.14 μmol), methanesulfonato(2-dicyclohexylphosphino-2′,6′-di-isopropoxy-1,1′-biphenyl)(2-amino-1,1′-biphenyl-2-yl)palladium (28.43 mg, 33.96 μmol), 2-dicyclohexylphosphino-2′,6′-di-isopropoxy-1,1′-biphenyl (31.69 mg, 67.91 μmol) and potassium phosphate (108.17 mg, 509.36 μmol) were added to a mixed solution of 1,4-dioxane (2 mL) and water (0.2 mL), subjected to argon gas displacement thrice, heated to 100° C., and reacted for 16 hours. After the reaction was completed, the reaction solution was concentrated under reduced pressure and separated on a C¹⁸ reversed phase 97 hromatographic column (C¹⁸ separation column 20-45 μm; mobile phase A: H₂O, mobile phase B: CH₃CN) to obtain 6-(4-amino-4-(pyridin-4-ylmethyl)piperidin-1-yl)-3-(2,3-dichlorophenyl)-1H-pyrazolo[3,4-d]pyrimidine-4-carbonitrile 35g (15 mg) with a yield of 18.43%.

MS m/z (ESI): 479.1 [M+1]

### Step 7

### 6-(4-amino-4-(pyridin-4-ylmethyl)piperidin-1-yl)-3-(2,3-dichlorophenyl)-1H-pyrazolo[3,4-d]pyrimidine-4-carboxamide

5 M sodium hydroxide solution (0.5 mL) was added to a solution of methanol (1 mL) containing 6-(4-amino-4-(pyridin-4-ylmethyl)piperidin-1-yl)-3-(2,3-dichlorophenyl)-1H-pyrazolo[3,4-d]pyrimidine-4-carbonitrile 35g (17.04 mg, 35.54 μmol), then added with hydrogen peroxide (0.5 mL), and stirred at room temperature for 1 hour. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-4-ylmethyl)piperidin-1-yl)-3-(2,3-dichlorophenyl)-1H-pyrazolo[3,4-d]pyrimidine-4-carboxamide 35 (1 mg) with a yield of 5.66%.

MS m/z (ESI): 497.1 [M+1]

¹H NMR (400 MHz, DMSO-d₆) δ 13.57 (s, 1H), 8.63 (d, J=4.9 Hz, 1H), 8.12 (s, 1H), 7.99 (s, 1H), 7.66 (d, J=5.2 Hz, 1H), 7.40 (d, J=4.8 Hz, 2H), 7.21 (s, 1H), 6.66 (s, 1H), 5.32 (s, 2H), 1.92-2.11 (m, 4H), 1.79 (d, J=30.8 Hz, 4H), 1.45 (s, 2H).

### Example 36

### 6-(4-amino-4-(pyridin-3-ylmethyl)piperidin-1-yl)-3-(2,3-dichlorophenyl)-1H-pyrazolo[3,4-d]pyrimidine-4-carboxamide

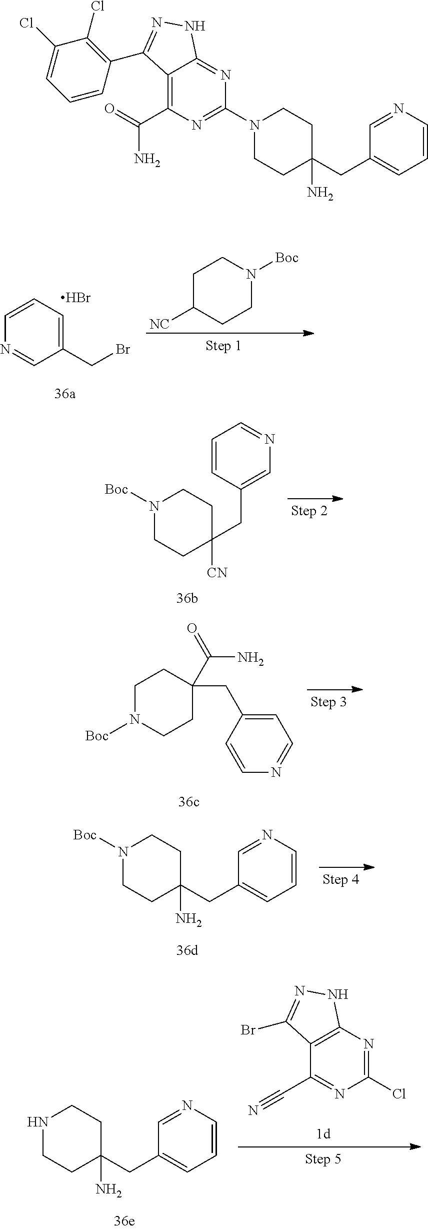

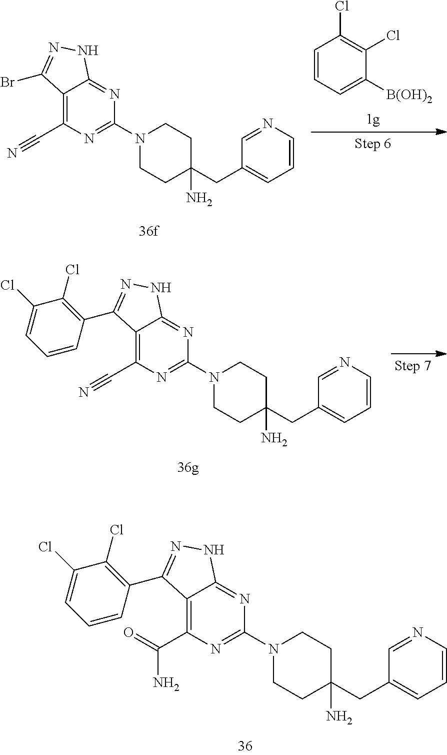 [SMILES: N#Cc1nc(N2CCC(N)(Cc3cccnc3)CC2)nc2[nH]nc(Br)c12 ; OB(O)c1cccc(Cl)c1Cl ; N#Cc1nc(N2CCC(N)(Cc3cccnc3)CC2)nc2[nH]nc(-c3cccc(Cl)c3Cl)c12 ; NC(=O)c1nc(N2CCC(N)(Cc3cccnc3)CC2)nc2[nH]nc(-c3cccc(Cl)c3Cl)c12]

### Step 1

### Tert-butyl 4-cyano-4-(pyridin-3-ylmethyl)piperidine-1-carboxylate

3-(bromomethyl)pyridine hydrobromide 36a (1.98 g, 7.85 mmol), tert-butyl 4-cyanopiperidine-1-carboxylate (1.5 g, 7.13 mmol) and N,N-diisopropylethylamine (1.20 g, 9.27 mmol) were added to 4 mL of toluene, stirred at room temperature for 15 minutes, cooled to 0° C., dropwise added with a 1 M tetrahydrofuran solution (1.57 g, 7.85 mmol) containing potassium bis(trimethylsilyl)amide, then heated to room temperature, and reacted for 16 hours. After reaction was completed, the reaction solution was added with 20 mL of saturated sodium chloride solution and extracted with ethyl acetate (20 mL×3), and then washed with a saturated sodium chloride solution (20 mL). Organic phases were combined, dried with anhydrous sodium sulfate, filtered, concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, and mobile phase B: CH₃CN) to obtain tert-butyl 4-cyano-4-(pyridin-3-ylmethyl)piperidine-1-carboxylate 36b (1.18 g) with a yield of 54.89%.

MS m/z (ESI): 302.0 [M+1]

### Step 2

### Tert-butyl 4-carbamoyl-4-(pyridin-3-ylmethyl)piperidine-1-carboxylate

Potassium hydroxide (439.37 mg, 7.83 mmol) and tert-butyl 4-cyano-4-(pyridin-3-ylmethyl)piperidine-1-carboxylate 36b (1.18 g, 3.92 mmol) were added to dimethyl sulfoxide (6.81 mL), and hydrogen peroxide (2.7 mL) was slowly added to the reaction solution in a water bath, then the reaction solution was heated to room temperature, and reacted for 30 minutes. After reaction was completed, the reaction solution was added with 100 mL of water, and extracted with ethyl acetate (20 mL×2), washed with a saturated sodium chloride solution (20 mL), dried with anhydrous sodium sulfate, and concentrated under reduced pressure to obtain tert-butyl 4-carbamoyl-4-(pyridin-3-ylmethyl)piperidine-1-carboxylate 36c (1.25 g) with a yield of 100%, which was directly used for the next reaction without purification.

MS m/z (ESI): 320.0 [M+1]

### Step 3

### Tert-butyl 4-amino-4-(pyridin-3-ylmethyl)piperidine-1-carboxylate

Potassium hydroxide (988.17 mg, 17.61 mmol) was added to a mixed solution of acetonitrile (3 mL) and water (3 mL) containing tert-butyl 4-carbamoyl-4-(pyridin-3-ylmethyl)piperidine-1-carboxylate 36c (1.25 g, 3.91 mmol), added with 1,3-dibromo-5,5-dimethylhydantoin (615.44 mg, 2.15 mmol) in a water bath in batches, and stirred at room temperature for 1 hour. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain tert-butyl 4-amino-4-(pyridin-3-ylmethyl)piperidine-1-carboxylate 36d (700 mg) with a yield of 61.38%.

MS m/z (ESI): 292.2 [M+1]

### Step 4

### 4-(pyridin-3-ylmethyl)piperidin-4-amine

Tert-butyl 4-amino-4-(pyridin-3-ylmethyl)piperidine-1-carboxylate 36d (200 mg, 686.37 μmol) and 0.5 mL of trifluoroacetic acid were added to 2 mL of dichloromethane, and reacted for 1 hour. After the reaction was completed, the reaction solution was directly concentrated under reduced pressure to obtain 4-(pyridin-3-ylmethyl)piperidin-4-amine 36e (131 mg) with a yield of 99.78%, which was directly used for the next reaction without purification.

MS m/z (ESI): 192.1 [M+1]

### Step 5

### 6-(4-amino-4-(pyridine-3-ylmethyl)piperidine-1-yl)-3-bromo-1H-pyrazolo[3,4-d]pyrimidine-4-nitrile

3-bromo-6-chloro-1H-pyrazolo[3,4-d]pyrimidine-4-carbonitrile 1d (147.51 mg, 570.74 μmol) was added to N,N-dimethylacetamide (3 mL), added with N,N-diisopropylethylamine (221.29 mg, 1.71 mmol) and 4-(pyridin-3-ylmethyl)piperidin-4-amine 36e (131.00 mg, 684.89 μmol),heated to 80° C., and reacted for 1 hour. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-3-ylmethyl)piperidin-1-yl)-3-bromo-1H-pyrazolo[3,4-d]pyrimidine-4-carbonitrile 36f (80 mg) with a yield of 33.92%.

MS m/z (ESI): 413.1 [M+1]

### Step 6

### 6-(4-amino-4-(pyridin-3-ylmethyl)piperidin-1-yl)-3-(2,3-dichlorophenyl)-1H-pyrazolo[3,4-d]pyrimidine-4-carbonitrile

6-(4-amino-4-(pyridin-3-ylmethyl)piperidin-1-yl)-3-bromo-1H-pyrazolo[3,4-d]pyrimidine-4-carbonitrile 36f (80.19 mg, 194.04 μmol), (2,3-dichlorophenyl)boronic acid 1g (148.11 mg, 776.16 μmol), methanesulfonato(2-dicyclohexylphosphino-2′,6′-di-isopropoxy-1,1′-biphenyl)(2-amino-1,1′-biphenyl-2-yl)palladium (32.50 mg, 38.81 μmol), 2-dicyclohexylphosphino-2′,6′-di-isopropoxy-1,1′-biphenyl (36.22 mg, 77.62 μmol) and potassium phosphate (123.62 mg, 582.12 μmol) were added to a mixed solution of 1,4-dioxane (10 mL) and water (1 mL), subjected to argon gas displacement thrice, heated to 100° C., and reacted for 16 hours. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-3-ylmethyl)piperidin-1-yl)-3-(2,3-dichlorophenyl)-1H-pyrazolo[3,4-d]pyrimidine-4-carbonitrile 36g (12 mg) with a yield of 12.90%.

MS m/z (ESI): 479.1 [M+1]

### Step 7

### 6-(4-amino-4-(pyridin-3-ylmethyl)piperidin-1-yl)-3-(2,3-dichlorophenyl)-1H-pyrazolo[3,4-d]pyrimidine-4-carboxamide

5 M sodium hydroxide solution (0.5 mL) was added to a solution of methanol (1 mL) containing 6-(4-amino-4-(pyridin-3-ylmethyl)piperidin-1-yl)-3-(2,3-dichlorophenyl)-1H-pyrazolo[3,4-d]pyrimidine-4-carbonitrile 36g (11.02 mg, 22.99 μmol), then added with hydrogen peroxide (0.5 mL), and stirred at room temperature for 1 hour. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-3-ylmethyl)piperidin-1-yl)-3-(2,3-dichlorophenyl)-1H-pyrazolo[3,4-d]pyrimidine-4-carboxamide 36 (1 mg) with a yield of 8.74%.

MS m/z (ESI): 497.1 [M+1]

### Example 37

### 6-(4-amino-4-(pyridin-2-ylmethyl)piperidin-1-yl)-3-(2,3-dichlorophenyl)-1H-pyrazolo[3,4-d]pyrimidine-4-carboxamide

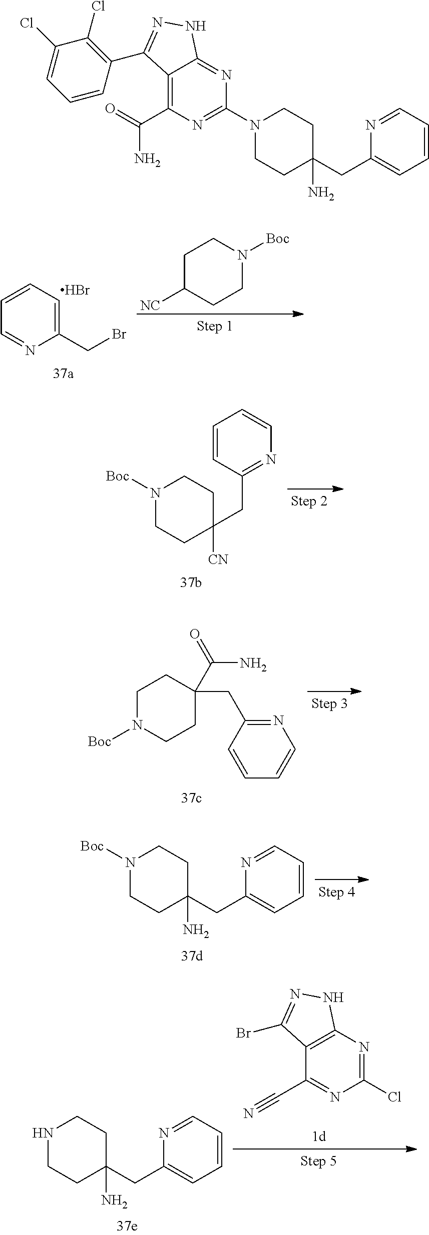

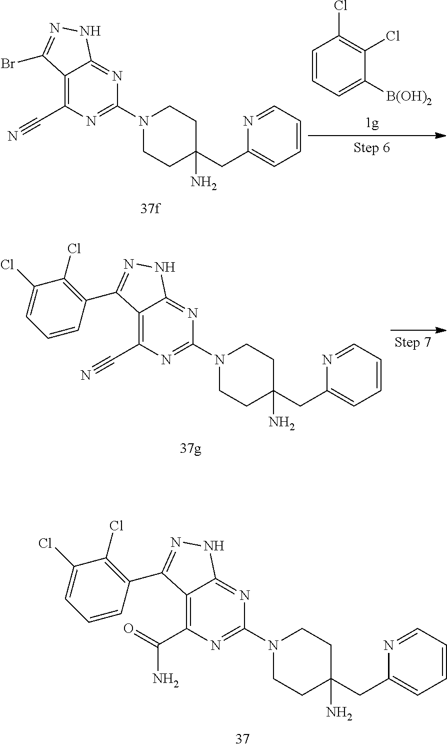

### Step 1

### Tert-butyl 4-cyano-4-(pyridin-2-ylmethyl)piperidine-1l-carboxylate

2-(bromomethyl)pyridine hydrobromide 37a (2.65 g, 10.46 mmol), tert-butyl 4-cyanopiperidine-1-carboxylate (2 g, 9.51 mmol) and N,N-diisopropylethylamine (1.60 g, 12.36 mmol) were added to 2 mL of toluene, stirred at room temperature for 15 minutes, cooled to 0° C., dropwise added with a 1 M tetrahydrofuran solution (2.09 g, 10.46 mmol) containing potassium bis(trimethylsilyl)amide, then heated to room temperature, and reacted for 1 hour. After the reaction was completed, the reaction solution was added with 20 mL of saturated sodium chloride solution and extracted with ethyl acetate (20 mL×3), and then washed with a saturated sodium chloride solution (20 mL). Organic phases were dried with anhydrous sodium sulfate, filtered, concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, and mobile phase B: CH₃CN) to obtain tert-butyl 4-cyano-4-(pyridin-2-ylmethyl)piperidine-1-carboxylate 37b (1.4 g) with a yield of 48.84%.

MS m/z (ESI): 302.0 [M+1]

### Step 2

### Tert-butyl 4-carbamoyl-4-(pyridin-2-ylmethyl)piperidine-1-carboxylate

Potassium hydroxide (703.74 mg, 12.54 mmol) and tert-butyl 4-cyano-4-(pyridin-2-ylmethyl)piperidine-1-carboxylate 37b (1.89 g, 6.27 mmol) were added to dimethyl sulfoxide (10.9 mL), and hydrogen peroxide (4.3 mL) was slowly added to the reaction solution in a water bath, then the reaction solution was heated to room temperature, and reacted for 30 minutes. After the reaction was completed, the reaction solution was added with 100 mL of water, and extracted with ethyl acetate (20 mL×2), washed with a saturated sodium chloride solution (20 mL), dried with anhydrous sodium sulfate, and concentrated under reduced pressure to obtain tert-butyl 4-carbamoyl-4-(pyridin-2-ylmethyl)piperidine-1-carboxylate 37c (2 g) with a yield of 99.85%, which was directly used for the next reaction without purification.

MS m/z (ESI): 320.0 [M+1]

### Step 3

### Tert-butyl 4-amino-4-(pyridin-2-ylmethyl)piperidine-1-carboxylate

Potassium hydroxide (1.58 g, 28.18 mmol) was added to a mixed solution of acetonitrile (6 mL) and water (6 mL) containing tert-butyl 4-carbamoyl-4-(pyridin-2-ylmethyl)piperidine-1-carboxylate 37c (2 g, 6.26 mmol), added with 1,3-dibromo-5,5-dimethylhydantoin (984.70 mg, 3.44 mmol) in a water bath in batches, and stirred at room temperature for 1 hour. The reaction solution was concentrated under reduced pressure and separated on a C₁₈ reversed phase chromatographic column (C₁₈ separation column 20-45 μm; mobile phase A: H₂O, mobile phase B: CH₃CN) to obtain tert-butyl 4-amino-4-(pyridin-2-ylmethyl)piperidine-1-carboxylate 37d (1.3 g) with a yield of 71.25%.

MS m/z (ESI): 292.2 [M+1]

### Step 4

### 4-(pyridin-2-ylmethyl)piperidin-4-amine

Tert-butyl 4-amino-4-(pyridin-2-ylmethyl)piperidine-1-carbonylate 37d (219 mg, 751.58 μmol) and 0.5 mL of trifluoroacetic acid were added to 2 mL of dichloromethane, and reacted for 1 hour. After the reaction was completed, the reaction solution was directly concentrated under reduced pressure to obtain 4-(pyridin-2-ylmethyl)piperidin-4-amine 37e (143.76 mg) with a yield of 100.00%, which was directly used for the next reaction without purification.

MS m/z (ESI): 192.1 [M+1]

### Step 5

### 6-(4-amino-4-(pyridin-2-ylmethyl)piperidin-1-yl)-3-bromo-1H-pyrazolo[3,4-d]pyrimidine-4-carbonitrile

3-bromo-6-chloro-1H-pyrazolo[3,4-d]pyrimidine-4-carbonitrile 1d (161.03 mg, 623.02 μmol) was added to N,N-dimethylacetamide (3 mL), added with N,N-diisopropylethylamine (241.56 mg, 1.87 mmol) and 4-(pyridin-2-ylmethyl)piperidin-4-amine 37e (143.0 mg, 747.63 μmol), heated to 80° C., and reacted for 1 hour. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-2-ylmethyl)piperidin-1-yl)-3-bromo-1H-pyrazolo[3,4-d]pyrimidine-4-carbonitrile 37f (200 mg) with a yield of 77.68%.

MS m/z (ESI): 413.1 [M+1]

### Step 6

### 6-(4-amino-4-(pyridin-2-ylmethyl)piperidin-1-yl)-3-(2,3-dichlorophenyl)-1H-pyrazolo[3,4-d]pyrimidine-4-carbonitrile

6-(4-amino-4-(pyridin-2-ylmethyl)piperidin-1-yl)-3-bromo-1H-pyrazolo[3,4-d]pyrimidine-4-carbonitrile 37f (200.48 mg, 485.10 μmol), (2,3-dichlorophenyl)boronic acid 1g (370.27 mg, 1.94 mmol), methanesulfonato(2-dicyclohexylphosphino-2′,6′-di-isopropoxy-1,1′-biphenyl)(2-amino-1,1′-biphenyl-2-yl)palladium (81.24 mg, 97.02 μmol), 2-dicyclohexylphosphino-2′,6′-di-isopropoxy-1,1′-biphenyl (90.54 mg, 194.04 μmol) and potassium phosphate (309.06 mg, 1.46 mmol) were added to a mixed solution of 1,4-dioxane (10 mL) and water (1 mL), subjected to argon gas displacement thrice, heated to 100° C., and reacted for 16 hours. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-2-ylmethyl)piperidin-1-yl)-3-(2,3-dichlorophenyl)-1H-pyrazolo[3,4-d]pyrimidine-4-carbonitrile 37g (30 mg) with a yield of 12.90%.

MS m/z (ESI): 479.1 [M+1]

### Step 7

### 6-(4-amino-4-(pyridin-2-ylmethyl)piperidin-1-yl)-3-(2,3-dichlorophenyl)-1H-pyrazolo[3,4-d]pyrimidine-4-carboxamide

5 M sodium hydroxide solution (0.5 mL) was added to a solution of methanol (1 mL) containing 6-(4-amino-4-(pyridin-2-ylmethyl)piperidin-1-yl)-3-(2,3-dichlorophenyl)-1H-pyrazolo[3,4-d]pyrimidine-4-carbonitrile 37g (30 mg, 62.58 μmol), then added with hydrogen peroxide (0.5 mL), and stirred at room temperature for 1 hour. After the reaction was completed, a trifluoroacetic acid was added to adjust the pH to be acidic, and then the reaction solution was concentrated under reduced pressure, and subjected to liquid phase separation (separation column AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-2-ylmethyl)piperidin-1-yl)-3-(2,3-dichlorophenyl)-1H-pyrazolo[3,4-d]pyrimidine-4-carboxamide 37 (1 mg) with a yield of 3.21%.

MS m/z (ESI): 497.1 [M+1]

### Example 38

### 6-(4-amino-4-(pyridin-2-yl)piperidin-1-yl)-3-(2,3-dichlorophenyl)-1H-pyrazolo[3,4-d]pyrimidine-4-carboxamide

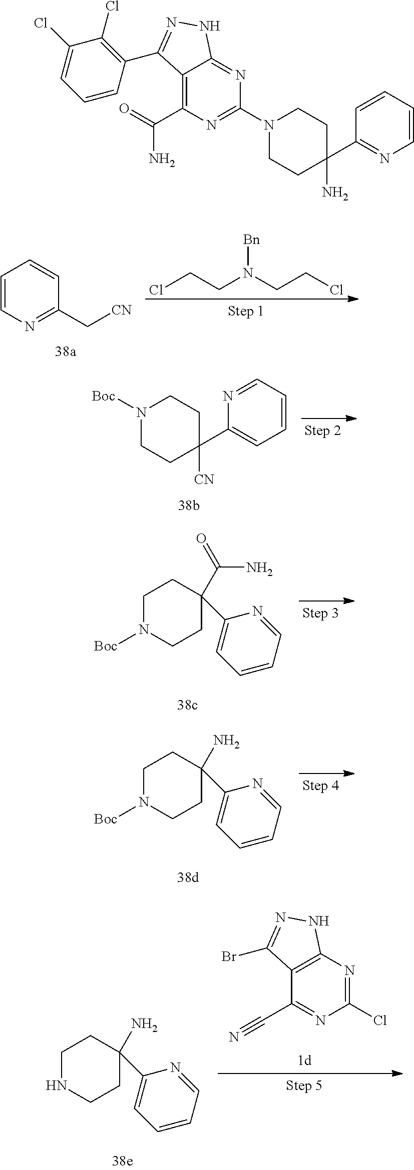

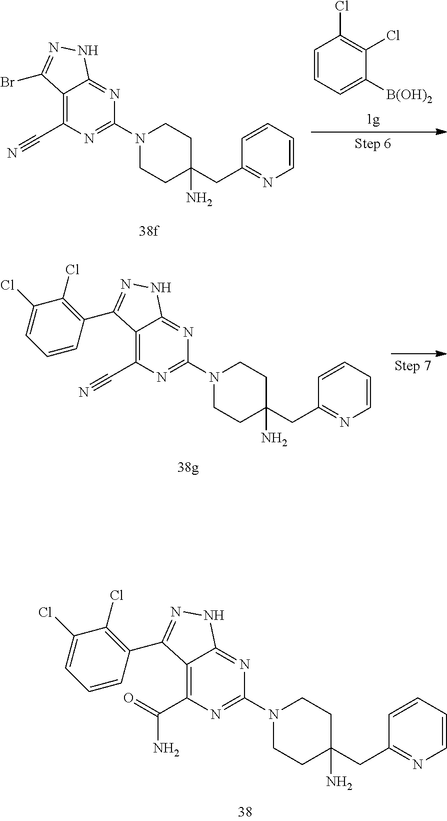 [SMILES: N#Cc1nc(N2CCC(N)(Cc3ccccn3)CC2)nc2[nH]nc(Br)c12 ; OB(O)c1cccc(Cl)c1Cl ; N#Cc1nc(N2CCC(N)(Cc3ccccn3)CC2)nc2[nH]nc(-c3cccc(Cl)c3Cl)c12 ; NC(=O)c1nc(N2CCC(N)(Cc3ccccn3)CC2)nc2[nH]nc(-c3cccc(Cl)c3Cl)c12]

### Step 1

### Tert-butyl 4-cyano-4-(pyridine-2-yl)piperidine-1-carboxylate

Under argon gas, sodium hydride (1.39 g, 53.45 mmol) was added to a solution of N,N-dimethylformamide (30 mL) containing 2-(pyridine-2-yl)acetonitrile 38a (1.80 g, 15.27 mmol), cooled to 0° C., added with tert-butyl bis(2-chloroethyl)carbamate (4.07 g, 16.80 mmol) in batches, continuously stirred for 1 hour, heated to 60° C., and reacted for 16 hours. After the reaction was completed, the reaction solution was cooled to room temperature, added with 40 mL of saturated aqueous ammonium chloride solution, extracted with ethyl acetate (50 mL×3), and washed with a saturated sodium chloride solution (30 mL). Organic phases were dried with anhydrous sodium sulfate, filtered, and concentrated under reduced pressure. The obtained residue was further analyzed and purified by silicagel column chromatography (eluent: system A) to obtain tert-butyl 4-cyano-4-(pyridin-2-yl)piperidine-1-carboxylate 38b (3.27 g) with a yield of 74.51%.

MS m/z (ESI): 231.9 [M−55]

### Step 2

### Tert-butyl 4-carbamoyl-4-(pyridin-2-yl)piperidine-1-carboxylate

Tert-butyl 4-cyano-4-(pyridin-2-yl)piperidine-1-carboxylate 38b (3.27 g, 11.38 mmol) and potassium hydroxide (1.28 g, 22.76 mmol) were added to dimethyl sulfoxide (8.12 mL), and hydrogen peroxide (8.12 mL) was slowly added to the reaction solution, then the reaction solution was heated to room temperature, and reacted for 30 minutes. After the reaction was completed, the reaction solution was added with 100 mL of water to precipitate a large amount of solids and filtered to obtain tert-butyl 4-carbamoyl-4-(pyridine-2-yl)piperidine-1-carboxylate 38c (1.3 g) with a yield of 37.41%.

MS m/z (ESI): 306.2 [M+1]

### Step 3

### Tert-butyl 4-amino-4-(pyridin-2-yl)piperidine-1-carboxylate

Potassium hydroxide (1.07 g, 19.16 mmol) was added to a mixed solution of acetonitrile (3.4 mL) and water (13.7 mL) containing tert-butyl 4-carbamoyl-4-(pyridin-2-yl)piperidine-1-carboxylate 38c (1.3 g, 4.26 mmol), added with 1,3-dibromo-5,5-dimethylhydantoin (669.46 mg, 2.34 mmol) in a water bath in batches, and stirred at room temperature for 16 hours. After the reaction was completed, the reaction solution was added with sodium sulphite and stirred at room temperature for 15 minutes, then added with 50 mL of ethyl acetate and potassium phosphate, and then concentrated under reduced pressure. The obtained residue was further analyzed and purified by silica gel column chromatography (eluent: system B) to obtain tert-butyl 4-amino-4-(pyridin-2-yl)piperidine-1-carboxylate 38d (1.18 g) with a yield of 100%, which was directly used for the next reaction without purification.

MS m/z (ESI): 278.1 [M+1]

### Step 4

### 4-(pyridin-2-yl)piperidine-4-amine

Tert-butyl 4-amino-4-(pyridin-2-yl)piperidine-1-carboxylate 38d (180 mg, 648.97 μmol) was added to dichloromethane (4 mL), and then added with trifluoroacetic acid (1 mL) and reacted for 1 hour. After the reaction was completed, the reaction solution was concentrated under reduced pressure to obtain 4-(pyridin-2-yl)piperidin-4-amine 38e (115.03 mg) with a yield of 100%, which was directly used for the next reaction without purification.

MS m/z (ESI): 178.1 [M+1]

### Step 5

### 6-(4-amino-4-(pyridin-2-yl)piperidin-1-yl)-3-bromo-1H-pyrazolo[3,4-d]pyrimidine-4-carbonitrile

3-bromo-6-chloro-1H-pyrazolo[3,4-d]pyrimidine-4-carbonitrile 1d (130 mg, 502.97 μmol) was added to N-methyl pyrrolidone (5 mL), then added with N,N-diisopropylethylamine (195.01 mg, 1.51 mmol) and 4-(pyridin-2-yl)piperidin-4-amine 38e (89.15 mg, 502.97 μmol), heated to 110° C., and reacted for 1 hour. After the reaction was completed, the reaction solution was concentrated under reduced pressure. The obtained residue was further analyzed and purified by silica gel column chromatography (eluent: system B) to obtain 6-(4-amino-4-(pyridin-2-yl)piperidin-1-yl)-3-bromo-1H-pyrazolo[3,4-d]pyrimidine-4-carbonitrile 38f (150 mg) with a yield of 74.70%.

MS m/z (ESI): 398.9 [M+1]

### Step 6

### 6-(4-amino-4-(pyridin-2-yl)piperidin-1-yl)-3-(2,3-dichlorophenyl)-1H-pyrazolo[3,4-d]pyrimidine-4-carbonitrile

6-(4-amino-4-(pyridin-2-yl)piperidin-1-yl)-3-bromo-1H-pyrazolo[3,4-d]pyrimidine-4-carbonitrile 38f (150 mg, 375.71 μmol), (2,3-dichlorophenyl)boronic acid 1g (286.77 mg, 1.50 mmol), methanesulfonato(2-dicyclohexylphosphino-2′,6′-di-isopropoxy-1,1′-biphenyl)(2-amino-1,1′-biphenyl-2-yl)palladium (62.92 mg, 75.14 μmol), 2-dicyclohexylphosphino-2′,6′-di-isopropoxy-1,1′-biphenyl (70.13 mg, 150.28 μmol) and potassium phosphate (239.37 mg, 1.13 mmol) were added to a mixed solution of 1,4-dioxane (10 mL) and water (1 mL), subjected to argon gas displacement thrice, heated to 100° C., and reacted for 16 hours. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-2-yl)piperidin-1-yl)-3-(2,3-dichlorophenyl)-1H-pyrazolo[3,4-d]pyrimidine-4-carbonitrile 38g (26 mg) with a yield of 14.87%.

MS m/z (ESI): 464.9 [M+1]

### Step 7

### 6-(4-amino-4-(pyridin-2-yl)piperidin-1-yl)-3-(2,3-dichlorophenyl)-1H-pyrazolo[3,4-d]pyrimidine-4-carboxamide

6-(4-amino-4-(pyridin-2-yl)piperidin-1-yl)-3-(2,3-dichlorophenyl)-1H-pyrazolo[3,4-d]pyrimidine-4-carbonitrile 38g (26.27 mg, 55.87 μmol), sodium hydroxide (0.5 mL) and hydrogen peroxide (0.5 mL) were added to a mixed solution of methanol (1 mL) in turn, and stirred at room temperature for 1 hour. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-2-yl)piperidin-1-yl)-3-(2,3-dichlorophenyl)-1H-pyrazolo[3,4-d]pyrimidine-4-carboxamide 38 (5 mg) with a yield of 18.51%.

MS m/z (ESI): 482.9 [M+1]

¹H NMR (400 MHz, CD₃OD) δ 8.75 (d, J=4.8 Hz, 1H), 7.97 (t, J=7.8 Hz, 1H), 7.75 (d, J=8.1 Hz, 1H), 7.61 (dd, J=7.4, 1.9 Hz, 1H), 7.48 (dd, J=7.6, 4.9 Hz, 1H), 7.34-7.44 (m, 2H), 4.21 (d, J=25.2 Hz, 4H), 2.58-2.69 (m, 2H), 2.17 (dd, J=13.9, 7.4 Hz, 2H).

### Example 39

### (S)-6-(5-amino-5,7-dihydrospiro[cyclopenta[b]pyridine-6,4′-piperidin]-1′-yl)-3-((2,3-dichlorophenyl)thio)-1H-pyrazolo[3,4-d]pyrimidine-4-carboxamide

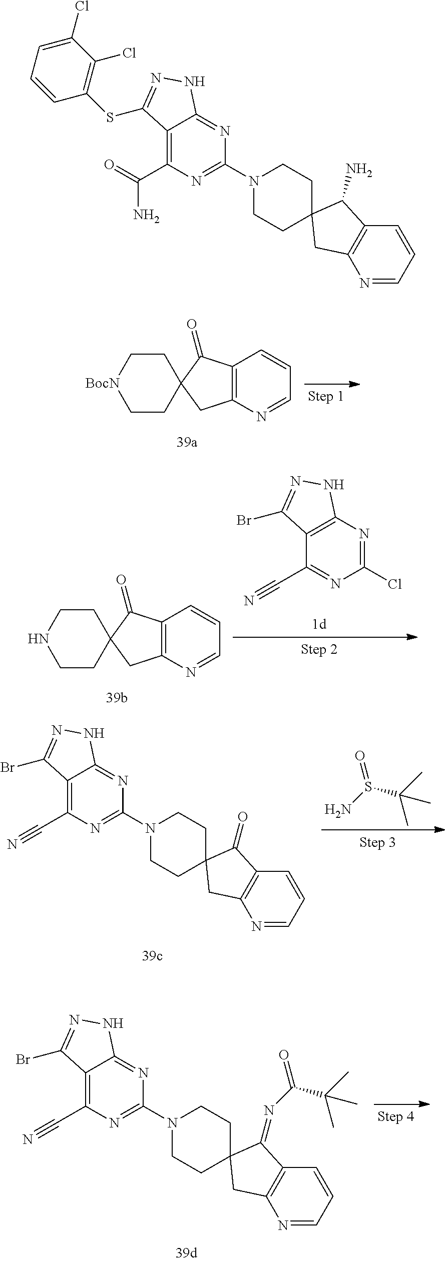

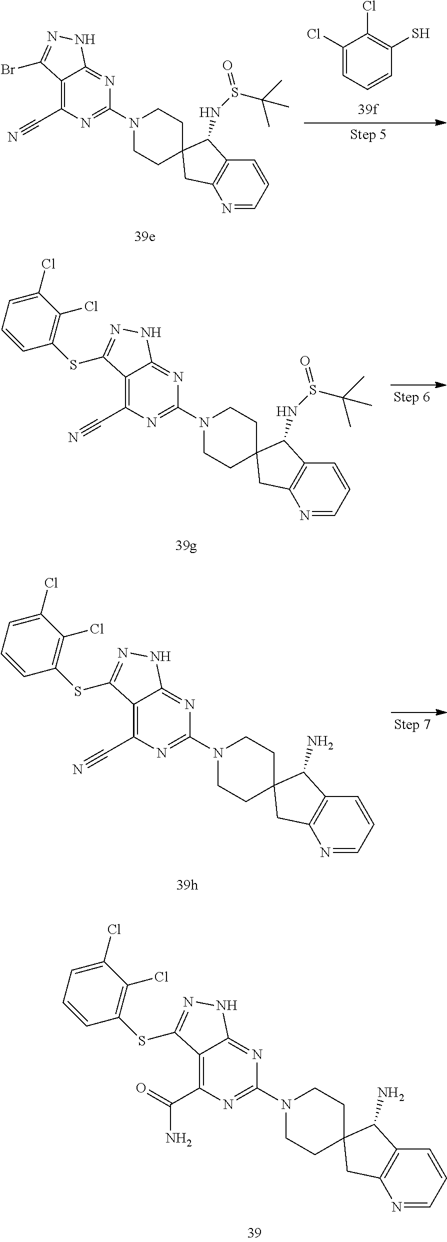 [SMILES: CC(C)(C)S(=O)N[C@@H]1c2cccnc2CC12CCN(c1nc(C#N)c3c(Br)n[nH]c3n1)CC2 ; Sc1cccc(Cl)c1Cl ; CC(C)(C)S(=O)N[C@@H]1c2cccnc2CC12CCN(c1nc(C#N)c3c(Sc4cccc(Cl)c4Cl)n[nH]c3n1)CC2 ; N#Cc1nc(N2CCC3(CC2)Cc2ncccc2[C@H]3N)nc2[nH]nc(Sc3cccc(Cl)c3Cl)c12 ; NC(=O)c1nc(N2CCC3(CC2)Cc2ncccc2[C@H]3N)nc2[nH]nc(Sc3cccc(Cl)c3Cl)c12]

### Step 1

### spiro[cyclopenta[b]pyridine-6,4′-piperidin]-5(7H)-one

tert-butyl 5-oxo-5,7-dihydrospiro[cyclopenta[b]pyridine 6,4′-piperidine]-1′-carboxylate 39a (600 mg, 1.98 mmol) was added to dichloromethane (4 mL), and continuously reacted for 1 hour. After the reaction was completed, the reaction solution was concentrated under reduced pressure to obtain spiro[cyclopenta[b]pyridine-6,4′-piperidin]-5(7H)-one 39b (401 mg) with a yield of 99.92%, which was directly used for the next reaction without purification.

MS m/z (ESI): 202.9 [M+1]

### Step 2

### 3-bromo-6-(5-oxo-5,7-dihydrospiro[cyclopenta[b]pyridine-6,4′-piperidin]-1′-yl)-1H-pyrazolo[3,4-d]pyrimidine-4-carbonitrile

3-bromo-6-chloro-1H-pyrazolo[3,4-d]pyrimidine-4-carbonitrile 1d (300 mg, 1.16 mmol), N,N-diisopropylethylamine (750.05 mg, 5.80 mmol) and spiro[cyclopenta[b]pyridine-6,4′-piperidin]-5(7H)-one 39b (399.08 mg, 1.97 mmol) were added to N,N-dimethylacetamide (2 mL), heated to 90° C., and reacted for 1 hour. After the reaction was completed, the reaction solution was concentrated under reduced pressure. The obtained residue was further analyzed and purified by silica gel column chromatography (eluent: system A) to obtain 3-bromo-6-(5-oxo-5,7-dihydrospiro[cyclopenta[b]pyridine-6,4′-piperidin]-1′-yl)-1H-pyrazolo[3,4-d]pyrimidine-4-carbonitrile 39c (350 mg) with a yield of 71.08%.

MS m/z (ESI): 424.0 [M+1]

### Step 3

### (R,Z)—N-(1′-(3-bromo-4-cyano-1H-pyrazolo[3,4-d]pyrimidin-6-yl)spiro[cyclopenta[b]pyridine-6,4′-piperidin]-5(7H)-ylidene)-2-methylpropane-2-sulfinamide

3-bromo-6-(5-oxo-5,7-dihydrospiro[cyclopenta[b]pyridine-6,4′-piperidin]-1′-yl)-1H-pyrazolo[3,4-d]pyrimidine-4-carbonitrile 39c (369 mg, 869.76 μmol) and (R)-2-methylpropane-2-sulfinamide (316.25 mg, 2.61 mmol) were added to tetrahydrofuran (2 mL), and reacted at 100° C. for 16 hours. After the reaction was completed, the reaction solution was added with water and extracted with ethyl acetate (30 mL×2) to separate an aqueous layer. Combined organic phases were washed with a saturated sodium chloride solution (30 mL×2) in turn, dried with anhydrous sodium sulfate, filtered, and concentrated under reduced pressure to obtain (R,Z)—N-(1′-(3-bromo-4-cyano-1H-pyrazolo[3,4-d]pyrimidin-6-yl)spiro[cyclopenta[b]pyridine-6,4′-piperidin]-5(7H)-ylidene)-2-methylpropane-2-sulfinamide 39d (458 mg) with a yield of 99.84%, which was directly used for the next reaction without purification.

MS m/z (ESI): 527.1 [M+1]

### Step 4

### (R)—N—((S)-1′-(3-bromo-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide

9-borabicyclo[3.3.1]nonane (0.5 M, 5.21 mL) was added to a solution of tetrahydrofuran (5 mL) containing (R,Z)—N-(1′-(3-bromo-4-cyano-1H-pyrazolo[3,4-d]pyrimidin-6-yl)spiro[cyclopenta[b]pyridine-6,4′-piperidin]-5(7H)-ylidene)-2-methylpropane-2-sulfinamide 39d (458 mg, 868.35 μmol), and stirred at room temperature for 2 hours. After the reaction was completed, the reaction solution was added with 50 mL of water, extracted with ethyl acetate (50 mL×3), and washed with a saturated sodium chloride solution (50 mL). Organic phases were dried with anhydrous sodium sulfate, filtered, and concentrated under reduced pressure. The obtained residue was further analyzed and purified by silica gel column chromatography (eluent: system A) to obtain (R)—N—((S)-1′-(3-bromo-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 39e (300 mg) with a yield of 65.25%.

MS m/z (ESI): 529.2 [M+1]

### Step 5

### (R)—N—((S)-1′-(3-((2,3-dichlorophenyl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide

(R)—N—((S)-1′-(3-bromo-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 39e (150 mg, 283.31 μmol), 2,3-dichlorobenzenethiol 39f (76.10 mg, 424.97 μmol), tris(dibenzylideneacetone)dipalladium (15.57 mg, 17.00 μmol), 4,5-bis(diphenylphosphino)-9,9-dimethylxanthene (16.39 mg, 28.33 μmol) and N,N-diisopropylethylamine (73.23 mg, 566.62 μmol) were added to 1,4-dioxane (4 mL), subjected to nitrogen gas displacement, heated to 100° C., and reacted for 3 hours. After the reaction was completed, the reaction solution was cooled to room temperature, and concentrated under reduced pressure. The obtained residue was further analyzed and purified by silica gel column chromatography (eluent: system B) to obtain (R)—N—((S)-1′-(3-((2,3-dichlorophenyl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 39g (125 mg) with a yield of 70.30%.

MS m/z (ESI): 627.1 [M+1]

### Step 6

### (S)-6-(5-amino-5,7-dihydrospiro[cyclopenta[b]pyridine-6,4′-piperidin]-1′-yl)-3-((2,3-dichlorophenyl)thio)-1H-pyrazolo[3,4-d]pyrimidine-4-carbonitrile

A solution of dichloromethane (4 mL) containing (R)—N—((S)-1′-(3-((2,3-dichlorophenyl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 39g (125 mg, 199.17 μmol) was slowly dropwise added to hydrochloric acid in methanol (4 M, 199.17 μL), and reacted for 1 hour. After the reaction was completed, the reaction solution was concentrated under reduced pressure to obtain (S)-6-(5-amino-5,7-dihydrospiro[cyclopenta[b]pyridine-6,4′-piperidin]-1′-yl)-3-((2,3-dichlorophenyl)thio)-1H-pyrazolo[3,4-d]pyrimidine-4-carbonitrile 39h (104 mg) with a yield of 99.76%, which was directly used for the next reaction without purification.

MS m/z (ESI): 522.8 [M+1]

### Step 7

### (S)-6-(5-amino-5,7-dihydrospiro[cyclopenta[b]pyridine-6,4′-piperidin]-1′-yl)-3-((2,3-dichlorophenyl)thio)-1H-pyrazolo[3,4-d]pyrimidine-4-carboxamide

5 M sodium hydroxide solution (0.25 mL) was added to a solution of methanol (2 mL) containing (S)-6-(5-amino-5,7-dihydrospiro[cyclopenta[b]pyridine-6,4′-piperidin]-1′-yl)-3-((2,3-dichlorophenyl)thio)-1H-pyrazolo[3,4-d]pyrimidine-4-carbonitrile 39h (104 mg, 198.69 μmol), then added with hydrogen peroxide (0.5 mL), and stirred at room temperature for 30 minutes. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain (S)-6-(5-amino-5,7-dihydrospiro[cyclopenta[b]pyridine-6,4′-piperidin]-1′-yl)-3-((2,3-dichlorophenyl)thio)-1H-pyrazolo[3,4-d]pyrimidine-4-carboxamide 39 (3 mg) with a yield of 2.13%.

MS m/z (ESI): 541.1 [M+1]

¹H NMR (400 MHz, DMSO-d₆) δ 13.34 (s, 1H), 8.54 (d, J=5.0 Hz, 1H), 8.30 (s, 2H), 8.24 (s, 1H), 7.91 (d, J=5.0 Hz, 1H), 7.88 (s, 1H), 7.63 (dd, J=8.0, 1.5 Hz, 1H), 7.44 (d, J=7.6 Hz, 1H), 7.35 (dd, J=7.7, 4.9 Hz, 2H), 4.46 (d, J=5.5 Hz, 1H), 3.27 (d, J=16.6 Hz, 2H), 3.07-3.21 (m, 2H), 1.73 (d, J=14.2 Hz, 2H), 1.54 (d, J=13.2 Hz, 2H).

### Example 40

### (S)-6-(5-amino-5,7-dihydrospiro[cyclopenta[b]pyridine-6,4′-piperidin]-1′-yl)-3-(2-(trifluoromethyl)pyridin-3-yl)-1H-pyrazolo[3,4-d]pyrimidine-4-carboxamide

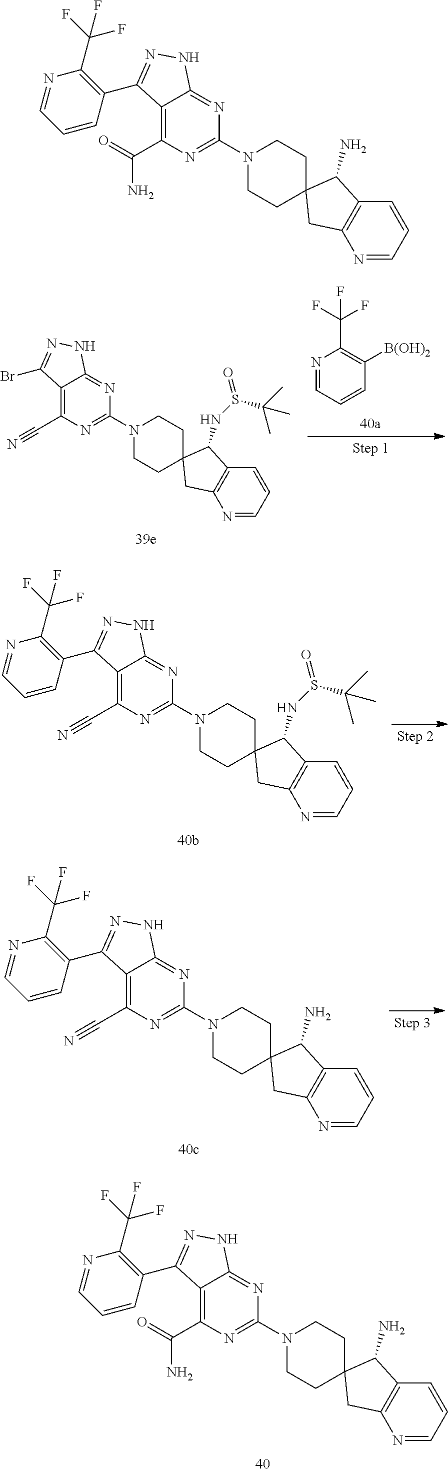

### Step 1

### (R)—N—((S)-1′-(3-(2-(trifluoromethyl)pyridin-3-yl)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide

(R)—N—((S)-1′-(3-bromo-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 39e (150 mg, 283.31 μmol), (2-(trifluoromethyl)pyridin-3-yl)boronic acid 40a (216.35 mg, 1.13 mmol), methanesulfonato(2-dicyclohexylphosphino-2′,6′-di-isopropoxy-1,1′-biphenyl)(2-amino-1,1′-biphenyl-2-yl)palladium (47.45 mg, 56.66 μmol), 2-dicyclohexylphosphino-2′,6′-di-isopropoxy-1,1′-biphenyl (52.88 mg, 113.32 μmol) and potassium phosphate (180.41 mg, 849.93 μmol) were added to 1,4-dioxane (3 mL), subjected to nitrogen gas displacement, heated to 100° C., and reacted for 7 hours. After the reaction was completed, the reaction solution was cooled to room temperature. The obtained residue was further analyzed and purified by silicagel column chromatography (eluent: system B) to obtain (R)—N—((S)-1′-(3-(2-(trifluoromethyl)pyridin-3-yl)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 40b (80 mg) with a yield of 47.41%.

MS m/z (ESI): 596.2 [M+1]

### Step 2

### (S)-6-(5-amino-5,7-dihydrospiro[cyclopenta[b]pyridine-6,4′-piperidin]-1′-yl)-3-(2-(trifluoromethyl)pyridin-3-yl)-1H-pyrazolo[3,4-d]pyrimidine-4-carbonitrile

A solution of dichloromethane (4 mL) containing (R)—N—((S)-1′-(3-(2-(trifluoromethyl)pyridin-3-yl)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 40b (80 mg, 134.31 μmol) was slowly dropwise added to hydrochloric acid in methanol (4 M, 134.31 μL), and reacted for 1 hour. After the reaction was completed, the reaction solution was concentrated under reduced pressure to obtain (S)-6-(5-amino-5,7-dihydrospiro[cyclopenta[b]pyridine-6,4′-piperidin]-1′-yl)-3-(2-(trifluoromethyl)pyridin-3-yl)-1H-pyrazolo[3,4-d]pyrimidine-4-carbonitrile 40c (66 mg) with a yield of 99.99%, which was directly used for the next reaction without purification.

MS m/z (ESI): 492.2 [M+1]

### Step 3

### (S)-6-(5-amino-5,7-dihydrospiro[cyclopenta[b]pyridine-6,4′-piperidin]-1′-yl)-3-(2-(trifluoromethyl)pyridin-3-yl)-1H-pyrazolo[3,4-d]pyrimidine-4-carboxamide

5 M sodium hydroxide solution (1.31 mL) was added to a solution of methanol (952.38 μL) containing (S)-6-(5-amino-5,7-dihydrospiro[cyclopenta[b]pyridine-6,4′-piperidin]-1′-yl)-3-(2-(trifluoromethyl)pyridine-3-yl)-1H-pyrazolo[3,4-d]pyrimidine-4-carbonitrile 40c (66 mg, 134.29 μmol), then added with hydrogen peroxide (238.10 μL), and stirred at room temperature for 30 minutes. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μM, 20 mL/min; mobile phase A: 0.05% TFA+H₂O, mobile phase B: CH₃CN) to obtain (S)-6-(5-amino-5,7-dihydrospiro[cyclopenta[b]pyridine-6,4′-piperidin]-1′-yl)-3-(2-(trifluoromethyl)pyridin-3-yl)-1H-pyrazolo[3,4-d]pyrimidine-4-carboxamide 40 (10 mg) with a yield of 14.6%.

MS m/z (ESI): 510.2 [M+1]

¹H NMR (400 MHz, DMSO-d₆) δ 8.76 (d, J=4.6 Hz, 1H), 8.55 (d, J=4.9 Hz, 1H), 8.28-8.43 (m, 3H), 8.13 (s, 1H), 7.91 (t, J=7.9 Hz, 2H), 7.73 (dd, J=7.8, 4.8 Hz, 1H), 7.58 (s, 1H), 7.36 (dd, J=7.6, 5.1 Hz, 1H), 4.74 (s, 2H), 4.47 (d, J=5.4 Hz, 1H), 3.26-3.34 (m, 3H), 3.16 (d, J=16.9 Hz, 1H), 1.76 (d, J=13.8 Hz, 2H), 1.57 (d, J=13.3 Hz, 2H).

### Example 41

### (S)-6-(5-amino-5,7-dihydrospiro[cyclopenta[b]pyridine-6,4′-piperidin]-1′-yl)-3-((3-chloro-2-cyclopropoxypyridin-4-yl)thio)-1H-pyrazolo[3,4-d]pyrimidine-4-carboxamide

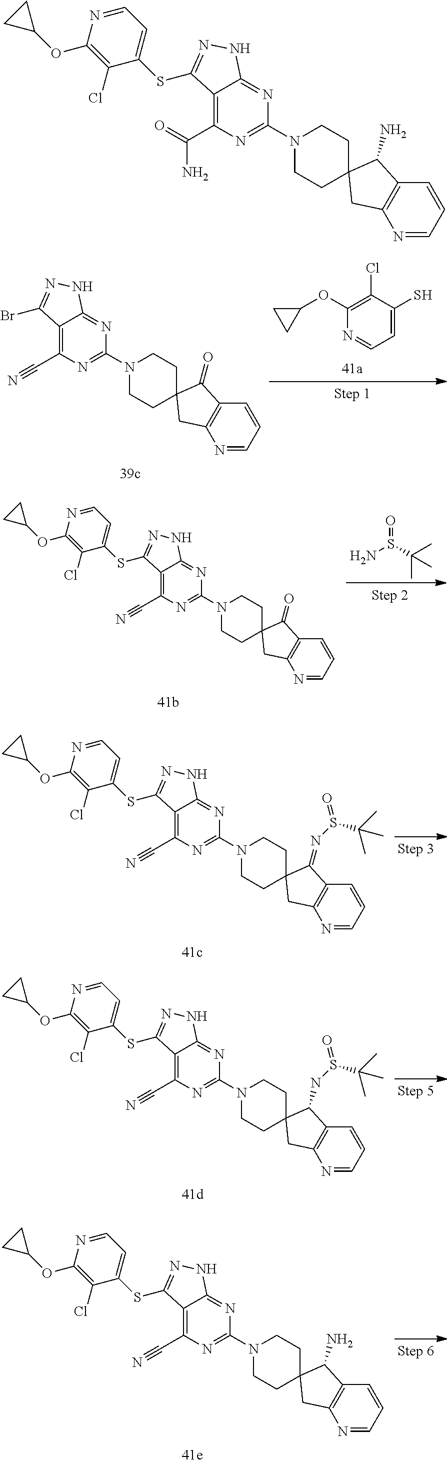

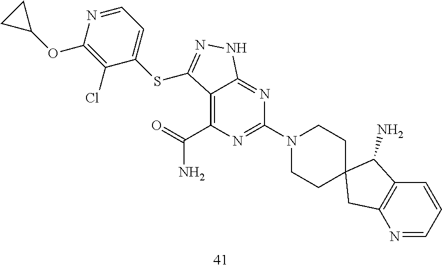 [SMILES: NC(=O)c1nc(N2CCC3(CC2)Cc2ncccc2[C@H]3N)nc2[nH]nc(Sc3ccnc(OC4CC4)c3Cl)c12]

### Step 1

### 3-((3-chloro-2-cyclopropoxypyridin-4-yl)thio)-6-(5-oxo-5,7-dihydrospiro[cyclopenta[b]pyridine-6,4′-piperidin]-1′-yl)-1H-pyrazolo[3,4-d]pyrimidine-4-carbonitrile

3-bromo-6-(5-oxo-5,7-dihydrospiro[cyclopenta[b]pyridine-6,4′-piperidin]-1′-yl)-1H-pyrazolo[3,4-d]pyrimidine-4-carbonitrile 39c (150 mg, 353.56 μmol), 3-chloro-2-(cyclopropoxy)pyridine-4-thiol 41a (142.61 mg, 707.12 μmol, self-prepared according to patent WO2018013597), cuprous iodide (33.67 mg, 176.78 μmol), copper (22.47 mg, 353.56 μmol) and potassium carbonate (146.60 mg, 1.06 mmol) were added to N,N-dimethylformamide (3 mL), subjected to nitrogen gas displacement, heated to 130° C., and reacted for 17 hours. After the reaction was completed, the reaction solution was cooled to room temperature, and concentrated under reduced pressure. The obtained residue was further analyzed and purified by silica gel column chromatography (eluent: system A) to obtain 3-((3-chloro-2-cyclopropoxypyridin-4-yl)thio)-6-(5-oxo-5,7-dihydrospiro[cyclopenta[b]pyridine-6,4′-piperidin]-1′-yl)-1H-pyrazolo[3,4-d]pyrimidine-4-carbonitrile 41b (60 mg) with a yield of 31.14%.

MS m/z (ESI): 545.2 [M+1]

### Step 2

### (R,Z)—N-(1′-(3-((3-chloro-2-cyclopropoxypyridin-4-yl)thio)-4-cyano-1H-pyrazolo[3,4-d]pyrimidin-6-yl)spiro[cyclopenta[b]pyridine-6,4′-piperidin]-5(7H)-ylidene)-2-methylpropane-2-sulfinamide

3-((3-chloro-2-cyclopropoxypyridin-4-yl)thio)-6-(5-oxo-5,7-dihydrospiro[cyclopenta[b]pyridine-6,4′-piperidin]-1′-yl)-1H-pyrazolo[3,4-d]pyrimidine-4-carbonitrile 41b (60 mg, 110.09 μmol) and (R)-2-methylpropane-2-sulfinamide (40.03 mg, 330.27 μmol) were added to tetraethyl titanate (2.00 mL), heated to 100° C., and reacted for 3 hours. After the reaction was completed, the reaction solution was added with 20 mL of water and extracted with ethyl acetate (30 mL×2). The aqueous layer was separated. Combined organic phases were washed with a saturated sodium chloride solution (30 mL×2) in turn, dried with anhydrous sodium sulfate, filtered, and concentrated under reduced pressure to obtain (R,Z)—N-(1′-(3-((3-chloro-2-cyclopropoxypyridin-4-yl)thio)-4-cyano-1H-pyrazolo[3,4-d]pyrimidin-6-yl)spiro[cyclopenta[b]pyridine-6,4′-piperidin]-5(7H)-ylidene)-2-methylpropane-2-sulfinamide 41c (71 mg) with a yield of 99.50%, which was directly used for the next reaction without purification.

MS m/z (ESI): 648.2 [M+1]

### Step 3

### (R)—N—((S)-1′-(3-((3-chloro-2-cyclopropoxypyridin-4-yl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide

9-borabicyclo[3.3.1]nonane (0.5 M, 657.20 μL) was added to a solution of tetrahydrofuran (1 mL) containing (R,Z)—N-(1′-(3-((3-chloro-2-cyclopropoxypyridin-4-yl)thio)-4-cyano-1H-pyrazolo[3,4-d]pyrimidin-6-yl)spiro[cyclopenta[b]pyridine-6,4′-piperidine]-5(7H)-ylidene)-2-methylpropane-2-sulfinamide 41c (71 mg, 109.53 μmol), and stirred at room temperature for 2 hours. After the reaction was completed, the reaction solution was added with 50 mL of water, extracted with ethyl acetate (50 mL×3), and washed with a saturated sodium chloride solution (50 mL). Organic phases were dried with anhydrous sodium sulfate, filtered, and concentrated under reduced pressure. The obtained residue was further analyzed and purified by silica gel column chromatography (eluent: system A) to obtain (R)—N—((S)-1′-(3-((3-chloro-2-cyclopropoxypyridin-4-yl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 41d (40 mg) with a yield of 56.16%.

MS m/z (ESI): 650.3 [M+1]

### Step 4

### (S)-6-(5-amino-5,7-dihydrospiro[cyclopenta[b]pyridine-6,4′-piperidin]-1′-yl)-3-((3-chloro-2-cyclopropoxypyridin-4-yl)thio)-1H-pyrazolo[3,4-d]pyrimidine-4-carbonitrile

A solution of dichloromethane (3.94 mL) containing (R)—N—((S)-1′-(3-((3-chloro-2-cyclopropoxypyridin-4-yl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 41d (40 mg, 61.52 μmol)) was slowly dropwise added to hydrochloric acid in methanol (4 M, 61.52 μL), and reacted for 1 hour. After the reaction was completed, the reaction solution was concentrated under reduced pressure to obtain (S)-6-(5-amino-5,7-dihydrospiro[cyclopenta[b]pyridine-6,4′-piperidin]-1′-yl)-3-((3-chloro-2-cyclopropoxypyridin-4-yl)thio)-1H-pyrazolo[3,4-d]pyrimidine-4-carbonitrile 41e (33 mg) with a yield of 98.24%, which was directly used for the next reaction without purification.

MS m/z (ESI): 546.2 [M+1]

### Step 5

### (S)-6-(5-amino-5,7-dihydrospiro[cyclopenta[b]pyridine-6,4′-piperidin]-1′-yl)-3-((3-chloro-2-cyclopropoxypyridin-4-yl)thio)-1H-pyrazolo[3,4-d]pyrimidine-4-carboxamide

Sodium hydroxide (0.15 mL) was added to a solution of methanol (1.5 mL) containing (S)-6-(5-amino-5,7-dihydrospiro[cyclopenta[b]pyridine-6,4′-piperidin]-1′-yl)-3-((3-chloro-2-cyclopropoxypyridin-4-yl)thio)-1H-pyrazolo[3,4-d]pyrimidine-4-carbonitrile 41e (33 mg, 60.43 μmol), then added with hydrogen peroxide (0.3 mL), and stirred at room temperature for 30 minutes. After the reaction was completed, a trifluoroacetic acid was added to adjust the pH to be acidic, and then the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH3CN) to obtain (S)-6-(5-amino-5,7-dihydrospiro[cyclopenta[b]pyridine-6,4′-piperidin]-1′-yl)-3-((3-chloro-2-cyclopropoxypyridin-4-yl)thio)-1H-pyrazolo[3,4-d]pyrimidine-4-carboxamide 41 (11 mg) with a yield of 32.2%.

MS m/z (ESI): 563.9 [M+1]

¹H NMR (400 MHz, DMSO-d₆) δ 8.54 (d, J=5.0 Hz, 1H), 8.29 (s, 2H), 8.12 (s, 1H), 7.86-7.97 (m, 2H), 7.81 (s, 1H), 7.35 (t, J=6.3 Hz, 1H), 6.55 (d, J=5.4 Hz, 1H), 4.46 (s, 1H), 4.32 (s, 1H), 3.31 (d, J=13.4 Hz, 2H), 3.25 (s, 1H), 3.14 (d, J=16.9 Hz, 2H), 2.67 (s, 1H), 2.33 (s, 1H), 1.73 (d, J=14.2 Hz, 2H), 1.55 (d, J=12.9 Hz, 2H), 0.67-0.84 (m, 4H).

### Example 42

### Ethyl (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxylate

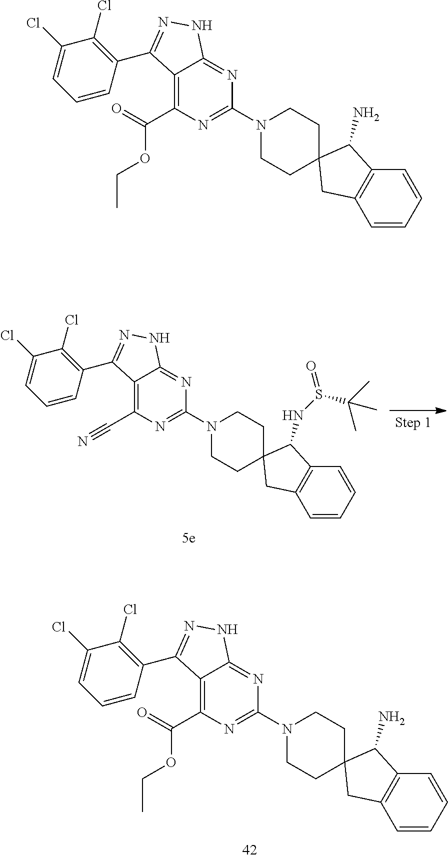 [SMILES: CCOC(=O)c1nc(N2CCC3(CC2)Cc2ccccc2[C@H]3N)nc2[nH]nc(-c3cccc(Cl)c3Cl)c12 ; CC(C)(C)[S@@](=O)N[C@@H]1c2ccccc2CC12CCN(c1nc(C#N)c3c(-c4cccc(Cl)c4Cl)n[nH]c3n1)CC2 ; CCOC(=O)c1nc(N2CCC3(CC2)Cc2ccccc2[C@H]3N)nc2[nH]nc(-c3cccc(Cl)c3Cl)c12]

### Step 1

### Ethyl (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxylate

(R)—N—((S)-1′-(4-cyano-3-(2,3-dichlorophenyl)-1H-pyrazolo[3,4-d]pyrimidin-6-yl)-1,3-dihydrospiro[indene-2,4′-piperidin]-1-yl)-2-methylpropane-2-sulfinamide 5e (130 mg, 218.65 μmol) was added to a concentrated hydrochloric acid (3 mL) and ethanol (3 mL), heated to 100° C., and reacted for 1 hour. After the reaction was completed, the reaction solution was concentrated under reduced pressure, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain ethyl (S)-6-(1-amino-1,3-dihydrospiro[indene-2,4′-piperidin]-1′-yl)-3-(2,3-dichlorophenyl)-1H-pyrazolo[3,4-d]pyrimidine-4-carboxylate 42 (20 mg) with a yield of 17%.

MS m/z (ESI): 537.2 [M+1]

¹H NMR (400 MHz, DMSO-d₆) δ 8.29 (s, 3H), 7.72-7.85 (m, 1H), 7.51 (d, J=5.0 Hz, 3H), 7.37 (d, J=4.8 Hz, 2H), 7.33 (d, J=6.6 Hz, 1H), 4.60 (d, J=25.5 Hz, 2H), 4.39 (d, J=6.1 Hz, 1H), 3.91 (d, J=7.3 Hz, 2H), 3.22 (d, J=16.4 Hz, 1H), 3.07 (s, 1H), 1.63-1.83 (m, 2H), 1.56 (s, 2H), 1.24 (s, 2H), 0.88 (t, J=7.2 Hz, 3H).

### Example 43

### 6-(4-amino-4-((methylsulfonyl)methyl)piperidin-1-yl)-3-(2,3-dichlorophenyl)-1H-pyrazolo[3,4-d]pyrimidine-4-carboxamide

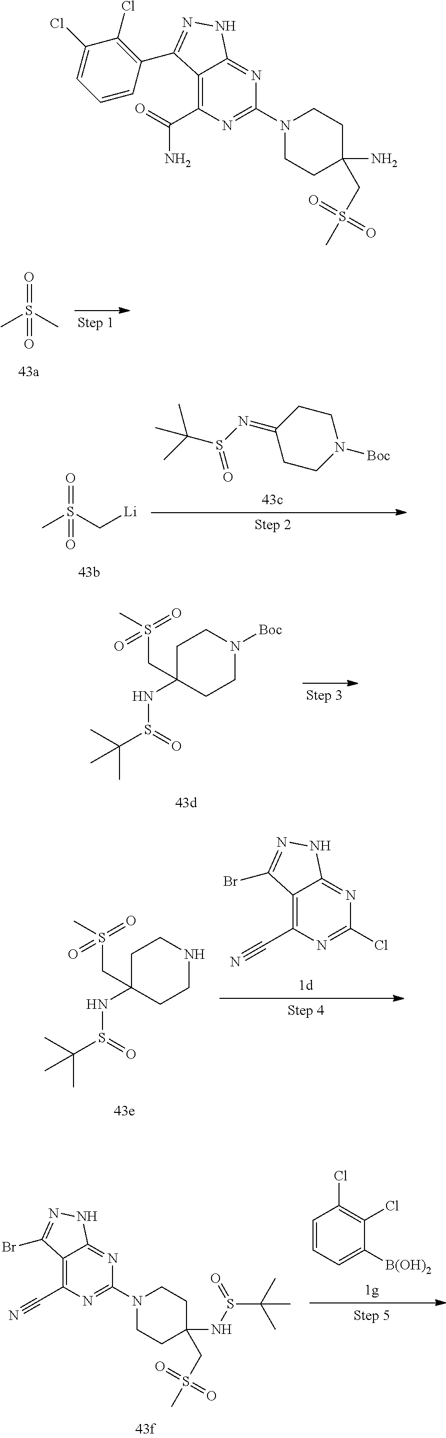

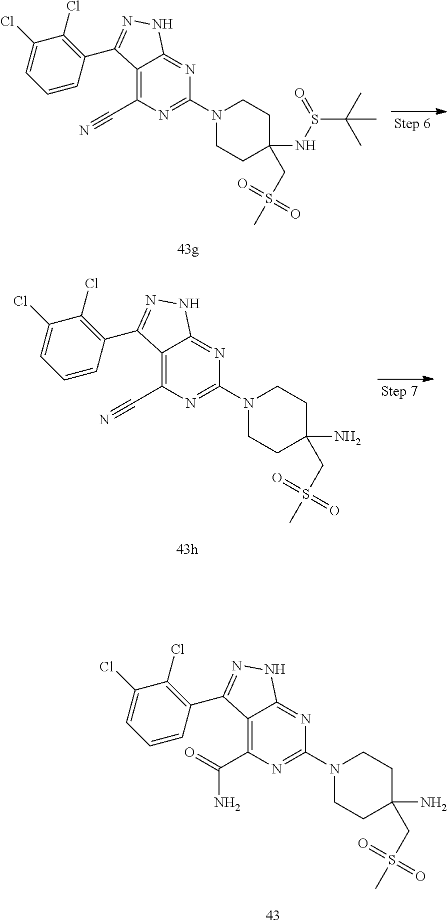 [SMILES: CC(C)(C)S(=O)NC1(CS(C)(=O)=O)CCN(c2nc(C#N)c3c(-c4cccc(Cl)c4Cl)n[nH]c3n2)CC1 ; CS(=O)(=O)CC1(N)CCN(c2nc(C#N)c3c(-c4cccc(Cl)c4Cl)n[nH]c3n2)CC1 ; CS(=O)(=O)CC1(N)CCN(c2nc(C(N)=O)c3c(-c4cccc(Cl)c4Cl)n[nH]c3n2)CC1]

### Step 1

### ((methylsulfonyl)methyl)lithium

(Methylsulfonyl)methane 43a (300 mg, 3.19 mmol) was added to tetrahydrofuran (4 mL), subjected to nitrogen gas displacement, dropwise added with a normal hexane solution (2.5 M, 1.66 mL) containing n-butyl lithium at −20° C., and continuously at −20° C. for 1 hour. After the reaction was completed, ((methylsulfonyl)methyl)lithium 43b was obtained, which was directly used for the next step without treatment.

### Step 2

### Tert-butyl 4-((tert-butylsulfinyl)amino)-4-((methylsulfonyl)methyl)piperidine-1-carboxylate

Tert-butyl 4-((tert-butylsulfinyl)imino)piperidine-1-carboxylate 43c (384 mg, 1.27 mmol, self-prepared according to patent WO 2007125321) was added to tetrahydrofuran (3 mL), then the reaction solution was dropwise added to the above-mentioned reaction system at −20° C., and continuously reacted for 1 hour. After the reaction was completed, the reaction solution was added with a saturated ammonium chloride solution and the reaction solution was extracted with ethyl acetate (30 mL×2) to separate an aqueous layer. Combined organic phases were washed with a saturated sodium chloride solution (30 mL×2) in turn, dried with anhydrous sodium sulfate, filtered, and concentrated under reduced pressure. The obtained residue was further analyzed and purified by silica gel column chromatography (eluent: system A) to obtain tert-butyl 4-((tert-butylsulfinyl)amino)-4-((methylsulfonyl)methyl)piperidine-1-carboxylate 43d (120 mg) with a yield of 23.83%.

MS m/z (ESI): 397.3 [M+1]

### Step 3

### 2-methyl-N-(4-((methylsulfonyl)methyl)piperidin-4-yl)propane-2-sulfinamide

Tert-butyl 4-((tert-butylsulfinyl)amino)-4-((methylsulfonyl)methyl)piperidine-1-carboxylate 43d (120 mg, 302.60 μmol) was added to dichloromethane (2.5 mL), then added with trifluoroacetic acid (0.5 mL) at room temperature, and reacted for 2 hours. After the reaction was completed, the reaction solution was concentrated under reduced pressure to obtain (2-methyl-N-(4-((methylsulfonyl)methyl)piperidin-4-yl)propane-2-sulfinamide 43e (89.71 mg) with a yield of 100.00%, which was directly used for the next reaction without purification.

MS m/z (ESI): 297.2 [M+1]

### Step 4

### N-(1-(3-bromo-4-cyano-1H-pyrazolo[3,4-d]pyrimidin-6-yl)-4-((methylsulfonyl)methyl)piperidin-4-yl)-2-methylpropane-2-sulfinamide

3-bromo-6-chloro-1H-pyrazolo[3,4-d]pyrimidine-4-carbonitrile 1d (76.67 mg, 296.63 μmol), 2-methyl-N-(4-((methylsulfonyl)methyl)piperidin-4-yl)propane-2-sulfinamide 43e (87.94 mg, 296.63 μmol) and N,N-diisopropylethylamine(191.68 mg, 1.48 mmol) were added to N,N-dimethylacetamide (2 mL), heated to 80° C., and reacted for 1 hour. After the reaction was completed, the reaction solution was concentrated under reduced pressure. The obtained residue was purified by silica gel column chromatography (eluent: system A) to obtain N-(1-(3-bromo-4-cyano-1H-pyrazolo[3,4-d]pyrimidin-6-yl)-4-((methylsulfonyl)methyl)piperidin-4-yl)-2-methylpropane-2-sulfinamide 43f (70 mg) with a yield of 45.52%.

MS m/z (ESI): 518.0 [M+1]

### Step 5

### N-(1-(3-(2,3-dichlorophenyl)-4-cyano-1H-pyrazolo[3,4-d]pyrimidin-6-yl)-4-((methylsulfonyl)methyl)piperidin-4-yl)-2-methylpropane-2-sulfinamide

N-(1-(3-bromo-4-cyano-1H-pyrazolo[3,4-d]pyrimidin-6-yl)-4-((methylsulfonyl)methyl)piperidin-4-yl)-2-methylpropane-2-sulfinamide 43f (70 mg, 135.02 μmol), (2,3-dichlorophenyl)boronic acid 1g (103.06 mg, 540.07 μmol), methanesulfonato(2-dicyclohexylphosphino-2′,6′-di-isopropoxy-1,1′-biphenyl)(2-amino-1,1′-biphenyl-2-yl)palladium (22.61 mg, 27.00 μmol), 2-dicyclohexylphosphino-2′,6′-di-isopropoxy-1,1′-biphenyl (25.20 mg, 54.01 μmol) and potassium phosphate (86.02 mg, 405.05 μmol) were added to a mixed solution of 1,4-dioxane (2 mL) and water (0.2 mL), subjected to argon gas displacement thrice, heated to 100° C., and reacted for 16 hours. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain N-(1-(3-(2,3-dichlorophenyl)-4-cyano-1H-pyrazolo[3,4-d]pyrimidin-6-yl)-4-((methylsulfonyl)methyl)piperidin-4-yl)-2-methylpropane-2-sulfinamide 43g (60 mg) with a yield of 76%.

MS m/z (ESI): 584.1 [M+1]

### Step 6

### 6-(4-amino-4-((methylsulfonyl)methyl)piperidin-1-yl)-3-(2,3-dichlorophenyl)-1H-pyrazolo[3,4-d]pyrimidine-4-carbonitrile

N-(1-(3-(2,3-dichlorophenyl)-4-cyano-1H-pyrazolo[3,4-d]pyrimidin-6-yl)-4-((methylsulfonyl)methyl)piperidin-4-yl)-2-methylpropane-2-sulfinamide 43g (10 mg, 17.11 μmol) was added to dichloromethane (2 mL), and dropwise added with hydrochloric acid in methanol (4 M, 17.11 μL), and reacted for 1 hour. After the reaction was completed, the reaction solution was concentrated under reduced pressure to obtain 6-(4-amino-4-((methylsulfonyl)methyl)piperidin-1-yl)-3-(2,3-dichlorophenyl)-1H-pyrazolo[3,4-d]pyrimidine-4-carbonitrile 43h (8.22 mg) with a yield of 100.00%, which was directly used for the next reaction without purification.

MS m/z (ESI): 479.8 [M+1]

### Step 7

### 6-(4-amino-4-((methylsulfonyl)methyl)piperidin-1-yl)-3-(2,3-dichlorophenyl)-1H-pyrazolo[3,4-d]pyrimidine-4-carboxamide

Sodium hydroxide solution (0.15 mL) was added to a solution of methanol (1 mL) containing 6-(4-amino-4-((methylsulfonyl)methyl)piperidin-1-yl)-3-(2,3-dichlorophenyl)-1H-pyrazolo[3,4-d]pyrimidine-4-carbonitrile 43h (8.22 mg, 17.11 μmol), then added with hydrogen peroxide (0.3 mL), and stirred at room temperature for 1 hour. After the reaction was completed, a trifluoroacetic acid was added to adjust the pH to be acidic, and then the reaction solution was subjected to liquid chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-((methylsulfonyl)methyl)piperidin-1-yl)-3-(2,3-dichlorophenyl)-1H-pyrazolo[3,4-d]pyrimidine-4-carboxamide 43 (7 mg) with a yield of 82.15%.

MS m/z (ESI): 498.1 [M+1]

¹H NMR (400 MHz, DMSO-d₆) δ 13.60 (s, 1H), 8.27 (s, 2H), 8.16 (s, 1H), 7.61-7.73 (m, 2H), 7.40 (d, J=4.9 Hz, 2H), 4.58 (s, 1H), 4.31 (s, 1H), 3.90 (s, 2H), 3.64 (s, 2H), 3.19 (s, 3H), 2.13 (d, J=13.6 Hz, 2H), 1.85 (s, 2H).

### Example 44

### 6-(4-amino-4-(6-methoxypyridin-3-yl)piperidin-1-yl)-3-(2,3-dichlorophenyl)-1H-pyrazolo[3,4-d]pyrimidine-4-carboxamide

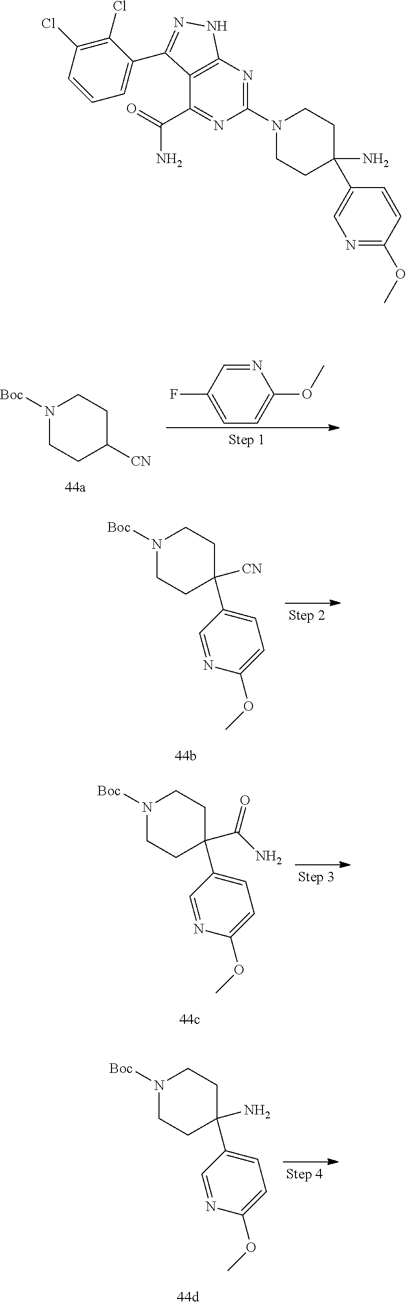

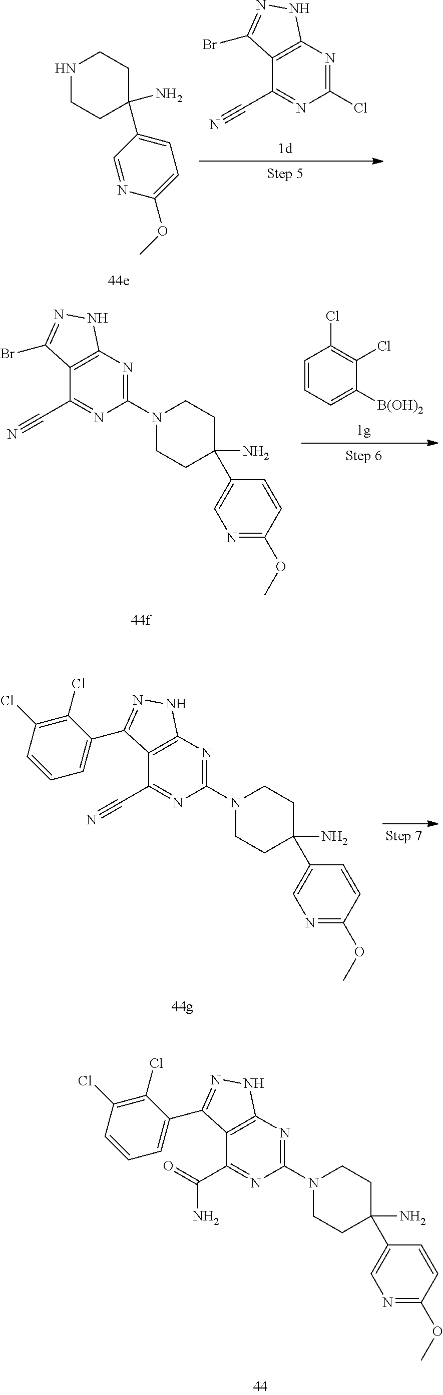 [SMILES: COc1ccc(C2(N)CCNCC2)cn1 ; N#Cc1nc(Cl)nc2[nH]nc(Br)c12 ; COc1ccc(C2(N)CCN(c3nc(C#N)c4c(Br)n[nH]c4n3)CC2)cn1 ; OB(O)c1cccc(Cl)c1Cl ; COc1ccc(C2(N)CCN(c3nc(C#N)c4c(-c5cccc(Cl)c5Cl)n[nH]c4n3)CC2)cn1 ; COc1ccc(C2(N)CCN(c3nc(C(N)=O)c4c(-c5cccc(Cl)c5Cl)n[nH]c4n3)CC2)cn1]

### Step 1

### Tert-butyl 4-(6-methoxypyridin-3-yl)-4-cyanopiperidine-1-carboxylate

At room temperature, tert-butyl 4-cyanopiperidine-1-carboxylate 44a (19.85 g, 94.4 mmol), 5-fluoro-2-methoxypyridine (3.0 g, 23.6 mmol) and 1 M tetrahydrofuran solution (35.4 mL) containing potassium bis(trimethylsilyl)amide were added to a solution of tetrahydrofuran (40 mL) in turn, and stirred for 1 hour under the protection of argon gas. After the reaction was completed, the reaction solution was cooled to room temperature, added with a saturated aqueous ammonium chloride solution (30 mL), concentrated under reduced pressure, then added with ethyl acetate (30 mL) for liquid separation, then aqueous phases were washed with ethyl acetate (30 mL×2), and organic phases were combined, and washed with saturated salt water, dried by anhydrous sodium sulfate, and concentrated under reduced pressure. The obtained residue was further separated and purified by silica gel column chromatography (eluent: system A) to obtain the product tert-butyl 4-(6-methoxypyridin-3-yl)-4-cyanopiperidine-1-carboxylate 44b (680 mg) with a yield of 9.1%.

MS m/z (ESI): 318.0 [M+1]

### Step 2

### Tert-butyl 4-carbamoyl-4-(6-methoxypyridin-3-yl)piperidine-1-carboxylate

Potassium hydroxide (240 mg, 4.3 mmol) and tert-butyl 4-cyano-4-(6-methoxypyridin-3-yl)piperidine-1-carboxylate 44b (680 mg, 2.14 mmol) were added to a solution of dimethyl sulfoxide (2 mL), and hydrogen peroxide (30%, 1 mL) was slowly added dropwise to the reaction solution. After the dropwise addition was completed, the reaction solution was stirred for 1 hour. After the reaction was completed, the reaction solution was added with 50 mL of water to precipitate a yellow solid, and filtered, then the filter cake was washed with water, and dried in vacuum to obtain the product tert-butyl 4-carbamoyl-4-(6-methoxypyridin-3-yl)piperidine-1-carboxylate 44c (370 mg) with a yield of 51.5%.

MS m/z (ESI): 335.9 [M+1]

### Step 3

### Tert butyl 4-amino-4-(6-methoxypyridin-3-yl)piperidine-1-carboxylate

[Bis(trifluoroacetoxy)iodo]benzene (522 mg, 1.21 mmol) was added to a solution of acetonitrile (2 mL) containing tert-butyl 4-carbamoyl-4-(6-methoxypyridin-3-yl)piperidine-1-carboxylate 44c (370 mg, 1.1 mmol), and stirred at room temperature for 2 hours. After the reaction was completed, the reaction solution was added with a saturated sodium bicarbonate solution (10 mL), and extracted with ethyl acetate (10 mL×3), then organic phases were combined, washed with a saturated sodium chloride solution (10 mL). The organic phases were dried with anhydrous sodium sulfate, filtered, and concentrated under reduced pressure. The obtained residue was purified by silica gel column chromatography (eluent: system A) to obtain the product tert-butyl 4-amino-4-(6-methoxypyridin-3-yl)piperidine-1-carboxylate 44d (170 mg) with a yield of 50.1%.

MS m/z (ESI): 307.8 [M+1]

### Step 4

### 4-(6-methoxypyridin-3-yl)piperidin-4-amine

A trifluoroacetic acid (1 mL) was dropwise added to 3 mL of dichloromethane solution containing tert-butyl 4-amino-4-(6-methoxypyridin-3-yl)piperidine-1-carboxylate 44d (170 mg, 553 μmol), and reacted at room temperature for 1 hour. The reaction solution was concentrated under reduced pressure to obtain 4-(6-methoxypyridin-3-yl)piperidin-4-amine 44e, which was directly used for the next reaction without purification.

MS m/z (ESI): 191.2 [M−16]

### Step 5

### 6-(4-amino-4-(6-methoxypyridin-3-yl)piperidin-1-yl)-3-bromo-1H-pyrazolo[3,4-d]pyrimidine-4-carbonitrile

N,N-diisopropylethylamine (225 mg, 1.74 mmol) and the above-mentioned crude product 4-(6-methoxypyridin-3-yl)piperidin-4-amine 44e were added to a solution of N-methyl pyrrolidone (5 mL) containing 3-bromo-6-chloro-1H-pyrazolo[3,4-d]pyrimidine-4-carbonitrile 1d (150 mg, 580 μmol), heated to 100° C., and stirred for 1 hour. After the reaction was completed, the reaction solution was subjected to reverse chromatographic purification (C₁₈ separation column 20-45 μm; mobile phase A: H₂O, mobile phase B: CH₃CN) to obtain the product 6-(4-amino-4-(6-methoxypyridin-3-yl)piperidin-1-yl)-3-bromo-1H-pyrazolo[3,4-d]pyrimidine-4-carbonitrile 44f (150 mg) with a yield of 63.2%.

MS m/z (ESI): 411.8 [M−16]

### Step 6

### 6-(4-amino-4-(6-methoxypyridin-3-yl)piperidin-1-yl)-3-(2,3-dichlorophenyl)-1H-pyrazolo[3,4-d]pyrimidine-4-carbonitrile

6-(4-amino-4-(6-methoxypyridin-3-yl)piperidin-1-yl)-3-bromo-1H-pyrazolo[3,4-d]pyrimidine-4-carbonitrile 44f (150 mg, 349 μmol), (2,3-dichlorophenyl)boronic acid 1g (266.7 mg, 1.4 mmol), methanesulfonato(2-dicyclohexylphosphino-2′,6′-di-isopropoxy-1,1′-biphenyl)(2-amino-1,1′-biphenyl-2-yl)palladium (58.5 mg, 69.9 μmol), 2-dicyclohexylphosphino-2′,6′-di-isopropoxy-1,1′-biphenyl (65.2 mg, 139.8 μmol) and potassium phosphate (222.5 mg, 1.05 mmol) were added to a mixed solution of 1,4-dioxane (5 mL) and water (1 mL), subjected to argon gas displacement thrice, heated to 100° C., and reacted overnight. After the reaction was completed, the reaction solution was concentrated under reduced pressure, and added with ethyl acetate (10 mL) and water (10 mL) for liquid separation, then aqueous phases were extracted with ethyl acetate (10 mL×2), and organic phases were combined, and washed with saturated salt water, dried by anhydrous sodium sulfate, and concentrated under reduced pressure. The obtained residue was purified by silica gel column chromatography (eluent: system A) to obtain 6-(4-amino-4-(6-methoxypyridin-3-yl)piperidin-1-yl)-3-(2,3-dichlorophenyl)-1H-pyrazolo[3,4-d]pyrimidine-4-carbonitrile 44g (30 mg) with a yield of 17.3%.

MS m/z (ESI): 477.8 [M−16]

### Step 7

### 6-(4-amino-4-(6-methoxypyridin-3-yl)piperidin-1-yl)-3-(2,3-dichlorophenyl)-1H-pyrazolo[3,4-d]pyrimidine-4-carboxamide

Potassium hydroxide (6.8 mg, 121 μmol) and 6-(4-amino-4-(6-methoxypyridin-3-yl)piperidin-1-yl)-3-(2,3-dichlorophenyl)-1H-pyrazolo[3,4-d]pyrimidine-4-carbonitrile 44g (30 mg, 61 μmol) were added to a solution of dimethyl sulfoxide (1 mL), and then hydrogen peroxide (30%, 0.5 mL) was slowly added dropwise to the reaction solution. After the dropwise addition was completed, the reaction solution was stirred for 1 hour. After the reaction was completed, a trifluoroacetic acid was dropwise added to adjust the pH to be 3-4, and then the reaction solution was subjected to liquid chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(6-methoxypyridin-3-yl)piperidin-1-yl)-3-(2,3-dichlorophenyl)-1H-pyrazolo[3,4-d]pyrimidine-4-carboxamide 44 (5 mg) with a yield of 16%.

MS m/z (ESI): 512.8 [M+1]

### Example 45

### 6-(4-amino-4-(6-chloropyridin-3-yl)piperidin-1-yl)-3-(2,3-dichlorophenyl)-1H-pyrazolo[3,4-d]pyrimidine-4-carboxamide

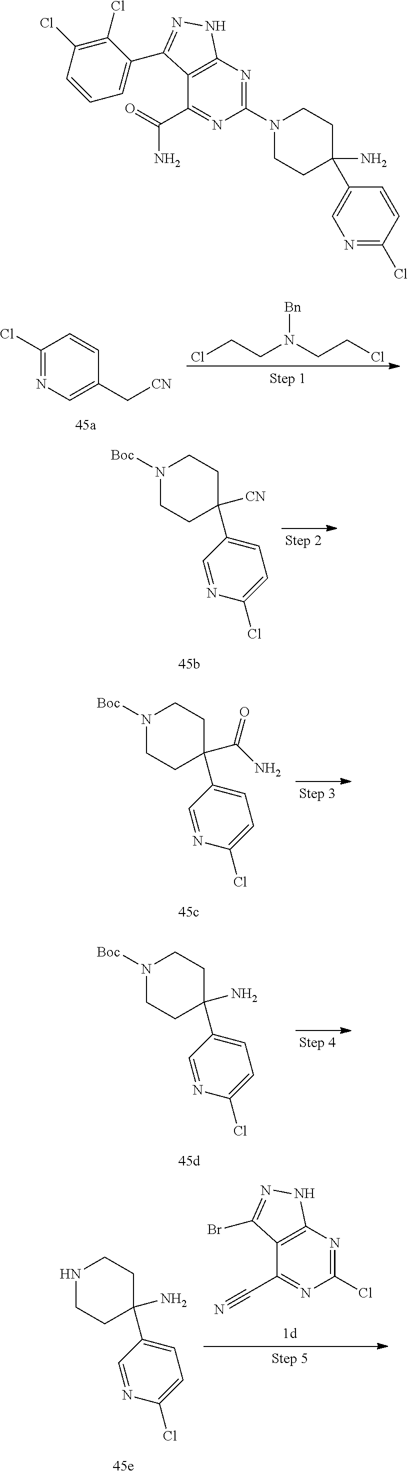

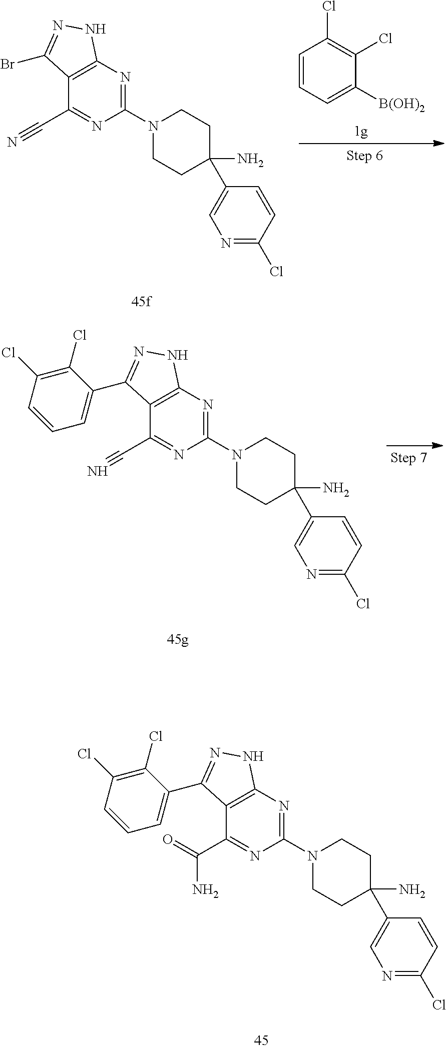 [SMILES: N#Cc1nc(N2CCC(N)(c3ccc(Cl)nc3)CC2)nc2[nH]nc(Br)c12 ; OB(O)c1cccc(Cl)c1Cl ; N#Cc1nc(N2CCC(N)(c3ccc(Cl)nc3)CC2)nc2[nH]nc(-c3cccc(Cl)c3Cl)c12 ; NC(=O)c1nc(N2CCC(N)(c3ccc(Cl)nc3)CC2)nc2[nH]nc(-c3cccc(Cl)c3Cl)c12]

### Step 1

### Tert-butyl 4-(6-chloropyridin-3-yl)-4-cyanopiperidine-1-carboxylate

In an ice water bath, sodium hydride (3.6 g, 90.0 mmol, 60%) was added to a solution of N,N-dimethylformamide (20 mL) containing 2-(6-chloropyridin-3-yl)acetonitrile 45a (2.29 g, 15 mmol) and tert-butyl bis(2-chloroethyl)carbamate (3.99 g, 16.5 mmol), stirred for 1 hour, heated to 60° C., and then stirred overnight. After the reaction was completed, the reaction solution was cooled to room temperature, quenched with a saturated aqueous ammonium chloride solution (30 mL), and added with ethyl acetate (30 mL) for liquid separation, then aqueous phases were washed with ethyl acetate (30 mL×2), and organic phases were combined, and washed with saturated salt water, dried with anhydrous sodium sulfate, and concentrated under reduced pressure. The obtained residue was purified by silica gel column chromatography (eluent: system A) to obtain the product tert-butyl 4-(6-chloropyridin-3-yl)-4-cyanopiperidine-1-carboxylate 45b (0.9 g) with a yield of 18.7%.

MS m/z (ESI): 322.0 [M+1]

### Step 2

### Tert-butyl 4-carbamoyl-4-(6-chloropyridin-3-yl)piperidine-1-carboxylate

Potassium hydroxide (314 mg, 5.6 mmol) and tert-butyl 4-(6-chloropyridin-3-yl)-4-cyanopiperidine-1-carboxylate 45b (0.9 g, 2.8 mmol) were added to a solution of dimethyl sulfoxide (4 mL), and hydrogen peroxide (30%, 2 mL) was slowly added dropwise to the reaction solution. After the dropwise addition was completed, the reaction solution was stirred for 1 hour. After the reaction was completed, the reaction solution was added with 50 mL of water to precipitate a yellow solid, and filtered, then the filter cake was washed with water, and dried in vacuum to obtain the product tert-butyl 4-carbamoyl-4-(6-chloropyridin-3-yl)piperidine-1-carboxylate 45c (0.9 g) with a yield of 94.7%.

MS m/z (ESI): 340.0 [M+1]

### Step 3

### Tert-butyl 4-amino-4-(6-chloropyridine-3-yl)piperidine-1-carboxylate

[Bis(trifluoroacetoxy)iodo]benzene (1.25 g, 2.91 mmol) was added to a solution of acetonitrile (10 mL) containing tert-butyl 4-carbamoyl-4-(6-chloropyridin-3-yl)piperidine-1-carboxylate 45c (0.9 g, 2.65 mmol), and stirred at room temperature for 2 hours. After the reaction was completed, the reaction solution was added with a saturated sodium bicarbonate solution (40 mL), and extracted with ethyl acetate (30 mL×3), then organic phases were combined, washed with a saturated sodium chloride solution (30 mL). The organic phases were dried with anhydrous sodium sulfate, filtered, and concentrated under reduced pressure. The obtained residue was purified by silica gel column chromatography (eluent: system A) to obtain the product tert-butyl 4-amino-4-(6-chloropyridin-3-yl)piperidine-1-carboxylate 45d (0.4 g) with a yield of 48.4%.

MS m/z (ESI): 311.9 [M+1]

### Step 4

### 4-(6-chloropyridin-3-yl)piperidin-4-amine

A trifluoroacetic acid (1 mL) was dropwise added to a solution of dichloromethane (3 mL) containing tert-butyl 4-amino-4-(6-chloropyridin-3-yl)piperidine-1-carboxylate 45d (300 mg, 962 μmol), and stirred at room temperature for 1 hour. The reaction solution was concentrated under reduced pressure to obtain 4-(6-chloropyridin-3-yl)piperidin-4-amine 45e, which was directly used for the next reaction without purification.

MS m/z (ESI): 212.0 [M+1]

### Step 5

### 6-(4-amino-4-(6-chloropyridin-3-yl)piperidin-1-yl)-3-bromo-1H-pyrazolo[3,4-d]pyrimidine-4-carbonitrile

N,N-diisopropylethylamine (300 mg, 2.32 mmol) and the above-mentioned crude product 4-(6-chloropyridin-3-yl)piperidin-4-amine 45e were added to a solution of N-methyl pyrrolidone (5 mL) containing 3-bromo-6-chloro-1H-pyrazolo[3,4-d]pyrimidine-4-carbonitrile 1d (200 mg, 774 μmol), heated to 100° C., and stirred for 1 hour. After the reaction was completed, the reaction solution was purified on a reversed phase chromatographic column (C₁₈ separation column 20-45 μm; mobile phase A: H₂O, mobile phase B: CH₃CN) to obtain 6-(4-amino-4-(6-chloropyridin-3-yl)piperidin-1-yl)-3-bromo-1H-pyrazolo[3,4-d]pyrimidine-4-carbonitrile 45f (280 mg) with a yield of 83.4%.

MS m/z (ESI): 415.8 [M−16]

### Step 6

### 6-(4-amino-4-(6-chloropyridin-3-yl)piperidin-1-yl)-3-(2,3-dichlorophenyl)-1H-pyrazolo[3,4-d]pyrimidine-4-carbonitrile

6-(4-amino-4-(6-chloropyridin-3-yl)piperidin-1-yl)-3-bromo-1H-pyrazolo[3,4-d]pyrimidine-4-carbonitrile 45f (280 mg, 646 μmol), (2,3-dichlorophenyl)boronic acid 1g (493 mg, 2.58 mmol), methanesulfonato(2-dicyclohexylphosphino-2′,6′-di-isopropoxy-1,1′-biphenyl)(2-amino-1,1′-biphenyl-2-yl)palladium (108 mg, 129 μmol), 2-dicyclohexylphosphino-2′,6′-di-isopropoxy-1,1′-biphenyl (121 mg, 258 μmol) and potassium phosphate (411 mg, 1.94 mmol) were added to a mixed solution of 1,4-dioxane (5 mL) and water (1 mL), subjected to argon gas displacement thrice, heated to 100° C., and reacted overnight. After the reaction was completed, the reaction solution was concentrated under reduced pressure, and added with ethyl acetate (10 mL) and water (10 mL) for liquid separation, then aqueous phases were extracted with ethyl acetate (10 mL×2), and organic phases were combined, and washed with saturated salt water, dried by anhydrous sodium sulfate, and concentrated under reduced pressure. The obtained residue was purified by silica gel column chromatography (eluent: system A) to obtain 6-(4-amino-4-(6-chloropyridin-3-yl)piperidin-1-yl)-3-(2,3-dichlorophenyl)-1H-pyrazolo[3,4-d]pyrimidine-4-carbonitrile 45g (120 mg) with a yield of 37.2%.

MS m/z (ESI): 481.8 [M−16]

### Step 7

### 6-(4-amino-4-(6-chloropyridin-3-yl)piperidin-1-yl)-3-(2,3-dichlorophenyl)-1H-pyrazolo[3,4-d]pyrimidine-4-carboxamide

Potassium hydroxide (8.1 mg, 144 μmol)) and 6-(4-amino-4-(6-chloropyridin-3-yl)piperidin-1-yl)-3-(2,3-dichlorophenyl)-1H-pyrazolo[3,4-d]pyrimidine-4-carbonitrile 45g (36 mg, 72 μmol) were added to a solution of dimethyl sulfoxide (1 mL), and then hydrogen peroxide (30%, 0.5 mL) was slowly added dropwise to the reaction solution. After the dropwise addition was completed, the reaction solution was stirred for 1 hour. After the reaction was completed, a trifluoroacetic acid was dropwise added to adjust the pH to be 3-4, and then the reaction solution was subjected to liquid chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(6-chloropyridin-3-yl)piperidin-1-yl)-3-(2,3-dichlorophenyl)-1H-pyrazolo[3,4-d]pyrimidine-4-carboxamide 45 (12 mg) with a yield of 32%.

MS m/z (ESI): 516.8 [M+1]

¹H NMR (400 MHz, DMSO-d₆) δ 8.76 (d, J=4.0 Hz, 1H), 8.54 (br, 3H), 8.09-8.20 (m, 2H), 7.62-7.76 (m, 3H), 7.35-7.44 (m, 2H), 4.11-4.67 (m, 2H), 3.40-3.70 (m, 2H), 2.55-2.72 (m, 2H), 2.02-2.20 (m, 2H).

### Example 46

### 6-(4-amino-4-(4-methoxyphenyl)piperidin-1-yl)-3-(2,3-dichlorophenyl)-1H-pyrazolo[3,4-d]pyrimidine-4-carboxamide

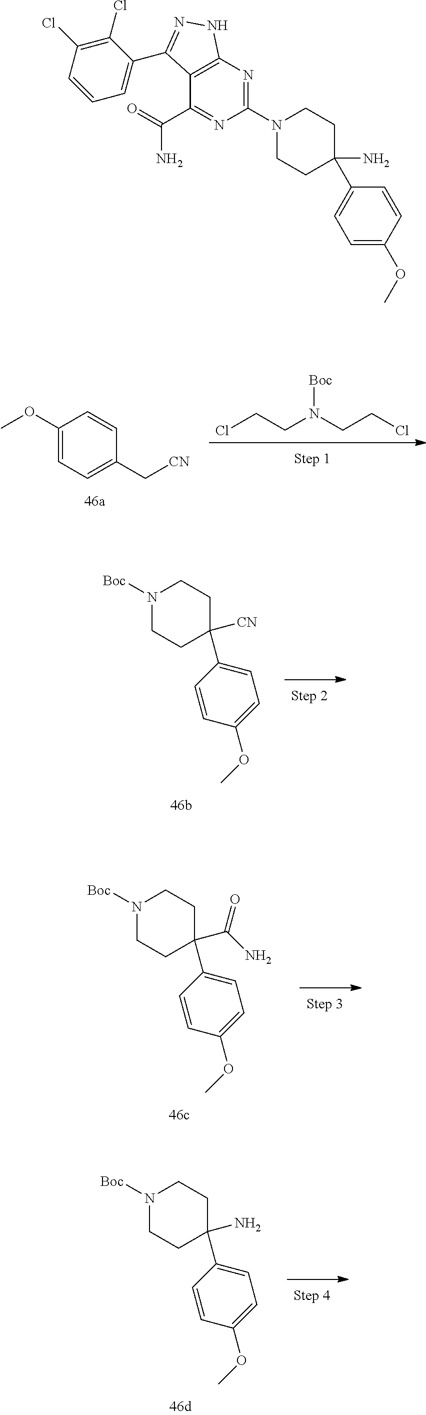 [SMILES: COc1ccc(C2(N)CCN(c3nc(C(N)=O)c4c(-c5cccc(Cl)c5Cl)n[nH]c4n3)CC2)cc1 ; COc1ccc(CC#N)cc1 ; CC(C)(C)OC(=O)N(CCCl)CCCl ; COc1ccc(C2(C#N)CCN(C(=O)OC(C)(C)C)CC2)cc1 ; COc1ccc(C2(C(N)=O)CCN(C(=O)OC(C)(C)C)CC2)cc1 ; COc1ccc(C2(N)CCN(C(=O)OC(C)(C)C)CC2)cc1]

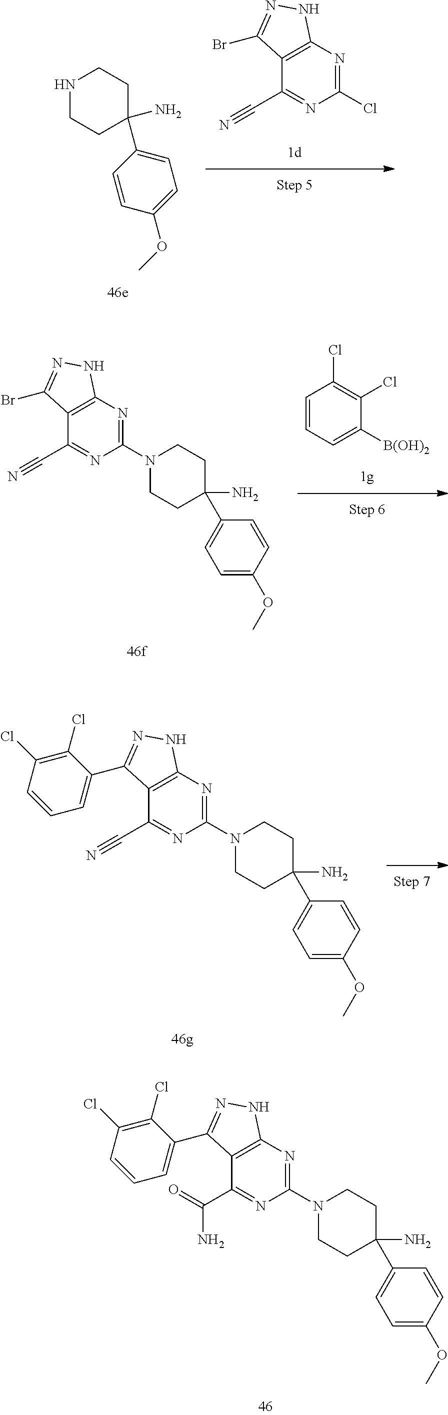 [SMILES: COc1ccc(C2(N)CCNCC2)cc1 ; N#Cc1nc(Cl)nc2[nH]nc(Br)c12 ; COc1ccc(C2(N)CCN(c3nc(C#N)c4c(Br)n[nH]c4n3)CC2)cc1 ; OB(O)c1cccc(Cl)c1Cl ; COc1ccc(C2(N)CCN(c3nc(C#N)c4c(-c5cccc(Cl)c5Cl)n[nH]c4n3)CC2)cc1 ; COc1ccc(C2(N)CCN(c3nc(C(N)=O)c4c(-c5cccc(Cl)c5Cl)n[nH]c4n3)CC2)cc1]

### Step 2

### Tert-butyl 4-cyano-4-(4-methoxyphenyl)piperidine-1-carboxylate

In an ice water bath, tert-butyl bis(2-chloroethyl)carbamate (4 g, 16.52 mmol) and 2-(4-methoxyphenyl)acetonitrile 46a (2.21 g, 15.02 mmol) were added to N,N-dimethylformamide (35 mL) in turn, added with 60% sodium hydride (3 g, 75.09 mmol) in batches into the above-mentioned mixed solution, and then heated to 60° C., and reacted for 5 hours. The reaction solution was cooled to room temperature, quenched with water (100 mL), and extracted with ethyl acetate (100 mL×2). Organic phases were combined, washed with a saturated sodium chloride solution (100 mL), dried with anhydrous sodium sulfate and concentrated under reduced pressure. The obtained residue was purified by silica gel column chromatography (eluent: system A) to obtain tert-butyl 4-cyano-4-(4-methoxyphenyl)piperidine-1-carboxylate 46b (1.78 g) with a yield of 37.5%.

MS m/z (ESI): 217.1 [M−99]

### Step 2

### Tert-butyl 4-carbamoyl-4-(4-methoxyphenyl)piperidine-1-carboxylate

Tert-butyl 4-cyano-4-(4-methoxyphenyl)piperidine-1-carboxylate 46b (1.78 g, 5.63 mmol) and potassium hydroxide (631.34 mg, 11.25 mmol) were added to dimethyl sulfoxide (8 mL) in turn, dropwise added with hydrogen peroxide (4 mL), and continuously stirred for 1 hour. The reaction solution was added with a large amount of water (50 mL) to precipitate a white solid, which was filtered and dried to obtain tert-butyl 4-carbamoyl-4-(4-methoxyphenyl)piperidine-1-carboxylate 46c (1.45 g) with a yield of 77.1%.

MS m/z (ESI): 279.0 [M−55]

### Step 3

### Tert-butyl 4-amino-4-(4-methoxyphenyl)piperidine-1-carboxylate

1,3-dibromo-5,5-dimethylhydantoin (619.88 mg, 2.17 mmol), potassium hydroxide (1.09 g, 19.51 mmol) and tert-butyl 4-carbamoyl-4-(4-methoxyphenyl)piperidine-1-carboxylate 46c (1.45 g, 4.34 mmol) were added to a mixed solution of acetonitrile (10 mL) and water (10 mL) in turn, and stirred at room temperature for 2 hours. The reaction solution was concentrated under reduced pressure and purified on a C₁₈ reversed phase chromatographic column (C₁₈ separation column 20-45 μm; mobile phase A: H₂O, mobile phase B: CH₃CN) to obtain tert-butyl 4-amino-4-(4-methoxyphenyl)piperidine-1-carboxylate 46d (1.18 g) with a yield of 88.8%.

MS m/z (ESI): 234.1 [M−72]

### Step 4

### 4-(4-methoxyphenyl)piperidin-4-amine

Tert-butyl 4-amino-4-(4-methoxyphenyl)piperidine-1-carboxylate 46d (818 mg, 2.67 mmol) was dissolved in dichloromethane (10 mL), slowly added with a trifluoroacetic acid (6.56 g, 57.51 mmol), and stirred overnight at room temperature. After the reaction was completed, the reaction solution was concentrated under reduced pressure to obtain 4-(4-methoxyphenyl)piperidin-4-amine 46e, which was directly used for the next reaction without purification.

MS m/z (ESI): 190.1 [M−16]

### Step 5

### 6-(4-amino-4-(4-methoxyphenyl)piperidin-1-yl)-3-bromo-1H-pyrazolo[3,4-d]pyrimidine-4-carbonitrile

The above-mentioned crude product 4-(4-methoxyphenyl)piperidin-4-amine 46e, 3-bromo-6-chloro-1H-pyrazolo[3,4-d]pyrimidine-4-carbonitrile 1d (689.12 mg, 2.67 mmol) and N,N-diisopropylethylamine (1.38 g, 10.66 mmol) were added to N-methyl pyrrolidone (8 mL) in turn, subjected to argon gas replacement thrice, and continuously stirred at 100° C. for 4 hours. The reaction solution was quenched with water (30 mL), and extracted with ethyl acetate (30 mL×3). Organic phases were combined, dried with anhydrous sodium sulfate and concentrated under reduced pressure. The obtained residue was purified by silica gel column chromatography (eluent: system B) to obtain 6-(4-amino-4-(4-methoxyphenyl)piperidin-1-yl)-3-bromo-1H-pyrazolo[3,4-d]pyrimidine-4-carbonitrile 46f (1.1 g) with a yield of 96.3%.

MS m/z (ESI): 411.0 [M−16]

### Step 6

### 6-(4-amino-4-(4-methoxyphenyl)piperidin-1-yl)-3-(2,3-dichlorophenyl)-1H-pyrazolo[3,4-d]pyrimidine-4-carbonitrile

6-(4-amino-4-(4-methoxyphenyl)piperidin-1-yl)-3-bromo-1H-pyrazolo[3,4-d]pyrimidine-4-carbonitrile 46f (1.1 g, 2.57 mmol), (2,3-dichlorophenyl)boronic acid 1g (1.96 g, 10.27 mmol), methanesulfonato(2-dicyclohexylphosphino-2′,6′-di-isopropoxy-1,1′-biphenyl)(2-amino-1,1′-biphenyl-2-yl)palladium (430.14 mg, 513.68 μmol), 2-dicyclohexylphosphino-2′,6′-di-isopropoxy-1,1′-biphenyl (479.39 mg, 1.03 mmol) and potassium phosphate (2.18 g, 10.27 mmol) were added to a mixed solution of 1,4-dioxane (12 mL) and water (1.2 mL) in turn, subjected to argon gas displacement thrice, heated to 100° C., and reacted overnight. After the reaction was completed, the reaction solution was concentrated under reduced pressure, added with water (10 mL) and extracted with ethyl acetate thrice (100 mL×3), then organic phases were combined, and washed with saturated salt water, dried by anhydrous sodium sulfate, and concentrated under reduced pressure. The obtained residue was purified by silica gel column chromatography (eluent: system B) to obtain 6-(4-amino-4-(4-methoxyphenyl)piperidin-1-yl)-3-(2,3-dichlorophenyl)-1H-pyrazolo[3,4-d]pyrimidine-4-carbonitrile 46g (400 mg) with a yield of 31.5%.

MS m/z (ESI): 476.9 [M−16]

### Step 7

### 6-(4-amino-4-(4-methoxyphenyl)piperidin-1-yl)-3-(2,3-dichlorophenyl)-1H-pyrazolo[3,4-d]pyrimidine-4-carboxamide

6-(4-amino-4-(4-methoxyphenyl)piperidin-1-yl)-3-(2,3-dichlorophenyl)-1H-pyrazolo[3,4-d]pyrimidine-4-carbonitrile 46g (300 mg, 606.83 μmol) and potassium hydroxide (68.10 mg, 1.21 mmol) were added to dimethyl sulfoxide (4 mL) in turn, and then dropwise added with hydrogen peroxide (1 mL). After the reaction solution was continuously stirred at room temperature for 1 hour, the reaction solution was added with water (20 mL) to precipitate a faint yellow solid, neutralized with a dilute hydrochloric acid, and extracted with ethyl acetate (20 mL×3), then organic phases were combined, and washed with saturated salt water, dried by anhydrous sodium sulfate, and concentrated under reduced pressure. The obtained residue was purified by silica gel column chromatography (eluent: system B) to obtain 6-(4-amino-4-(4-methoxyphenyl)piperidin-1-yl)-3-(2,3-dichlorophenyl)-1H-pyrazolo[3,4-d]pyrimidine-4-carboxamide 46 (200 mg) with a yield of 64.6%.

MS m/z (ESI): 495.1 [M−16]

¹H NMR (400 MHz, CD₃OD) δ 7.62-7.66 (m, 2H), 7.58 (dd, J=7.6, 2.0 Hz, 1H), 7.33-7.40 (m, 2H), 7.08-7.12 (m, 2H), 4.72-4.75 (m, 2H), 3.85 (s, 3H), 3.33-3.40 (m, 2H), 2.72-2.76 (m, 2H), 2.08-2.16 (m, 2H).

### Example 47

### 6-(4-amino-4-(4-hydroxyphenyl)piperidin-1-yl)-3-(2,3-dichlorophenyl)-1H-pyrazolo[3,4-d]pyrimidine-4-carboxamide

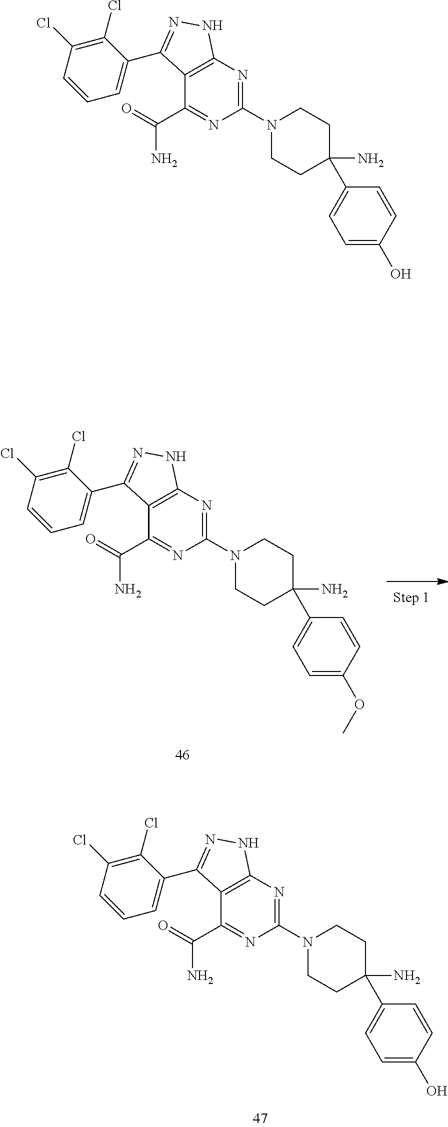 [SMILES: NC(=O)c1nc(N2CCC(N)(c3ccc(O)cc3)CC2)nc2[nH]nc(-c3cccc(Cl)c3Cl)c12 ; COc1ccc(C2(N)CCN(c3nc(C(N)=O)c4c(-c5cccc(Cl)c5Cl)n[nH]c4n3)CC2)cc1 ; NC(=O)c1nc(N2CCC(N)(c3ccc(O)cc3)CC2)nc2[nH]nc(-c3cccc(Cl)c3Cl)c12]

### Step 1

### 6-(4-amino-4-(4-hydroxyphenyl)piperidin-1-yl)-3-(2,3-dichlorophenyl)-1H-pyrazolo[3,4-d]pyrimidine-4-carboxamide

6-(4-amino-4-(4-methoxyphenyl)piperidin-1-yl)-3-(2,3-dichlorophenyl)-1H-pyrazolo[3,4-d]pyrimidine-4-carboxamide 46 (50 mg, 97.58 μmol) was dissolved in dichloromethane (3 mL), slowly added dropwise with boron tribromide (5 mL, 1.0 M dichloromethane solution), and reacted at room temperature for 2 hours. After the reaction was completed, the reaction solution was added with ice water (100 mL), and extracted with dichloromethane (100 mL×3). Organic phases were combined, concentrated under reduced pressure, and subjected to liquid chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(4-hydroxyphenyl)piperidin-1-yl)-3-(2,3-dichlorophenyl)-1H-pyrazolo[3,4-d]pyrimidine-4-carboxamide 47 (15 mg) with a yield of 30.9%.

MS m/z (ESI): 481.1 [M−16]

¹H NMR (400 MHz, CD₃OD) δ 7.58 (dd, J=7.6, 2.0 Hz, 1H), 7.52-7.55 (m, 2H), 7.33-7.40 (m, 2H), 6.94-6.96 (m, 2H), 4.72-4.76 (m, 2H), 3.32-3.38 (m, 2H), 2.71-2.75 (m, 2H), 2.06-2.13 (m, 2H).

### Example 48

### 6-(4-amino-4-(2-methoxyphenyl)piperidin-1-yl)-3-(2,3-dichlorophenyl)-1H-pyrazolo[3,4-d]pyrimidine-4-carboxamide

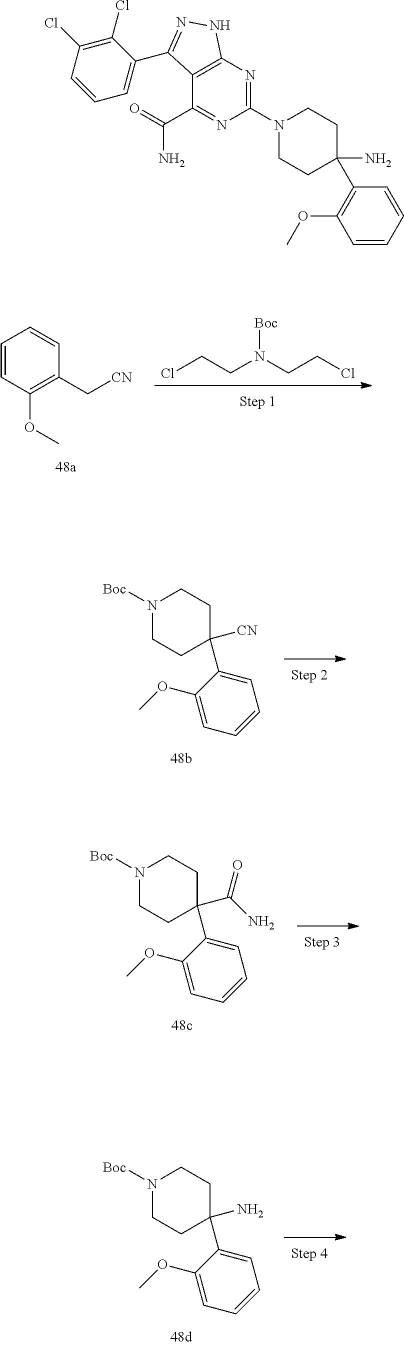 [SMILES: COc1ccccc1C1(N)CCN(c2nc(C(N)=O)c3c(-c4cccc(Cl)c4Cl)n[nH]c3n2)CC1 ; COc1ccccc1CC#N ; CC(C)(C)OC(=O)N(CCCl)CCCl ; COc1ccccc1C1(C#N)CCN(C(=O)OC(C)(C)C)CC1 ; COc1ccccc1C1(C(N)=O)CCN(C(=O)OC(C)(C)C)CC1 ; COc1ccccc1C1(N)CCN(C(=O)OC(C)(C)C)CC1]

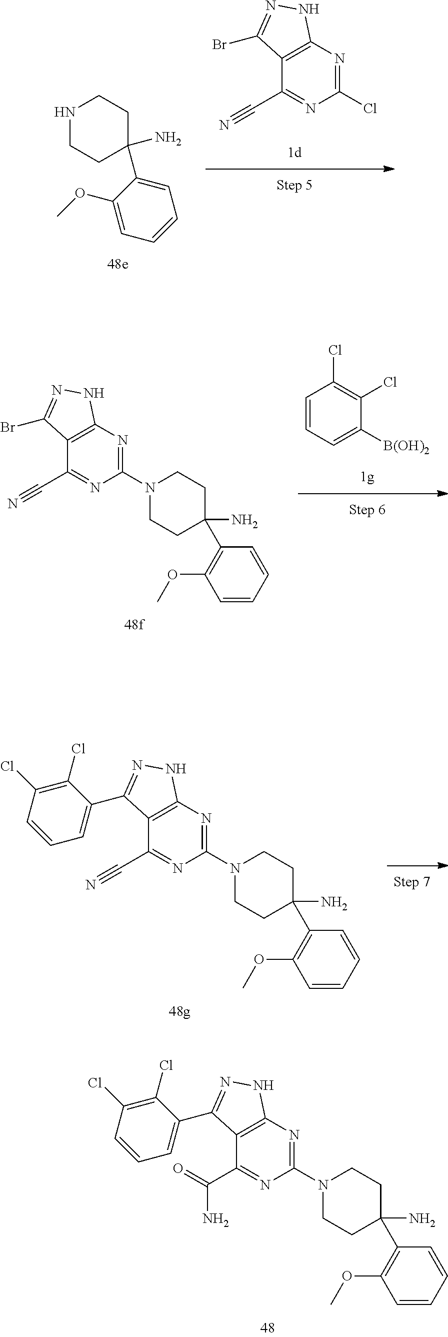 [SMILES: COc1ccccc1C1(N)CCNCC1 ; N#Cc1nc(Cl)nc2[nH]nc(Br)c12 ; COc1ccccc1C1(N)CCN(c2nc(C#N)c3c(Br)n[nH]c3n2)CC1 ; OB(O)c1cccc(Cl)c1Cl ; COc1ccccc1C1(N)CCN(c2nc(C#N)c3c(-c4cccc(Cl)c4Cl)n[nH]c3n2)CC1 ; COc1ccccc1C1(N)CCN(c2nc(C(N)=O)c3c(-c4cccc(Cl)c4Cl)n[nH]c3n2)CC1]

### Step 1

### Tert-butyl 4-cyano-4-(2-methoxyphenyl)piperidine-1-carboxylate

2-(2-methoxyphenyl)acetonitrile 48a (1 g, 6.79 mmol) and tert-butyl bis(2-chloroethyl)carbamate (1.81 g, 7.47 mmol) were dissolved in N,N-dimethylformamide (6 mL), added with 60% sodium hydride (1.06 g, 26.51 mmol) in batches, stirred for 40 minutes, heated to 70° C., and reacted overnight. The reaction solution was cooled to room temperature, quenched with water (100 mL), extracted with ethyl acetate (100 mL×3). Organic phases were combined, washed with a saturated sodium chloride solution (100 mL), dried with anhydrous sodium sulfate and concentrated under reduced pressure. The obtained residue was purified by silica gel column chromatography (eluent: system A) to obtain tert-butyl 4-cyano-4-(2-methoxyphenyl)piperidine-1-carboxylate 48b (1.7 g) with a yield of 79.1%.

MS m/z (ESI): 217.0 [M−99]

### Step 2

### Tert-butyl 4-carbamoyl-4-(2-methoxyphenyl)piperidine-1-carboxylate

Potassium hydroxide (283.75 mg, 5.06 mmol) and tert-butyl 4-cyano-4-(2-methoxyphenyl)piperidine-1-carboxylate 48b (0.8 g, 2.53 mmol) were dissolved in dimethyl sulfoxide (5 mL), slowly added dropwise with hydrogen peroxide (5 mL), and reacted at room temperature overnight. The reaction solution was added with a large amount of water to precipitate a yellow solid, which was filtered, then the filter cake was washed with water and dried to obtain tert-butyl 4-carbamoyl-4-(2-methoxyphenyl)piperidine-1-carboxylate 48c (400 mg) with a yield of 47.3%.

MS m/z (ESI): 279.0 [M−55]

### Step 3

### Tert-butyl 4-amino-4-(2-methoxyphenyl)piperidine-1-carboxylate

Potassium hydroxide (302.02 mg, 5.38 mmol) was added to a mixed solution of acetonitrile (2 mL) and water (3 mL) containing tert-butyl 4-carbamoyl-4-(2-methoxyphenyl)piperidine-1-carboxylate 48c (400 mg, 1.20 mmol), added with 1,3-dibromo-5,5-dimethylhydantoin (188.10 mg, 657.88 μmol) in batches, and stirred at room temperature for 1 hour. The reaction solution was added with water (100 mL) and potassium phosphate (279.30 mg, 1.32 mmol) and stirred for 15 minutes, then added with ethyl acetate (20 mL) and sodium sulphite (15.07 mg, 119.61 μmol) for liquid separation, aqueous phases were extracted with ethyl acetate (20 mL×2), organic phases were combined and washed with a sodium chloride solution (20 mL), dried, and concentrated under reduced pressure to obtain tert-butyl 4-amino-4-(2-methoxyphenyl)piperidine-1-carboxylate 48d (366 m) with a yield of 99.6%, which was directly used for the next reaction without purification.

MS m/z (ESI): 234.1 [M−72]

### Step 4

### 4-(2-methoxyphenyl)piperidin-4-amine

Tert-butyl 4-amino-4-(2-methoxyphenyl)piperidine-1-carboxylate 48d (366 mg, 1.19 mmol) was dissolved in dichloromethane (15 mL), slowly added with a trifluoroacetic acid (3 g, 26.31 mmol), and stirred overnight at room temperature. After the reaction was completed, the reaction solution was concentrated under reduced pressure to obtain 4-(2-methoxyphenyl)piperidin-4-amine 48e, which was directly used for the next reaction without purification.

MS m/z (ESI): 190.1 [M−16]

### Step 5

### 6-(4-amino-4-(2-methoxyphenyl)piperidin-1-yl)-3-bromo-1H-pyrazolo[3,4-d]pyrimidine-4-carbonitrile

3-bromo-6-chloro-1H-pyrazolo[3,4-d]pyrimidine-4-carbonitrile 1d (283.17 mg, 1.10 mmol), the above-mentioned crude product 4-(2-methoxyphenyl)piperidin-4-amine 48e and N,N-diisopropylethylamine (566.37 mg, 4.38 mmol) were added to N-methyl pyrrolidone (3 mL) in turn, subjected to argon gas replacement, and continuously stirred at 100° C. for 2 hours. The reaction solution was quenched with water (30 mL), and extracted with ethyl acetate (30 mL×3). Organic phases were combined, dried with anhydrous sodium sulfate and concentrated under reduced pressure. The obtained residue was purified by silica gel column chromatography (eluent: system B) to obtain 6-(4-amino-4-(2-methoxyphenyl)piperidin-1-yl)-3-bromo-1H-pyrazolo[3,4-d]pyrimidine-4-carbonitrile 48f (360 mg) with a yield of 70.8%.

MS m/z (ESI): 411.0 [M−16]

### Step 6

### 6-(4-amino-4-(2-methoxyphenyl)piperidin-1-yl)-3-(2,3-dichlorophenyl)-1H-pyrazolo[3,4-d]pyrimidine-4-carbonitrile

6-(4-amino-4-(2-methoxyphenyl)piperidin-1-yl)-3-bromo-1H-pyrazolo[3,4-d]pyrimidine-4-carbonitrile 48f (360 mg, 840.56 μmol), (2,3-dichlorophenyl)boronic acid 1g (641.58 mg, 3.36 mmol), methanesulfonato(2-dicyclohexylphosphino-2′,6′-di-isopropoxy-1,1′-biphenyl)(2-amino-1,1′-biphenyl-2-yl)palladium (140.77 mg, 168.11 μmol), 2-dicyclohexylphosphino-2′,6′-di-isopropoxy-1,1′-biphenyl (156.89 mg, 336.22 μmol) and potassium phosphate (713.70 mg, 3.36 mmol) were added to a mixed solution of 1,4-dioxane (4 mL) and water ((0.4 mL) in turn, subjected to argon gas displacement thrice, heated to 100° C., and reacted overnight. After the reaction was completed, the reaction solution was cooled to room temperature, added with water (20 mL) and extracted with ethyl acetate (50 mL×3), then organic phases were combined, and washed with saturated salt water, dried by anhydrous sodium sulfate, and concentrated under reduced pressure. The obtained residue was purified by silica gel column chromatography (eluent: system B) to obtain 6-(4-amino-4-(2-methoxyphenyl)piperidin-1-yl)-3-(2,3-dichlorophenyl)-1H-pyrazolo[3,4-d]pyrimidine-4-carbonitrile 48g (160 mg) with a yield of 38.5%.

MS m/z (ESI): 477.1 [M−16]

### Step 7

### 6-(4-amino-4-(2-methoxyphenyl)piperidin-1-yl)-3-(2,3-dichlorophenyl)-1H-pyrazolo[3,4-d]pyrimidine-4-carboxamide

6-(4-amino-4-(2-methoxyphenyl)piperidin-1-yl)-3-(2,3-dichlorophenyl)-1H-pyrazolo[3,4-d]pyrimidine-4-carbonitrile 48g (160 mg, 323.64 μmol) and potassium hydroxide (36.32 mg, 647.28 μmol) were added to dimethyl sulfoxide (5 mL) in turn, and then dropwise added with hydrogen peroxide (0.5 mL). After the reaction solution was continuously stirred at room temperature for 1 hour, the reaction solution was added with water (20 mL) to precipitate a faint yellow solid, neutralized with a dilute hydrochloric acid, and extracted with ethyl acetate (20 mL×3), then organic phases were combined, and washed with saturated salt water, dried by anhydrous sodium sulfate, and concentrated under reduced pressure. The obtained residue was purified by silica gel column chromatography (eluent: system B) to obtain 6-(4-amino-4-(2-methoxyphenyl)piperidin-1-yl)-3-(2,3-dichlorophenyl)-1H-pyrazolo[3,4-d]pyrimidine-4-carboxamide 48 (90 mg) with a yield of 54.4%.

MS m/z (ESI): 495.1 [M−16]

### Example 49

### 6-(4-amino-4-(2-hydroxyphenyl)piperidin-1-yl)-3-(2,3-dichlorophenyl)-1H-pyrazolo[3,4-d]pyrimidine-4-carboxamide

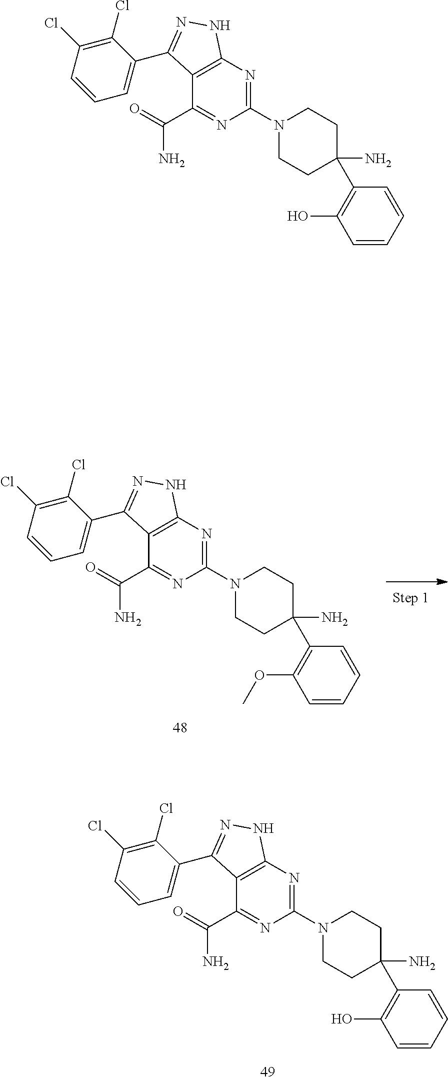 [SMILES: NC(=O)c1nc(N2CCC(N)(c3ccccc3O)CC2)nc2[nH]nc(-c3cccc(Cl)c3Cl)c12 ; COc1ccccc1C1(N)CCN(c2nc(C(N)=O)c3c(-c4cccc(Cl)c4Cl)n[nH]c3n2)CC1 ; NC(=O)c1nc(N2CCC(N)(c3ccccc3O)CC2)nc2[nH]nc(-c3cccc(Cl)c3Cl)c12]

### Step 1

### 6-(4-amino-4-(2-hydroxyphenyl)piperidin-1-yl)-3-(2,3-dichlorophenyl)-1H-pyrazolo[3,4-d]pyrimidine-4-carboxamide

6-(4-amino-4-(2-methoxyphenyl)piperidin-1-yl)-3-(2,3-dichlorophenyl)-1H-pyrazolo[3,4-d]pyrimidine-4-carboxamide 48 (20 mg, 39 μmol) was dissolved in dichloromethane (6 mL), and dropwise added with boron tribromide (488.92 mg, 1.95 mmol). After the reaction was completed, the reaction solution was added with dichloromethane (40 mL) and water (50 mL) for extraction. Aqueous phases were extracted with dichloromethane (40 mL×3). Organic phases were combined, concentrated under reduced pressure, and subjected to liquid chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(2-hydroxyphenyl)piperidin-1-yl)-3-(2,3-dichlorophenyl)-1H-pyrazolo[3,4-d]pyrimidine-4-carboxamide 49 (3 mg) with a yield of 15.4%.

MS m/z (ESI): 481.0 [M−16]

### Example 50

### 6-(4-amino-4-(3-methoxyphenyl)piperidin-1-yl)-3-(2,3-dichlorophenyl)-1H-pyrazolo[3,4-d]pyrimidine-4-carboxamide

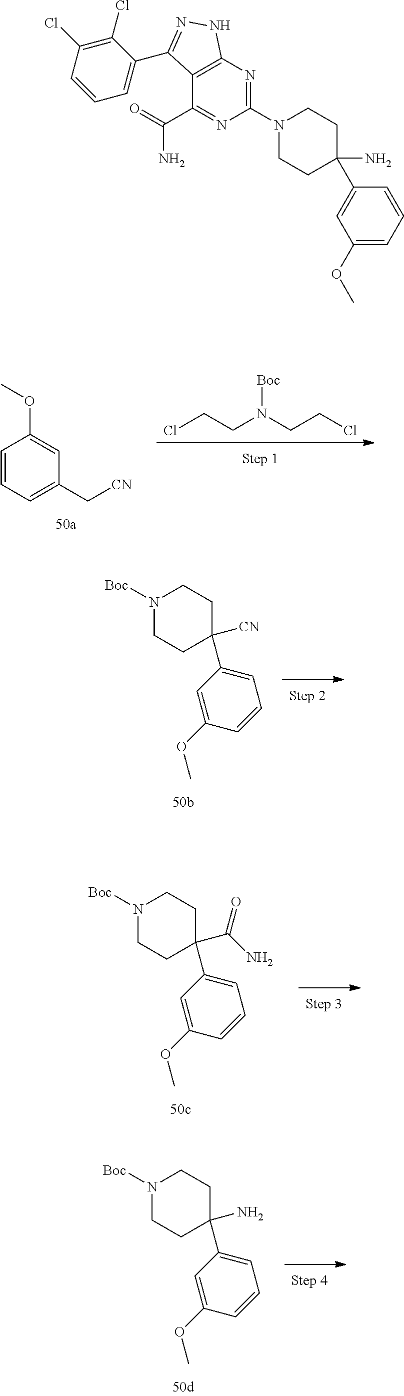 [SMILES: COc1cccc(C2(N)CCN(c3nc(C(N)=O)c4c(-c5cccc(Cl)c5Cl)n[nH]c4n3)CC2)c1 ; COc1cccc(CC#N)c1 ; CC(C)(C)OC(=O)N(CCCl)CCCl ; COc1cccc(C2(C#N)CCN(C(=O)OC(C)(C)C)CC2)c1 ; COc1cccc(C2(C(N)=O)CCN(C(=O)OC(C)(C)C)CC2)c1 ; COc1cccc(C2(N)CCN(C(=O)OC(C)(C)C)CC2)c1]

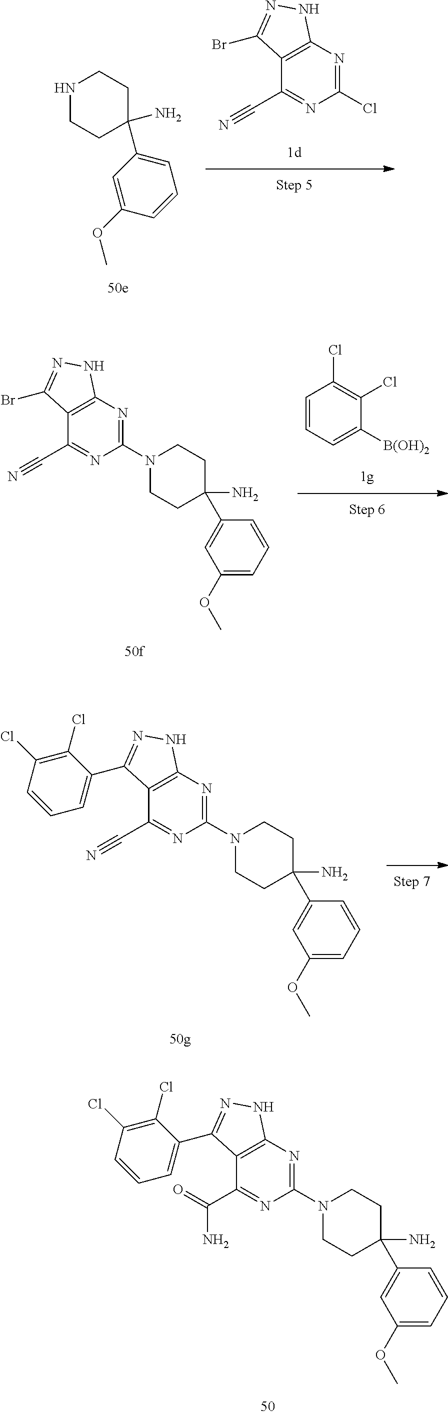 [SMILES: COc1cccc(C2(N)CCNCC2)c1 ; N#Cc1nc(Cl)nc2[nH]nc(Br)c12 ; COc1cccc(C2(N)CCN(c3nc(C#N)c4c(Br)n[nH]c4n3)CC2)c1 ; OB(O)c1cccc(Cl)c1Cl ; COc1cccc(C2(N)CCN(c3nc(C#N)c4c(-c5cccc(Cl)c5Cl)n[nH]c4n3)CC2)c1 ; COc1cccc(C2(N)CCN(c3nc(C(N)=O)c4c(-c5cccc(Cl)c5Cl)n[nH]c4n3)CC2)c1]

### Step 1

### Tert-butyl 4-cyano-4-(3-methoxyphenyl)piperidine-1-carboxylate

2-(3-methoxyphenyl)acetonitrile 50a (2 g, 13.59 mmol) and tert-butyl bis(2-chloroethyl)carbamate (3.62 g, 14.95 mmol) were dissolved in N,N-dimethylformamide (12 mL), added with 60% sodium hydride (2.17 g, 54.36 mmol) in batches, stirred for 40 minutes, heated to 70° C., and reacted overnight. The reaction solution was cooled to room temperature, quenched with water (100 mL), and extracted with ethyl acetate (100 mL×3). Organic phases were combined, washed with a saturated sodium chloride solution (100 mL), dried with anhydrous sodium sulfate and concentrated under reduced pressure. The obtained residue was purified by silica gel column chromatography (eluent: system A) to obtain tert-butyl 4-cyano-4-(3-methoxyphenyl)piperidine-1-carboxylate 50b (3.8 g) with a yield of 88.3%.

MS m/z (ESI): 217.0 [M−99]

### Step 2

### Tert-butyl 4-carbamoyl-4-(3-methoxyphenyl)piperidine-1-carboxylate

Potassium hydroxide (709.37 mg, 12.64 mmol) and tert-butyl 4-cyano-4-(3-methoxyphenyl)piperidine-1-carboxylate 50b (2.00 g, 6.32 mmol) were dissolved in dimethyl sulfoxide (10 mL), slowly added dropwise with hydrogen peroxide (2 mL), and reacted at room temperature overnight. After the reaction was completed, the reaction solution was added with water (50 mL) to precipitate a yellow solid, and filtered, then the filter cake was washed with water, and dried to obtain tert-butyl 4-carbamoyl-4-(3-methoxyphenyl)piperidine-1-carboxylate 50c (1.7 g) with a yield of 80.4%.

MS m/z (ESI): 279.0 [M−55]

### Step 3

### Tert-butyl 4-amino-4-(3-methoxyphenyl)piperidine-1-carboxylate

Potassium hydroxide (1.28 g, 22.88 mmol) was added to a mixed solution of acetonitrile (6 mL) and water (9 mL) containing tert-butyl 4-carbamoyl-4-(3-methoxyphenyl)piperidine-1-carboxylate 50c (1.7 g, 5.08 mmol), added with 1,3-dibromo-5,5-dimethylhydantoin (799.43 mg, 2.80 mmol) in batches, and stirred at room temperature for 1 hour. The reaction solution was added with water (20 mL) and potassium phosphate (1.19 g, 5.59 mmol) and stirred for 15 minutes, then added with ethyl acetate (50 mL) and sodium sulphite (64.05 mg, 508.36 μmol) for liquid separation, aqueous phases were extracted with ethyl acetate (50 mL×2), organic phases were combined and washed with a sodium chloride solution (20 mL), dried and concentrated under reduced pressure. The obtained residue was further analyzed and purified by silica gel column chromatography (eluent: system A) to obtain tert-butyl 4-amino-4-(3-methoxyphenyl)piperidine-1-carboxylate 50d (1.0 g) with a yield of 64.2%.

MS m/z (ESI): 234.1 [M−72]

### Step 4

### 4-(3-methoxyphenyl)piperidin-4-amine

Tert-butyl 4-amino-4-(3-methoxyphenyl)piperidine-1-carboxylate 50d (1 g, 3.26 mmol) was dissolved in dichloromethane (10 mL), slowly added with a trifluoroacetic acid (5 g, 43.85 mmol), and stirred overnight at room temperature. After the reaction was completed, the reaction solution was concentrated under reduced pressure to obtain 4-(3-methoxyphenyl)piperidin-4-amine 50e, which was directly used for the next reaction without purification.

MS m/z (ESI): 190.1 [M−16]

### Step 5

### 6-(4-amino-4-(3-methoxyphenyl)piperidin-1-yl)-3-bromo-1H-pyrazolo[3,4-d]pyrimidine-4-carbonitrile

4-(3-methoxyphenyl)piperidin-4-amine 50e (300 mg, 1.45 mmol), 3-bromo-6-chloro-1H-pyrazolo[3,4-d]pyrimidine-4-carbonitrile 1d (375.88 mg, 1.45 mmol) and N,N-diisopropylethylamine (751.82 mg, 5.82 mmol) were added to N-methyl pyrrolidone (5 mL) in turn, subjected to argon gas replacement, and continuously stirred at 100° C. for 4 hours. After the reaction was completed, the reaction solution was quenched with water (30 mL), and extracted with ethyl acetate (30 mL×3). Organic phases were combined, dried with anhydrous sodium sulfate and concentrated under reduced pressure. The obtained residue was purified by silica gel column chromatography (eluent: system B) to obtain 6-(4-amino-4-(3-methoxyphenyl)piperidin-1-yl)-3-bromo-1H-pyrazolo[3,4-d]pyrimidine-4-carbonitrile 50f (550 mg) with a yield of 88.3%.

MS m/z (ESI): 411.0 [M−16]

### Step 6

### 6-(4-amino-4-(3-methoxyphenyl)piperidin-1-yl)-3-(2,3-dichlorophenyl)-1H-pyrazolo[3,4-d]pyrimidine-4-carbonitrile

6-(4-amino-4-(3-methoxyphenyl)piperidin-1-yl)-3-bromo-1H-pyrazolo[3,4-d]pyrimidine-4-carbonitrile 50f (0.55 g, 1.28 mmol), (2,3-dichlorophenyl)boronic acid 1g (980.19 mg, 5.14 mmol), methanesulfonato(2-dicyclohexylphosphino-2′,6′-di-isopropoxy-1,1′-biphenyl)(2-amino-1,1′-biphenyl-2-yl)palladium (215.07 mg, 256.84 μmol), 2-dicyclohexylphosphino-2′,6′-di-isopropoxy-1,1′-biphenyl (239.70 mg, 513.68 μmol) and potassium phosphate (1.09 g, 5.14 mmol) were added to a mixed solution of 1,4-dioxane (7 mL) and water ((0.7 mL) in turn, subjected to argon gas displacement thrice, heated to 100° C., and reacted overnight. After the reaction was completed, the reaction solution was cooled to room temperature, added with water (20 mL) and extracted with ethyl acetate (20 mL×3), then organic phases were combined, and washed with saturated brine, dried by anhydrous sodium sulfate, and concentrated under reduced pressure. The obtained residue was purified by silica gel column chromatography (eluent: system B) to obtain 6-(4-amino-4-(3-methoxyphenyl)piperidin-1-yl)-3-(2,3-dichlorophenyl)-1H-pyrazolo[3,4-d]pyrimidine-4-carbonitrile 50g (290 mg) with a yield of 46.0%.

MS m/z (ESI): 477.1 [M−16]

### Step 7

### 6-(4-amino-4-(3-methoxyphenyl)piperidin-1-yl)-3-(2,3-dichlorophenyl)-1H-pyrazolo[3,4-d]pyrimidine-4-carboxamide

6-(4-amino-4-(3-methoxyphenyl)piperidin-1-yl)-3-(2,3-dichlorophenyl)-1H-pyrazolo[3,4-d]pyrimidine-4-carbonitrile 50g (290.00 mg, 586.60 μmol) and potassium hydroxide (65.83 mg, 1.17 mmol) were added to dimethyl sulfoxide (4 mL) in turn, and then dropwise added with hydrogen peroxide (1 mL). After the reaction solution was continuously stirred at room temperature for 1 hour, the reaction solution was added with water (20 mL) to precipitate a faint yellow solid, neutralized with a dilute hydrochloric acid, and extracted with ethyl acetate (20 mL×3), then organic phases were combined, and washed with saturated salt water, dried by anhydrous sodium sulfate, and concentrated under reduced pressure. The obtained residue was purified by silica gel column chromatography (eluent: system B) to obtain 6-(4-amino-4-(3-methoxyphenyl)piperidin-1-yl)-3-(2,3-dichlorophenyl)-1H-pyrazolo[3,4-d]pyrimidine-4-carboxamide 50 (210 mg) with a yield of 70%.

MS m/z (ESI): 495.1 [M−16]

¹H NMR (400 MHz, CD₃OD) δ 7.58 (dd, J=7.6, 2.0 Hz, 1H), 7.50 (t, J=8.0 Hz, 1H), 7.33-7.40 (m, 2H), 7.26-7.29 (m, 1H), 7.24 (t, J=2.0 Hz, 1H), 7.08 (dd, J=8.0, 2.0 Hz, 1H), 4.66-4.69 (m, 2H), 3.87 (s, 3H), 3.46-3.51 (m, 2H), 2.72-2.75 (m, 2H), 2.10-2.19 (m, 2H).

### Example 51

### 6-(4-amino-4-(3-hydroxyphenyl)piperidin-1-yl)-3-(2,3-dichlorophenyl)-1H-pyrazolo[3,4-d]pyrimidine-4-carboxamide

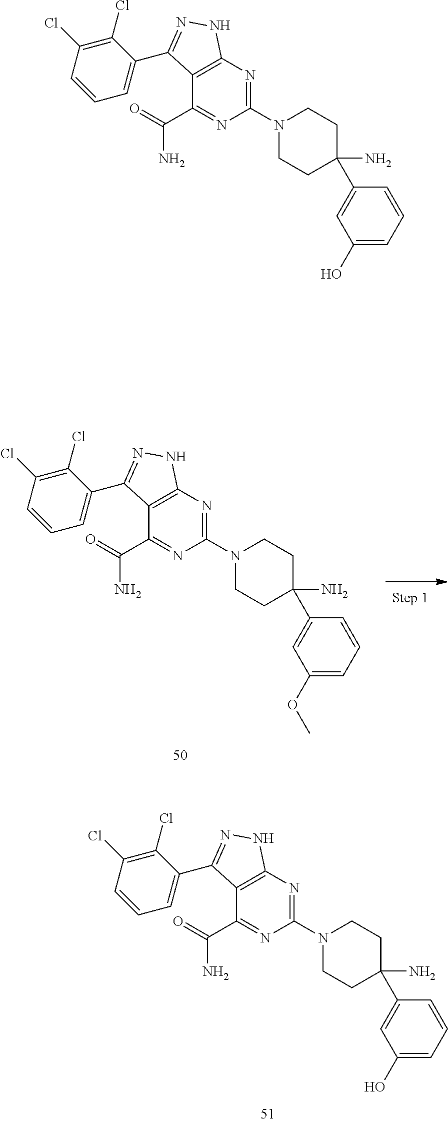 [SMILES: NC(=O)c1nc(N2CCC(N)(c3cccc(O)c3)CC2)nc2[nH]nc(-c3cccc(Cl)c3Cl)c12 ; COc1cccc(C2(N)CCN(c3nc(C(N)=O)c4c(-c5cccc(Cl)c5Cl)n[nH]c4n3)CC2)c1 ; NC(=O)c1nc(N2CCC(N)(c3cccc(O)c3)CC2)nc2[nH]nc(-c3cccc(Cl)c3Cl)c12]

### Step 1

### 6-(4-amino-4-(3-hydroxyphenyl)piperidin-1-yl)-3-(2,3-dichlorophenyl)-1H-pyrazolo[3,4-d]pyrimidine-4-carboxamide

6-(4-amino-4-(3-methoxyphenyl)piperidin-1-yl)-3-(2,3-dichlorophenyl)-1H-pyrazolo[3,4-d]pyrimidine-4-carbonitrile 50 (80 mg, 156.13 μmol)) was dissolved in dichloromethane (4 mL), slowly added dropwise with boron tribromide (5 mL, 1.0 M dichloromethane solution) in an ice bath, and reacted at room temperature for 2 hours. The reaction solution was added with ice water (20 mL), and extracted with dichloromethane (20 mL×3). Organic phases were combined, concentrated under reduced pressure, and subjected to liquid chromatogramatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(3-hydroxyphenyl)piperidin-1-yl)-3-(2,3-dichlorophenyl)-1H-pyrazolo[3,4-d]pyrimidine-4-carboxamide 51 (15 mg) with a yield of 19.3%.

MS m/z (ESI): 481.1 [M−16]

¹H NMR (400 MHz, CD₃OD) δ 7.57-7.59 (m, 1H), 7.33-7.40 (m, 3H), 7.12-7.17 (m, 2H), 6.90 (d J=8.0 Hz, 1H), 4.67-4.71 (m, 2H), 3.47 (t, J=12.0 Hz, 2H), 2.69-2.73 (m, 2H), 2.09-2.16 (m, 2H).

### Example 52

### 6-(4-amino-4-cyclopropylpiperidin-1-yl)-3-(2,3-dichlorophenyl)-1H-pyrazolo[3,4-d]pyrimidine-4-carboxamide

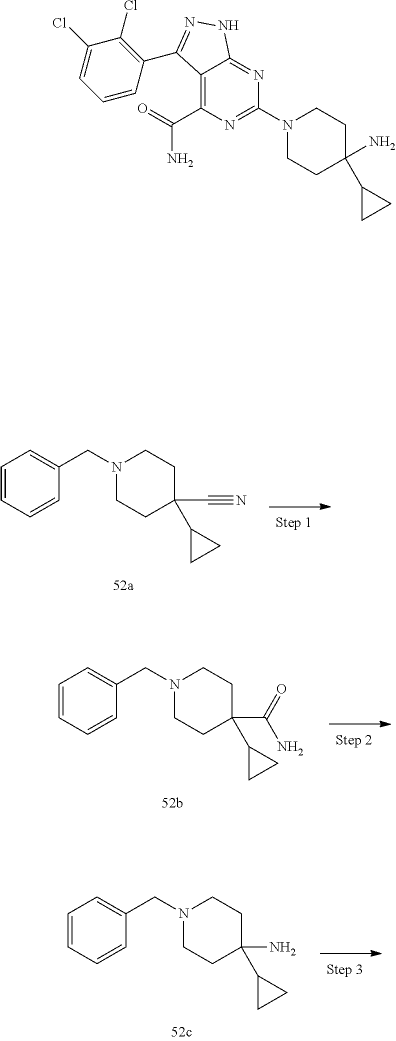 [SMILES: NC(=O)c1nc(N2CCC(N)(C3CC3)CC2)nc2[nH]nc(-c3cccc(Cl)c3Cl)c12 ; N#CC1(C2CC2)CCN(Cc2ccccc2)CC1 ; NC(=O)C1(C2CC2)CCN(Cc2ccccc2)CC1 ; NC1(C2CC2)CCN(Cc2ccccc2)CC1]

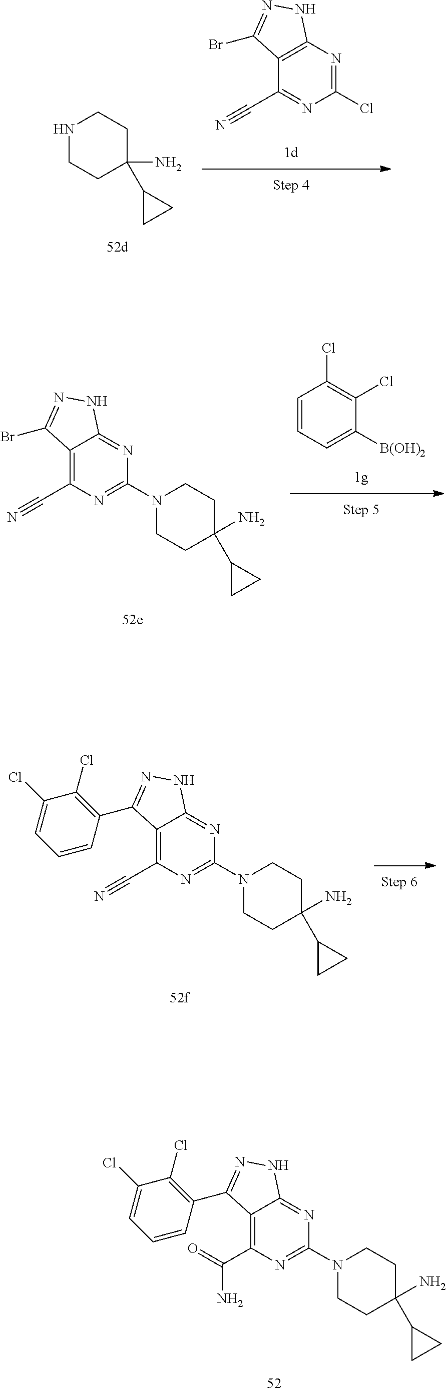 [SMILES: NC1(C2CC2)CCNCC1 ; N#Cc1nc(Cl)nc2[nH]nc(Br)c12 ; N#Cc1nc(N2CCC(N)(C3CC3)CC2)nc2[nH]nc(Br)c12 ; OB(O)c1cccc(Cl)c1Cl ; N#Cc1nc(N2CCC(N)(C3CC3)CC2)nc2[nH]nc(-c3cccc(Cl)c3Cl)c12 ; NC(=O)c1nc(N2CCC(N)(C3CC3)CC2)nc2[nH]nc(-c3cccc(Cl)c3Cl)c12]

### Step 1

### 1-benzyl-4-cyclopropylpiperidine-4-carbonitrile

At room temperature, potassium hydroxide (1.31 g, 23.30 mmol) was added to a solution of tertiary butanol (20 mL) containing 1-benzyl-4-cyclopropylpiperidine-4-carbonitrile 52a (280 mg, 1.17 mmol, self-prepared according to patent WO 2003042174), heated to 110° C., and reacted overnight. The reaction solution was concentrated under reduced pressure, added with 20 mL of water, extracted with dichloromethane (30 mL×3), and washed with a saturated sodium chloride solution (20 mL). Organic phases were dried with anhydrous sodium sulfate, filtered, and concentrated under reduced pressure to obtain 1-benzyl-4-cyclopropylpiperidine-4-carboxamide 52b (300 mg) with a yield of 99.1%, which was directly used for the next reaction without purification.

MS m/z (ESI): 259.2 [M+1]

### Step 2

### 1-benzyl-4-cyclopropylpiperidin-4-amine

Potassium hydroxide (293.19 mg, 5.23 mmol) was added to a mixed solution of acetonitrile (2 mL) and water (6 mL) containing 1-benzyl-4-cyclopropylpiperidine-4-carboxamide 52b (300 mg, 1.16 mmol), added with 1,3-dibromo-5,5-dimethylhydantoin (249.00 mg, 870.89 μmol), and stirred at room temperature for 1.5 hours. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 1-benzyl-4-cyclopropylpiperidin-4-amine 52c (200 mg) with a yield of 74.77%.

MS m/z (ESI): 231.2 [M+1]

### Step 3

### 4-cyclopropylpiperidin-4-amine

Palladium on carbon (72.66 mg, 520.95 μmol) was added to a solution of methanol (20 mL) containing 1-benzyl-4-cyclopropylpiperidin-4-amine 52c (200 mg, 868.25 μmol), subjected to hydrogen gas replacement, and reacted at room temperature for 3 hours. After the reaction was completed, the reaction solution was filtered with diatomite to obtain 4-cyclopropylpiperidin-4-amine 52d (121.7 mg) with a yield of 99.96%, which was directly used for the next reaction without purification.

MS m/z (ESI): 141.1 [M+1]

### Step 4

### 6-(4-amino-4-cyclopropylpiperidin-1-yl)-3-bromo-1H-pyrazolo[3,4-d]pyrimidine-4-carbonitrile

3-bromo-6-chloro-1H-pyrazolo[3,4-d]pyrimidine-4-carbonitrile 1d (224.32 mg, 867.89 μmol), 4-cyclopropylpiperidin-4-amine 52d (121.7 mg, 867.89 μmol) and N,N-diisopropylethylamine (336.50 mg, 2.60 mmol) were added to N,N-dimethylacetamide (2.5 mL) in turn, heated to 100° C., and reacted for 1 hour. After the reaction was completed, the reaction solution was separated on a C₁₈ reversed phase chromatographic column (C₁₈ separation column 20-45 μm; mobile phase A: H₂O, mobile phase B: CH₃CN) to 6-(4-amino-4-cyclopropylpiperidin-1-yl)-3-bromo-1H-pyrazolo[3,4-d]pyrimidine-4-carbonitrile 52e (180 mg) with a yield of 57.26%.

MS m/z (ESI): 345.0 [M−16]

### Step 5

### 6-(4-amino-4-cyclopropylpiperidin-1-yl)-3-(2,3-dichlorophenyl)-1H-pyrazolo[3,4-d]pyrimidine-4-carbonitrile

6-(4-amino-4-cyclopropylpiperidin-1-yl)-3-bromo-1H-pyrazolo[3,4-d]pyrimidine-4-carbonitrile 52e (90 mg, 248.46 μmol), (2,3-dichlorophenyl)boronic acid 1g (165.94 mg, 869.62 μmol), methanesulfonato(2-dicyclohexylphosphino-2′,6′-di-isopropoxy-1,1′-biphenyl)(2-amino-1,1′-biphenyl-2-yl)palladium (20.81 mg, 24.85 μmol), 2-dicyclohexylphosphino-2′,6′-di-isopropoxy-1,1′-biphenyl (23.19 mg, 49.69 μmol) and potassium phosphate (158.22 mg, 745.39 μmol) were added to a mixed solution of 1,4-dioxane (6 mL) and water (0.6 mL), subjected to argon gas displacement thrice, heated to 110° C., and reacted overnight. After the reaction was completed, the reaction solution was added with 20 mL of water, extracted with ethyl acetate (20 mL×3), and washed with a saturated sodium chloride solution (20 mL), then organic phases were dried with anhydrous sodium sulfate, filtered, and concentrated under reduced pressure. The obtained residue was purified by silica gel column chromatography (eluent: system A) to obtain 6-(4-amino-4-cyclopropylpiperidin-1-yl)-3-(2,3-dichlorophenyl)-1H-pyrazolo[3,4-d]pyrimidine-4-carbonitrile 52f (15 mg) with a yield of 14.09%.

MS m/z (ESI): 411.1 [M−16]

### Step 6

### 6-(4-amino-4-cyclopropylpiperidin-1-yl)-3-(2,3-dichlorophenyl)-1H-pyrazolo[3,4-d]pyrimidine-4-carboxamide

Sodium hydroxide (7.00 mg, 175.10 μmol) was added to a solution of methanol (2 mL) containing 6-(4-amino-4-cyclopropylpiperidin-1-yl)-3-(2,3-dichlorophenyl)-1H-pyrazolo[3,4-d]pyrimidine-4-carbonitrile 52f (15 mg, 35.02 μmol), then added with hydrogen peroxide (0.2 mL), and stirred at room temperature for 1 hour. After the reaction was completed, the reaction solution was concentrated under reduced pressure, and subjected to liquid phase chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-cyclopropylpiperidin-1-yl)-3-(2,3-dichlorophenyl)-1H-pyrazolo[3,4-d]pyrimidine-4-carboxamide 52 (2.04 mg) with a yield of 13%.

MS m/z (ESI): 446.1 [M+1]

¹H NMR (400 MHz, DMSO-d₆) δ 8.10 (s, 1H), 7.96 (s, 3H), 7.57-7.73 (m, 2H), 7.39 (d, J=4.9 Hz, 2H), 4.20 (s, 2H), 3.80 (s, 2H), 1.68 (s, 4H), 1.23 (s, 1H), 0.43-0.65 (m, 4H).

### Example 53

### 2-(4-amino-4-phenylpiperidin-1-yl)-5-(2,3-dichlorophenyl)-7H-pyrrolo[2,3-d]pyrimidine-4-carboxamide

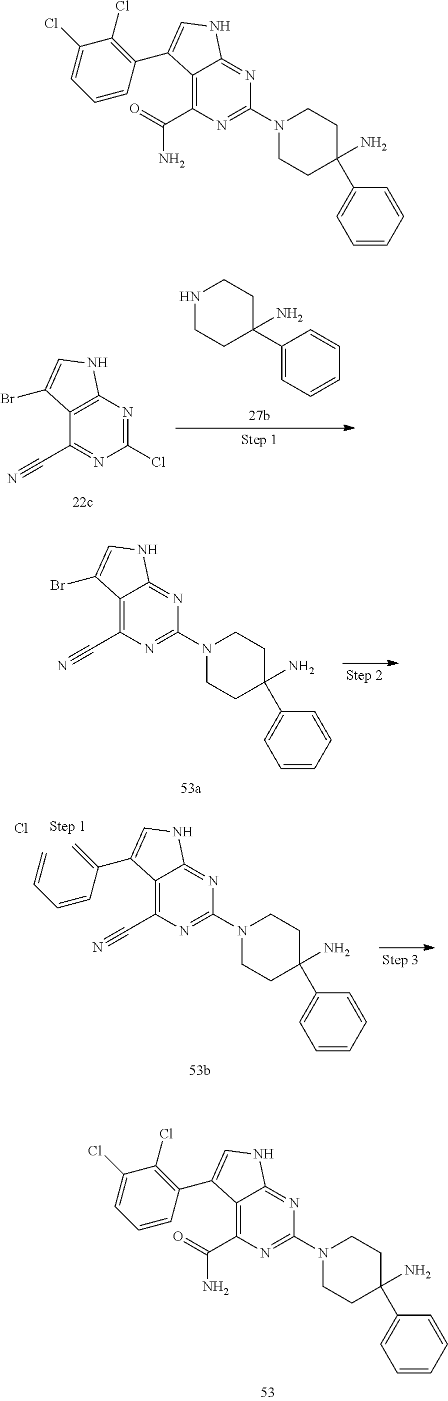

### Step 1

### 2-(4-amino-4-phenylpiperidin-1-yl)-5-bromo-7H-pyrrolo[2,3-d]pyrimidine-4-carbonitrile

N,N-diisopropylethylamine (1.40 g, 10.83 mmol) was added to a solution of N,N-dimethylacetamide (5 mL) containing 4-phenylpiperidin-4-amine 27b (318 mg, 1.80 mmol), stirred for 30 seconds, added with 5-bromo-2-chloro-7H-pyrrolo[2,3-d]pyrimidine-4-carbonitrile 22c (464.53 mg, 1.80 mmol), heated to 120° C., and reacted overnight. The reaction solution was concentrated under reduced pressure. The obtained residue was purified by silicagel column chromatography (eluent: system B) to obtain 2-(4-amino-4-phenylpiperidin-1-yl)-5-bromo-7H-pyrrolo[2,3-d]pyrimidine-4-carbonitrile 53a (50 mg) with a yield of 6.98%.

MS m/z (ESI): 380.2 [M−16]

### Step 2

### 2-(4-amino-4-phenylpiperidin-1-yl)-5-(2,3-dichlorophenyl)-7H-pyrrolo[2,3-d]pyrimidine-4-carbonitrile

2-(4-amino-4-phenylpiperidin-1-yl)-5-bromo-7H-pyrrolo[2,3-d]pyrimidine-4-carbonitrile 53a (50 mg, 125.86 μmol), (2,3-dichlorophenyl)boronic acid 1g (84.06 mg, 440.51 μmol), methanesulfonato(2-dicyclohexylphosphino-2′,6′-di-isopropoxy-1,1′-biphenyl)(2-amino-1,1′-biphenyl-2-yl)palladium (10.54 mg, 12.59 μmol), 2-dicyclohexylphosphino-2′,6′-di-isopropoxy-1,1′-biphenyl (11.75 mg, 25.17 μmol) and potassium phosphate (80.15 mg, 377.58 μmol) were added to a mixed solution of 1,4-dioxane (3 mL) and water (0.3 mL) in turn, subjected to argon gas displacement thrice, heated to 110° C., and reacted overnight. After the reaction was completed, the reaction solution was concentrated under reduced pressure. The obtained residue was purified by silica gel column chromatography (eluent: system B) to obtain 2-(4-amino-4-phenylpiperidin-1-yl)-5-(2,3-dichlorophenyl)-7H-pyrrolo[2,3-d]pyrimidine-4-carbonitrile 53b (50 mg) with a yield of 85.74%.

MS m/z (ESI): 446.1 [M−16]

### Step 3

### 2-(4-amino-4-phenylpiperidin-1-yl)-5-(2,3-dichlorophenyl)-7H-pyrrolo[2,3-d]pyrimidine-4-carboxamide

Sodium hydroxide (21.58 mg, 539.54 μmol) was added to a solution of methanol (3 mL) containing 2-(4-amino-4-phenylpiperidin-1-yl)-5-(2,3-dichlorophenyl)-7H-pyrrolo[2,3-d]pyrimidine-4-carbonitrile 53b (50 mg, 107.91 μmol), then added with hydrogen peroxide (0.3 mL), and stirred at room temperature for 1 hour. After the reaction was completed, the reaction solution was concentrated under reduced pressure, and subjected to liquid phase chromatography purification (separation column: AKZONOBEL Kromasil; 250×21.2 mm I.D.; 5 μm, 20 ml/min; mobile phase A: 0.05% TFA+H₂O, mobile phase B: CH₃CN) to obtain 2-(4-amino-4-phenylpiperidin-1-yl)-5-(2,3-dichlorophenyl)-7H-pyrrolo[2,3-d]pyrimidine-4-carboxamide 53 (13.14 mg) with a yield of 25%.

MS m/z (ESI): 481.1 [M+1]

¹H NMR (400 MHz, DMSO-d₆) δ 11.85 (s, 1H), 8.33 (s, 3H), 7.93 (s, 1H), 7.70 (d, J=7.7 Hz, 2H), 7.39-7.62 (m, 5H), 7.30 (d, J=5.5 Hz, 3H), 4.33 (d, J=13.7 Hz, 2H), 3.90 (s, 2H), 3.44 (t, J=11.5 Hz, 2H), 2.08 (t, J=11.3 Hz, 2H).

### Example 54

### (S)-6-(5-amino-5,7-dihydrospiro[cyclopenta[b]pyridine-6,4′-piperidin]-1′-yl)-3-((2-aminopyrimidine-4-yl)thio)-1H-pyrazolo[3,4-d]pyrimidine-4-carboxamide

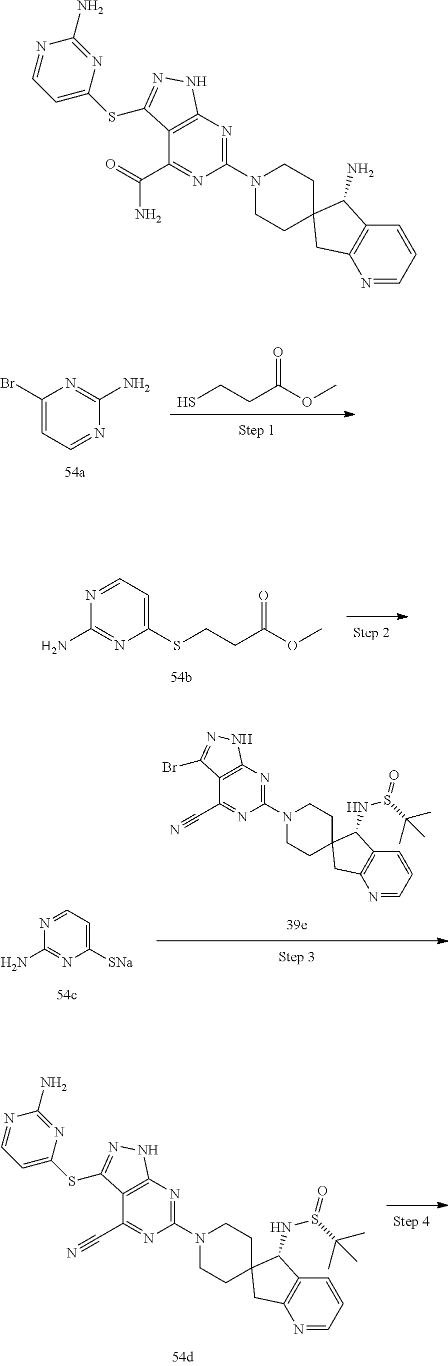

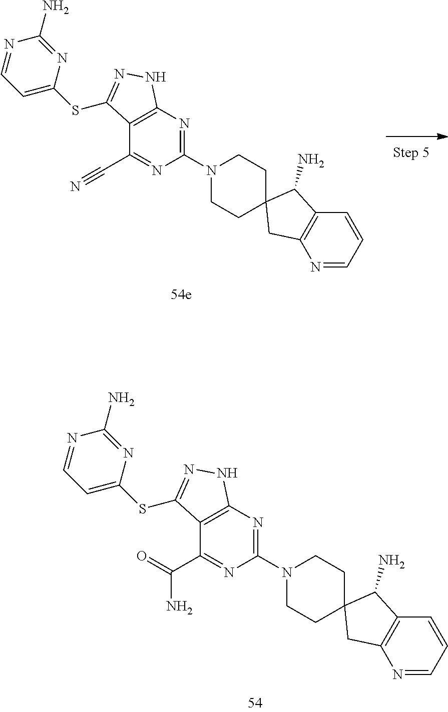 [SMILES: N#Cc1nc(N2CCC3(CC2)Cc2ncccc2[C@H]3N)nc2[nH]nc(Sc3ccnc(N)n3)c12 ; NC(=O)c1nc(N2CCC3(CC2)Cc2ncccc2[C@H]3N)nc2[nH]nc(Sc3ccnc(N)n3)c12]

### Step 1

### Methyl 3-((2-aminopyrimidin-4-yl)thio)propanoate

Tris(dibenzylideneacetone)dipalladium (473.65 mg, 517.25 μmol), 4,5-bis(diphenylphosphino)-9,9-dimethylxanthene (598.58 mg, 1.03 mmol), N,N-diisopropylethylamine (4.01 g, 31.03 mmol) and 4-bromopyrimidin-2-amine 54a (1.8 g, 10.34 mmol) were added to a solution of 1,4-dioxane (30 mL), subjected to argon gas displacement, then added with methyl 3-mercaptopropanoate, subjected to argon gas displacement once, heated to 85° C., and reacted for 3 hours. After the reaction was completed, the reaction solution was concentrated under reduced pressure. The obtained residue was purified by silica gel column chromatography (eluent: system A) to obtain methyl 3-((2-aminopyrimidin-4-yl)thio)propanoate 54b (2.1 g) with a yield of 95.19%.

MS m/z (ESI): 214.1 [M+1]

### Step 2

### Sodium 2-aminopyrimidine-4-thiolate

At room temperature, sodium methylate (506.62 mg, 9.38 mmol) was added to a solution of methanol (6 mL) containing methyl 3-((2-aminopyrimidin-4-yl)thio)propanoate 54b (1 g, 4.69 mmol), and stirred at room temperature for 1 hour. After the reaction was completed, the reaction solution was concentrated under reduced pressure to obtain sodium 2-aminopyrimidine-4-thiolate 54c (670 mg) with a yield of 96.08%.

MS m/z (ESI): 128.1 [M+1]

### Step 3

### (R)—N—((S)-1′-(3-((2-aminopyrimidin-4-yl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide

Sodium 2-aminopyrimidine-4-thiolate 54c (120 mg, 804.56 μmol), (R)—N—((S)-1′-(3-bromo-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 39e (340.78 mg, 643.65 μmol), tris(dibenzylideneacetone)dipalladium (73.68 mg, 80.46 μmol), 4,5-bis(diphenylphosphino)-9,9-dimethylxanthene (93.11 mg, 160.91 μmol) and N,N-diisopropylethylamine (311.95 mg, 2.41 mmol) were added to a solution of 1,4-dioxane (3 mL), subjected to argon gas displacement, heated to 100° C., and reacted overnight. After the reaction was completed, the reaction solution was added with 20 mL of water, extracted with dichloromethane (30 mL×3), and washed with a saturated sodium chloride solution (20 mL). Organic phases were dried with anhydrous sodium sulfate, filtered, and concentrated under reduced pressure. The obtained residue was further analyzed and purified by silica gel column chromatography (eluent: system B) to obtain (R)—N—((S)-1′-(3-((2-aminopyrimidin-4-yl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 54d (140 mg) with a yield of 37%.

MS m/z (ESI): 576.2 [M+1]

### Step 4

### (S)-6-(5-amino-5,7-dihydrospiro[cyclopenta[b]pyridine-6,4′-piperidin]-1′-yl)-3-((2-aminopyrimidin-4-yl)thio)-1H-pyrazolo[3,4-d]pyrimidine-4-carbonitrile

A concentrated hydrochloric acid (17.73 mg, 486.36 μmol) was added to a solution of methanol (3 mL) containing (R)—N—((S)-1′-(3-((2-aminopyrimidin-4-yl)thio)-4-cyano-1H-pyrazolo[3,4-d]pyrimidin-6-yl)-5,7-dihydrospiro[cyclopenta[b]pyridine-6,4′-piperidin]-5-yl)-2-methylpropane-2-sulfinamide 54d (70 mg, 121.59 μmol),and stirred at room temperature for 2 hours. The reaction solution was concentrated under reduced pressure to obtain (S)-6-(5-amino-5,7-dihydrospiro[cyclopenta[b]pyridine-6,4′-piperidin]-1′-yl)-3-((2-aminopyrimidin-4-yl)thio)-1H-pyrazolo[3,4-d]pyrimidine-4-carbonitrile 54e (57 mg) with a yield of 99.42%, which was directly used for the next reaction without purification.

MS m/z (ESI): 472.2 [M+1]

### Step 5

### (S)-6-(5-amino-5,7-dihydrospiro[cyclopenta[b]pyridine-6,4′-piperidin]-1′-yl)-3-((2-aminopyrimidin-4-yl)thio)-1H-pyrazolo[3,4-d]pyrimidine-4-carboxamide

Sodium hydroxide (24.18 mg, 604.40 μmol) was added to a solution of methanol (3 mL) containing (S)-6-(5-amino-5,7-dihydrospiro[cyclopenta[b]pyridine-6,4′-piperidin]-1′-yl)-3-((2-aminopyrimidin-4-yl)thio)-1H-pyrazolo[3,4-d]pyrimidine-4-carbonitrile 54e (57 mg, 120.88 μmol), then added with hydrogen peroxide (0.4 mL), and stirred at room temperature for 1 hour. After the reaction was completed, the reaction solution was concentrated under reduced pressure, and subjected to liquid phase chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain (S)-6-(5-amino-5,7-dihydrospiro[cyclopenta[b]pyridine-6,4′-piperidin]-1′-yl)-3-((2-aminopyrimidin-4-yl)thio)-1H-pyrazolo[3,4-d]pyrimidine-4-carboxamide 54 (7.2 mg) with a yield of 12%.

MS m/z (ESI): 490.2 [M+1]

¹H NMR (400 MHz, CD₃OD) δ 8.48 (d, J=5.2 Hz, 1H), 7.91 (d, J=7.8 Hz, 1H), 7.78 (d, J=6.8 Hz, 1H), 7.25-7.41 (m, 1H), 6.59 (d, J=6.8 Hz, 1H), 4.81-4.85 (m, 2H), 4.45 (s, 1H), 3.33 (q, J=14.9, 14.3 Hz, 2H), 3.16 (s, 2H), 1.65-1.81 (m, 2H), 1.57 (d, J=13.3 Hz, 1H), 1.34-1.48 (m, 1H).

### Example 55

### 6-(4-amino-4-(pyridin-3-yl)piperidin-1-yl)-3-(2,3-dichlorophenyl)-1H-pyrazolo[3,4-d]pyrimidine-4-carboxamide

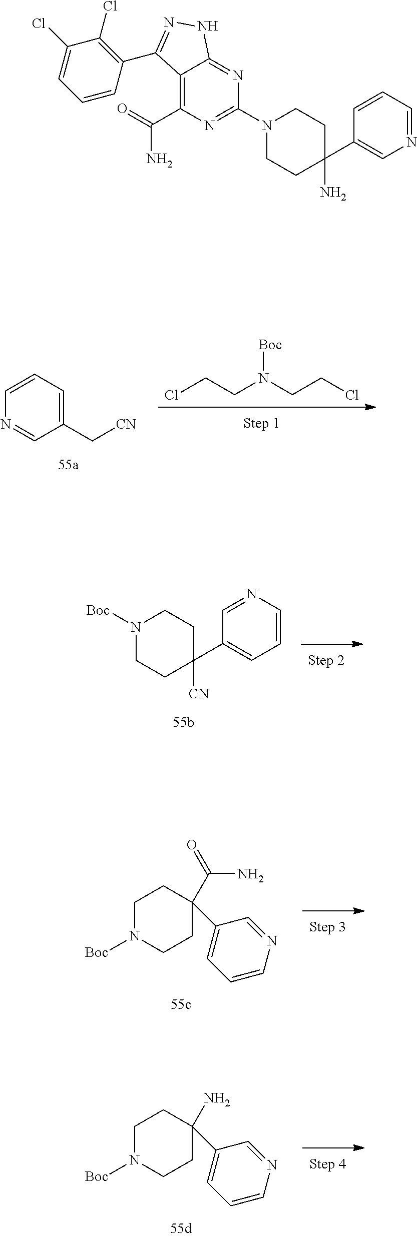 [SMILES: NC(=O)c1nc(N2CCC(N)(c3cccnc3)CC2)nc2[nH]nc(-c3cccc(Cl)c3Cl)c12 ; N#CCc1cccnc1 ; CC(C)(C)OC(=O)N(CCCl)CCCl ; CC(C)(C)OC(=O)N1CCC(C#N)(c2cccnc2)CC1 ; CC(C)(C)OC(=O)N1CCC(C(N)=O)(c2cccnc2)CC1 ; CC(C)(C)OC(=O)N1CCC(N)(c2cccnc2)CC1]

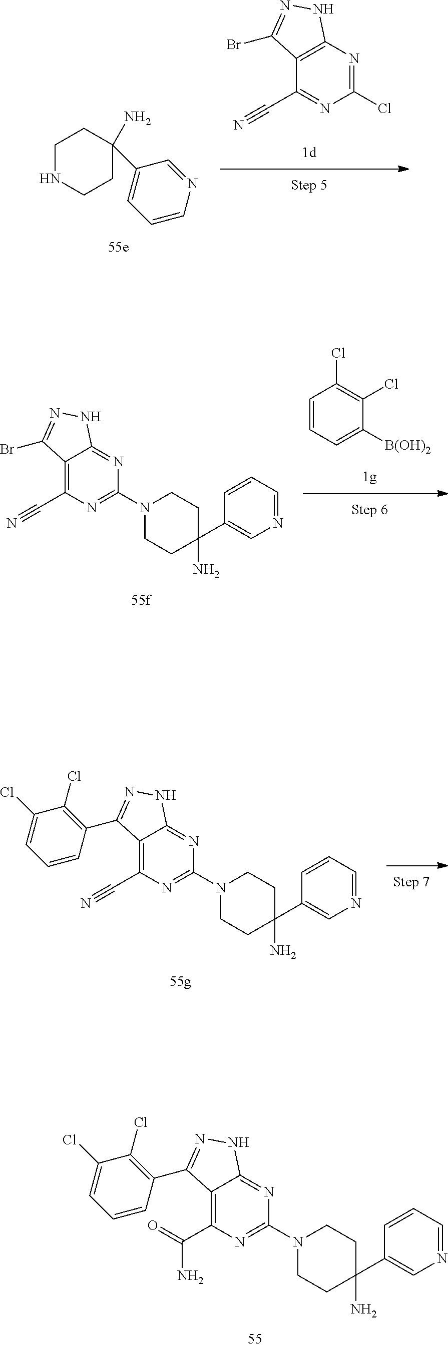 [SMILES: NC1(c2cccnc2)CCNCC1 ; N#Cc1nc(Cl)nc2[nH]nc(Br)c12 ; N#Cc1nc(N2CCC(N)(c3cccnc3)CC2)nc2[nH]nc(Br)c12 ; OB(O)c1cccc(Cl)c1Cl ; N#Cc1nc(N2CCC(N)(c3cccnc3)CC2)nc2[nH]nc(-c3cccc(Cl)c3Cl)c12 ; NC(=O)c1nc(N2CCC(N)(c3cccnc3)CC2)nc2[nH]nc(-c3cccc(Cl)c3Cl)c12]

### Step 1

### Tert-butyl 4-cyano-4-(pyridin-3-yl)piperidine-1-carboxylate

Sodium hydride (3.56 g, 88.88 mmol) was added to a solution of N,N-dimethylformamide (35 mL) containing 2-(pyridin-3-yl)acetonitrile 55a (3 g, 25.39 mmol) and tert-butyl bis(2-chloroethyl)carbamate (6.76 g, 27.93 mmol), heated to 60° C., and reacted overnight. After the reaction was completed, the reaction solution was quenched with 40 mL of saturated ammonium chloride, extracted with ethyl acetate (40 mL×3), and washed with a saturated sodium chloride solution (30 mL). Organic phases were dried with anhydrous sodium sulfate, filtered, and concentrated under reduced pressure. The obtained residue was further analyzed and purified by silica gel column chromatography (eluent: system A) to obtain tert-butyl 4-cyano-4-(pyridin-3-yl)piperidine-1-carboxylate 55b (6.6 g) with a yield of 90.44%.

MS m/z (ESI): 288.2 [M+1]

### Step 2

### Tert-butyl 4-carbamoyl-4-(pyridin-3-yl)piperidine-1-carboxylate

Potassium hydroxide (781.05 mg, 13.92 mmol) was added to a solution of dimethyl sulfoxide (12 mL) containing tert-butyl 4-cyano-4-(pyridin-3-yl)piperidine-1-carboxylate 55b (2 g, 6.96 mmol), and slowly added dropwise with hydrogen peroxide (5 mL). After the reaction was completed, the reaction solution was added with 10 mL of water, continuously stirred for 1 hour, and then filtered to obtain tert-butyl 4-carbamoyl-4-(pyridin-3-yl)piperidine-1-carboxylate 55c (1.8 g) with a yield of 84.69%.

MS m/z (ESI): 306.2 [M+1]

### Step 3

### Tert-butyl 4-amino-4-(pyridin-3-yl)piperidine-1-carboxylate

[Bis(trifluoroacetoxy)iodo]benzene (1.24 g, 2.88 mmol) was added to a solution of acetonitrile (12 mL) and water (12 mL) containing tert-butyl 4-carbamoyl-4-(pyridin-3-yl)piperidine-1-carboxylate 55c (800.00 mg, 2.62 mmol), and reacted at room temperature overnight. After the reaction was completed, the reaction solution was added with 40 mL of sodium bicarbonate solution, extracted with ethyl acetate (30 mL×3), and washed with a saturated sodium chloride solution (30 mL). Organic phases were dried with anhydrous sodium sulfate, filtered, and concentrated under reduced pressure to obtain tert-butyl 4-amino-4-(pyridin-3-yl)piperidine-1-carboxylate 55d (726 mg) with a yield of 99.91%.

MS m/z (ESI): 278.2 [M+1]

### Step 4

### 4-(pyridin-3-yl)piperidine-4-amine

A trifluoroacetic acid (1 mL) was added to a solution of dichloromethane (4 mL) containing tert-butyl 4-amino-4-(pyridin-3-yl)piperidine-1-carboxylate 55d (150 mg, 540.81 μmol), and stirred at room temperature for 40 minutes. The reaction solution was concentrated under reduced pressure to obtain 4-(pyridin-3-yl)piperidin-4-amine 55e (95.86 mg) with a yield of 100.00%, which was directly used for the next reaction without purification.

MS m/z (ESI): 178.1 [M+1]

### Step 5

### 6-(4-amino-4-(pyridin-3-yl)piperidin-1-yl)-3-bromo-1H-pyrazolo[3,4-d]pyrimidine-4-carbonitrile

N,N-diisopropylethylamine (314.86 mg, 2.44 mmol) and 3-bromo-6-chloro-1H-pyrazolo[3,4-d]pyrimidine-4-carbonitrile 1d (125.94 mg, 487.25 μmol) were added to a solution of N-methyl pyrrolidone (5 mL) containing 4-(pyridin-3-yl)piperidin-4-amine 55e (95 mg, 535.98 μmol), heated to 110° C., and reacted for 1 hour. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-3-yl)piperidin-1-yl)-3-bromo-1H-pyrazolo[3,4-d]pyrimidine-4-carbonitrile 55f (130 mg) with a yield of 66.83%.

MS m/z (ESI): 399.0 [M+1]

### Step 6

### 6-(4-amino-4-(pyridin-3-yl)piperidin-1-yl)-3-(2,3-dichlorophenyl)-1H-pyrazolo[3,4-d]pyrimidine-4-carbonitrile

6-(4-amino-4-(pyridin-3-yl)piperidin-1-yl)-3-bromo-1H-pyrazolo[3,4-d]pyrimidine-4-carbonitrile 55f (130 mg, 325.61 μmol), (2,3-dichlorophenyl)boronic acid 1g (248.53 mg, 1.30 mmol), methanesulfonato(2-dicyclohexylphosphino-2′,6′-di-isopropoxy-1,1′-biphenyl)(2-amino-1,1′-biphenyl-2-yl)palladium (54.53 mg, 65.12 μmol), 2-dicyclohexylphosphino-2′,6′-di-isopropoxy-1,1′-biphenyl (60.78 mg, 130.25 μmol) and potassium phosphate (207.45 mg, 976.84 μmol) were added to a mixed solution of 1,4-dioxane (8 mL) and water (0.8 mL) in turn, subjected to argon gas displacement thrice, heated to 110° C., and reacted overnight. After the reaction was completed, the reaction solution was concentrated under reduced pressure, and subjected to liquid phase chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-3-yl)piperidin-1-yl)-3-(2,3-dichlorophenyl)-1H-pyrazolo[3,4-d]pyrimidine-4-carbonitrile 55g (10 mg) with a yield of 6.60%.

MS m/z (ESI): 465.1 [M+1]

### Step 7

### 6-(4-amino-4-(pyridin-3-yl)piperidin-1-yl)-3-(2,3-dichlorophenyl)-1H-pyrazolo[3,4-d]pyrimidine-4-carboxamide

Sodium hydroxide solution (0.5 mL) and hydrogen peroxide (0.5 mL) were added to a solution of methanol (1 mL) containing 6-(4-amino-4-(pyridin-3-yl)piperidin-1-yl)-3-(2,3-dichlorophenyl)-1H-pyrazolo[3,4-d]pyrimidine-4-carbonitrile 55g (10 mg, 21.49 μmol) in turn, and stirred at room temperature for 1 hour. After the reaction was completed, the reaction solution was concentrated under reduced pressure, and subjected to liquid phase chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-3-yl)piperidin-1-yl)-3-(2,3-dichlorophenyl)-1H-pyrazolo[3,4-d]pyrimidine-4-carboxamide 55 (2.19 mg) with a yield of 21%.

MS m/z (ESI): 483.1 [M+1]

¹H NMR (400 MHz, CD₃OD) δ 8.99 (s, 1H), 8.74 (s, 1H), 8.28 (d, J=8.3 Hz, 1H), 7.71 (s, 1H), 7.61 (dd, J=7.5, 2.0 Hz, 1H), 7.32-7.50 (m, 2H), 4.67 (d, J=14.2 Hz, 2H), 3.57 (t, J=12.0 Hz, 2H), 2.82 (d, J=13.8 Hz, 2H), 2.15-2.37 (m, 2H).

### Example 56

### 6-(4-amino-4-(pyridin-4-yl)piperidin-1-yl)-3-(2,3-dichlorophenyl)-1H-pyrazolo[3,4-d]pyrimidine-4-carboxamide

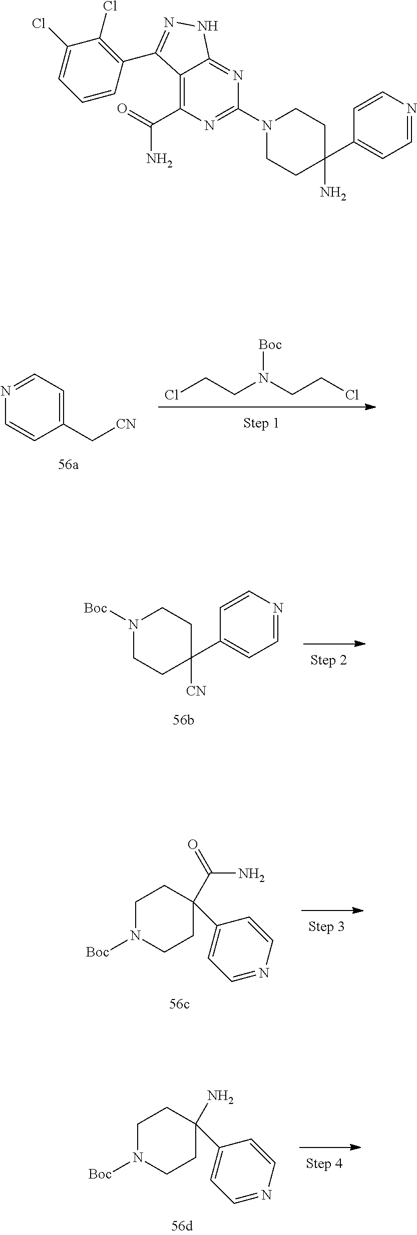 [SMILES: NC(=O)c1nc(N2CCC(N)(c3ccncc3)CC2)nc2[nH]nc(-c3cccc(Cl)c3Cl)c12 ; N#CCc1ccncc1 ; CC(C)(C)OC(=O)N(CCCl)CCCl ; CC(C)(C)OC(=O)N1CCC(C#N)(c2ccncc2)CC1 ; CC(C)(C)OC(=O)N1CCC(C(N)=O)(c2ccncc2)CC1 ; CC(C)(C)OC(=O)N1CCC(N)(c2ccncc2)CC1]

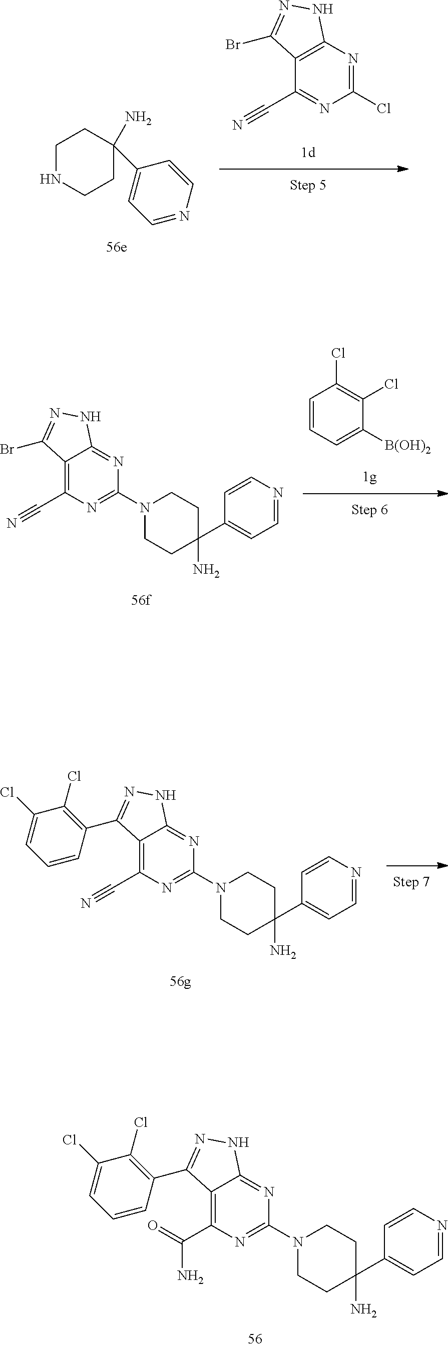 [SMILES: NC1(c2ccncc2)CCNCC1 ; N#Cc1nc(Cl)nc2[nH]nc(Br)c12 ; N#Cc1nc(N2CCC(N)(c3ccncc3)CC2)nc2[nH]nc(Br)c12 ; OB(O)c1cccc(Cl)c1Cl ; N#Cc1nc(N2CCC(N)(c3ccncc3)CC2)nc2[nH]nc(-c3cccc(Cl)c3Cl)c12 ; NC(=O)c1nc(N2CCC(N)(c3ccncc3)CC2)nc2[nH]nc(-c3cccc(Cl)c3Cl)c12]

### Step 1

### Tert-butyl 4-cyano-4-(pyridin-4-yl)piperidine-1-carboxylate Sodium hydride (1.36 g, 33.96 mmol) was added to a solution of N,N-dimethylformamide

(25 mL) containing 2-(pyridin-4-yl)acetonitrile 56a (1.5 g, 9.70 mmol) in batches, heated to 60° C., and reacted overnight. After the reaction was completed, the reaction solution was quenched with 40 mL of saturated ammonium chloride, extracted with ethyl acetate (40 mL×3), and washed with a saturated sodium chloride solution (30 mL). Organic phases were dried with anhydrous sodium sulfate, filtered, and concentrated under reduced pressure. The obtained residue was further analyzed and purified by silica gel column chromatography (eluent: system A) to obtain tert-butyl 4-cyano-4-(pyridin-4-yl)piperidine-1-carboxylate 56b (1.65 g) with a yield of 59.18%.

MS m/z (ESI): 288.2 [M+1]

### Step 2

### Tert-butyl 4-carbamoyl-4-(pyridin-4-yl)piperidine-1-carboxylate

Potassium hydroxide (644.37 mg, 11.48 mmol) was added to a solution of dimethyl sulfoxide (15 mL) containing tert-butyl 4-cyano-4-(pyridin-4-yl)piperidine-1-carboxylate 56b (1.65 g, 5.74 mmol), slowly dropwise added with hydrogen peroxide (5 mL) to release heat violently. The reaction solution may be partially cooled with ice water, and reacted at room temperature for 1 hour. After the reaction was completed, a large amount of solids were precipitated and filtered to obtain the product tert-butyl 4-carbamoyl-4-(pyridin-4-yl)piperidine-1-carboxylate 56c (1.08 g) with a yield of 61.59%.

MS m/z (ESI): 306.2 [M+1]

### Step 3

### Tert-butyl 4-amino-4-(pyridin-4-yl)piperidine-1-carboxylate Potassium hydroxide (892.99 mg, 15.92 mmol) was added to a mixed solution of acetonitrile

(2.5 mL) and water (10 mL) containing tert-butyl 4-carbamoyl-4-(pyridin-4-yl)piperidine-1-carboxylate 56c (1.08 g, 3.54 mmol), added with 1,3-dibromo-5,5-dimethylhydantoin (758.41 mg, 2.65 mmol) in a water bath in batches, and stirred at room temperature for 1 hour. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain tert-butyl 4-amino-4-(pyridin-4-yl)piperidine-1-carboxylate 56d (980 mg) with a yield of 99%.

MS m/z (ESI): 278.2 [M+1]

### Step 4

### 4-(pyridin-4-yl)piperidin-4-amine

A trifluoroacetic acid (1 mL) was added to a solution of dichloromethane (4 mL) containing tert-butyl 4-amino-4-(pyridin-4-yl)piperidine-1-carboxylate 56d (250 mg, 901.35 μmol),and stirred at room temperature for 40 minutes. After the reaction was completed, the reaction solution was concentrated under reduced pressure to obtain 4-(pyridin-4-yl)piperidin-4-amine 56e (159.76 mg) with a yield of 100.00%, which was directly used for the next reaction without purification.

MS m/z (ESI): 178.1 [M+1]

### Step 5

### 6-(4-amino-4-(pyridin-4-yl)piperidin-1-yl)-3-bromo-1H-pyrazolo[3,4-d]pyrimidine-4-carbonitrile

N,N-diisopropylethylamine (486.11 mg, 3.76 mmol) was added to a solution of N,N-dimethylacetamide (3 mL) containing 4-(pyridin-4-yl)piperidin-4-amine 56e (160 mg, 902.70 μmol), added with 3-bromo-6-chloro-1H-pyrazolo[3,4-d]pyrimidine-4-carbonitrile 1d (194.43 mg, 752.25 μmol), heated to 80° C., and reacted for 1.5 hours. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-4-yl)piperidin-1-yl)-3-bromo-1H-pyrazolo[3,4-d]pyrimidine-4-carbonitrile 56f (274 mg) with a yield of 91.23%.

MS m/z (ESI): 399.1 [M+1]

### Step 6

### 6-(4-amino-4-(pyridin-4-yl)piperidin-1-yl)-3-(2,3-dichlorophenyl)-1H-pyrazolo[3,4-d]pyrimidine-4-carbonitrile

6-(4-amino-4-(pyridin-4-yl)piperidin-1-yl)-3-bromo-1H-pyrazolo[3,4-d]pyrimidine-4-carbonitrile 56f (122 mg, 305.58 μmol), (2,3-dichlorophenyl)boronic acid 1g (291.55 mg, 1.53 mmol), methanesulfonato(2-dicyclohexylphosphino-2′,6′-di-isopropoxy-1,1′-biphenyl)(2-amino-1,1′-biphenyl-2-yl)palladium (51.18 mg, 61.12 μmol), 2-dicyclohexylphosphino-2′,6′-di-isopropoxy-1,1′-biphenyl (57.04 mg, 122.23 μmol) and potassium phosphate (194.68 mg, 916.73 μmol) were added to a mixed solution of 1,4-dioxane (8 mL) and water (0.8 mL) in turn, subjected to argon gas displacement thrice, heated to 100° C., and reacted overnight. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-4-(pyridin-4-yl)piperidin-1-yl)-3-(2,3-dichlorophenyl)-1H-pyrazolo[3,4-d]pyrimidine-4-carbonitrile 56g (60 mg) with a yield of 42.2%.

MS m/z (ESI): 465.1 [M+1]

### Step 7

### 6-(4-amino-4-(pyridin-4-yl)piperidin-1-yl)-3-(2,3-dichlorophenyl)-1H-pyrazolo[3,4-d]pyrimidine-4-carboxamide

Sodium hydroxide solution (0.5 mL) and hydrogen peroxide (0.5 mL) were added to a solution of methanol (1 mL) containing 6-(4-amino-4-(pyridin-4-yl)piperidin-1-yl)-3-(2,3-dichlorophenyl)-1H-pyrazolo[3,4-d]pyrimidine-4-carbonitrile 56g (60 mg, 128.94 μmol) in turn, and stirred at room temperature for 1.5 hours. After the reaction was completed, the reaction solution was concentrated under reduced pressure, and subjected to liquid phase chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-(pyridin-4-yl)piperidin-1-yl)-3-(2,3-dichlorophenyl)-1H-pyrazolo[3,4-d]pyrimidine-4-carboxamide 56 (16.22 mg) with a yield of 26%.

MS m/z (ESI): 483.1 [M+1]

1H NMR (400 MHz, DMSO-d₆) δ 8.74 (d, J=5.2 Hz, 2H), 8.58 (s, 3H), 8.14 (s, 1H), 7.61-7.78 (m, 4H), 7.40 (d, J=4.8 Hz, 2H), 4.27 (s, 2H), 3.81 (s, 2H), 2.47 (s, 2H), 2.09 (s, 2H).

### Example 57

### 6-(4-amino-3-phenylpiperidin-1-yl)-3-(2,3-dichlorophenyl)-1H-pyrazolo[3,4-d]pyrimidine-4-carboxamide

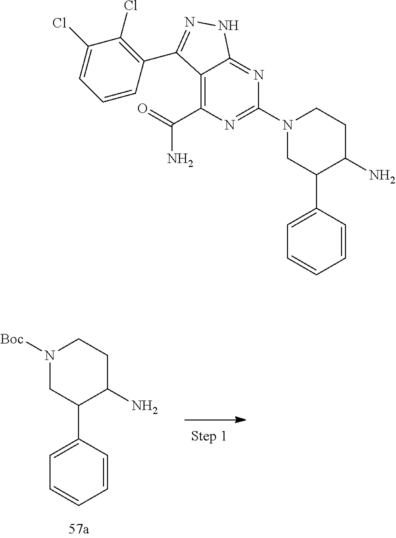 [SMILES: NC(=O)c1nc(N2CCC(N)C(c3ccccc3)C2)nc2[nH]nc(-c3cccc(Cl)c3Cl)c12 ; CC(C)(C)OC(=O)N1CCC(N)C(c2ccccc2)C1]

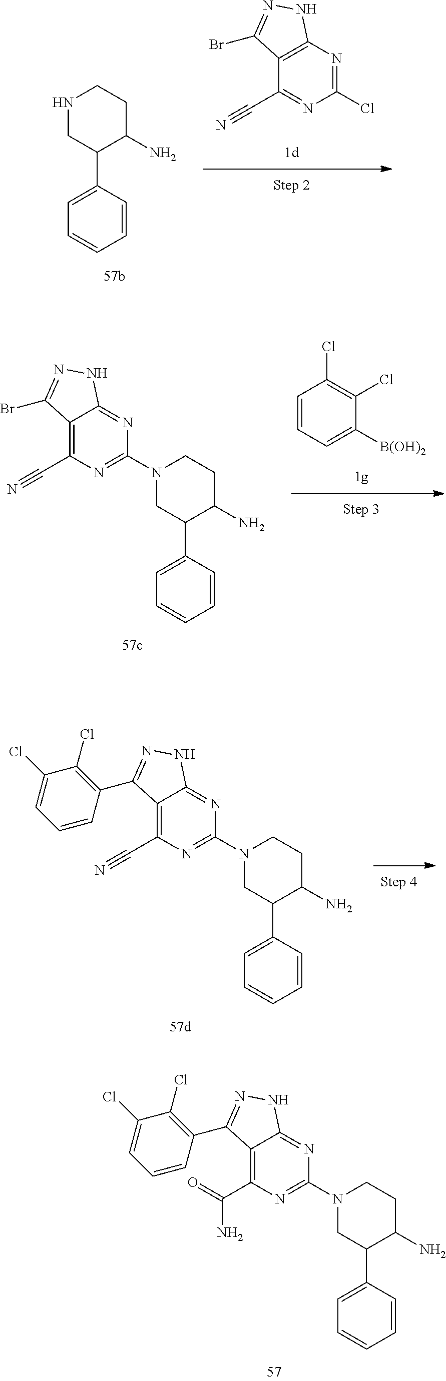 [SMILES: NC1CCNCC1c1ccccc1 ; N#Cc1nc(Cl)nc2[nH]nc(Br)c12 ; N#Cc1nc(N2CCC(N)C(c3ccccc3)C2)nc2[nH]nc(Br)c12 ; OB(O)c1cccc(Cl)c1Cl ; N#Cc1nc(N2CCC(N)C(c3ccccc3)C2)nc2[nH]nc(-c3cccc(Cl)c3Cl)c12 ; NC(=O)c1nc(N2CCC(N)C(c3ccccc3)C2)nc2[nH]nc(-c3cccc(Cl)c3Cl)c12]

### Step 1

### 3-phenylpiperidin-4-amine

A trifluoroacetic acid (1.5 mL) and tert-butyl 4-amino-3-phenylpiperidine-1-carboxylate 57a (350 mg, 1.27 mmol, self-prepared according to patent WO2019169153) were added to a solution of dichloromethane (6 mL), and stirred at room temperature for 40 minutes. The reaction solution was concentrated under reduced pressure to obtain 3-phenylpiperidin-4-amine 57b (223.21 mg) with a yield of 99%, which was directly used for the next reaction without purification.

MS m/z (ESI): 177.1 [M+1]

### Step 2

### 6-(4-amino-3-phenylpiperidin-1-yl)-3-bromo-1H-pyrazolo[3,4-d]pyrimidine-4-carbonitrile

N,N-diisopropylethylamine (400.03 mg, 3.10 mmol) was added to a solution of N,N-dimethylacetamide (3 mL) containing 3-phenylpiperidin-4-amine 57b (223 mg, 1.26 mmol), added with 3-bromo-6-chloro-1H-pyrazolo[3,4-d]pyrimidine-4-carbonitrile 1d (200 mg, 773.81 μmol), heated to 70° C., and reacted for 1.5 hours. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-3-phenylpiperidin-1-yl)-3-bromo-1H-pyrazolo[3,4-d]pyrimidine-4-carbonitrile 57c (180 mg) with a yield of 58.41%.

MS m/z (ESI): 398.1 [M+1]

### Step 3

### 6-(4-amino-3-phenylpiperidin-1-yl)-3-(2,3-dichlorophenyl)-1H-pyrazolo[3,4-d]pyrimidine-4-carbonitrile

6-(4-amino-3-phenylpiperidin-1-yl)-3-bromo-1H-pyrazolo[3,4-d]pyrimidine-4-carbonitrile 57c (180 mg, 451.97 μmol), (2,3-dichlorophenyl)boronic acid 1g (344.98 mg, 1.81 mmol), methanesulfonato(2-dicyclohexylphosphino-2′,6′-di-isopropoxy-1,1′-biphenyl)(2-amino-1,1′-biphenyl-2-yl)palladium (75.69 mg, 90.39 μmol), 2-dicyclohexylphosphino-2′,6′-di-isopropoxy-1,1′-biphenyl (84.36 mg, 180.79 μmol) and potassium phosphate (287.95 mg, 1.36 mmol) were added to a mixed solution of 1,4-dioxane (10 mL) and water (1 mL) in turn, subjected to argon gas displacement thrice, heated to 120° C., and reacted overnight. After the reaction was completed, the reaction solution was concentrated under reduced pressure and separated on a C₁₈ reversed phase chromatographic column (C₁₈ separation column 20-45 μm; mobile phase A: H₂O, mobile phase B: CH₃CN) to obtain 6-(4-amino-3-phenylpiperidin-1-yl)-3-(2,3-dichlorophenyl)-1H-pyrazolo[3,4-d]pyrimidine-4-carbonitrile 57d (21 mg) with a yield of 10.01%.

MS m/z (ESI): 464.1 [M+1]

### Step 4

### 6-(4-amino-3-phenylpiperidin-1-yl)-3-(2,3-dichlorophenyl)-1H-pyrazolo[3,4-d]pyrimidine-4-carboxamide

A sodium hydroxide solution (0.5 mL) was added to a solution of methanol (2 mL) containing 6-(4-amino-3-phenylpiperidin-1-yl)-3-(2,3-dichlorophenyl)-1H-pyrazolo[3,4-d]pyrimidine-4-carbonitrile 57d (21 mg, 45.22 μmol), then added with hydrogen peroxide (0.5 mL), and stirred at room temperature for 1.5 hours. After the reaction was completed, the reaction solution was concentrated under reduced pressure, and subjected to liquid phase chromatography purification (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-3-phenylpiperidin-1-yl)-3-(2,3-dichlorophenyl)-1H-pyrazolo[3,4-d]pyrimidine-4-carboxamide 57 (3.6 mg) with a yield of 16.5%.

MS m/z (ESI): 482.1 [M+1]

¹H NMR (400 MHz, DMSO-d₆) δ 8.11 (s, 1H), 7.86 (s, 1H), 7.58-7.77 (m, 2H), 7.41 (dt, J=20.8, 8.0 Hz, 4H), 4.26 (s, 1H), 3.77 (s, 1H), 3.20 (q, J=13.4, 12.6 Hz, 2H), 2.80 (s, 1H), 2.17 (d, J=12.4 Hz, 1H), 1.98 (s, 1H), 1.59 (s, 1H).

### Example 58

### 6-(4-amino-4-ethylpiperidin-1-yl)-3-(2,3-dichlorophenyl)-1H-pyrazolo[3,4-d]pyrimidine-4-carboxamide

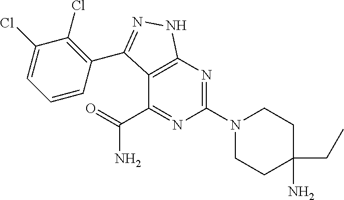 [SMILES: CCC1(N)CCN(c2nc(C(N)=O)c3c(-c4cccc(Cl)c4Cl)n[nH]c3n2)CC1]

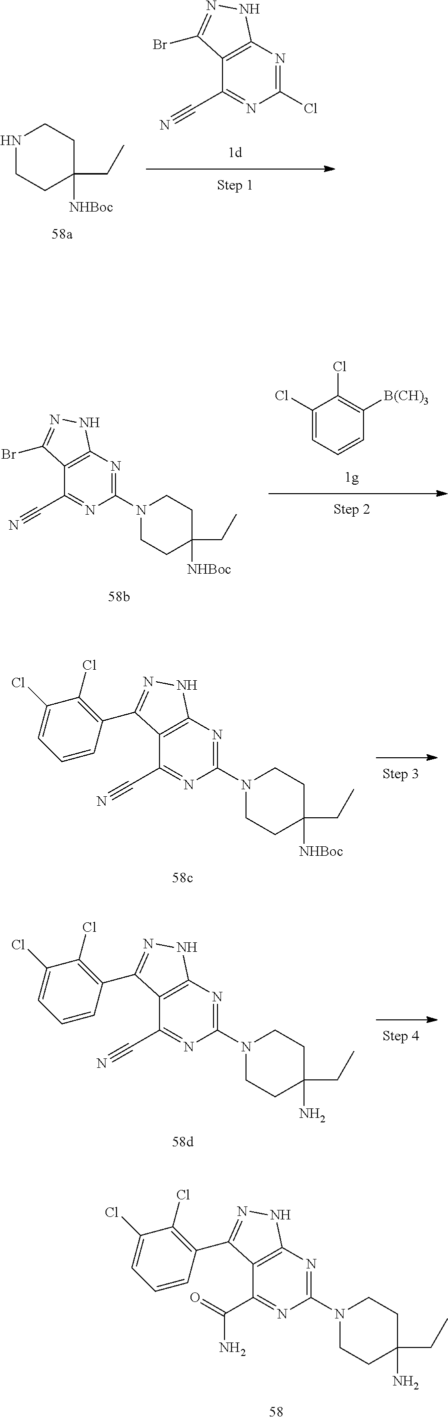

### Step 1

### Tert-butyl (1-(3-bromo-4-cyano-1H-pyrazolo[3,4-d]pyrimidin-6-yl)-4-ethylpiperidin-4-yl)carbamate

3-bromo-6-chloro-1H-pyrazolo[3,4-d]pyrimidine-4-carbonitrile 1d (150 mg, 580 μmol), tert-butyl (4-ethylpiperidin-4-yl) carbamate 58a (133 mg, 580 μmol) and N,N-diisopropylethylamine (300 mg, 2.32 mmol, 385 μL) were added to N-methyl pyrrolidone (3 mL) in turn, subjected to argon gas displacement, heated to 100° C., and reacted for 4 hours. After the reaction was completed, the reaction solution was cooled to room temperature, quenched with a saturated aqueous ammonium chloride solution (30 mL), then added with ethyl acetate (30 mL), and aqueous phases were washed with ethyl acetate (30 mL×2). Organic phases were combined, and washed with saturated salt water, dried by anhydrous sodium sulfate, and concentrated under reduced pressure. The obtained residue was further separated and purified by silica gel column chromatography (eluent: system A) to obtain tert-butyl (1-(3-bromo-4-cyano-1H-pyrazolo[3,4-d]pyrimidin-6-yl)-4-ethylpiperidin-4-yl)carbamate 58b (250 mg) with a yield of 96%.

MS m/z (ESI): 449.9 [M+1]

### Step 2

### Tert-butyl (1-(3-(2,3-dichlorophenyl)-4-cyano-1H-pyrazolo[3,4-d]pyrimidine-6-yl)-4-ethylpiperidine-4-yl) carbamate

Tert-butyl (1-(3-bromo-4-cyano-1H-pyrazolo[3,4-d]pyrimidin-6-yl)-4-ethylpiperidin-4-yl)carbamate 58b (250 mg, 555 μmol), (2,3-dichlorophenyl)boronic acid 1g (424 mg, 2.22 mmol), methanesulfonato(2-dicyclohexylphosphino-2′,6′-di-isopropoxy-1,1′-biphenyl)(2-amino-1,1′-biphenyl-2-yl)palladium (93 mg, 111 μmol), 2-dicyclohexylphosphino-2′,6′-di-isopropoxy-1,1′-biphenyl (104 mg, 222 μmol) and potassium phosphate (471 mg, 2.22 mmol) were added to a mixed solution of 1,4-dioxane (3 mL) and water (0.3 mL), subjected to argon gas displacement thrice, heated to 130° C., and reacted for 24 hours. After the reaction was completed, the reaction solution was concentrated under reduced pressure, quenched with a saturated aqueous ammonium chloride solution (30 mL), then added with ethyl acetate (30 mL), and aqueous phases were washed with ethyl acetate (30 mL×2). Organic phases were combined, and washed with saturated salt water, dried by anhydrous sodium sulfate, and concentrated under reduced pressure. The obtained residue was further separated and purified by silica gel column chromatography (eluent: system A) to obtain tert-butyl (1-(3-(2,3-dichlorophenyl)-4-cyano-1H-pyrazolo[3,4-d]pyrimidine-6-yl)-4-ethylpiperidine-4-yl) carbamate 58c (250 mg) with a yield of 87%.

MS m/z (ESI): 516.2 [M+1]

### Step 3

### 6-(4-amino-4-ethylpiperidin-1-yl)-3-(2,3-dichlorophenyl)-1H-pyrazolo[3,4-d]pyrimidine-4-carbonitrile

Tert-butyl (1-(3-(2,3-dichlorophenyl)-4-cyano-1H-pyrazolo[3,4-d]pyrimidine-6-yl)-4-ethylpiperidine-4-yl) carbamate 58c (250 mg, 484 μmol) was dissolved in dichloromethane (5 mL), slowly added with a trifluoroacetic acid (5.00 g, 43.9 mmol), and stirred overnight at room temperature. After the reaction was completed, the reaction solution was added with ethyl acetate (80 mL), and extracted with saturated sodium bicarbonate (100 mL×3). Organic phases were dried with anhydrous sodium sulfate, filtered, and concentrated under reduced pressure to obtain 6-(4-amino-4-ethylpiperidin-1-yl)-3-(2,3-dichlorophenyl)-1H-pyrazolo[3,4-d]pyrimidine-4-carbonitrile 58d (140 mg) with a yield of 69%, which was directly used for the next reaction without purification.

MS m/z (ESI): 416.1 [M+1]

### Step 4

### 6-(4-amino-4-ethylpiperidin-1-yl)-3-(2,3-dichlorophenyl)-1H-pyrazolo[3,4-d]pyrimidine-4-carboxamide

6-(4-amino-4-ethylpiperidin-1-yl)-3-(2,3-dichlorophenyl)-1H-pyrazolo[3,4-d]pyrimidine-4-carbonitrile 58d (140 mg, 336 μmol) and potassium hydroxide (38 mg, 673 μmol) were added to dimethyl sulfoxide (4 mL) in turn, and then slowly dropwise added with hydrogen peroxide(30⁰/o, 1 mL), and continuously stirred at room temperature for 1 hour. After the reaction was completed, a small amount of water was added to precipitate a faint yellow solid, a solid was obtained by filtration, and subjected to liquid phase separation (separation column: AKZONOBEL Kromasil; 250×21.2 mm I.D.; 5 μm, 20 mL/min; mobile phase A: 0.05% TFA+H₂O, mobile phase B: CH₃CN) to obtain 6-(4-amino-4-ethylpiperidin-1-yl)-3-(2,3-dichlorophenyl)-1H-pyrazolo[3,4-d]pyrimidine-4-carboxamide 58 (15 mg) with a yield of 10.3%.

MS m/z (ESI): 433.9 [M+1]

¹H NMR (400 MHz, CD₃OD) δ 7.57-7.59 (m, 1H), 7.33-7.40 (m, 2H), 4.37-4.41 (m, 2H), 3.76-3.82 (m, 2H), 2.66 (s, 4H), 1.90-1.99 (m, 4H), 1.79-1.86 (m, 2H), 1.06 (t, J=7.6 Hz, 3H).

### Biological Evaluation

### Test Example 1 Determination of Allosteric Inhibition Activities of the Compounds of the Present Invention on SHP2

The following method was used to determine the inhibition degrees of the compounds of the present invention on the activity of recombinant human full-length SHP2 in vitro. SHP2 was allosterically activated by the binding of di-tyrosyl-phosphorylated peptide to a Src homologous 2(SH2) domain of SHP2. An activation step of the latter leads to the self-inhibitory interface release of SHP2, and then activates SHP2 protein tyrosine phosphatase (PTP), which might be used for substrate recognition and reaction catalysis.

The experimental process was briefly described as follows: Test compounds were first dissolved in DMSO to prepare storage solutions. The reaction was carried out in a 384-well Small Volume™ HiBase microplate (Greiner, 784075). Firstly, SHP2 (signalchem, P38-20G-10ug) and SHP-2 Activating Peptide (IRS1_pY1172(dPEG8)pY1222) BPS, 79319-1) were added to the wells till the final concentrations were 0.5 nM and 0.5 uM, respectively. Then, the compounds to be tested were added in a concentration range of 0.00004-10 uM and incubated at 25° C. for 60 minutes. Then DiFMUP (Thermo, D6567) was added in the reaction and incubated at 25° C. for 30 minutes. After incubation, readings were taken using a microplate reader (BMG) with excitation and emission wavelengths of 340 nm and 450 nm, respectively. Compared with the fluorescence intensity ratio of a control group (0.1% DMSO), the percentage inhibition rates of the compounds at each concentration were calculated, and the IC₅₀ values of the compounds were obtained by performing nonlinear regression analysis with logarithmic value of the compound concentration—inhibition rate by GraphPad Prism 5 software, which was shown in Table 1.

TABLE 1
IC₅₀ data of the compounds of the present invention
on activity inhibition of full-length SHP2 enzyme

| Compound No. | SHP2/IC₅₀ (nM) |
|---|---|
| SHP-099 | 128 |
| 2 | 29 |
| 5 | 1 |
| 6 | 4 |
| 7 | 14 |
| 8 | 5 |
| 9 | 1 |
| 10 | 7 |
| 11 | 17 |
| 12 | 8 |
| 13 | 10 |
| 15 | 24 |
| 17 | 4 |
| 23 | 2 |
| 24 | 5 |
| 25 | 5 |
| 27 | 7 |
| 29 | 3 |
| 30 | 3 |
| 32 | 10 |
| 38 | 27 |
| 46 | 3 |
| 51 | 3 |
| 53 | 11 |

Conclusion: it can be seen from Table 1 that the compounds of the present invention have preferable allosteric inhibition effects on SHP2 enzyme.

Remarks: the structure of SHP-099 (prepared according to WO2015107493) is as follows:

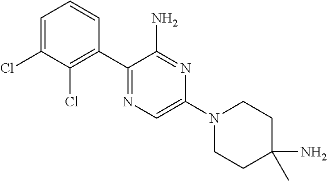 [SMILES: CC1(N)CCN(c2cnc(-c3cccc(Cl)c3Cl)c(N)n2)CC1]

### Test Example 2 Determination of the Compounds of the Present Invention on Inhibiting NCI-H23 Cell Proliferation

The following method was used to determine the effects of the compounds of the present invention on NCI-H23 cell proliferation. NCI-H23 cells (containing KRAS G12C mutation) were purchased from Cell Resource Center of Shanghai Institutes for Biological Sciences, Chinese Academy of Sciences, and cultured in RPMI 1640 mediums containing 10% fetal bovine serum, 100 U penicillin, 100 μg/mL streptomycin and 1 mM Sodium Pyruvate. The activities of the cells were determined by CellTiter-Glo® Luminescent Cell Viability Assay kit (Promega, article number: G7573).

The experimental method was operated according to the steps in the kit instruction, and was briefly described as follows: test compounds were first dissolved in DMSO to prepare storage solutions of 10 mM, and then diluted in mediums, prepared as test samples. The final concentrations of the test compounds were 10,000 nM to 1.52 nM. The cells in logarithmic phase were inoculated in a 96-well cell culture plate with 1,000 cells per well, after cultured overnight in 5% CO₂ incubator at 37° C., the test compounds were added to the incubator to continue the culture for 120 hours. After the culture, 50 μL of CellTiter-Glo detection solution was added to each well, shaken for 5 minutes, and then stood for 10 minutes. Then, a Luminescence mode was used to read the luminescence values of each well of the samples on a microplate reader. Compared with the numerical value of a control group (0.3% DMSO), the percentage inhibition rates of the compounds at each concentration were calculated, and the IC₅₀ values of the compounds inhibiting cell proliferation were obtained by performing nonlinear regression analysis with logarithmic values of the compound concentration—inhibition rate by GraphPad Prism 5 software, which was shown in Table 2.

TABLE 2
IC₅₀ data of the compounds of the present invention
on inhibiting NCI-H23 cell proliferation

| Example No. | IC₅₀(nM)/NCI-H23 |
|---|---|
| RMC-4550 | 240 |
| 5 | 143 |
| 6 | 61 |
| 9 | 13 |
| 27 | 167 |

It can be seen from Table 2 that the compounds of the present invention have preferable inhibition effects on NCI-H23.

Remarks: the structure of RMC-4550 (prepared according to WO2018013597) is as follows:

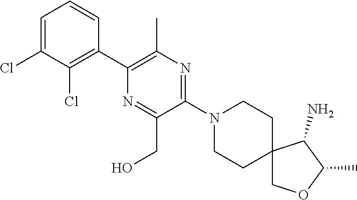 [SMILES: Cc1nc(N2CCC3(CC2)CO[C@@H](C)[C@H]3N)c(CO)nc1-c1cccc(Cl)c1Cl]

### Test Example 3 Determination of the Compounds of the Present Invention on Inhibiting NCI-H358 Cell Proliferation

The following method was used to determine the effects of the compounds of the present invention on NCI-H358 cell proliferation. NCI-H358 cells (containing KRAS G12C mutation) were purchased from Cell Resource Center of Shanghai Institutes for Biological Sciences, Chinese Academy of Sciences, and cultured in RPMI 1640 mediums containing 10% fetal bovine serum, 100 U penicillin, 100 μg/mL streptomycin and 1 mM Sodium Pyruvate. The activities of the cells were determined by CellTiter-Glo® Luminescent Cell Viability Assay kit (Promega, article number: G7573).

The experimental method was operated according to the steps in the kit instruction, and was briefly described as follows: test compounds were first dissolved in DMSO to prepare storage solutions of 10 mM, and then diluted in mediums, prepared as test samples. The final concentrations of the test compounds were 10,000 nM to 1.52 nM. The cells in logarithmic phase were inoculated in a 96-well cell culture plate with 1,000 cells per well, after cultured overnight in 5% CO₂ incubator at 37° C., the test compounds were added to the incubator to continue the culture for 120 hours. After the culture, 50 μL of CellTiter-Glo detection solution was added to each well, shaken for 5 minutes, and then stood for 10 minutes. Then, a Luminescence mode was used to read the luminescence values of each well of the samples on a microplate reader. Compared with the numerical value of the control group (0.3% DMSO), the percentage inhibition rates of the compounds at each concentration were calculated, and the IC₅₀ values of the compounds inhibiting cell proliferation were obtained by performing nonlinear regression analysis with logarithmic values of the compound concentration—inhibition rate by GraphPad Prism 5 software, which was shown in Table 3.

TABLE 3
IC₅₀ data of the compounds of the present invention
on inhibiting NCI-H358 cell proliferation

| Example No. | IC₅₀(nM)/NCI-H358 |
|---|---|
| RMC-4550 | 80 |
| 5 | 27 |
| 6 | 19 |
| 27 | 37 |

It can be seen from Table 3 that the compounds of the present invention have preferable inhibition effects on NCI-H358.

### Test Example 4 Pharmacokinetic Test

### 1. Experimental Purpose

ICR mice were used as test animals, and LC/MS/MS methods was used to determine the drug concentrations at different moments in plasma of mice administered by intragastric injection with the compound 5 and the compound 6 of the present invention, and to study the pharmacokinetic characteristics of the compounds of the present invention in mice.

### 2. Experimental Solution

2.1 Experimental Drugs and Animals:

Compound 5 and compound 6

ICR mice, male, 29.0 g to 33.8 g, purchased from Beijing Charles River Laboratory Animal Technology Co., Ltd.

2.2 Drug Preparation

An appropriate amount of drug was weighed, added with an appropriate amount of sodium carboxymethylcellulose (CMC-Na, containing 0.5% Tween 80), vortexed, and ultrasonically prepared into 1 mg/kg suspension.

2.3 Administration

The ICR mice in the intragastric group of each compound to be tested (9 mice in each group) were fasted overnight and then administered by intragastric injection (PO, administration dose of 1 mg/kg, and administration volume of 10 mL/kg), and ate 4 hours after administration.

### 3. Operation

About 0.2 mL of blood was collected via jugular vein before administration and 0.083 hour, 0.25 hour, 0.5 hour, 1 hour, 2 hours, 4 hours, 8 hours and 24 hours after administration, and heparin sodium was used for anticoagulation. The collected blood samples were placed on ice, and plasma was separated by centrifugation (centrifugation condition: 1,500 g, 10 minutes). The collected plasma samples were stored at −40° C. to −20° C. before analysis.

LC-MS/MS was used to determine the contents of the compounds to be tested in mouse plasma after intragastric administration.

### 4. Results of Pharmacokinetic Parameters

The pharmacokinetic parameters of the compounds of the present invention were shown in Table 4.

TABLE 4
Pharmacokinetic parameters of mice administrated with compounds

| | Pharmacokinetic experiment | | |
|---|---|---|---|
| | Administration mode | Blood | Area under |
| | and administration | concentration | curve AUC_{0-∞} |
| Compound No. | dose | Cₘₐₓ (ng/mL) | (ng · h/mL) |
| Compound 5 | PO | 2,113 | 31,264 |
| | (10 mg/kg) | | |
| Compound 6 | PO | 1,192 | 18,471 |
| | (10 mg/kg) | | |

Conclusion: the compound 5 and the compound 6 of the present invention have high blood concentrations and areas under curve, and have good pharmacokinetic properties.

### Test Example 5 Pharmacokinetic Test of Mice

### 1. Experimental Purpose

ICR mice were used as test animals, and LC/MS/MS methods was used to determine the drug concentrations at different moments in plasma of mice administered by intragastric injectiion with the compound 27 of the present invention, and to study the pharmacokinetic characteristics of the compound of the present invention in mice.

### 2. Experimental Solution

2.1 Experimental Drugs and Animals:

Compound 27

ICR mice, male, 29.0 g to 33.8 g, purchased from Beijing Charles River Laboratory Animal Technology Co., Ltd.

2.2 Drug Preparation

An appropriate amount of drug compound 27 was weighed, added with an appropriate amount of sodium carboxymethylcellulose (CMC-Na, containing 0.5% Tween 80), vortexed, and ultrasonically prepared into 0.5 mg/kg suspension.

2.3 Administration

The ICR mice in the intragastric group of each compound to be tested (9 mice in each group) were fasted overnight and then administered by intragastric injection (PO, administration dose of the compound 27 was 5 mg/kg, and administration volume of the compound 27 was 10 mL/kg), and ate 4 hours after administration.

### 3. Operation

About 0.2 mL of blood was collected via jugular vein before administration and 0.083 hour, 0.25 hour, 0.5 hour, 1 hour, 2 hours, 4 hours, 8 hours, 12 hours and 24 hours after administration, and heparin sodium was used for anticoagulation. The collected blood samples were placed on ice, and plasma was separated by centrifugation (centrifugation condition: 1,500 g, 10 minutes). The collected plasma samples were stored at −40° C. to −20° C. before analysis.

LC-MS/MS was used to determine the content of the compound to be tested in mouse plasma after intragastric administration.

### 4. Results of Pharmacokinetic Parameters

The pharmacokinetic parameters of the compound of the present invention were shown in Table 5.

TABLE 5
Pharmacokinetic parameters of mice administrated with compound

| | Pharmacokinetic experiment | | | |
|---|---|---|---|---|
| | Administration mode | Blood | Area under | |
| | and administration | concentration | curve AUC_{0-∞} | Peak time |
| Compound No. | dose | Cₘₐₓ (ng/mL) | (ng · h/mL) | Tmax (h) |
| Compound 27 | PO | 285 | 2,720 | 8 |
| | (5 mg/kg) | | | |

Conclusion: the compound 27 of the present invention has high blood concentration and area under curve, and has good pharmacokinetic properties.

### Test Example 6 Test of Growth Inhibition Effect of the Compounds of the Present Invention in Subcutaneous Transplanted Tumors of NCI-H358 Tumor-Bearing BALB/c Nude Mice

### 1. Experimental Purpose

To evaluate antitumor effects and safety of the compounds of Example 27 in an animal model of BALB/c nude mice with subcutaneous transplanted NCI-H358 cell lines

### 2. Subject Preparation

Solvent control group given DMA: Solutol HS 15: Saline=5:10:85 (v/v/v);

An approximate amount of the compound of Example 27 was weighed, added with an approximate amount of DMA (dimethylacetamide) to fully dissolve the compound, then added with Solutol HS 15 and Saline (DMA: Solutol HS 15: Saline=5:10:85 (v/v/v)) in turn, and mixed evenly by vortex, and the configuration concentration was 3 mg/mL.

### 3. Experimental Animals

BALB/c nude mice, female, 6-7 weeks old (the age of mice when tumor cells were inoculated), 12 mice, purchased from Jiangsu GemPharmatech.

### 4. Cell Culture

NCI-H358 cells were cultured in RPMI 1640 mediums containing 10% fetal bovine serum, 1% sodium pyruvate and 1% glutamine. NCI-H358 cells in exponential growth period were collected, and resuspend in PBS to a suitable concentration for subcutaneous tumor inoculation in nude mice.

### 5. Animal Modeling and Random Grouping

12 female BALB/c nude mice were subcutaneously inoculated with about 3×10⁶ NCI-H358 cells on the right sides of their backs. When the average volume of tumors reaches about 100 mm³ to 200 mm³, the mice were randomly divided according to tumor sizes with 6 mice in each group. The animals in each group were given the subjects once a day (qd) according to the animal body weights at a fixed time every day according to the table below, administered orally (po) for 10 consecutive days, and the daily body weights were recorded.

### 6. Animal Administration and Observation

After tumor inoculation, routine monitoring included the effects of tumor growth and treatment on the normal behaviors of the animals, specifically the mobility, feeding and drinking, weight gain or loss, eyes, coat and other abnormalities of the experimental animals.

Calculation formulae of a relative tumor volume (RTV), a relative tumor inhibition rate (T/C) and a tumor inhibition percentage (IR) were as follows:

(1) TV (tumor volume)=½×a×b², wherein a and b represent the length and the width of the tumor respectively;

(2) RTV (relative tumor volume)=Vₜ/V₀, wherein V₀ is the tumor volume measured at the time of grouping administration (i.e., d0), and Vₜ is the tumor volume at each measurement;

(3) Relative tumor proliferation rate T/C (%)=T_{RTV}/C_{RTV}×100%, wherein T_{RTV} is the RTV of the treatment group and C_{RTV} is the RTV of the control group;

(4) Tumor growth inhibition rate TGI (%)=(1−T/C)×100%; wherein, T and C are the relative tumor volumes of the treatment group and the control group at a specific time point.

### 7. Results

TABLE 6
Inhibition rate (TGI %) of the compound of the present
invention on tumor growth of human non-small cell
lung cancer cell NCI-H358 tumor-bearing mice

| | Tumor volume | | |
|---|---|---|---|
| | (mm³, mean value ± standard error) | | TGI % |
| Group | Day 0 | Day 10 | Day 10 |
| Solvent control group | 138 ± 14 | 347 ± 38 | — |
| Example 27 30 mg/kg | 138 ± 14 | 155 ± 22 | 55.6% |

At the dose of 30 mg/kg, the compound of Example 27 of the present invention has obvious growth inhibition effects on NCI-H358 tumor-bearing BALB/c nude mice, with no obvious change in body weight, and has high safety.

1. A compound represented by a general formula (AI) or a stereoisomer or a tautomer thereof, or a pharmaceutically acceptable salt thereof: 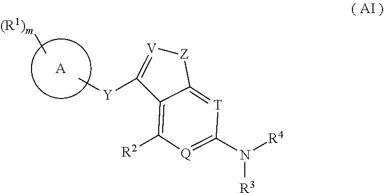
wherein:
Y is selected from a chemical bond or —S—;
when Z is selected from —NH—, V is selected from —N— or —CH—; alternatively, when Z is selected from —O—, V is selected from —N—;
Q and T are each independently selected from N or CH; wherein at least one of Q and T is selected from N;
ring A is selected from aryl, heteroaryl or bicyclic fused ring, wherein the aryl is monocyclic aryl, the heteroaryl is a 5-6 membered monocyclic heteroaryl, and the bicyclic fused ring is preferably a fused ring of aryl or heteroaryl with monocyclic heterocyclyl or monocyclic cycloalkyl;
R¹ are the same or different, and are each independently selected from hydrogen atom, alkyl, alkenyl, alkynyl, cyano, halogen, nitro, cycloalkyl, heterocyclyl, —OR⁵, —C(O)R⁵, —SO₂R⁵, —NR⁶R⁷, —SO₂NR⁶R⁷, —NHSO₂R⁵ or —C(O)NR⁶R⁷, wherein the alkyl, alkenyl, alkynyl, cycloalkyl or heterocyclyl is optionally further substituted by one or more substituents selected from halogen, nitro, cyano, alkyl, cycloalkyl, heterocyclyl, aryl, heteroaryl, —OR⁵, —C(O)R⁵, —C(O)OR⁵, —OC(O)R⁵, —SO₂R⁵, —NR⁶R⁷, —SO₂NR⁶R⁷, —NHSO₂R⁵ or —C(O)NR⁶R⁷;
R² is selected from cyano, tetrazolyl, —C(O)R⁵, —C(O)OR⁵ or —C(O)NR⁶R⁷;
R³ and R⁴ together with the N atom bound therewith form a 4-11 membered heterocyclyl, preferably a 5-11 membered heterocyclyl, wherein the heterocyclyl internally contains one or more N, O, S or SO₂ atoms, and the heterocyclyl is optionally further substituted by one or more substituents selected from halogen, nitro, cyano, alkyl, alkenyl, alkynyl, cycloalkyl, heterocyclyl, aryl, heteroaryl, —CH₂R⁵, —CH(OH)R⁵, —CH₂OR⁵, ═O, —OR⁵, —SR⁵, —SOR³, —C(O)R³, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷, wherein the alkyl, alkenyl, alkynyl, cycloalkyl, heterocyclyl, aryl or heteroaryl is optionally further substituted by one or more substituents selected from hydroxy, amino, halogen, nitro, cyano, alkyl, haloalkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
alternatively, R³ and R⁴ together with the N atom bound therewith form a group: 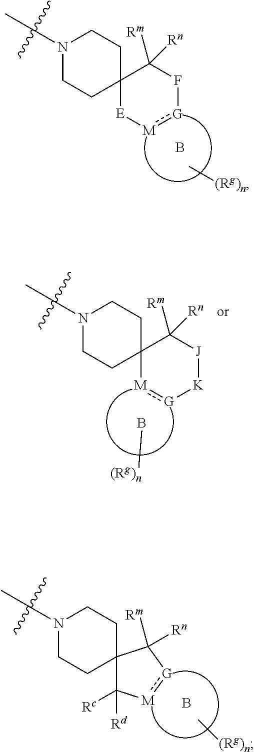 [symbol]
is a single bond or double bond;
when [symbol] represents a single bond, G and M are each independently selected from N or CR^{j};
when [symbol] represents a double bond, G and M are each independently selected from C;
ring B is selected from cycloalkyl, heterocyclyl aryl or heteroaryl;
E is selected from NR^{k}, (CR^{p}R^{q})ₚ, O or S;
F is selected from (CR^{p}R^{p})_{q};
the condition is that when E is selected from (CR^{p}R^{q})ₚ, p is 1 and q is 1; alternatively, p is 2 and q is 0; and when E is selected from NR^{k}, O or S, q is 1;
J is selected from CR^{p}R^{q};
K is selected from NRk, (CR^{p}R^{q})ᵣ, O or S;
r is 0 or 1;
R^{m}, Rⁿ, R^{p} and R^{q} are the same or different, and are each independently selected from R^{A};
alternatively, R^{p} and R^{q} together with the carbon atom bound therewith form R^{B};
R^{c} and R^{d} are the same or different, and are each independently selected from hydrogen atom, halogen, alkyl or —OR⁵, wherein the alkyl is optionally further substituted by a substituent of hydroxy, halogen, alkoxy, cycloalkyl or —NR⁶R⁷;
alternatively, R^{c} and R^{d} together with the carbon atom bound therewith form R^{B};
R^{g} are the same or different, and are each independently selected from hydrogen atom, halogen, nitro, alkyl, alkenyl, alkynyl, cyano, cycloalkyl, heterocyclyl, aryl, heteroaryl, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷, wherein the alkyl, alkenyl, alkynyl, cycloalkyl, heterocyclyl, aryl or heteroaryl is optionally further substituted by a substituent of hydroxy, halogen, alkyl, alkoxy, cycloalkyl or —NR⁶R⁷;
alternatively, two R^{g} together with the same carbon atom bound therewith form C═O;
R^{j} and R^{k} are the same or different, and are each independently selected from hydrogen atom or alkyl;
R^{A} are the same or different, and are each independently selected from hydrogen atom, halogen, nitro, alkyl, alkenyl, alkynyl, cyano, cycloalkyl, heterocyclyl, aryl, heteroaryl, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷, wherein the alkyl, alkenyl, alkynyl, cycloalkyl, heterocyclyl, aryl or heteroaryl is optionally further substituted by a substituent of hydroxy, halogen, alkyl, alkoxy, cycloalkyl or —NR⁶R⁷;
R^{B} are the same or different, and are each independently selected from 3-10 membered cycloalkyl or 3-10 membered heterocyclyl, wherein the cycloalkyl or heterocyclyl is optionally further substituted by one or more substituents selected from halogen, cyano, nitro, alkyl, cycloalkyl, heterocyclyl, aryl, heteroaryl, ═O, —OR⁵, —C(O)R⁵, —C(O)OR⁵, —OC(O)R⁵, —SO₂R⁵, —NR⁶R⁷, —SO₂NR⁶R⁷, —NHC(═NH)NH₂, —NHSO₂R⁵ or —C(O)NR⁶R⁷;
R⁵, R⁶ and R⁷ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, amino, halogen, nitro, cyano, alkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
alternatively, R⁶ and R⁷ together with the N atom bound therewith form a 3-8 membered heterocyclyl, wherein the 3-8 membered heterocyclyl internally contains one or more N, O, S or SO₂ atoms, and the 3-8 membered heterocyclyl is optionally further substituted by one or more substituents selected from hydroxy, halogen, amino, alkyl or alkoxy;
R⁸, R⁹ and R¹⁰ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, halogen, nitro, cyano, alkyl, alkoxy, cycloalkyl, heterocyclyl, aryl, heteroaryl, carboxyl or carboxylate;
m is 0, 1, 2, 3, 4 or 5;
n is selected from 0, 1, 2, 3 or 4; and
p is selected from 1 or 2.

2. The compound or the stereoisomer or the tautomer thereof, or the pharmaceutically acceptable salt thereof according to claim 1, which is a compound represented by general formula (AII) or a stereoisomer or a tautomer thereof, or a pharmaceutically acceptable salt thereof: 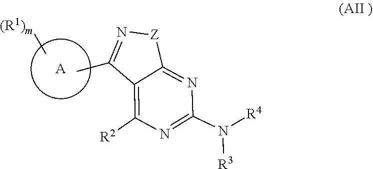
wherein: ring A, m, Z and R¹-R⁴ are defined as in claim 1.

3. The compound or the stereoisomer or the tautomer thereof, or the pharmaceutically acceptable salt thereof according to claim 1, which is a compound represented by general formula (I) or a stereoisomer or a tautomer thereof, or a pharmaceutically acceptable salt thereof: 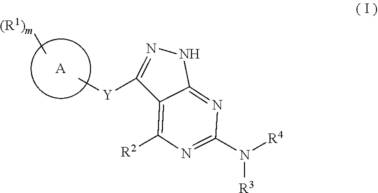
wherein: ring A, Y, m and R¹-R⁴ are defined as in claim 1.

4. The compound or the stereoisomer or the tautomer thereof, or the pharmaceutically acceptable salt thereof according to claim 1, which is a compound represented by general formula (II) or a stereoisomer or a tautomer thereof, or a pharmaceutically acceptable salt thereof: 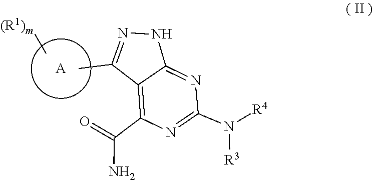
wherein: ring A, m, R¹, R³ and R⁴ are defined as in claim 1.

5. The compound or the stereoisomer or the tautomer thereof, or the pharmaceutically acceptable salt thereof according to claim 1, wherein:
R³ and R⁴ together with the N atom bound therewith form a 4-8 membered monocyclic heterocyclyl, preferably a 5-6 membered monocyclic heterocyclyl, more preferably piperidinyl, wherein the monocyclic heterocyclyl is optionally further substituted by one or more substituents selected from methyl, amino, cycloalkyl, phenyl, halophenyl, heteroaryl, —CH₂NH₂, —CH₂OH, —NHC(═NH)NH₂, ═O or —OR⁵; wherein the methyl, cycloalkyl, phenyl or heteroaryl is optionally further substituted by substituents selected from one or more of mesyl, hydroxy, amino, halogen, haloalkyl, alkoxy, haloalkoxy, pyridinyl, or pyrimidinyl; wherein the heteroaryl is preferably pyridinyl, pyrimidinomethylbenzopyrazolyl, pyrrolyl, furanyl, thienyl, pyrazolyl, imidazolyl, thiazolyl, benzimidazolyl, benzofuranyl or benzoxazolyl; and
R⁵ is defined as in claim 1;
alternatively, R³ and R⁴ together with the N atom bound therewith form a 7-11 membered bridged heterocyclyl, wherein the bridged heterocyclyl is optionally further substituted by one or more substituents selected from methyl, amino, —CH₂NH₂, —CH₂OH, —NHC(═NH)NH₂, ═O or —OR⁵; and
R⁵ is defined as in claim 1;
alternatively, R³ and R⁴ together with the N atom bound therewith form a 7-11 membered fused heterocyclyl, wherein the fused heterocyclyl is optionally further substituted by one or more substituents selected from methyl, amino, —CH₂NH₂, —CH₂OH, —NHC(═NH)NH₂, ═O or —OR⁵; and
R⁵ is defined as in claim 1;
alternatively, R³ and R⁴ together with the N atom bound therewith form a 7-11 membered spiroheterocyclyl, wherein the spiroheterocyclyl is optionally further substituted by one or more substituents selected from methyl, amino, —CH₂NH₂, —CH₂OH, —NHC(═NH)NH₂, ═O or —OR⁵; and
R⁵ is defined as in claim 1;
Preferably, the spiroheterocyclyl is selected from: 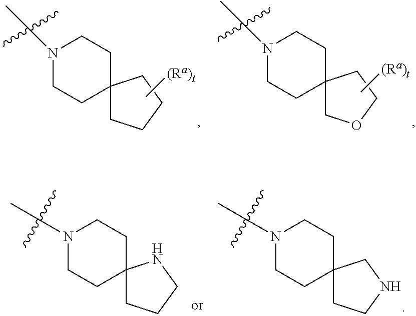
R^{a} are the same or different, and are each independently selected from methyl, amino, —CH₂NH₂, —CH₂OH, —NHC(═NH)NH₂ or —OR⁵;
alternatively, two R^{a} together with the same carbon atom bound therewith form C═O;
t is 1, 2 or 3.

6. (canceled)

7. (canceled)

8. (canceled)

9. (canceled)

10. The compound or the stereoisomer or the tautomer thereof, or the pharmaceutically acceptable salt thereof according to claim 3, which is a compound represented by general formula (III) or a stereoisomer or a tautomer thereof, or a pharmaceutically acceptable salt thereof: 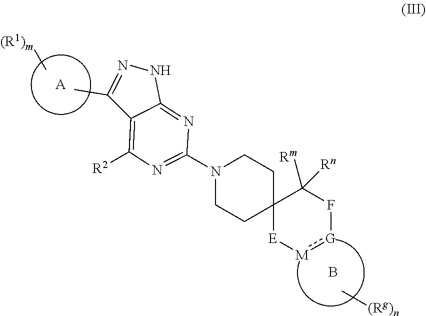
wherein:
ring B is selected from phenyl, 3-8 membered cycloalkyl, 4-8 membered heterocyclyl or 5-6 membered heteroaryl;
E is selected from NR^{k}, (CR^{p}R^{q})ₚ, O or S;
F is selected from ((CR^{p}R^{q})_{q};
the condition is that when E is selected from (CR^{p}R^{q})ₚ, p is 1 and q is 1; alternatively, p is 2 and q is 0; and when E is selected from NR^{k}, O or S, q is 1;
R^{m} is selected from amino, —CH₂NH₂ or —NHC(═NH)NH₂;
Rⁿ is selected from hydrogen atom, methyl or —CH₂OH;
R^{p} and R^{q} are each independently selected from hydrogen atom, halogen, amino, C₁-C₄ alkyl, hydroxy C₁-C₄ alkyls, amino C₁-C₄ alkyls or —OR⁵; and [symbol]
, ring A, G, M, m, n, R¹-R², R⁵, R^{k} and R^{g} are defined as in claim 3;
preferably, R² is selected from —C(O)NH₂ or —C(O)OH;
more preferably, R^{g} are the same or different, and are each independently selected from hydrogen atom, F, Cl, Br, amino, hydroxy, cyano, nitro, methoxy, ethoxy, methyl, ethyl, ethynyl, ethenyl, —NHCH₃ or —N(CH₃)₂; and
alternatively, two R^{g} together with the same carbon atom bound therewith form C═O.

11. The compound or the stereoisomer or the tautomer thereof, or the pharmaceutically acceptable salt thereof according to claim 3, which is a compound represented by general formula (IV) or a stereoisomer or a tautomer thereof, or a pharmaceutically acceptable salt thereof: 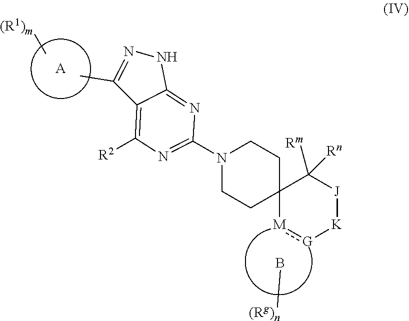
wherein:
ring B is selected from phenyl, 3-8 membered cycloalkyl, 4-8 membered heterocyclyl or 5-6 membered heteroaryl;
J is selected from CR^{p}R^{q};
K is selected from NR^{k}, (CR^{p}R^{q})ᵣ, O or S;
r is 0 or 1;
R^{m} is selected from amino, —CH₂NH₂ or —NHC(═NH)NH₂;
Rⁿ is selected from hydrogen atom, methyl or —CH₂OH;
R^{p} and R^{q} are each independently selected from hydrogen atom, halogen, amino, C₁-C₄ alkyls, hydroxy C₁-C₄ alkyls, amino C₁-C₄ alkyls or —OR⁵; and [symbol]
, ring A, G, M, m, n, R¹-R², R⁵, R^{k} and R^{g} are defined as in claim 3,
preferably, R² is selected from —C(O)NH₂ or —C(O)OH;
more preferably, R^{g} are the same or different, and are each independently selected from hydrogen atom, F, Cl, Br, amino, hydroxy, cyano, nitro, methoxy, ethoxy, methyl, ethyl, ethynyl, ethenyl, —NHCH₃ or —N(CH₃)₂; and
alternatively, two R^{g} together with the same carbon atom bound therewith form C═O.

12. The compound or the stereoisomer or the tautomer thereof, or the pharmaceutically acceptable salt thereof according to claim 3, which is a compound represented by general formula (V) or a stereoisomer or a tautomer thereof, or a pharmaceutically acceptable salt thereof: 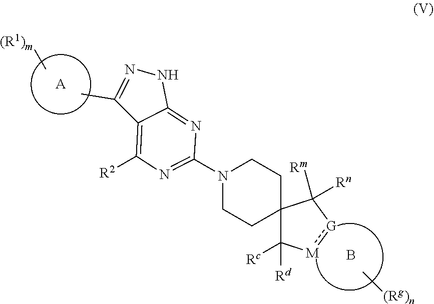
wherein:
ring B is selected from phenyl, 3-8 membered cycloalkyl, 4-8 membered heterocyclyl or 5-6 membered heteroaryl;
R^{c} and R^{d} together with the atom bound therewith form a 3-8 membered cycloalkyl;
R^{m} is selected from amino, —CH₂NH₂ or —NHC(═NH)NH₂;
Rⁿ is selected from hydrogen atom, methyl or —CH₂OH; and [symbol]
, ring A, G, M, m, n, R¹-R² and R^{g} are defined as in claim 3;
preferably, R² is selected from —C(O)NH₂ or —C(O)OH;
more preferably, R^{g} are the same or different, and are each independently selected from hydrogen atom, F, Cl, Br, amino, hydroxy, cyano, nitro, methoxy, ethoxy, methyl, ethyl, ethynyl, ethenyl, —NHCH₃ or —N(CH₃); and
alternatively, two R^{g} together with the same carbon atom bound therewith form C═O.

13. The compound or the stereoisomer or the tautomer thereof, or the pharmaceutically acceptable salt thereof according to claim 1, which is a compound represented by general formula (VI) or a stereoisomer or a tautomer thereof, or a pharmaceutically acceptable salt thereof: 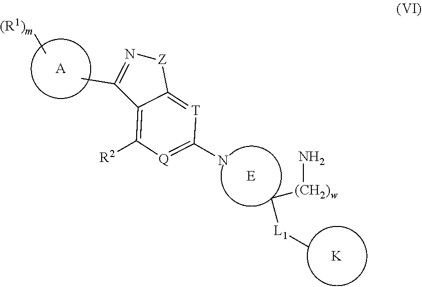
wherein:
L₁ is absent, or selected from —(C═O)— and —(CR^{w}R^{v})ᵤ—, wherein any one of —(CR^{W}R^{v})— is optionally further replaced by —N(R^{z})—, —O—, —S—, —SO— and —SO₂—;
each R^{w} and R^{v} are the same or different, and are each independently selected from hydrogen atom, halogen, hydroxy, alkyl or alkoxy;
each R^{z} are the same or different, and are each independently selected from hydrogen atom or alkyl;
ring E is selected from 4-11 membered monocyclic heterocyclyl containing N, 4-11 membered fused heterocyclyl containing N or 4-11 membered bridged heterocyclyl containing N, wherein the monocyclic heterocyclyl, fused heterocyclyl or bridged heterocyclyl is optionally further substituted by one or more substituents selected from halogen, alkyl, —OR⁵ or ═O;
ring K is absent, or selected from cycloalkyl, aryl or heteroaryl, wherein the cycloalkyl, aryl or heteroaryl is optionally further substituted by one or more substituents selected from hydroxy, amino, halogen, nitro, cyano, alkyl, alkoxy, haloalkyl, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
wherein -L₁-ring K and —(CH₂)_{w}—NH₂ are bound to the same carbon atom of ring E;
w is 0, 1 or 2;
u is 0, 1, 2 or 3; and
ring A, Z, Q, T, m, n, R¹-R², R⁵, and R⁸-R¹⁰ are defined as in claim;
preferably, R² is selected from —C(O)NH₂ or —C(O)OH;
more preferably, ring E is selected from:

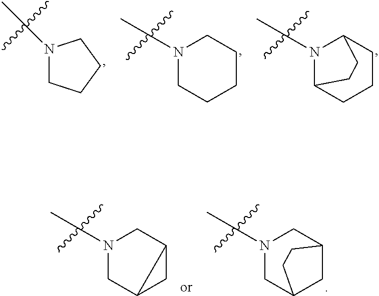 [SMILES: *N1CCCC1 ; *N1CCCCC1 ; *N1C2CCCC1CC2 ; *N1CC2CC2C1 ; *N1CC2CCC(C2)C1]

14. The compound or the stereoisomer or the tautomer thereof, or the pharmaceutically acceptable salt thereof according to claim 1, wherein R¹ is selected from hydrogen atom, F, Cl, Br, amino, hydroxy, cyano, nitro, methoxy, ethoxy, methyl, ethyl, trifluoromethyl, cyclopropyloxy, ethynyl, ethenyl, —NHCH₃ or —N(CH₃)₂.

15. (canceled)

16. The compound or the stereoisomer or the tautomer thereof, or the pharmaceutically acceptable salt thereof according to claim 1, wherein R⁵ is selected from hydrogen atom or alkyl.

17. The compound or the stereoisomer or the tautomer thereof, or the pharmaceutically acceptable salt thereof according to claim 1, wherein ring A is selected from phenyl, pyridinyl or pyrimidinyl.

18. The compound or the stereoisomer or the tautomer thereof, or the pharmaceutically acceptable salt thereof according to claim 10, wherein ring B is selected from:

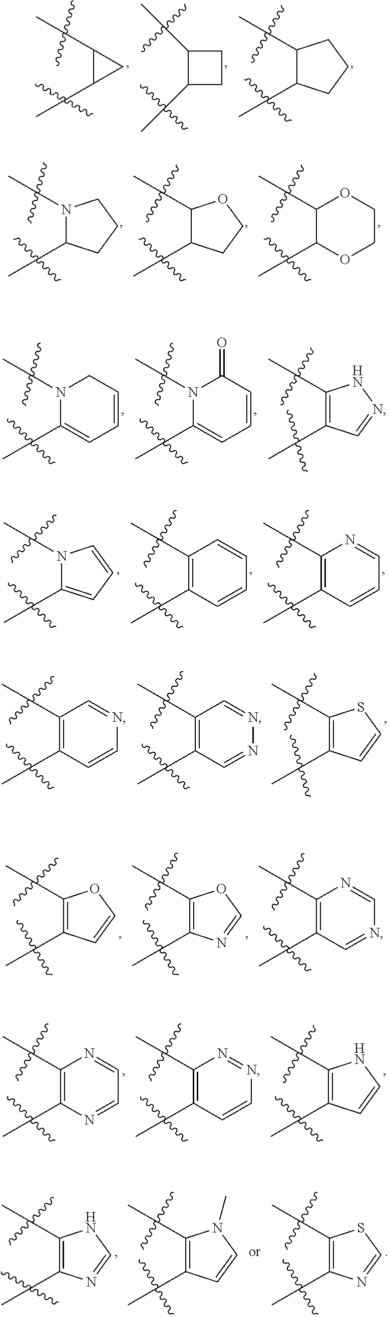

19. (canceled)

20. (canceled)

21. The compound or the stereoisomer or the tautomer thereof, or the pharmaceutically acceptable salt thereof according to claim 1, wherein the compound is selected from:

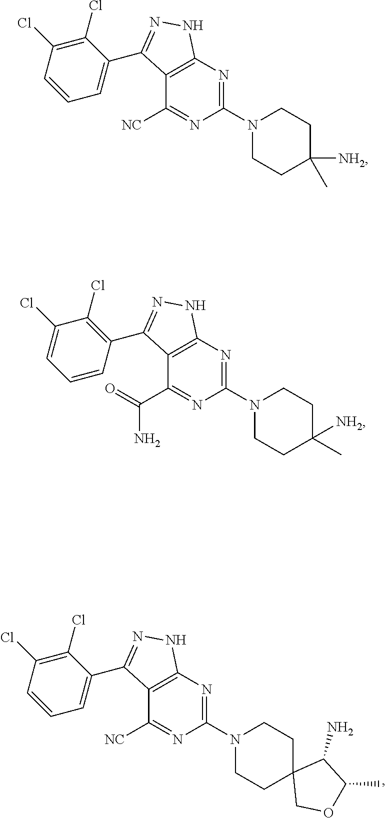 [SMILES: CC1(N)CCN(c2nc(C#N)c3c(-c4cccc(Cl)c4Cl)n[nH]c3n2)CC1 ; CC1(N)CCN(c2nc(C(N)=O)c3c(-c4cccc(Cl)c4Cl)n[nH]c3n2)CC1 ; C[C@@H]1OCC2(CCN(c3nc(C#N)c4c(-c5cccc(Cl)c5Cl)n[nH]c4n3)CC2)[C@@H]1N]

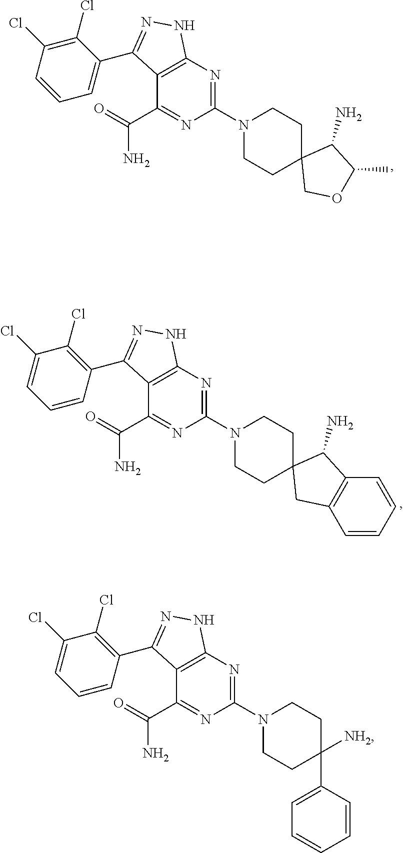 [SMILES: C[C@@H]1OCC2(CCN(c3nc(C(N)=O)c4c(-c5cccc(Cl)c5Cl)n[nH]c4n3)CC2)[C@@H]1N ; NC(=O)c1nc(N2CCC3(CC2)Cc2ccccc2[C@H]3N)nc2[nH]nc(-c3cccc(Cl)c3Cl)c12 ; NC(=O)c1nc(N2CCC(N)(c3ccccc3)CC2)nc2[nH]nc(-c3cccc(Cl)c3Cl)c12]

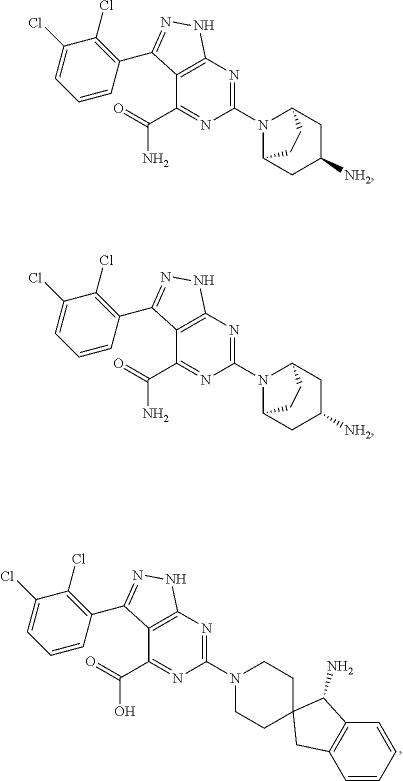 [SMILES: NC(=O)c1nc(N2[C@@H]3CC[C@H]2C[C@H](N)C3)nc2[nH]nc(-c3cccc(Cl)c3Cl)c12 ; NC(=O)c1nc(N2[C@@H]3CC[C@H]2C[C@@H](N)C3)nc2[nH]nc(-c3cccc(Cl)c3Cl)c12 ; N[C@@H]1c2ccccc2CC12CCN(c1nc(C(=O)O)c3c(-c4cccc(Cl)c4Cl)n[nH]c3n1)CC2]

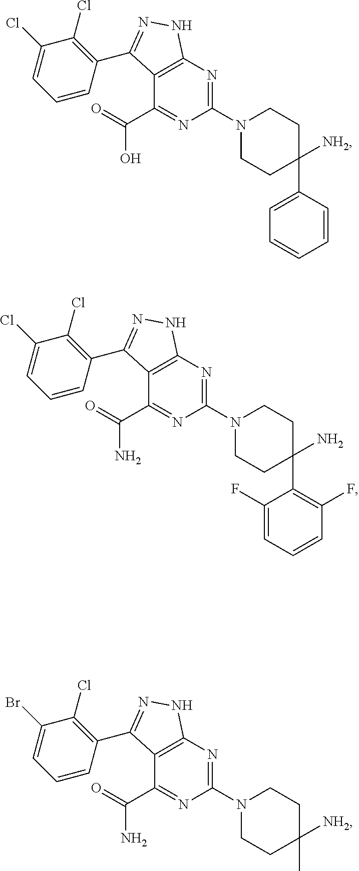 [SMILES: NC1(c2ccccc2)CCN(c2nc(C(=O)O)c3c(-c4cccc(Cl)c4Cl)n[nH]c3n2)CC1 ; NC(=O)c1nc(N2CCC(N)(c3c(F)cccc3F)CC2)nc2[nH]nc(-c3cccc(Cl)c3Cl)c12 ; CC1(N)CCN(c2nc(C(N)=O)c3c(-c4cccc(Br)c4Cl)n[nH]c3n2)CC1]

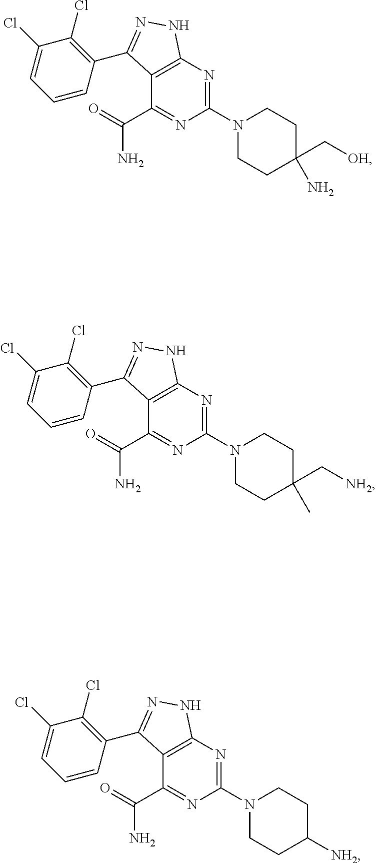 [SMILES: NC(=O)c1nc(N2CCC(N)(CO)CC2)nc2[nH]nc(-c3cccc(Cl)c3Cl)c12 ; CC1(CN)CCN(c2nc(C(N)=O)c3c(-c4cccc(Cl)c4Cl)n[nH]c3n2)CC1 ; NC(=O)c1nc(N2CCC(N)CC2)nc2[nH]nc(-c3cccc(Cl)c3Cl)c12]

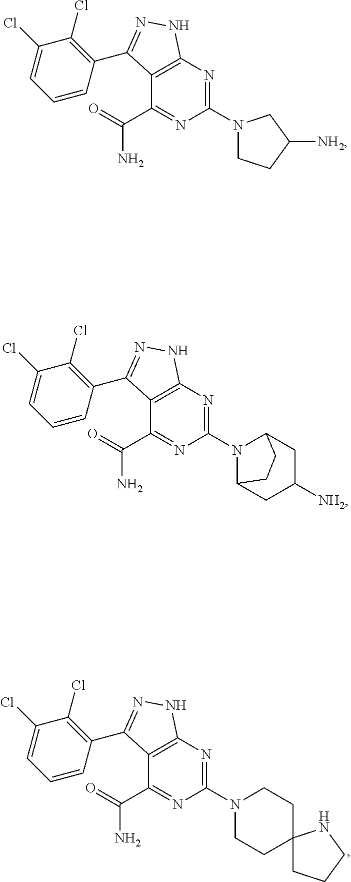 [SMILES: NC(=O)c1nc(N2CCC(N)C2)nc2[nH]nc(-c3cccc(Cl)c3Cl)c12 ; NC(=O)c1nc(N2C3CCC2CC(N)C3)nc2[nH]nc(-c3cccc(Cl)c3Cl)c12 ; NC(=O)c1nc(N2CCC3(CCCN3)CC2)nc2[nH]nc(-c3cccc(Cl)c3Cl)c12]

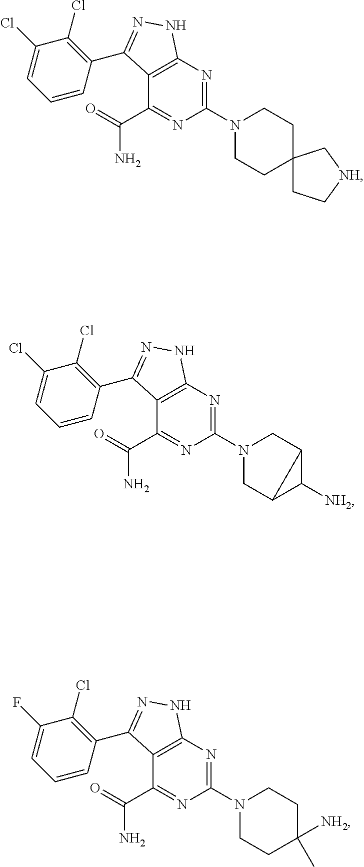 [SMILES: NC(=O)c1nc(N2CCC3(CCNC3)CC2)nc2[nH]nc(-c3cccc(Cl)c3Cl)c12 ; NC(=O)c1nc(N2CC3C(N)C3C2)nc2[nH]nc(-c3cccc(Cl)c3Cl)c12 ; CC1(N)CCN(c2nc(C(N)=O)c3c(-c4cccc(F)c4Cl)n[nH]c3n2)CC1]

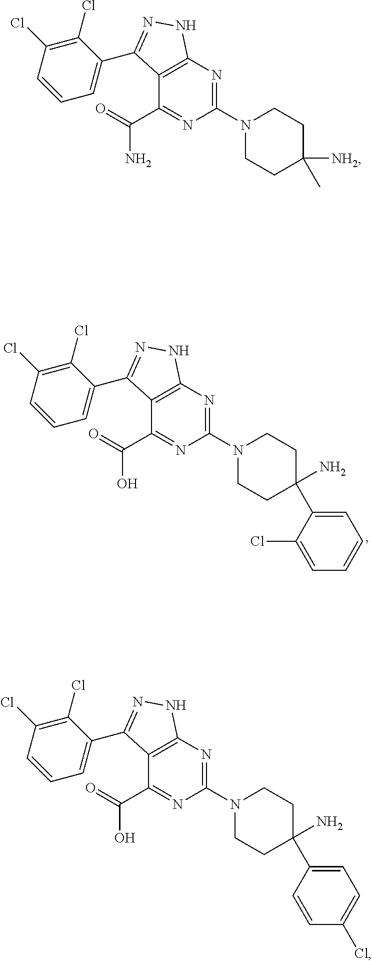 [SMILES: CC1(N)CCN(c2nc(C(N)=O)c3c(-c4cccc(Cl)c4Cl)n[nH]c3n2)CC1 ; NC1(c2ccccc2Cl)CCN(c2nc(C(=O)O)c3c(-c4cccc(Cl)c4Cl)n[nH]c3n2)CC1 ; NC1(c2ccc(Cl)cc2)CCN(c2nc(C(=O)O)c3c(-c4cccc(Cl)c4Cl)n[nH]c3n2)CC1]

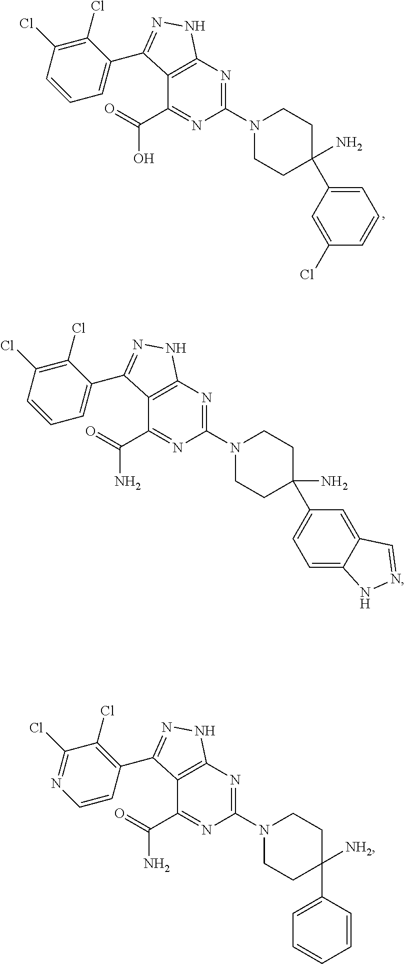 [SMILES: NC1(c2cccc(Cl)c2)CCN(c2nc(C(=O)O)c3c(-c4cccc(Cl)c4Cl)n[nH]c3n2)CC1 ; NC(=O)c1nc(N2CCC(N)(c3ccc4[nH]ncc4c3)CC2)nc2[nH]nc(-c3cccc(Cl)c3Cl)c12 ; NC(=O)c1nc(N2CCC(N)(c3ccccc3)CC2)nc2[nH]nc(-c3ccnc(Cl)c3Cl)c12]

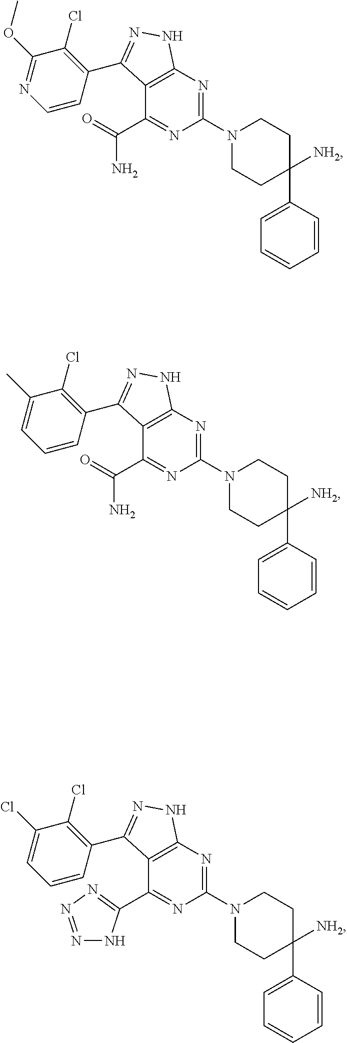 [SMILES: COc1nccc(-c2n[nH]c3nc(N4CCC(N)(c5ccccc5)CC4)nc(C(N)=O)c23)c1Cl ; Cc1cccc(-c2n[nH]c3nc(N4CCC(N)(c5ccccc5)CC4)nc(C(N)=O)c23)c1Cl ; NC1(c2ccccc2)CCN(c2nc(-c3nnn[nH]3)c3c(-c4cccc(Cl)c4Cl)n[nH]c3n2)CC1]

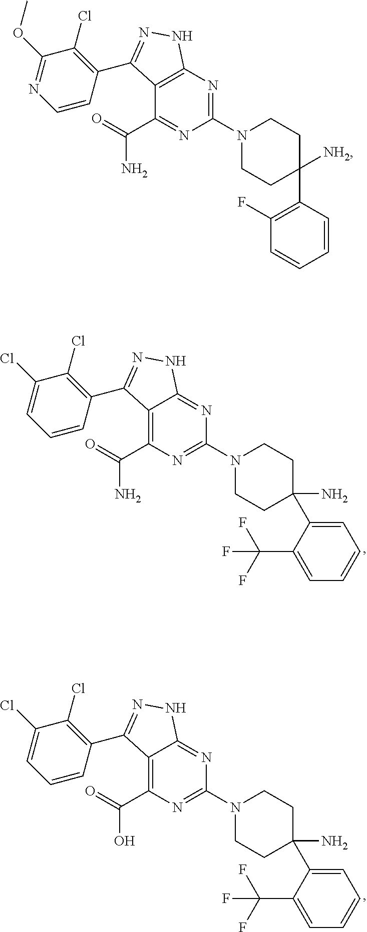 [SMILES: COc1nccc(-c2n[nH]c3nc(N4CCC(N)(c5ccccc5F)CC4)nc(C(N)=O)c23)c1Cl ; NC(=O)c1nc(N2CCC(N)(c3ccccc3C(F)(F)F)CC2)nc2[nH]nc(-c3cccc(Cl)c3Cl)c12 ; NC1(c2ccccc2C(F)(F)F)CCN(c2nc(C(=O)O)c3c(-c4cccc(Cl)c4Cl)n[nH]c3n2)CC1]

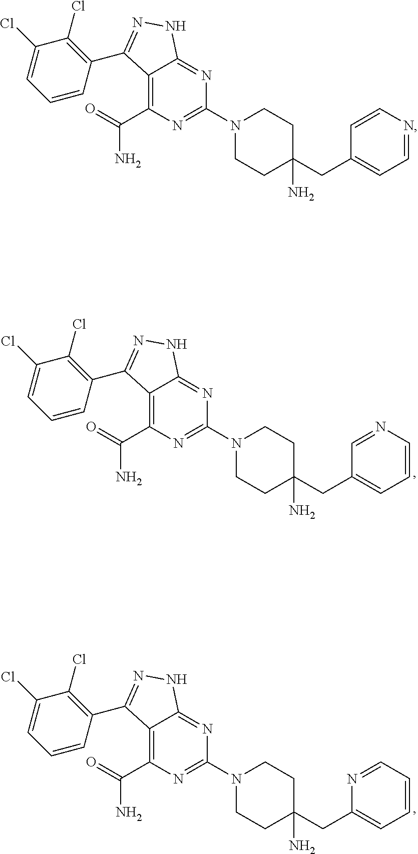 [SMILES: NC(=O)c1nc(N2CCC(N)(Cc3ccncc3)CC2)nc2[nH]nc(-c3cccc(Cl)c3Cl)c12 ; NC(=O)c1nc(N2CCC(N)(Cc3cccnc3)CC2)nc2[nH]nc(-c3cccc(Cl)c3Cl)c12 ; NC(=O)c1nc(N2CCC(N)(Cc3ccccn3)CC2)nc2[nH]nc(-c3cccc(Cl)c3Cl)c12]

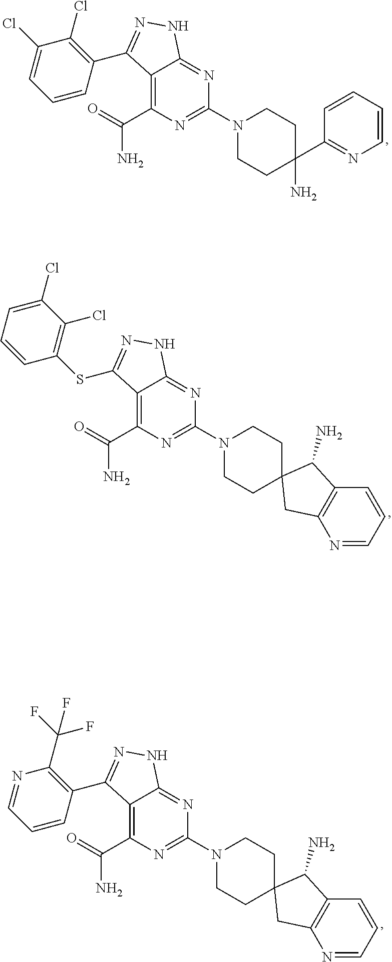 [SMILES: NC(=O)c1nc(N2CCC(N)(c3ccccn3)CC2)nc2[nH]nc(-c3cccc(Cl)c3Cl)c12 ; NC(=O)c1nc(N2CCC3(CC2)Cc2ncccc2[C@H]3N)nc2[nH]nc(Sc3cccc(Cl)c3Cl)c12 ; NC(=O)c1nc(N2CCC3(CC2)Cc2ncccc2[C@H]3N)nc2[nH]nc(-c3cccnc3C(F)(F)F)c12]

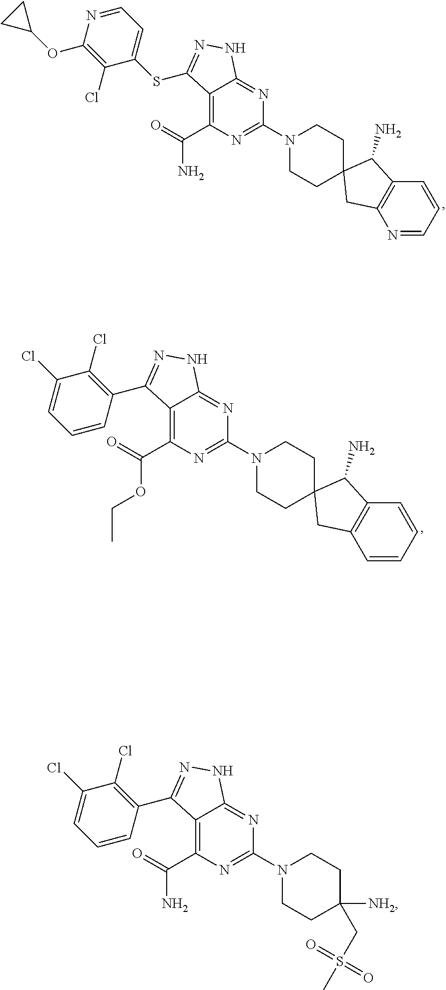 [SMILES: NC(=O)c1nc(N2CCC3(CC2)Cc2ncccc2[C@H]3N)nc2[nH]nc(Sc3ccnc(OC4CC4)c3Cl)c12 ; CCOC(=O)c1nc(N2CCC3(CC2)Cc2ccccc2[C@H]3N)nc2[nH]nc(-c3cccc(Cl)c3Cl)c12 ; CS(=O)(=O)CC1(N)CCN(c2nc(C(N)=O)c3c(-c4cccc(Cl)c4Cl)n[nH]c3n2)CC1]

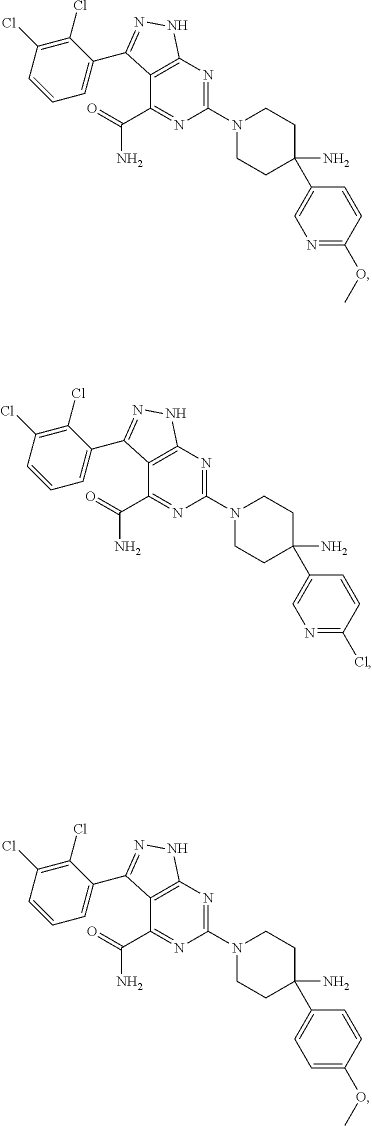 [SMILES: COc1ccc(C2(N)CCN(c3nc(C(N)=O)c4c(-c5cccc(Cl)c5Cl)n[nH]c4n3)CC2)cn1 ; NC(=O)c1nc(N2CCC(N)(c3ccc(Cl)nc3)CC2)nc2[nH]nc(-c3cccc(Cl)c3Cl)c12 ; COc1ccc(C2(N)CCN(c3nc(C(N)=O)c4c(-c5cccc(Cl)c5Cl)n[nH]c4n3)CC2)cc1]

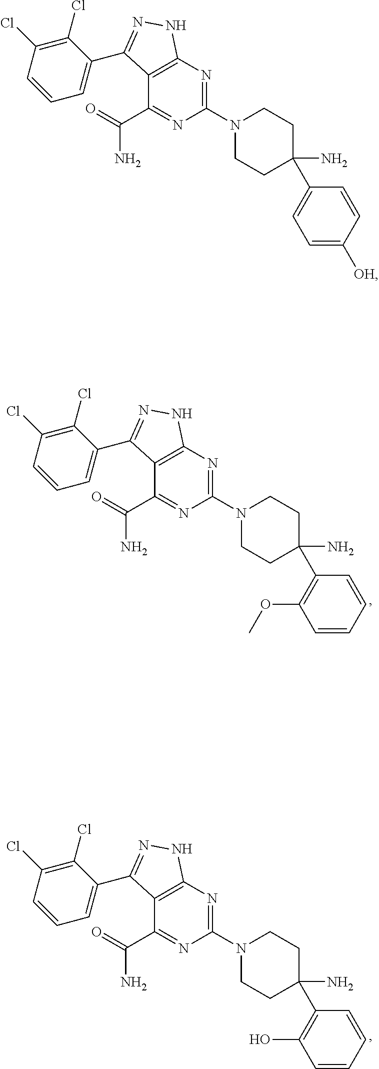 [SMILES: NC(=O)c1nc(N2CCC(N)(c3ccc(O)cc3)CC2)nc2[nH]nc(-c3cccc(Cl)c3Cl)c12 ; COc1ccccc1C1(N)CCN(c2nc(C(N)=O)c3c(-c4cccc(Cl)c4Cl)n[nH]c3n2)CC1 ; NC(=O)c1nc(N2CCC(N)(c3ccccc3O)CC2)nc2[nH]nc(-c3cccc(Cl)c3Cl)c12]

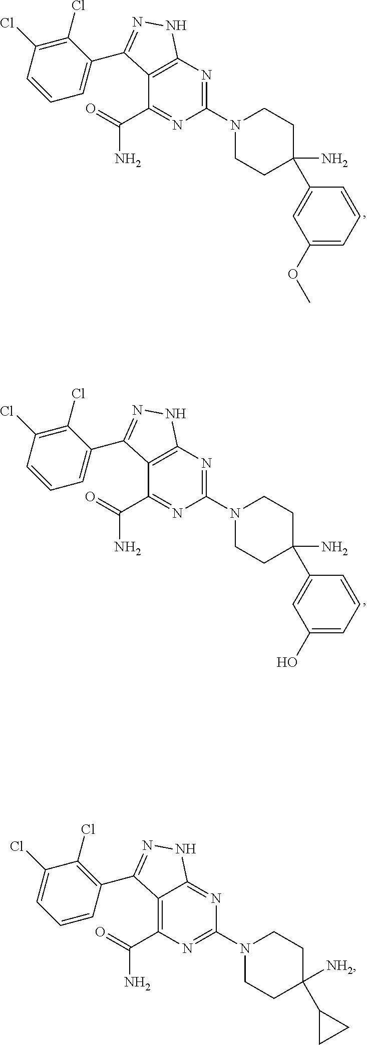 [SMILES: COc1cccc(C2(N)CCN(c3nc(C(N)=O)c4c(-c5cccc(Cl)c5Cl)n[nH]c4n3)CC2)c1 ; NC(=O)c1nc(N2CCC(N)(c3cccc(O)c3)CC2)nc2[nH]nc(-c3cccc(Cl)c3Cl)c12 ; NC(=O)c1nc(N2CCC(N)(C3CC3)CC2)nc2[nH]nc(-c3cccc(Cl)c3Cl)c12]

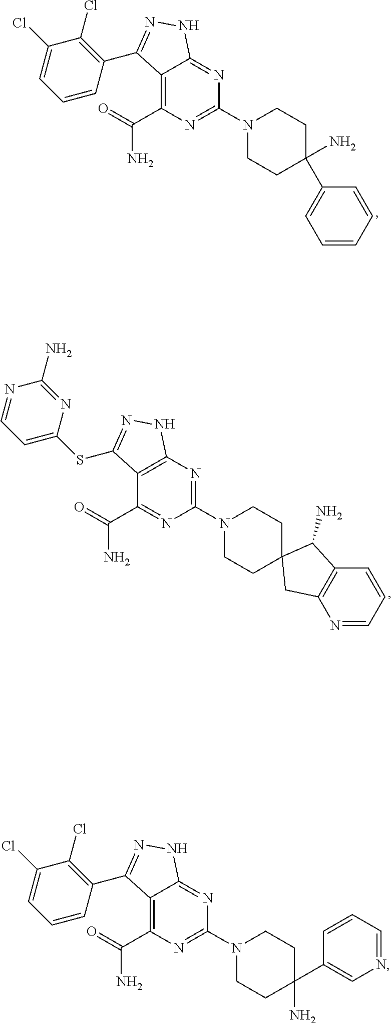 [SMILES: NC(=O)c1nc(N2CCC(N)(c3ccccc3)CC2)nc2[nH]nc(-c3cccc(Cl)c3Cl)c12 ; NC(=O)c1nc(N2CCC3(CC2)Cc2ncccc2[C@H]3N)nc2[nH]nc(Sc3ccnc(N)n3)c12 ; NC(=O)c1nc(N2CCC(N)(c3cccnc3)CC2)nc2[nH]nc(-c3cccc(Cl)c3Cl)c12]

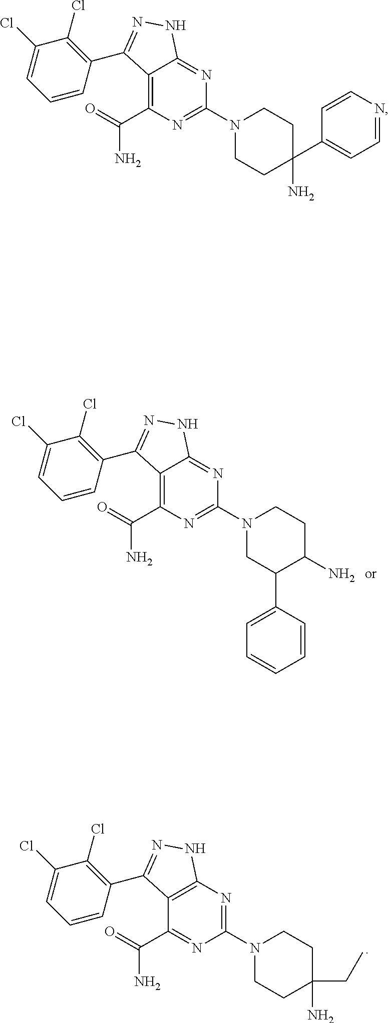 [SMILES: NC(=O)c1nc(N2CCC(N)(c3ccncc3)CC2)nc2[nH]nc(-c3cccc(Cl)c3Cl)c12 ; NC(=O)c1nc(N2CCC(N)C(c3ccccc3)C2)nc2[nH]nc(-c3cccc(Cl)c3Cl)c12 ; CCC1(N)CCN(c2nc(C(N)=O)c3c(-c4cccc(Cl)c4Cl)n[nH]c3n2)CC1]

22. A preparation method for the compound represented by general formula (I) or the stereoisomer or the tautomer thereof according to claim 3, wherein the method comprises: 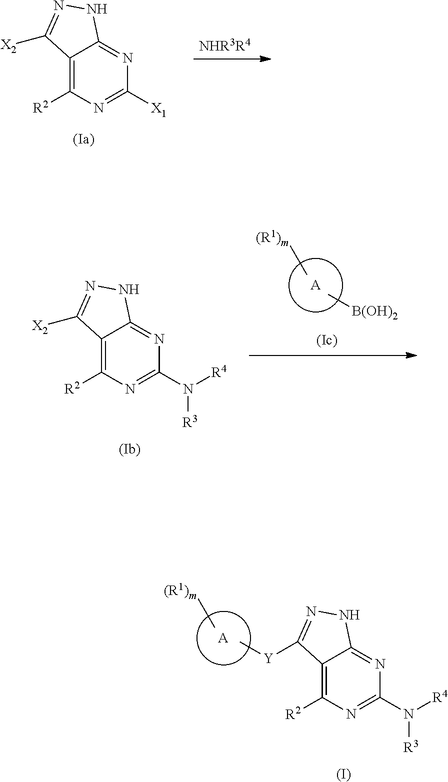
subjecting the compound represented by general formula (Ia) and NHR³R⁴ to a nucleophilic substitution reaction under alkaline condition to obtain the compound represented by general formula (Ib); and subjecting the compound represented by general formula (Ib) and the compound represented by general formula (Ic) to a Suzuki reaction in the presence of palladium catalyst and alkaline condition, and optionally further removing a protecting group of the obtained compound to obtain the compound represented by general formula (I);
wherein:
Y is selected from chemical bond;
X₁ is selected from leaving group, wherein the leaving group is selected from halogen or —SO₂R^{t};
X₂ is selected from halogen;
R^{t} is selected from alkyl; and
ring A, m, and R¹-R⁴ are defined as in claim 3.

23. A preparation method for the compound represented by general formula (I) or the stereoisomer or the tautomer thereof according to claim 3, wherein the method comprises: 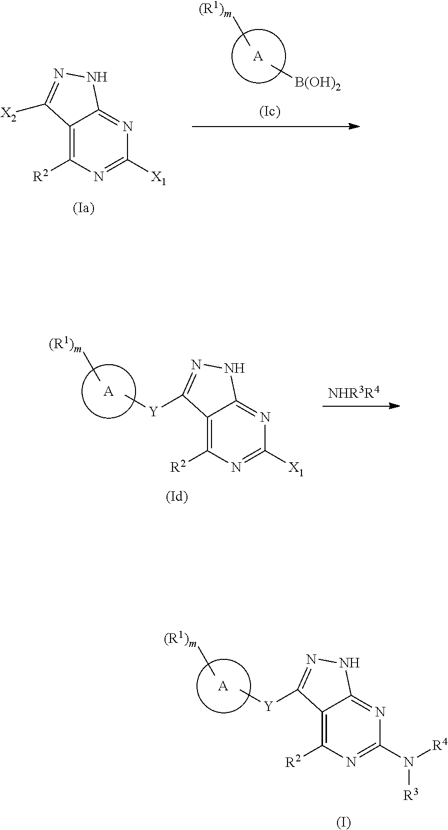
subjecting the compound represented by general formula (Ia) and the compound represented by general formula (Ic) to a Suzuki reaction in the presence of palladium catalyst and alkaline condition, to obtain the compound represented by general formula (Id); and subjecting the compound represented by general formula (Id) and NHR³R⁴ to a nucleophilic substitution reaction under alkaline condition to obtain the compound represented by general formula (I);
wherein:
Y is selected from chemical bond;
X₁ is selected from leaving group, wherein the leaving group is selected from halogen or SO₂R^{t};
X₂ is selected from halogen;
R^{t} is selected from alkyl; and
ring A, m, and R¹-R⁴ are defined as in claim 3.

24. A compound represented by general formula (Ia) or a stereoisomer or a tautomer thereof, which is an intermediate for preparing a compound represented by general formula (I): 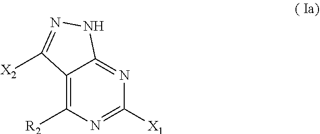
wherein:
X₁ is selected from leaving group, wherein the leaving group is selected from halogen or SO₂R^{t};
X₂ is selected from halogen;
R^{t} is selected from alkyl; and
R² is selected from cyano, tetrazolyl, —C(O)R⁵, —C(O)OR⁵ or —C(O)NR⁶R⁷;
R⁵, R⁶ and R⁷ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, amino, halogen, nitro, cyano, alkyl, alkoxy, cycloalkyl, heterocyclyl, aryl, heteroaryl, —C(O)R⁸, —C(O)OR⁸, —OC(O)R⁸, —SO₂R⁸, —NR⁹R¹⁰, —C(O)NR⁹R¹⁰, —SO₂NR⁹R¹⁰ or —NR⁹C(O)R¹⁰;
alternatively, R⁶ and R⁷ together with the N atom bound therewith form a 3-8 membered heterocyclyl, wherein the 3-8 membered heterocyclyl internally contains one or more N, O, S or SO₂ atoms, and the 3-8 membered heterocyclyl is optionally further substituted by one or more substituents selected from hydroxy, halogen, amino, alkyl or alkoxy; and
R⁸, R⁹ and R¹⁰ are each independently selected from hydrogen atom, alkyl, cycloalkyl, heterocyclyl, aryl or heteroaryl, wherein the alkyl, cycloalkyl, heterocyclyl, aryl or heteroaryl is optionally further substituted by one or more substituents selected from hydroxy, halogen, nitro, cyano, alkyl, alkoxy, cycloalkyl, heterocyclyl, aryl, heteroaryl, carboxyl or carboxylate.

25. A preparation method for the compound represented by general formula (Ia) or the stereoisomer or the tautomer thereof according to claim 24, wherein the method comprises: 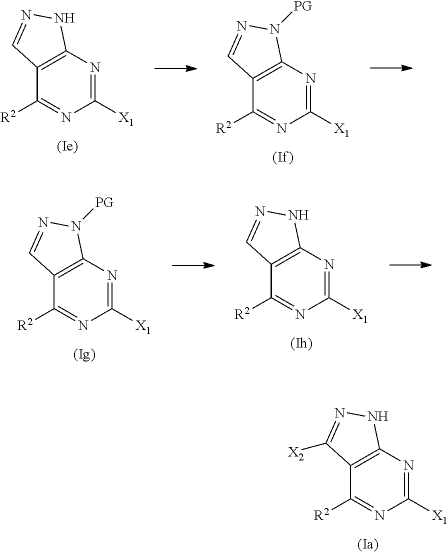
protecting the amino of the compound represented by general formula (Ie) to obtain the compound represented by general formula (If); subjecting the compound represented by general formula (If) to a coupling reaction under the action of palladium catalysts to obtain the compound represented by general formula (Ig); removing the protecting group PG from the compound represented by general formula (Ig) to obtain the compound represented by general formula (Ih); and
subjecting the compound represented by general formula (Ih) to a halogenating reaction to obtain the compound represented by general formula (Ia);
wherein:
PG is the protecting group, preferably 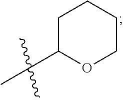 [SMILES: *C1CCCCO1]
X₃ is selected from halogen; and
X₁, X₂ and R² are as defined in claim 24.

26. A pharmaceutical composition, wherein the pharmaceutical composition comprises an effective dose of the compound or the stereoisomer or the tautomer thereof, or the pharmaceutically acceptable salt thereof according to claim 1, and a pharmaceutically acceptable carrier, an excipient or a combination thereof.

27. A method for inhibiting SHP2 allosterism, comprising administering a therapeutically effective amount of the compound or the stereoisomer or the tautomer thereof, or the pharmaceutically acceptable salt thereof according to claim 1 to a patient in need thereof.

28. A method for treating a disease mediated by SHP2, comprising administering a therapeutically effective amount of the compound or the stereoisomer or the tautomer thereof, or the pharmaceutically acceptable salt thereof according to claim 1, wherein the disease mediated by SHP2 is preferably cancer, cancerometastasis, cardiovascular disease, immune disorder, fibrosis or visual disorder; more preferably, the disease mediated by SHP2 is selected from Noonan syndrome, Leopard spot syndrome, juvenile myelomonocytic leukemia, neuroblastoma, melanoma, acute myeloid leukemia, breast cancer, esophagus cancer, lung cancer, colon cancer, head cancer, neuroblastoma, squamous cell carcinoma of head and neck, gastric cancer, anaplastic large cell lymphoma and glioblastoma.

29. (canceled)
